(19) United States
(12) Patent Application Publication
(10) Pub. No.: US 2025/0177391 A1
(43) Pub. Date: Jun. 5, 2025

(54) INHIBITORS OF DDR1 AND DDR2 FOR THE TREATMENT OF ARTHRITIS

(71) Applicant: President and Fellows of Harvard College, Cambridge, MA (US)

(72) Inventors: Yefu Li, Cambridge, MA (US); Lin Xu, Cambridge, MA (US)

(73) Assignee: President and Fellows of Harvard College, Cambridge, MA (US)

(21) Appl. No.: 18/840,802

(22) PCT Filed: Feb. 22, 2023
(86) PCT No.: PCT/US2023/063009

§ 371 Date: Aug. 22, 2024

Related U.S. Application Data
(60) Provisional application No. 63/313,102, filed on Feb. 23, 2022.

(51) Int. Cl. A61K 31/505; A61P 19/02; C07D 239/48

(52) U.S. Cl. CPC A61K 31/505; A61P 19/02; C07D 239/48

(57) ABSTRACT

Provided herein are compounds, such as compounds of Formulae (I) and (II), and pharmaceutically acceptable salts, hydrates, solvates, polymorphs, co-crystals, tautomers, stereoisomers, isotopically labeled derivatives, and prodrugs thereof, and compositions, methods, uses, and kits thereof. The compounds provided herein are DDR1, DDR2, or p38 MAPK (e.g., p38-alpha) inhibitors and are therefore useful for the treatment and/or prevention of various diseases and conditions (e.g., inflammatory diseases, joint diseases, proliferative diseases, fibrosis, or pain).

### RELATED APPLICATIONS

This application claims priority under 35 U.S.C. § 119(e) to U.S. Provisional Application No. 63/313,102 filed Feb. 23, 2022, the contents of which are incorporated herein by reference.

### GOVERNMENT SUPPORT

This invention was made with government support under W81XWH1810097 awarded by the U.S. Army Medical Research and Development Command. The government has certain rights in the invention.

### REFERENCE TO AN ELECTRONIC SEQUENCE LISTING

The contents of the electronic sequence listing (H082470408WO00-SEQ-PJH.xml; Size: 2,830 bytes; and Date of Creation: Feb. 21, 2023) are herein incorporated by reference in their entirety.

### BACKGROUND

Discoidin Domain Receptors 1 and 2 (DDR1 and DDR2, respectively) are receptor tyrosine kinases. Kinases in this class contribute to regulation of cell growth, differentiation, and metabolism. Both DDR1 and DDR2 bind collagens as ligands at their extracellular discoidin domains, promoting phosphorylation and signal transduction. DDR1 binds and signals in response to both fibrillar and non-fibrillar collagens whereas DDR2 is largely activated by fibrillar collagens. DDR1 and DDR2 can therefore be a target for treating cancer (e.g., ovarian, lung, head, neck, pancreatic, and breast cancer), inflammation, arthritis (e.g., osteoarthritis, rheumatoid arthritis), fibrosis, and related pain. As many current treatments for such diseases remain suboptimal, small molecule compounds and related methods are desired for inhibiting the activity of DDR1 or DDR2 and treating associated diseases.

Mitogen-activated protein kinases (MAPK) are also involved in signaling pathways. MAPKs operate as a series of modules engaged in phosphorylation.¹ The MAPK families include extracellular-regulated protein kinases, c-Jun NH₂-terminal kinase, and p38. The p38 family can upregulate cytokine production by various mechanisms and includes alpha, beta, gamma, and delta family members.¹ These MAPKs may also play a role in arthritis (e.g., osteoarthritis) and related pain.¹ Accordingly, small molecule inhibitors are desired to block cytokine signaling pathways, such as the p38 MAPK pathway, and attenuate synthesis of inflammatory cytokines.

### SUMMARY OF THE INVENTION

The present disclosure relates in part to new compounds (e.g., compounds of Formulae (I) and (II)) that can inhibit DDR1, DDR2, and/or p38 MAP kinase activity, and compositions and methods of using and preparing such compounds.

In one aspect, provided herein are compounds (e.g., compounds of Formulae (I) and (II)), and pharmaceutically acceptable salts, solvates, hydrates, polymorphs, co-crystals, tautomers, stereoisomers, isotopically labeled derivatives, and prodrugs thereof, and pharmaceutical compositions thereof. In certain embodiments, the compounds provided herein are DDR2 inhibitors and can therefore be used for the treatment and/or prevention of diseases (e.g., inflammatory disease, joint disease, proliferative disease, fibrosis, pain). In certain embodiments, the compounds provided herein are DDR1 inhibitors and can therefore be used for the treatment and/or prevention of diseases (e.g., inflammatory disease, joint disease, proliferative disease, fibrosis, pain). In certain embodiments, the compounds provided herein are p38 MAP kinase (e.g., p38-alpha) inhibitors and can therefore be used for the treatment and/or prevention of diseases (e.g., inflammatory disease, joint disease, proliferative disease, fibrosis, pain).

The present disclosure also provides methods of using the compounds and compositions provided herein, e.g., for treating and/or preventing an inflammatory disease, joint disease, proliferative disease, fibrosis, or pain in a subject. In certain embodiments, the present disclosure provides methods of using compounds and compositions provided herein for inhibiting DDR2 activity in a cell of a subject or a biological sample. In certain embodiments, the present disclosure provides methods of using compounds and compositions provided herein for inhibiting DDR1 activity in a cell of a subject or a biological sample. In certain embodiments, the present disclosure provides methods of using compounds and compositions provided herein for inhibiting p38 MAP kinase (e.g., p38-alpha) activity in a cell of a subject or a biological sample. In another aspect, the present invention provides kits comprising a compound provided herein, or a pharmaceutically acceptable salt, solvate, hydrate, polymorph, co-crystal, tautomer, stereoisomer, isotopically labeled derivative, or prodrug thereof, or a pharmaceutical composition thereof.

In one aspect, the disclosure provides compounds of Formula (I):

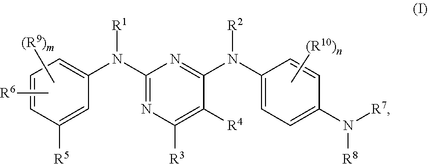

and pharmaceutically acceptable salts, solvates, hydrates, polymorphs, co-crystals, tautomers, stereoisomers, isotopically labeled derivatives, and prodrugs thereof, wherein R¹, R², R³, R⁴, R⁵, R⁶, R⁷, R⁸, R⁹, R¹⁰, m, and n are as defined herein.

In certain embodiments, a compound of Formula (I) is of Formula (I′):

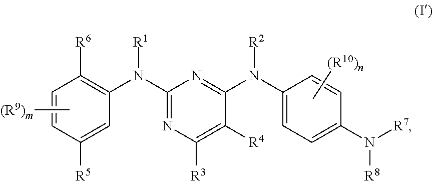

or a pharmaceutically acceptable salt, solvate, hydrate, polymorph, co-crystal, tautomer, stereoisomer, isotopically labeled derivative, or prodrug thereof, wherein R¹, R², R³, R⁴, R⁵, R⁶, R⁷, R⁸, R⁹, R¹⁰, m, and n are as defined herein.

In certain embodiments, a compound of Formula (I) is of Formula (Ia):

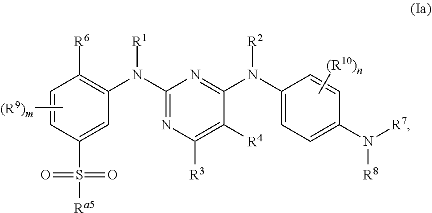

or a pharmaceutically acceptable salt, solvate, hydrate, polymorph, co-crystal, tautomer, stereoisomer, isotopically labeled derivative, or prodrug thereof, wherein R¹, R², R³, R⁴, R^{a5}, R⁶, R⁷, R⁸, R⁹, R¹⁰, m, and n are as defined herein.

In certain embodiments, a compound of Formula (I) is of Formula (Ib):

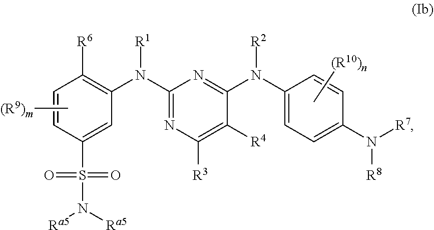

or a pharmaceutically acceptable salt, solvate, hydrate, polymorph, co-crystal, tautomer, stereoisomer, isotopically labeled derivative, or prodrug thereof, wherein R¹, R², R³, R⁴, R^{a5}, R⁶, R⁷, R⁸, R⁹, R¹⁰, m, and n are as defined herein.

In certain embodiments, a compound of Formula (I) is of Formula (Ic):

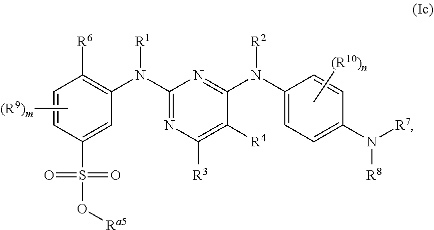

or a pharmaceutically acceptable salt, solvate, hydrate, polymorph, co-crystal, tautomer, stereoisomer, isotopically labeled derivative, or prodrug thereof, wherein R¹, R², R³, R⁴, R^{a5}, R⁶, R⁷, R⁸, R⁹, R¹⁰, m, and n are as defined herein.

In certain embodiments, a compound of Formula (I) is of Formula (Id):

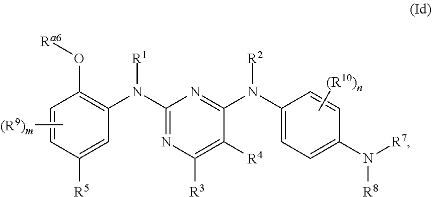

or a pharmaceutically acceptable salt, solvate, hydrate, polymorph, co-crystal, tautomer, stereoisomer, isotopically labeled derivative, or prodrug thereof, wherein R¹, R², R³, R⁴, R⁵, R^{a6}, R⁷, R⁸, R⁹, R¹⁰, m, and n are as defined herein.

In certain embodiments, a compound of Formula (I) is of Formula (Ie):

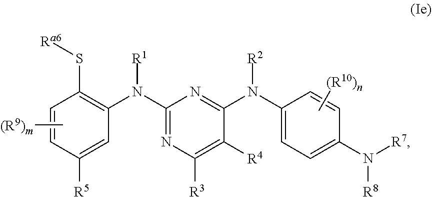

or a pharmaceutically acceptable salt, solvate, hydrate, polymorph, co-crystal, tautomer, stereoisomer, isotopically labeled derivative, or prodrug thereof, wherein R¹, R², R³, R⁴, R⁵, R^{a6}, R⁷, R⁸, R⁹, R¹⁰, m, and n are as defined herein.

In certain embodiments, a compound of Formula (I) is of Formula (If):

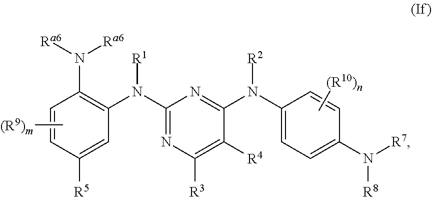

or a pharmaceutically acceptable salt, solvate, hydrate, polymorph, co-crystal, tautomer, stereoisomer, isotopically labeled derivative, or prodrug thereof, wherein R¹, R², R³, R⁴, R⁵, R^{a6}, R⁷, R⁸, R⁹, R¹⁰, m, and n are as defined herein.

In certain embodiments, a compound of Formula (I) is of Formula (Ij):

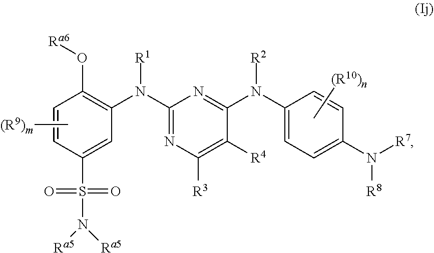

or a pharmaceutically acceptable salt, solvate, hydrate, polymorph, co-crystal, tautomer, stereoisomer, isotopically labeled derivative, or prodrug thereof, wherein R¹, R², R³, R⁴, R^{a5}, R^{a6}, R⁷, R⁸, R⁹, R¹⁰, m, and n are as defined herein.

In certain embodiments, a compound of Formula (I) is of Formula (Ik):

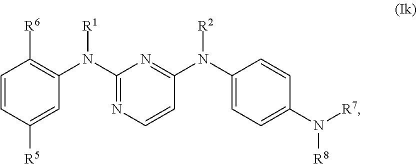

or a pharmaceutically acceptable salt, solvate, hydrate, polymorph, co-crystal, tautomer, stereoisomer, isotopically labeled derivative, or prodrug thereof, wherein R¹, R², R⁵, R⁶, R⁷, and R⁸ are as defined herein.

In certain embodiments, a compound of Formula (I) is of Formula (Ii):

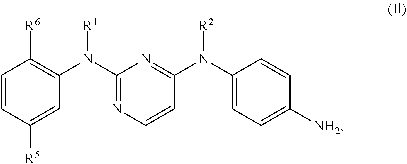

or a pharmaceutically acceptable salt, solvate, hydrate, polymorph, co-crystal, tautomer, stereoisomer, isotopically labeled derivative, or prodrug thereof, wherein R¹, R², R⁵, and R⁶ are as defined herein.

In certain embodiments, a compound of Formula (I) is of Formula (Im):

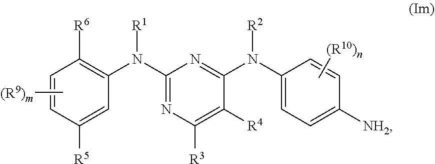

or a pharmaceutically acceptable salt, solvate, hydrate, polymorph, co-crystal, tautomer, stereoisomer, isotopically labeled derivative, or prodrug thereof, wherein R¹, R², R³, R⁴, R⁵, R⁶, R⁹, R¹⁰, m, and n are as defined herein.

In certain embodiments, a compound of Formula (I) is of Formula (In):

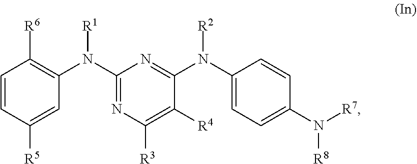

or a pharmaceutically acceptable salt, solvate, hydrate, polymorph, co-crystal, tautomer, stereoisomer, isotopically labeled derivative, or prodrug thereof, wherein R¹, R², R³, R⁴, R⁵, R⁶, R⁷, and R⁸ are as defined herein.

In certain embodiments, a compound of Formula (I) is of Formula (I″):

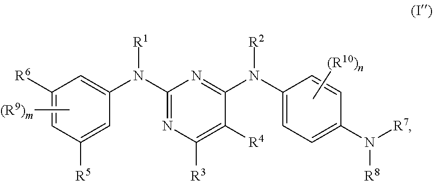

or a pharmaceutically acceptable salt, solvate, hydrate, polymorph, co-crystal, tautomer, stereoisomer, isotopically labeled derivative, or prodrug thereof, wherein R¹, R², R³, R⁴, R⁵, R⁶, R⁷, R⁸, R⁹, R¹⁰, m, and n are as defined herein.

In certain embodiments, a compound of Formula (I) is of Formula (Ig):

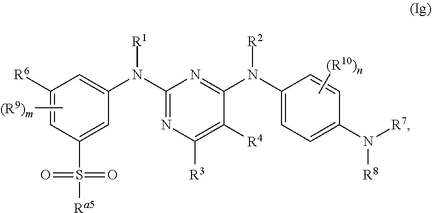

or a pharmaceutically acceptable salt, solvate, hydrate, polymorph, co-crystal, tautomer, stereoisomer, isotopically labeled derivative, or prodrug thereof, wherein R¹, R², R³, R⁴, R^{a5}, R⁶, R⁷, R⁸, R⁹, R¹⁰, m, and n are as defined herein.

In certain embodiments, a compound of Formula (I) is of Formula (Ih):

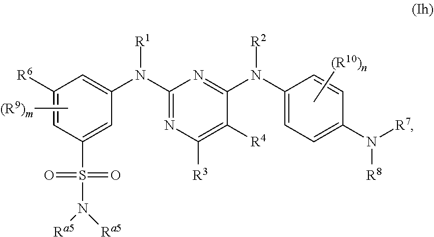

or a pharmaceutically acceptable salt, solvate, hydrate, polymorph, co-crystal, tautomer, stereoisomer, isotopically labeled derivative, or prodrug thereof, wherein R¹, R², R³, R⁴, R^{a5}, R⁶, R⁷, R⁸, R⁹, R¹⁰, m, and n are as defined herein.

In certain embodiments, a compound of Formula (I) is of Formula (Ii):

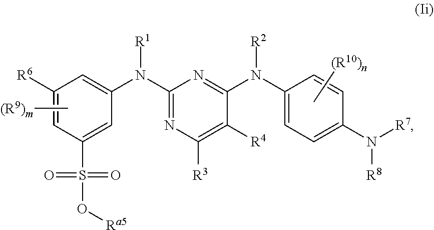

or a pharmaceutically acceptable salt, solvate, hydrate, polymorph, co-crystal, tautomer, stereoisomer, isotopically labeled derivative, or prodrug thereof, wherein R¹, R², R³, R⁴, R^{a5}, R⁶, R⁷, R⁸, R⁹, R¹⁰, m, and n are as defined herein.

In certain embodiments, a compound of Formula (I) is of Formula (Io):

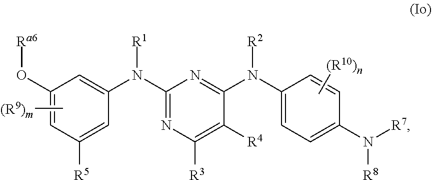

or a pharmaceutically acceptable salt, solvate, hydrate, polymorph, co-crystal, tautomer, stereoisomer, isotopically labeled derivative, or prodrug thereof, wherein R¹, R², R³, R⁴, R⁵, R^{a6}, R⁷, R⁸, R⁹, R¹⁰, m, and n are as defined herein.

In certain embodiments, a compound of Formula (I) is of Formula (Ip):

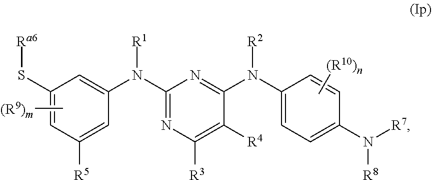

or a pharmaceutically acceptable salt, solvate, hydrate, polymorph, co-crystal, tautomer, stereoisomer, isotopically labeled derivative, or prodrug thereof, wherein R¹, R², R³, R⁴, R⁵, R^{a6}, R⁷, R⁸, R⁹, R¹⁰, m, and n are as defined herein.

In certain embodiments, a compound of Formula (I) is of Formula (Iq):

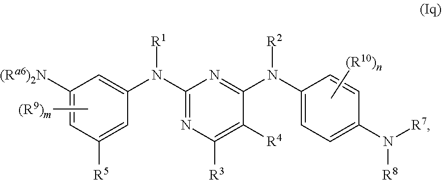

or a pharmaceutically acceptable salt, solvate, hydrate, polymorph, co-crystal, tautomer, stereoisomer, isotopically labeled derivative, or prodrug thereof, wherein R¹, R², R³, R⁴, R⁵, R^{a6}, R⁷, R⁸, R⁹, R¹⁰, m, and n are as defined herein.

In certain embodiments, a compound of Formula (I) is of Formula (Ir):

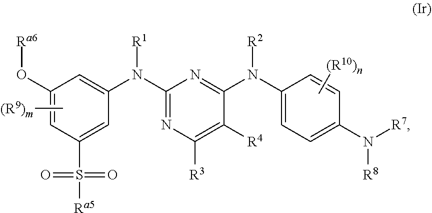

or a pharmaceutically acceptable salt, solvate, hydrate, polymorph, co-crystal, tautomer, stereoisomer, isotopically labeled derivative, or prodrug thereof, wherein R¹, R², R³, R⁴, R^{a5}, R^{a6}, R⁷, R⁸, R⁹, R¹⁰, m, and n are as defined herein.

In certain embodiments, a compound of Formula (I) is of Formula (Is):

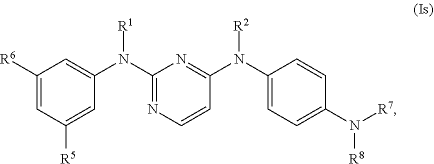

or a pharmaceutically acceptable salt, solvate, hydrate, polymorph, co-crystal, tautomer, stereoisomer, isotopically labeled derivative, or prodrug thereof, wherein R¹, R², R⁵, R⁶, R⁷, and R⁸ are as defined herein.

In certain embodiments, a compound of Formula (I) is of Formula (It):

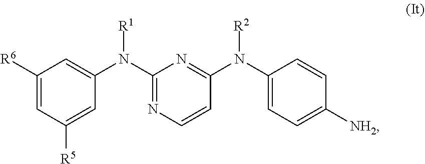

or a pharmaceutically acceptable salt, solvate, hydrate, polymorph, co-crystal, tautomer, stereoisomer, isotopically labeled derivative, or prodrug thereof, wherein R¹, R², R⁵, and R⁶ are as defined herein.

In certain embodiments, a compound of Formula (I) is of Formula (Iu):

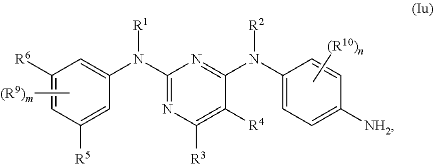

or a pharmaceutically acceptable salt, solvate, hydrate, polymorph, co-crystal, tautomer, stereoisomer, isotopically labeled derivative, or prodrug thereof, wherein R¹, R², R³, R⁴, R⁵, R⁶, R⁹, R¹⁰, m, and n are as defined herein.

In certain embodiments, a compound of Formula (I) is of Formula (Iv):

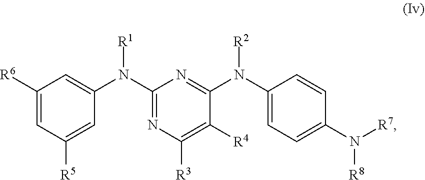

or a pharmaceutically acceptable salt, solvate, hydrate, polymorph, co-crystal, tautomer, stereoisomer, isotopically labeled derivative, or prodrug thereof, wherein R¹, R², R³, R⁴, R⁵, R⁶, R⁷, and R⁸ are as defined herein.

In certain embodiments, a compound of Formula (I) is of Formula (I′″):

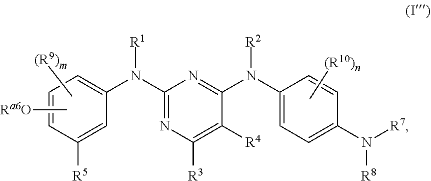

or a pharmaceutically acceptable salt, solvate, hydrate, polymorph, co-crystal, tautomer, stereoisomer, isotopically labeled derivative, or prodrug thereof, wherein R¹, R², R³, R⁴, R⁵, R^{a6}, R⁷, R⁸, R⁹, R¹⁰, m, and n are as defined herein.

In certain embodiments, a compound of Formula (I) is of Formula (Iw):

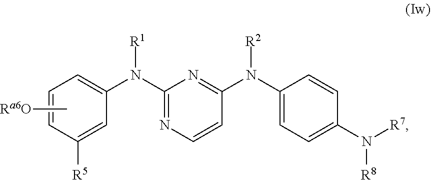

or a pharmaceutically acceptable salt, solvate, hydrate, polymorph, co-crystal, tautomer, stereoisomer, isotopically labeled derivative, or prodrug thereof, wherein R¹, R², R⁵, R^{a6}, R⁷, and R⁸ are as defined herein.

In certain embodiments, a compound of Formula (I) is of Formula (Ix):

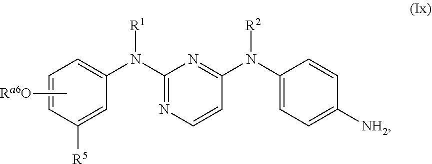

or a pharmaceutically acceptable salt, solvate, hydrate, polymorph, co-crystal, tautomer, stereoisomer, isotopically labeled derivative, or prodrug thereof, wherein R¹, R², R⁵, and R^{a6} are as defined herein.

In certain embodiments, a compound of Formula (I) is of Formula (Iy):

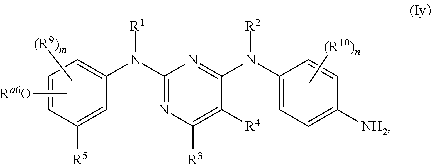

or a pharmaceutically acceptable salt, solvate, hydrate, polymorph, co-crystal, tautomer, stereoisomer, isotopically labeled derivative, or prodrug thereof, wherein R¹, R², R³, R⁴, R⁵, R^{a6}, R⁹, R¹⁰, m, and n are as defined herein.

In certain embodiments, a compound of Formula (I) is of Formula (Iz):

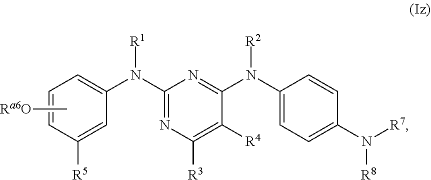

or a pharmaceutically acceptable salt, solvate, hydrate, polymorph, co-crystal, tautomer, stereoisomer, isotopically labeled derivative, or prodrug thereof, wherein R¹, R², R³, R⁴, R⁵, R^{a6}, R⁷, and R⁸ are as defined herein.

In certain embodiments, a compound of Formula (I) is the following:

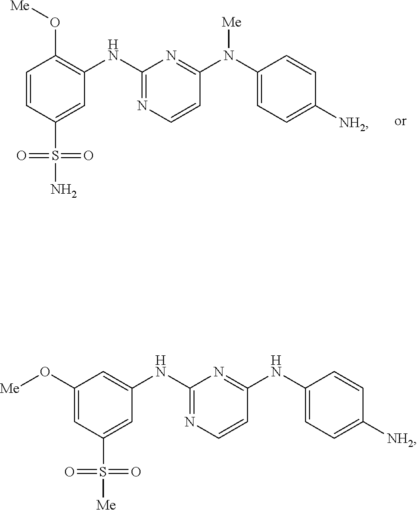 [SMILES: COc1ccc(S(N)(=O)=O)cc1Nc1nccc(N(C)c2ccc(N)cc2)n1 ; "or" ; COc1cc(Nc2nccc(Nc3ccc(N)cc3)n2)cc(S(C)(=O)=O)c1]

or a pharmaceutically acceptable salt, solvate, hydrate, polymorph, co-crystal, tautomer, stereoisomer, isotopically labeled derivative, or prodrug thereof.

In another aspect, the disclosure provides compounds of Formula (II):

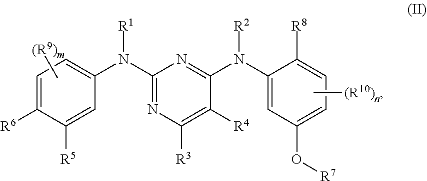

and pharmaceutically acceptable salts, solvates, hydrates, polymorphs, co-crystals, tautomers, stereoisomers, isotopically labeled derivatives, and prodrugs thereof, wherein R¹, R², R³, R⁴, R⁵, R⁶, R⁷, R⁸, R⁹, R¹⁰, m, and n are as defined herein.

In certain embodiments, a compound of Formula (II) is of Formula (IIa):

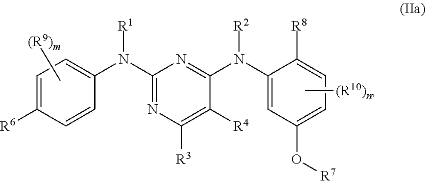

or a pharmaceutically acceptable salt, solvate, hydrate, polymorph, co-crystal, tautomer, stereoisomer, isotopically labeled derivative, or prodrug thereof, wherein R¹, R², R³, R⁴, R⁶, R⁷, R⁸, R⁹, R¹⁰, m, and n are as defined herein.

In certain embodiments, a compound of Formula (II) is of Formula (IIb):

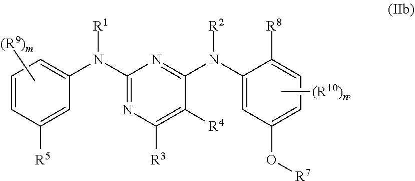

or a pharmaceutically acceptable salt, solvate, hydrate, polymorph, co-crystal, tautomer, stereoisomer, isotopically labeled derivative, or prodrug thereof, wherein R¹, R², R³, R⁴, R⁵, R⁷, R⁸, R⁹, R¹⁰, m, and n are as defined herein.

In certain embodiments, a compound of Formula (II) is of Formula (IIa-i):

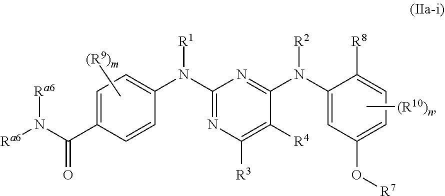

or a pharmaceutically acceptable salt, solvate, hydrate, polymorph, co-crystal, tautomer, stereoisomer, isotopically labeled derivative, or prodrug thereof, wherein R¹, R², R³, R⁴, R^{a6}, R⁷, R⁸, R⁹, R¹⁰, m, and n are as defined herein.

In certain embodiments, a compound of Formula (II) is of Formula (IIa-ii):

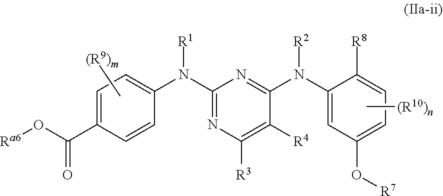

or a pharmaceutically acceptable salt, solvate, hydrate, polymorph, co-crystal, tautomer, stereoisomer, isotopically labeled derivative, or prodrug thereof, wherein R¹, R², R³, R⁴, R^{a6}, R⁷, R⁸, R⁹, R¹⁰, m, and n are as defined herein.

In certain embodiments, a compound of Formula (II) is of Formula (IIa-iii):

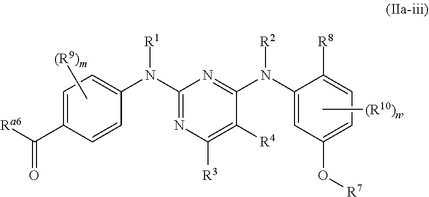

or a pharmaceutically acceptable salt, solvate, hydrate, polymorph, co-crystal, tautomer, stereoisomer, isotopically labeled derivative, or prodrug thereof, wherein R¹, R², R³, R⁴, R^{a6}, R⁷, R⁸, R⁹, R¹⁰, m, and n are as defined herein.

In certain embodiments, a compound of Formula (II) is of Formula (IIIa-iv):

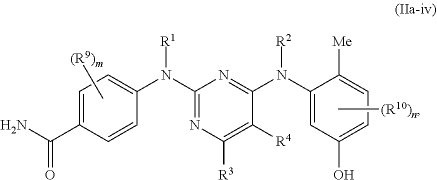

or a pharmaceutically acceptable salt, solvate, hydrate, polymorph, co-crystal, tautomer, stereoisomer, isotopically labeled derivative, or prodrug thereof, wherein R¹, R², R³, R⁴, R⁹, R¹⁰, m, and n are as defined herein.

In certain embodiments, a compound of Formula (II) is of Formula (IIIa-v):

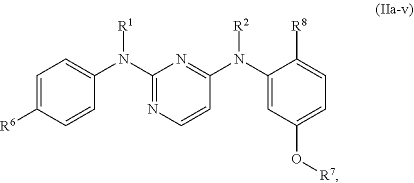

or a pharmaceutically acceptable salt, solvate, hydrate, polymorph, co-crystal, tautomer, stereoisomer, isotopically labeled derivative, or prodrug thereof, wherein R¹, R², R⁶, R⁷, and R⁸ are as defined herein.

In certain embodiments, a compound of Formula (II) is of Formula (IIa-vi):

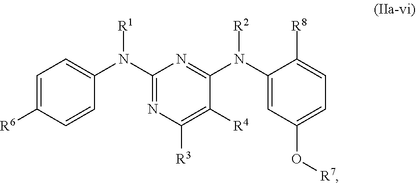

or a pharmaceutically acceptable salt, solvate, hydrate, polymorph, co-crystal, tautomer, stereoisomer, isotopically labeled derivative, or prodrug thereof, wherein R¹, R², R³, R⁴, R⁶, R⁷, and R⁸ are as defined herein.

In certain embodiments, a compound of Formula (II) is of Formula (IIb-i):

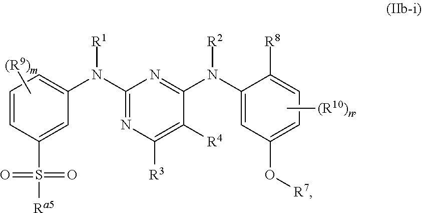

or a pharmaceutically acceptable salt, solvate, hydrate, polymorph, co-crystal, tautomer, stereoisomer, isotopically labeled derivative, or prodrug thereof, wherein R¹, R², R³, R⁴, R^{a5}, R⁷, R⁸, R⁹, R¹⁰, m, and n are as defined herein.

In certain embodiments, a compound of Formula (II) is of Formula (IIb-ii):

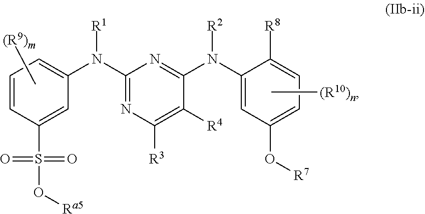

or a pharmaceutically acceptable salt, solvate, hydrate, polymorph, co-crystal, tautomer, stereoisomer, isotopically labeled derivative, or prodrug thereof, wherein R¹, R², R³, R⁴, R^{a5}, R⁷, R⁸, R⁹, R¹⁰, m, and n are as defined herein.

In certain embodiments, a compound of Formula (II) is of Formula (IIb-iii):

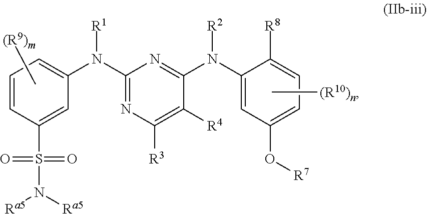

or a pharmaceutically acceptable salt, solvate, hydrate, polymorph, co-crystal, tautomer, stereoisomer, isotopically labeled derivative, or prodrug thereof, wherein R¹, R², R³, R⁴, R^{a5}, R⁷, R⁸, R⁹, R¹⁰, m, and n are as defined herein.

In certain embodiments, a compound of Formula (II) is of Formula (IIb-iv):

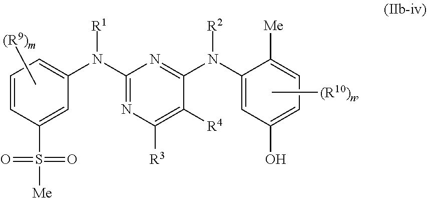

or a pharmaceutically acceptable salt, solvate, hydrate, polymorph, co-crystal, tautomer, stereoisomer, isotopically labeled derivative, or prodrug thereof, wherein R¹, R², R³, R⁴, R⁹, R¹⁰, m, and n are as defined herein.

In certain embodiments, a compound of Formula (II) is of Formula (IIb-v):

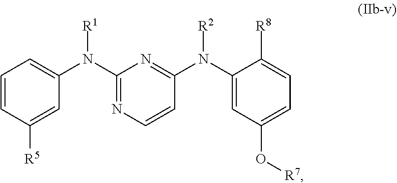

or a pharmaceutically acceptable salt, solvate, hydrate, polymorph, co-crystal, tautomer, stereoisomer, isotopically labeled derivative, or prodrug thereof, wherein R¹, R², R⁵, R⁷, and R⁸ are as defined herein.

In certain embodiments, a compound of Formula (II) is of Formula (IIb-vi):

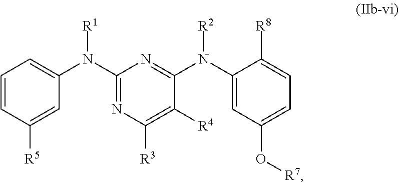

or a pharmaceutically acceptable salt, solvate, hydrate, polymorph, co-crystal, tautomer, stereoisomer, isotopically labeled derivative, or prodrug thereof, wherein R¹, R², R³, R⁴, R⁵, R⁷, and R⁸ are as defined herein.

In certain embodiments, a compound of Formula (II) is one of the following:

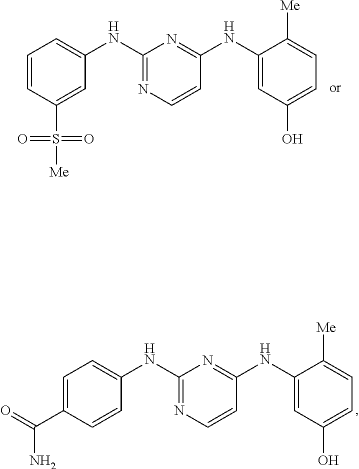 [SMILES: Cc1ccc(O)cc1Nc1ccnc(Nc2cccc(S(C)(=O)=O)c2)n1 ; Cc1ccc(O)cc1Nc1ccnc(Nc2ccc(C(N)=O)cc2)n1]

or a pharmaceutically acceptable salt, solvate, hydrate, polymorph, co-crystal, tautomer, stereoisomer, isotopically labeled derivative, or prodrug thereof.

In certain embodiments, the present disclosure provides pharmaceutical compositions comprising a compound provided herein (e.g., a compound of Formula (I) or Formula (II)), or a pharmaceutically acceptable salt, solvate, hydrate, polymorph, co-crystal, tautomer, stereoisomer, isotopically labeled derivative, or prodrug thereof; and a pharmaceutically acceptable carrier and/or excipient. In some embodiments, the pharmaceutical composition further comprises an additional pharmaceutical agent. In certain embodiments, a pharmaceutical composition provided herein is formulated for intraarticular injection.

In another aspect, the present disclosure provides methods for treating and/or preventing a disease or condition (e.g., an inflammatory disease, joint disease, proliferative disease, fibrosis, or pain) in a subject comprising administering to the subject an effective amount of a compound provided herein (e.g., a compound of Formula (I) or Formula (II)), or a pharmaceutically acceptable salt, solvate, hydrate, polymorph, co-crystal, tautomer, stereoisomer, isotopically labelled derivative, or prodrug thereof, or a pharmaceutical composition thereof.

In certain embodiments, the disease is an inflammatory disease. In some embodiments, the disease is a joint disease. In certain embodiments, the disease is a proliferative disease. In some embodiments, the disease is fibrosis. In certain embodiments, the condition is pain.

In yet another aspect, the present disclosure provides methods for inhibiting DDR2 activity (e.g., in a cell, tissue, or biological sample) comprising contacting a DDR2 protein with a compound provided herein (e.g., a compound of Formula (I) or Formula (II)), or a pharmaceutically acceptable salt, solvate, hydrate, polymorph, co-crystal, tautomer, stereoisomer, isotopically labeled derivative, or prodrug thereof, or a pharmaceutical composition thereof.

In yet another aspect, the present disclosure provides methods for inhibiting DDR1 activity (e.g., in a cell, tissue, or biological sample) comprising contacting a DDR1 protein with a compound provided herein (e.g., a compound of Formula (I) or Formula (II)), or a pharmaceutically acceptable salt, solvate, hydrate, polymorph, co-crystal, tautomer, stereoisomer, isotopically labeled derivative, or prodrug thereof, or a pharmaceutical composition thereof.

In yet another aspect, the present disclosure provides methods for inhibiting p38 MAPK (e.g., p38-alpha) activity (e.g., in a cell, tissue, or biological sample) comprising contacting a p38 MAPK protein (e.g., p38-alpha) with a compound provided herein (e.g., a compound of Formula (I) or Formula (II)), or a pharmaceutically acceptable salt, solvate, hydrate, polymorph, co-crystal, tautomer, stereoisomer, isotopically labeled derivative, or prodrug thereof, or a pharmaceutical composition thereof.

In further embodiments, the present disclosure provides kits comprising a compound provided herein (e.g., a compound of Formula (I) or Formula (II)), or a pharmaceutically acceptable salt, solvate, hydrate, polymorph, co-crystal, tautomer, stereoisomer, isotopically labeled derivative, or prodrug thereof; or a pharmaceutical composition provided herein; and optionally instructions for using the compound, pharmaceutically acceptable salt, solvate, hydrate, polymorph, co-crystal, tautomer, stereoisomer, isotopically labeled derivative, prodrug, or pharmaceutical composition (e.g., for treating and/or preventing a disease or condition in a subject, e.g., an inflammatory disease, joint disease, proliferative disease, fibrosis, or pain).

The details of certain embodiments of the invention are set forth in the Detailed Description of Certain Embodiments, as described below. Other features, objects, and advantages of the invention will be apparent from the Definitions, Examples, and Claims. It should be understood that the aspects described herein are not limited to specific embodiments, methods, or configurations, and as such can, of course, vary. It is also to be understood that the terminology used herein is for the purpose of describing particular aspects only and, unless specifically defined herein, is not intended to be limiting.

### BRIEF DESCRIPTION OF THE DRAWINGS

The accompanying drawings, which constitute a part of this specification, illustrate several embodiments of the invention and together with the description provide non-limiting examples of the invention.

FIG. 1 depicts morphology of articular cartilages of mouse knee joints after DMM surgery. Fibrillation was seen in the articular cartilage of mice at 8 weeks after DMM surgery without treatment with compound 3 (the PBS control group). In mice treated with compound 3, there was the appearance of localized chondrocyte clusters, but no fibrillation, in the articular cartilage after DMM surgery. At 12 weeks following DMM surgery, loss of articular cartilage was seen in mice without treatment with compound 3, whereas only localized absence of the proteoglycans was observed in mice treated with compound 3, see the arrows. There was no overt pathological morphology in sham control groups. (Bar=100 μm).

FIGS. 2A-2C show evaluation of the condition of articular cartilages. The condition of articular cartilages was evaluated by a modified scoring system for mice. As shown in the histogram, the severity of the cartilage damage was significantly different between mice with or without treatment with compound 3 at both 8 (FIG. 2A) and 12 weeks (FIG. 2B) following DMM. The average numbers with standard deviations are indicated in FIG. 2C. Inhibitor=compound 3.

FIG. 3 shows protein expression of DDR2 and Mmp-13 in the articular cartilage of mouse knee joints at 8 weeks following DMM surgery. Stained cells (insets), which represent the protein expression of DDR2 or Mmp-13, were detected in mice after DMM surgery without the treatment with compound 3. In mice treated with compound 3, the stained cells were dramatically reduced. There were no stained cells detected in sham control groups. (AC: articular cartilage; SB: subchondral bone, the dotted line: indicating the separation between articular cartilage and subchondral bone). Inhibitor=compound 3.

### DEFINITIONS

### Chemical Definitions

Definitions of specific functional groups and chemical terms are described in more detail below. The chemical elements are identified in accordance with the Periodic Table of the Elements, CAS version, Handbook of Chemistry and Physics, 75^{th} Ed., inside cover, and specific functional groups are generally defined as described therein. Additionally, general principles of organic chemistry, as well as specific functional moieties and reactivity, are described in Thomas Sorrell, Organic Chemistry, University Science Books, Sausalito, 1999; Michael B. Smith, March's Advanced Organic Chemistry, 7^{th} Edition, John Wiley & Sons, Inc., New York, 2013; Richard C. Larock, Comprehensive Organic Transformations, John Wiley & Sons, Inc., New York, 2018; and Carruthers, Some Modern Methods of Organic Synthesis, 3^{rd} Edition, Cambridge University Press, Cambridge, 1987.

Compounds described herein can comprise one or more asymmetric centers, and thus can exist in various stereoisomeric forms, e.g., enantiomers and/or diastereomers. For example, the compounds described herein can be in the form of an individual enantiomer, diastereomer or geometric isomer, or can be in the form of a mixture of stereoisomers, including racemic mixtures and mixtures enriched in one or more stereoisomer. Isomers can be isolated from mixtures by methods known to those skilled in the art, including chiral high pressure liquid chromatography (HPLC) and the formation and crystallization of chiral salts; or preferred isomers can be prepared by asymmetric syntheses. See, for example, Jacques et al., Enantiomers, Racemates and Resolutions (Wiley Interscience, New York, 1981); Wilen et al., Tetrahedron 33:2725 (1977); Eliel, E. L. Stereochemistry of Carbon Compounds (McGraw-Hill, NY, 1962); and Wilen, S. H., Tables of Resolving Agents and Optical Resolutions p. 268 (E. L. Eliel, Ed., Univ. of Notre Dame Press, Notre Dame, IN 1972). The invention additionally encompasses compounds as individual isomers substantially free of other isomers, and alternatively, as mixtures of various isomers.

Unless otherwise provided, formulae and structures depicted herein include compounds that do not include isotopically enriched atoms, and also include compounds that include isotopically enriched atoms. For example, compounds having the present structures except for the replacement of hydrogen by deuterium or tritium, replacement of ¹⁹F with ¹⁸F, or the replacement of a carbon by a ¹³C- or ¹⁴C-enriched carbon are within the scope of the disclosure. Such compounds are useful, for example, as analytical tools or probes in biological assays.

When a range of values is listed, it is intended to encompass each value and sub-range within the range. A range is inclusive of the values at the two ends of the range unless otherwise provided. For example “C₁₋₆ alkyl” encompasses, C₁, C₂, C₃, C₄, C₅, C₆, C₁₋₆, C₁₋₅, C₁₋₄, C₁₋₃, C₁₋₂, C₂₋₆, C₂₋₅, C₂₋₄, C₂₋₃, C₃₋₆, C₃₋₅, C₃₋₄, C₄₋₆, C₄₋₅, and C₅₋₆ alkyl.

The term “aliphatic” refers to alkyl, alkenyl, alkynyl, and carbocyclic groups. Likewise, the term “heteroaliphatic” refers to heteroalkyl, heteroalkenyl, heteroalkynyl, and heterocyclic groups.

The term “alkyl” refers to a radical of a straight-chain or branched saturated hydrocarbon group having from 1 to 20 carbon atoms (“C₁₋₂₀ alkyl”). In some embodiments, an alkyl group has 1 to 12 carbon atoms (“C₁₋₁₂ alkyl”). In some embodiments, an alkyl group has 1 to 10 carbon atoms (“C₁₋₁₀ alkyl”). In some embodiments, an alkyl group has 1 to 9 carbon atoms (“C₁₋₉ alkyl”). In some embodiments, an alkyl group has 1 to 8 carbon atoms (“C₁₋₈ alkyl”). In some embodiments, an alkyl group has 1 to 7 carbon atoms (“C₁₋₇ alkyl”). In some embodiments, an alkyl group has 1 to 6 carbon atoms (“C₁₋₆ alkyl”). In some embodiments, an alkyl group has 1 to 5 carbon atoms (“C₁₋₅ alkyl”). In some embodiments, an alkyl group has 1 to 4 carbon atoms (“C₁₋₄ alkyl”). In some embodiments, an alkyl group has 1 to 3 carbon atoms (“C₁₋₃ alkyl”). In some embodiments, an alkyl group has 1 to 2 carbon atoms (“C₁₋₂ alkyl”). In some embodiments, an alkyl group has 1 carbon atom (“C₁ alkyl”). In some embodiments, an alkyl group has 2 to 6 carbon atoms (“C₂₋₆ alkyl”). Examples of C₁₋₆ alkyl groups include methyl (C₁), ethyl (C₂), propyl (C₃) (e.g., n-propyl, isopropyl), butyl (C₄) (e.g., n-butyl, tert-butyl, sec-butyl, isobutyl), pentyl (C₅) (e.g., n-pentyl, 3-pentanyl, amyl, neopentyl, 3-methyl-2-butanyl, tert-amyl), and hexyl (C₆) (e.g., n-hexyl). Additional examples of alkyl groups include n-heptyl (C₇), n-octyl (C₈), n-dodecyl (C₁₂), and the like. Unless otherwise specified, each instance of an alkyl group is independently unsubstituted (an “unsubstituted alkyl”) or substituted (a “substituted alkyl”) with one or more substituents (e.g., halogen, such as F). In certain embodiments, the alkyl group is an unsubstituted C₁₋₁₂ alkyl (such as unsubstituted C₁₋₆ alkyl, e.g., —CH₃ (Me), unsubstituted ethyl (Et), unsubstituted propyl (Pr, e.g., unsubstituted n-propyl (n-Pr), unsubstituted isopropyl (i-Pr)), unsubstituted butyl (Bu, e.g., unsubstituted n-butyl (n-Bu), unsubstituted tert-butyl (tert-Bu or t-Bu), unsubstituted sec-butyl (sec-Bu or s-Bu), unsubstituted isobutyl (i-Bu)). In certain embodiments, the alkyl group is a substituted C₁₋₁₂ alkyl (such as substituted C₁₋₆ alkyl, e.g., —CH₂F, —CHF₂, —CF₃, —CH₂CH₂F, —CH₂CHF₂, —CH₂CF₃, or benzyl (Bn)).

The term “haloalkyl” is a substituted alkyl group, wherein one or more of the hydrogen atoms are independently replaced by a halogen, e.g., fluoro, bromo, chloro, or iodo. “Perhaloalkyl” is a subset of haloalkyl, and refers to an alkyl group wherein all of the hydrogen atoms are independently replaced by a halogen, e.g., fluoro, bromo, chloro, or iodo. In some embodiments, the haloalkyl moiety has 1 to 20 carbon atoms (“C₁₋₂₀ haloalkyl”). In some embodiments, the haloalkyl moiety has 1 to 10 carbon atoms (“C₁₋₁₀ haloalkyl”). In some embodiments, the haloalkyl moiety has 1 to 9 carbon atoms (“C₁₋₉ haloalkyl”). In some embodiments, the haloalkyl moiety has 1 to 8 carbon atoms (“C₁₋₈ haloalkyl”). In some embodiments, the haloalkyl moiety has 1 to 7 carbon atoms (“C₁₋₇ haloalkyl”). In some embodiments, the haloalkyl moiety has 1 to 6 carbon atoms (“C₁₋₆ haloalkyl”). In some embodiments, the haloalkyl moiety has 1 to 5 carbon atoms (“C₁₋₅ haloalkyl”). In some embodiments, the haloalkyl moiety has 1 to 4 carbon atoms (“C₁₋₄ haloalkyl”). In some embodiments, the haloalkyl moiety has 1 to 3 carbon atoms (“C₁₋₃ haloalkyl”). In some embodiments, the haloalkyl moiety has 1 to 2 carbon atoms (“C₁₋₂ haloalkyl”). In some embodiments, all of the haloalkyl hydrogen atoms are independently replaced with fluoro to provide a “perfluoroalkyl” group. In some embodiments, all of the haloalkyl hydrogen atoms are independently replaced with chloro to provide a “perchloroalkyl” group. Examples of haloalkyl groups include —CHF₂, —CH₂F, —CF₃, —CH₂CF₃, —CF₂CF₃, —CF₂CF₂CF₃, —CCl₃, —CFCl₂, —CF₂Cl, and the like.

The term “heteroalkyl” refers to an alkyl group, which further includes at least one heteroatom (e.g., 1, 2, 3, or 4 heteroatoms) selected from oxygen, nitrogen, or sulfur within (e.g., inserted between adjacent carbon atoms of) and/or placed at one or more terminal position(s) of the parent chain. In certain embodiments, a heteroalkyl group refers to a saturated group having from 1 to 20 carbon atoms and 1 or more heteroatoms within the parent chain (“heteroC₁₋₂₀ alkyl”). In certain embodiments, a heteroalkyl group refers to a saturated group having from 1 to 12 carbon atoms and 1 or more heteroatoms within the parent chain (“heteroC₁₋₁₂ alkyl”). In some embodiments, a heteroalkyl group is a saturated group having 1 to 11 carbon atoms and 1 or more heteroatoms within the parent chain (“heteroC₁₋₁₁ alkyl”). In some embodiments, a heteroalkyl group is a saturated group having 1 to 10 carbon atoms and 1 or more heteroatoms within the parent chain (“heteroC₁₋₁₀ alkyl”). In some embodiments, a heteroalkyl group is a saturated group having 1 to 9 carbon atoms and 1 or more heteroatoms within the parent chain (“heteroC₁₋₉ alkyl”). In some embodiments, a heteroalkyl group is a saturated group having 1 to 8 carbon atoms and 1 or more heteroatoms within the parent chain (“heteroC₁₋₈ alkyl”). In some embodiments, a heteroalkyl group is a saturated group having 1 to 7 carbon atoms and 1 or more heteroatoms within the parent chain (“heteroC₁₋₇ alkyl”). In some embodiments, a heteroalkyl group is a saturated group having 1 to 6 carbon atoms and 1 or more heteroatoms within the parent chain (“heteroC₁₋₆ alkyl”). In some embodiments, a heteroalkyl group is a saturated group having 1 to 5 carbon atoms and 1 or 2 heteroatoms within the parent chain (“heteroC₁₋₅ alkyl”). In some embodiments, a heteroalkyl group is a saturated group having 1 to 4 carbon atoms and 1 or 2 heteroatoms within the parent chain (“heteroC₁₋₄ alkyl”). In some embodiments, a heteroalkyl group is a saturated group having 1 to 3 carbon atoms and 1 heteroatom within the parent chain (“heteroC₁₋₃ alkyl”). In some embodiments, a heteroalkyl group is a saturated group having 1 to 2 carbon atoms and 1 heteroatom within the parent chain (“heteroC₁₋₂ alkyl”). In some embodiments, a heteroalkyl group is a saturated group having 1 carbon atom and 1 heteroatom (“heteroC₁ alkyl”). In some embodiments, a heteroalkyl group is a saturated group having 2 to 6 carbon atoms and 1 or 2 heteroatoms within the parent chain (“heteroC₂₋₆ alkyl”). Unless otherwise specified, each instance of a heteroalkyl group is independently unsubstituted (an “unsubstituted heteroalkyl”) or substituted (a “substituted heteroalkyl”) with one or more substituents. In certain embodiments, the heteroalkyl group is an unsubstituted heteroC₁₋₁₂ alkyl. In certain embodiments, the heteroalkyl group is a substituted heteroC₁₋₁₂ alkyl.

The term “alkenyl” refers to a radical of a straight-chain or branched hydrocarbon group having from 1 to 20 carbon atoms and one or more carbon-carbon double bonds (e.g., 1, 2, 3, or 4 double bonds). In some embodiments, an alkenyl group has 1 to 20 carbon atoms (“C₁₋₂₀ alkenyl”). In some embodiments, an alkenyl group has 1 to 12 carbon atoms (“C₁₋₁₂ alkenyl”). In some embodiments, an alkenyl group has 1 to 11 carbon atoms (“C₁₋₁₁ alkenyl”). In some embodiments, an alkenyl group has 1 to 10 carbon atoms (“C₁₋₁₀ alkenyl”). In some embodiments, an alkenyl group has 1 to 9 carbon atoms (“C₁₋₉ alkenyl”). In some embodiments, an alkenyl group has 1 to 8 carbon atoms (“C₁₋₈ alkenyl”). In some embodiments, an alkenyl group has 1 to 7 carbon atoms (“C₁₋₇ alkenyl”). In some embodiments, an alkenyl group has 1 to 6 carbon atoms (“C₁₋₆ alkenyl”). In some embodiments, an alkenyl group has 1 to 5 carbon atoms (“C₁₋₅ alkenyl”). In some embodiments, an alkenyl group has 1 to 4 carbon atoms (“C₁₋₄ alkenyl”). In some embodiments, an alkenyl group has 1 to 3 carbon atoms (“C₁₋₃ alkenyl”). In some embodiments, an alkenyl group has 1 to 2 carbon atoms (“C₁₋₂ alkenyl”). In some embodiments, an alkenyl group has 1 carbon atom (“C₁ alkenyl”). The one or more carbon-carbon double bonds can be internal (such as in 2-butenyl) or terminal (such as in 1-butenyl). Examples of C₁₋₄ alkenyl groups include methylidenyl (C₁), ethenyl (C₂), 1-propenyl (C₃), 2-propenyl (C₃), 1-butenyl (C₄), 2-butenyl (C₄), butadienyl (C₄), and the like. Examples of C₁₋₆ alkenyl groups include the aforementioned C₂₋₄ alkenyl groups as well as pentenyl (C₅), pentadienyl (C₅), hexenyl (C₆), and the like. Additional examples of alkenyl include heptenyl (C₇), octenyl (C₈), octatrienyl (C₈), and the like. Unless otherwise specified, each instance of an alkenyl group is independently unsubstituted (an “unsubstituted alkenyl”) or substituted (a “substituted alkenyl”) with one or more substituents. In certain embodiments, the alkenyl group is an unsubstituted C₁₋₂₀ alkenyl. In certain embodiments, the alkenyl group is a substituted C₁₋₂₀ alkenyl. In an alkenyl group, a C═C double bond for which the stereochemistry is not specified (e.g., —CH═CHCH₃ or

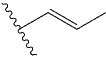 [SMILES: */C=C/C]

may be in the (E)- or (Z)-configuration.

The term “heteroalkenyl” refers to an alkenyl group, which further includes at least one heteroatom (e.g., 1, 2, 3, or 4 heteroatoms) selected from oxygen, nitrogen, or sulfur within (e.g., inserted between adjacent carbon atoms of) and/or placed at one or more terminal position(s) of the parent chain. In certain embodiments, a heteroalkenyl group refers to a group having from 1 to 20 carbon atoms, at least one double bond, and 1 or more heteroatoms within the parent chain (“heteroC₁₋₂₀ alkenyl”). In certain embodiments, a heteroalkenyl group refers to a group having from 1 to 12 carbon atoms, at least one double bond, and 1 or more heteroatoms within the parent chain (“heteroC₁₋₁₂ alkenyl”). In certain embodiments, a heteroalkenyl group refers to a group having from 1 to 11 carbon atoms, at least one double bond, and 1 or more heteroatoms within the parent chain (“heteroC₁₋₁₁ alkenyl”). In certain embodiments, a heteroalkenyl group refers to a group having from 1 to 10 carbon atoms, at least one double bond, and 1 or more heteroatoms within the parent chain (“heteroC₁₋₁₀ alkenyl”). In some embodiments, a heteroalkenyl group has 1 to 9 carbon atoms at least one double bond, and 1 or more heteroatoms within the parent chain (“heteroC₁₋₉ alkenyl”). In some embodiments, a heteroalkenyl group has 1 to 8 carbon atoms, at least one double bond, and 1 or more heteroatoms within the parent chain (“heteroC₁₋₈ alkenyl”). In some embodiments, a heteroalkenyl group has 1 to 7 carbon atoms, at least one double bond, and 1 or more heteroatoms within the parent chain (“heteroC₁₋₇ alkenyl”). In some embodiments, a heteroalkenyl group has 1 to 6 carbon atoms, at least one double bond, and 1 or more heteroatoms within the parent chain (“heteroC₁₋₆ alkenyl”). In some embodiments, a heteroalkenyl group has 1 to 5 carbon atoms, at least one double bond, and 1 or 2 heteroatoms within the parent chain (“heteroC₁₋₅ alkenyl”). In some embodiments, a heteroalkenyl group has 1 to 4 carbon atoms, at least one double bond, and 1 or 2 heteroatoms within the parent chain (“heteroC₁₋₄ alkenyl”). In some embodiments, a heteroalkenyl group has 1 to 3 carbon atoms, at least one double bond, and 1 heteroatom within the parent chain (“heteroC₁₋₃ alkenyl”). In some embodiments, a heteroalkenyl group has 1 to 2 carbon atoms, at least one double bond, and 1 heteroatom within the parent chain (“heteroC₁₋₂ alkenyl”). In some embodiments, a heteroalkenyl group has 1 to 6 carbon atoms, at least one double bond, and 1 or 2 heteroatoms within the parent chain (“heteroC₁₋₆ alkenyl”). Unless otherwise specified, each instance of a heteroalkenyl group is independently unsubstituted (an “unsubstituted heteroalkenyl”) or substituted (a “substituted heteroalkenyl”) with one or more substituents. In certain embodiments, the heteroalkenyl group is an unsubstituted heteroC₁₋₂₀ alkenyl. In certain embodiments, the heteroalkenyl group is a substituted heteroC₁₋₂₀ alkenyl.

The term “alkynyl” refers to a radical of a straight-chain or branched hydrocarbon group having from 1 to 20 carbon atoms and one or more carbon-carbon triple bonds (e.g., 1, 2, 3, or 4 triple bonds) (“C₁₋₂₀ alkynyl”). In some embodiments, an alkynyl group has 1 to 10 carbon atoms (“C₁₋₁₀ alkynyl”). In some embodiments, an alkynyl group has 1 to 9 carbon atoms (“C₁₋₉ alkynyl”). In some embodiments, an alkynyl group has 1 to 8 carbon atoms (“C₁₋₈ alkynyl”). In some embodiments, an alkynyl group has 1 to 7 carbon atoms (“C₁₋₇ alkynyl”). In some embodiments, an alkynyl group has 1 to 6 carbon atoms (“C₁₋₆ alkynyl”). In some embodiments, an alkynyl group has 1 to 5 carbon atoms (“C₁₋₅ alkynyl”). In some embodiments, an alkynyl group has 1 to 4 carbon atoms (“C₁₋₄ alkynyl”). In some embodiments, an alkynyl group has 1 to 3 carbon atoms (“C₁₋₃ alkynyl”). In some embodiments, an alkynyl group has 1 to 2 carbon atoms (“C₁₋₂ alkynyl”). In some embodiments, an alkynyl group has 1 carbon atom (“C₁ alkynyl”). The one or more carbon-carbon triple bonds can be internal (such as in 2-butynyl) or terminal (such as in 1-butynyl). Examples of C₁₋₄ alkynyl groups include, without limitation, methylidynyl (C₁), ethynyl (C₂), 1-propynyl (C₃), 2-propynyl (C₃), 1-butynyl (C₄), 2-butynyl (C₄), and the like. Examples of C₁₋₆ alkenyl groups include the aforementioned C₂₋₄ alkynyl groups as well as pentynyl (C₅), hexynyl (C₆), and the like. Additional examples of alkynyl include heptynyl (C₇), octynyl (C₈), and the like. Unless otherwise specified, each instance of an alkynyl group is independently unsubstituted (an “unsubstituted alkynyl”) or substituted (a “substituted alkynyl”) with one or more substituents. In certain embodiments, the alkynyl group is an unsubstituted C₁₋₂₀ alkynyl. In certain embodiments, the alkynyl group is a substituted C₁₋₂₀ alkynyl.

The term “heteroalkynyl” refers to an alkynyl group, which further includes at least one heteroatom (e.g., 1, 2, 3, or 4 heteroatoms) selected from oxygen, nitrogen, or sulfur within (e.g., inserted between adjacent carbon atoms of) and/or placed at one or more terminal position(s) of the parent chain. In certain embodiments, a heteroalkynyl group refers to a group having from 1 to 20 carbon atoms, at least one triple bond, and 1 or more heteroatoms within the parent chain (“heteroC₁₋₂₀ alkynyl”). In certain embodiments, a heteroalkynyl group refers to a group having from 1 to 10 carbon atoms, at least one triple bond, and 1 or more heteroatoms within the parent chain (“heteroC₁₋₁₀ alkynyl”). In some embodiments, a heteroalkynyl group has 1 to 9 carbon atoms, at least one triple bond, and 1 or more heteroatoms within the parent chain (“heteroC₁₋₉ alkynyl”). In some embodiments, a heteroalkynyl group has 1 to 8 carbon atoms, at least one triple bond, and 1 or more heteroatoms within the parent chain (“heteroC₁₋₈ alkynyl”). In some embodiments, a heteroalkynyl group has 1 to 7 carbon atoms, at least one triple bond, and 1 or more heteroatoms within the parent chain (“heteroC₁₋₇ alkynyl”). In some embodiments, a heteroalkynyl group has 1 to 6 carbon atoms, at least one triple bond, and 1 or more heteroatoms within the parent chain (“heteroC₁₋₆ alkynyl”). In some embodiments, a heteroalkynyl group has 1 to 5 carbon atoms, at least one triple bond, and 1 or 2 heteroatoms within the parent chain (“heteroC₁₋₅ alkynyl”). In some embodiments, a heteroalkynyl group has 1 to 4 carbon atoms, at least one triple bond, and 1 or 2 heteroatoms within the parent chain (“heteroC₁₋₄ alkynyl”). In some embodiments, a heteroalkynyl group has 1 to 3 carbon atoms, at least one triple bond, and 1 heteroatom within the parent chain (“heteroC₁₋₃ alkynyl”). In some embodiments, a heteroalkynyl group has 1 to 2 carbon atoms, at least one triple bond, and 1 heteroatom within the parent chain (“heteroC₁₋₂ alkynyl”). In some embodiments, a heteroalkynyl group has 1 to 6 carbon atoms, at least one triple bond, and 1 or 2 heteroatoms within the parent chain (“heteroC₁₋₆ alkynyl”). Unless otherwise specified, each instance of a heteroalkynyl group is independently unsubstituted (an “unsubstituted heteroalkynyl”) or substituted (a “substituted heteroalkynyl”) with one or more substituents. In certain embodiments, the heteroalkynyl group is an unsubstituted heteroC₁₋₂₀ alkynyl. In certain embodiments, the heteroalkynyl group is a substituted heteroC₁₋₂₀ alkynyl.

The term “carbocyclyl” or “carbocyclic” refers to a radical of a non-aromatic cyclic hydrocarbon group having from 3 to 14 ring carbon atoms (“C₃₋₁₄ carbocyclyl”) and zero heteroatoms in the non-aromatic ring system. In some embodiments, a carbocyclyl group has 3 to 14 ring carbon atoms (“C₃₋₁₄ carbocyclyl”). In some embodiments, a carbocyclyl group has 3 to 13 ring carbon atoms (“C₃₋₁₃ carbocyclyl”). In some embodiments, a carbocyclyl group has 3 to 12 ring carbon atoms (“C₃₋₁₂ carbocyclyl”). In some embodiments, a carbocyclyl group has 3 to 11 ring carbon atoms (“C₃₋₁₁ carbocyclyl”). In some embodiments, a carbocyclyl group has 3 to 10 ring carbon atoms (“C₃₋₁₀ carbocyclyl”). In some embodiments, a carbocyclyl group has 3 to 8 ring carbon atoms (“C₃₋₈ carbocyclyl”). In some embodiments, a carbocyclyl group has 3 to 7 ring carbon atoms (“C₃₋₇ carbocyclyl”). In some embodiments, a carbocyclyl group has 3 to 6 ring carbon atoms (“C₃₋₆ carbocyclyl”). In some embodiments, a carbocyclyl group has 4 to 6 ring carbon atoms (“C₄₋₆ carbocyclyl”). In some embodiments, a carbocyclyl group has 5 to 6 ring carbon atoms (“C₅₋₆ carbocyclyl”). In some embodiments, a carbocyclyl group has 5 to 10 ring carbon atoms (“C₅₋₁₀ carbocyclyl”). Exemplary C₃₋₆ carbocyclyl groups include cyclopropyl (C₃), cyclopropenyl (C₃), cyclobutyl (C₄), cyclobutenyl (C₄), cyclopentyl (C₅), cyclopentenyl (C₅), cyclohexyl (C₆), cyclohexenyl (C₆), cyclohexadienyl (C₆), and the like. Exemplary C₃₋₈ carbocyclyl groups include the aforementioned C₃₋₆ carbocyclyl groups as well as cycloheptyl (C₇), cycloheptenyl (C₇), cycloheptadienyl (C₇), cycloheptatrienyl (C₇), cyclooctyl (C₈), cyclooctenyl (C₈), bicyclo[2.2.1]heptanyl (C₇), bicyclo[2.2.2]octanyl (C₈), and the like. Exemplary C₃₋₁₀ carbocyclyl groups include the aforementioned C₃₋₈ carbocyclyl groups as well as cyclononyl (C₉), cyclononenyl (C₉), cyclodecyl (C₁₀), cyclodecenyl (C₁₀), octahydro-1H-indenyl (C₉), decahydronaphthalenyl (C₁₀), spiro[4.5]decanyl (C₁₀), and the like. Exemplary C₃₋₈ carbocyclyl groups include the aforementioned C₃₋₁₀ carbocyclyl groups as well as cycloundecyl (C₁₁), spiro[5.5]undecanyl (C₁₁), cyclododecyl (C₁₂), cyclododecenyl (C₁₂), cyclotridecane (C₁₃), cyclotetradecane (C₁₄), and the like. As the foregoing examples illustrate, in certain embodiments, the carbocyclyl group is either monocyclic (“monocyclic carbocyclyl”) or polycyclic (e.g., containing a fused, bridged or spiro ring system such as a bicyclic system (“bicyclic carbocyclyl”) or tricyclic system (“tricyclic carbocyclyl”)) and can be saturated or can contain one or more carbon-carbon double or triple bonds. “Carbocyclyl” also includes ring systems wherein the carbocyclyl ring, as defined above, is fused with one or more aryl or heteroaryl groups wherein the point of attachment is on the carbocyclyl ring, and in such instances, the number of carbons continue to designate the number of carbons in the carbocyclic ring system. Unless otherwise specified, each instance of a carbocyclyl group is independently unsubstituted (an “unsubstituted carbocyclyl”) or substituted (a “substituted carbocyclyl”) with one or more substituents. In certain embodiments, the carbocyclyl group is an unsubstituted C₃₋₁₄ carbocyclyl. In certain embodiments, the carbocyclyl group is a substituted C₃₋₁₄ carbocyclyl.

In some embodiments, “carbocyclyl” is a monocyclic, saturated carbocyclyl group having from 3 to 14 ring carbon atoms (“C₃₋₁₄ cycloalkyl”). In some embodiments, a cycloalkyl group has 3 to 10 ring carbon atoms (“C₃₋₁₀ cycloalkyl”). In some embodiments, a cycloalkyl group has 3 to 8 ring carbon atoms (“C₃₋₈ cycloalkyl”). In some embodiments, a cycloalkyl group has 3 to 6 ring carbon atoms (“C₃₋₆ cycloalkyl”). In some embodiments, a cycloalkyl group has 4 to 6 ring carbon atoms (“C₄₋₆ cycloalkyl”). In some embodiments, a cycloalkyl group has 5 to 6 ring carbon atoms (“C₅₋₆ cycloalkyl”). In some embodiments, a cycloalkyl group has 5 to 10 ring carbon atoms (“C₅₋₁₀ cycloalkyl”). Examples of C₅₋₆ cycloalkyl groups include cyclopentyl (C₅) and cyclohexyl (C₅). Examples of C₃₋₆ cycloalkyl groups include the aforementioned C₅₋₆ cycloalkyl groups as well as cyclopropyl (C₃) and cyclobutyl (C₄). Examples of C₃₋₈ cycloalkyl groups include the aforementioned C₃₋₆ cycloalkyl groups as well as cycloheptyl (C₇) and cyclooctyl (C₈). Unless otherwise specified, each instance of a cycloalkyl group is independently unsubstituted (an “unsubstituted cycloalkyl”) or substituted (a “substituted cycloalkyl”) with one or more substituents. In certain embodiments, the cycloalkyl group is an unsubstituted C₃₋₁₄ cycloalkyl. In certain embodiments, the cycloalkyl group is a substituted C₃₋₁₄ cycloalkyl. In certain embodiments, the carbocyclyl includes 0, 1, or 2 C═C double bonds in the carbocyclic ring system, as valency permits.

The term “heterocyclyl” or “heterocyclic” refers to a radical of a 3- to 14-membered non-aromatic ring system having ring carbon atoms and 1 to 4 ring heteroatoms, wherein each heteroatom is independently selected from nitrogen, oxygen, and sulfur (“3-14 membered heterocyclyl”). In heterocyclyl groups that contain one or more nitrogen atoms, the point of attachment can be a carbon or nitrogen atom, as valency permits. A heterocyclyl group can either be monocyclic (“monocyclic heterocyclyl”) or polycyclic (e.g., a fused, bridged or spiro ring system such as a bicyclic system (“bicyclic heterocyclyl”) or tricyclic system (“tricyclic heterocyclyl”)), and can be saturated or can contain one or more carbon-carbon double or triple bonds. Heterocyclyl polycyclic ring systems can include one or more heteroatoms in one or both rings. “Heterocyclyl” also includes ring systems wherein the heterocyclyl ring, as defined above, is fused with one or more carbocyclyl groups wherein the point of attachment is either on the carbocyclyl or heterocyclyl ring, or ring systems wherein the heterocyclyl ring, as defined above, is fused with one or more aryl or heteroaryl groups, wherein the point of attachment is on the heterocyclyl ring, and in such instances, the number of ring members continue to designate the number of ring members in the heterocyclyl ring system. Unless otherwise specified, each instance of heterocyclyl is independently unsubstituted (an “unsubstituted heterocyclyl”) or substituted (a “substituted heterocyclyl”) with one or more substituents. In certain embodiments, the heterocyclyl group is an unsubstituted 3-14 membered heterocyclyl. In certain embodiments, the heterocyclyl group is a substituted 3-14 membered heterocyclyl. In certain embodiments, the heterocyclyl is substituted or unsubstituted, 3- to 7-membered, monocyclic heterocyclyl, wherein 1, 2, or 3 atoms in the heterocyclic ring system are independently oxygen, nitrogen, or sulfur, as valency permits.

In some embodiments, a heterocyclyl group is a 5-10 membered non-aromatic ring system having ring carbon atoms and 1-4 ring heteroatoms, wherein each heteroatom is independently selected from nitrogen, oxygen, and sulfur (“5-10 membered heterocyclyl”). In some embodiments, a heterocyclyl group is a 5-8 membered non-aromatic ring system having ring carbon atoms and 1-4 ring heteroatoms, wherein each heteroatom is independently selected from nitrogen, oxygen, and sulfur (“5-8 membered heterocyclyl”). In some embodiments, a heterocyclyl group is a 5-6 membered non-aromatic ring system having ring carbon atoms and 1-4 ring heteroatoms, wherein each heteroatom is independently selected from nitrogen, oxygen, and sulfur (“5-6 membered heterocyclyl”). In some embodiments, the 5-6 membered heterocyclyl has 1-3 ring heteroatoms selected from nitrogen, oxygen, and sulfur. In some embodiments, the 5-6 membered heterocyclyl has 1-2 ring heteroatoms selected from nitrogen, oxygen, and sulfur. In some embodiments, the 5-6 membered heterocyclyl has 1 ring heteroatom selected from nitrogen, oxygen, and sulfur.

Exemplary 3-membered heterocyclyl groups containing 1 heteroatom include azirdinyl, oxiranyl, and thiiranyl. Exemplary 4-membered heterocyclyl groups containing 1 heteroatom include azetidinyl, oxetanyl, and thietanyl. Exemplary 5-membered heterocyclyl groups containing 1 heteroatom include tetrahydrofuranyl, dihydrofuranyl, tetrahydrothiophenyl, dihydrothiophenyl, pyrrolidinyl, dihydropyrrolyl, and pyrrolyl-2,5-dione. Exemplary 5-membered heterocyclyl groups containing 2 heteroatoms include dioxolanyl, oxathiolanyl and dithiolanyl. Exemplary 5-membered heterocyclyl groups containing 3 heteroatoms include triazolinyl, oxadiazolinyl, and thiadiazolinyl. Exemplary 6-membered heterocyclyl groups containing 1 heteroatom include piperidinyl, tetrahydropyranyl, dihydropyridinyl, and thianyl. Exemplary 6-membered heterocyclyl groups containing 2 heteroatoms include piperazinyl, morpholinyl, dithianyl, and dioxanyl. Exemplary 6-membered heterocyclyl groups containing 3 heteroatoms include triazinyl. Exemplary 7-membered heterocyclyl groups containing 1 heteroatom include azepanyl, oxepanyl and thiepanyl. Exemplary 8-membered heterocyclyl groups containing 1 heteroatom include azocanyl, oxecanyl and thiocanyl. Exemplary bicyclic heterocyclyl groups include indolinyl, isoindolinyl, dihydrobenzofuranyl, dihydrobenzothienyl, tetrahydrobenzothienyl, tetrahydrobenzofuranyl, tetrahydroindolyl, tetrahydroquinolinyl, tetrahydroisoquinolinyl, decahydroquinolinyl, decahydroisoquinolinyl, octahydrochromenyl, octahydroisochromenyl, decahydronaphthyridinyl, decahydro-1,8-naphthyridinyl, octahydropyrrolo[3,2-b]pyrrole, indolinyl, phthalimidyl, naphthalimidyl, chromanyl, chromenyl, 1H-benzo[e][1,4]diazepinyl, 1,4,5,7-tetrahydropyrano[3,4-b]pyrrolyl, 5,6-dihydro-4H-furo[3,2-b]pyrrolyl, 6,7-dihydro-5H-furo[3,2-b]pyranyl, 5,7-dihydro-4H-thieno[2,3-c]pyranyl, 2,3-dihydro-1H-pyrrolo[2,3-b]pyridinyl, 2,3-dihydrofuro[2,3-b]pyridinyl, 4,5,6,7-tetrahydro-1H-pyrrolo[2,3-b]pyridinyl, 4,5,6,7-tetrahydrofuro[3,2-c]pyridinyl, 4,5,6,7-tetrahydrothieno[3,2-b]pyridinyl, 1,2,3,4-tetrahydro-1,6-naphthyridinyl, and the like.

The term “aryl” refers to a radical of a monocyclic or polycyclic (e.g., bicyclic or tricyclic) 4n+2 aromatic ring system (e.g., having 6, 10, or 14 π electrons shared in a cyclic array) having 6-14 ring carbon atoms and zero heteroatoms provided in the aromatic ring system (“C₆₋₁₄ aryl”). In some embodiments, an aryl group has 6 ring carbon atoms (“C₆ aryl”; e.g., phenyl). In some embodiments, an aryl group has 10 ring carbon atoms (“C₁₀ aryl”; e.g., naphthyl such as 1-naphthyl and 2-naphthyl). In some embodiments, an aryl group has 14 ring carbon atoms (“C₁₄ aryl”; e.g., anthracyl). “Aryl” also includes ring systems wherein the aryl ring, as defined above, is fused with one or more carbocyclyl or heterocyclyl groups wherein the radical or point of attachment is on the aryl ring, and in such instances, the number of carbon atoms continue to designate the number of carbon atoms in the aryl ring system. Unless otherwise specified, each instance of an aryl group is independently unsubstituted (an “unsubstituted aryl”) or substituted (a “substituted aryl”) with one or more substituents. In certain embodiments, the aryl group is an unsubstituted C₆₋₁₄ aryl. In certain embodiments, the aryl group is a substituted C₆₋₁₄ aryl.

“Aralkyl” is a subset of “alkyl” and refers to an alkyl group substituted by an aryl group, wherein the point of attachment is on the alkyl moiety.

The term “heteroaryl” refers to a radical of a 5-14 membered monocyclic or polycyclic (e.g., bicyclic, tricyclic) 4n+2 aromatic ring system (e.g., having 6, 10, or 14 π electrons shared in a cyclic array) having ring carbon atoms and 1-4 ring heteroatoms provided in the aromatic ring system, wherein each heteroatom is independently selected from nitrogen, oxygen, and sulfur (“5-14 membered heteroaryl”). In heteroaryl groups that contain one or more nitrogen atoms, the point of attachment can be a carbon or nitrogen atom, as valency permits. Heteroaryl polycyclic ring systems can include one or more heteroatoms in one or both rings. “Heteroaryl” includes ring systems wherein the heteroaryl ring, as defined above, is fused with one or more carbocyclyl or heterocyclyl groups wherein the point of attachment is on the heteroaryl ring, and in such instances, the number of ring members continue to designate the number of ring members in the heteroaryl ring system. “Heteroaryl” also includes ring systems wherein the heteroaryl ring, as defined above, is fused with one or more aryl groups wherein the point of attachment is either on the aryl or heteroaryl ring, and in such instances, the number of ring members designates the number of ring members in the fused polycyclic (aryl/heteroaryl) ring system. Polycyclic heteroaryl groups wherein one ring does not contain a heteroatom (e.g., indolyl, quinolinyl, carbazolyl, and the like) the point of attachment can be on either ring, e.g., either the ring bearing a heteroatom (e.g., 2-indolyl) or the ring that does not contain a heteroatom (e.g., 5-indolyl). In certain embodiments, the heteroaryl is substituted or unsubstituted, 5- or 6-membered, monocyclic heteroaryl, wherein 1, 2, 3, or 4 atoms in the heteroaryl ring system are independently oxygen, nitrogen, or sulfur. In certain embodiments, the heteroaryl is substituted or unsubstituted, 9- or 10-membered, bicyclic heteroaryl, wherein 1, 2, 3, or 4 atoms in the heteroaryl ring system are independently oxygen, nitrogen, or sulfur.

In some embodiments, a heteroaryl group is a 5-10 membered aromatic ring system having ring carbon atoms and 1-4 ring heteroatoms provided in the aromatic ring system, wherein each heteroatom is independently selected from nitrogen, oxygen, and sulfur (“5-10 membered heteroaryl”). In some embodiments, a heteroaryl group is a 5-8 membered aromatic ring system having ring carbon atoms and 1-4 ring heteroatoms provided in the aromatic ring system, wherein each heteroatom is independently selected from nitrogen, oxygen, and sulfur (“5-8 membered heteroaryl”). In some embodiments, a heteroaryl group is a 5-6 membered aromatic ring system having ring carbon atoms and 1-4 ring heteroatoms provided in the aromatic ring system, wherein each heteroatom is independently selected from nitrogen, oxygen, and sulfur (“5-6 membered heteroaryl”). In some embodiments, the 5-6 membered heteroaryl has 1-3 ring heteroatoms selected from nitrogen, oxygen, and sulfur. In some embodiments, the 5-6 membered heteroaryl has 1-2 ring heteroatoms selected from nitrogen, oxygen, and sulfur. In some embodiments, the 5-6 membered heteroaryl has 1 ring heteroatom selected from nitrogen, oxygen, and sulfur. Unless otherwise specified, each instance of a heteroaryl group is independently unsubstituted (an “unsubstituted heteroaryl”) or substituted (a “substituted heteroaryl”) with one or more substituents. In certain embodiments, the heteroaryl group is an unsubstituted 5-14 membered heteroaryl. In certain embodiments, the heteroaryl group is a substituted 5-14 membered heteroaryl.

Exemplary 5-membered heteroaryl groups containing 1 heteroatom include pyrrolyl, furanyl, and thiophenyl. Exemplary 5-membered heteroaryl groups containing 2 heteroatoms include imidazolyl, pyrazolyl, oxazolyl, isoxazolyl, thiazolyl, and isothiazolyl. Exemplary 5-membered heteroaryl groups containing 3 heteroatoms include triazolyl, oxadiazolyl, and thiadiazolyl. Exemplary 5-membered heteroaryl groups containing 4 heteroatoms include tetrazolyl. Exemplary 6-membered heteroaryl groups containing 1 heteroatom include pyridinyl. Exemplary 6-membered heteroaryl groups containing 2 heteroatoms include pyridazinyl, pyrimidinyl, and pyrazinyl. Exemplary 6-membered heteroaryl groups containing 3 or 4 heteroatoms include triazinyl and tetrazinyl, respectively. Exemplary 7-membered heteroaryl groups containing 1 heteroatom include azepinyl, oxepinyl, and thiepinyl. Exemplary 5,6-bicyclic heteroaryl groups include indolyl, isoindolyl, indazolyl, benzotriazolyl, benzothiophenyl, isobenzothiophenyl, benzofuranyl, benzoisofuranyl, benzimidazolyl, benzoxazolyl, benzisoxazolyl, benzoxadiazolyl, benzthiazolyl, benzisothiazolyl, benzthiadiazolyl, indolizinyl, and purinyl. Exemplary 6,6-bicyclic heteroaryl groups include naphthyridinyl, pteridinyl, quinolinyl, isoquinolinyl, cinnolinyl, quinoxalinyl, phthalazinyl, and quinazolinyl. Exemplary tricyclic heteroaryl groups include phenanthridinyl, dibenzofuranyl, carbazolyl, acridinyl, phenothiazinyl, phenoxazinyl, and phenazinyl.

“Heteroaralkyl” is a subset of “alkyl” and refers to an alkyl group substituted by a heteroaryl group, wherein the point of attachment is on the alkyl moiety.

Affixing the suffix “-ene” to a group indicates the group is a divalent moiety, e.g., alkylene is the divalent moiety of alkyl, alkenylene is the divalent moiety of alkenyl, alkynylene is the divalent moiety of alkynyl, heteroalkylene is the divalent moiety of heteroalkyl, heteroalkenylene is the divalent moiety of heteroalkenyl, heteroalkynylene is the divalent moiety of heteroalkynyl, carbocyclylene is the divalent moiety of carbocyclyl, heterocyclylene is the divalent moiety of heterocyclyl, arylene is the divalent moiety of aryl, and heteroarylene is the divalent moiety of heteroaryl.

A group is optionally substituted unless expressly provided otherwise. The term “optionally substituted” refers to being substituted or unsubstituted. In certain embodiments, alkyl, alkenyl, alkynyl, heteroalkyl, heteroalkenyl, heteroalkynyl, carbocyclyl, heterocyclyl, aryl, and heteroaryl groups are optionally substituted. “Optionally substituted” refers to a group which is substituted or unsubstituted (e.g., “substituted” or “unsubstituted” alkyl, “substituted” or “unsubstituted” alkenyl, “substituted” or “unsubstituted” alkynyl, “substituted” or “unsubstituted” heteroalkyl, “substituted” or “unsubstituted” heteroalkenyl, “substituted” or “unsubstituted” heteroalkynyl, “substituted” or “unsubstituted” carbocyclyl, “substituted” or “unsubstituted” heterocyclyl, “substituted” or “unsubstituted” aryl or “substituted” or “unsubstituted” heteroaryl group). In general, the term “substituted” means that at least one hydrogen present on a group is replaced with a permissible substituent, e.g., a substituent which upon substitution results in a stable compound, e.g., a compound which does not spontaneously undergo transformation such as by rearrangement, cyclization, elimination, or other reaction. Unless otherwise indicated, a “substituted” group has a substituent at one or more substitutable positions of the group, and when more than one position in any given structure is substituted, the substituent is either the same or different at each position. The term “substituted” is contemplated to include substitution with all permissible substituents of organic compounds, and includes any of the substituents described herein that results in the formation of a stable compound. The present invention contemplates any and all such combinations in order to arrive at a stable compound. For purposes of this invention, heteroatoms such as nitrogen may have hydrogen substituents and/or any suitable substituent as described herein which satisfy the valencies of the heteroatoms and results in the formation of a stable moiety. The invention is not limited in any manner by the exemplary substituents described herein.

Exemplary carbon atom substituents include halogen, —CN, —NO₂, —N₃, —SO₂H, —SO₃H, —OH, —OR^{aa}, —ON(R^{bb})₂, —N(R^{bb})₂, —N(R^{bb})₃⁺X⁻, —N(OR^{cc})R^{bb}, —SH, —SR^{aa}, —SSR^{cc}, —C(═O)R^{aa}, —CO₂H, —CHO, —C(OR^{cc})₂, —CO₂R^{aa}, —OC(═O)R^{aa}, —OCO₂R^{aa}, —C(═O)N(R^{bb})₂, —OC(═O)N(R^{bb})₂, —NR^{bb}C(═O)R^{aa}, —NR^{bb}CO₂R^{aa}, —NR^{bb}C(═O)N(R^{bb})₂, —C(═NR^{bb})R^{aa}, —C(═NR^{bb})OR^{aa}, —OC(═NR^{bb})R^{aa}, —OC(═NR^{bb})OR^{aa}, —C(═NR^{bb})N(R^{bb})₂, —OC(═NR^{bb})N(R^{bb})₂, —NR^{bb}C(═NR^{bb})N(R^{bb})₂, —C(═O)NR^{bb}SO₂R^{aa}, —NR^{bb}SO₂R^{aa}, —SO₂N(R^{bb})₂, —SO₂R^{aa}, —SO₂OR^{aa}, —OSO₂R^{aa}, —S(═O)R^{aa}, —OS(═O)R^{aa}, —Si(R^{aa})₃, —OSi(R^{aa})₃—C(═S)N(R^{bb})₂, —C(═O)SR^{aa}, —C(═S)SR^{aa}, —SC(═S)SR^{aa}, —SC(═O)SR^{aa}, —OC(═O)SR^{aa}, —SC(═O)OR^{aa}, —SC(═O)R^{aa}, —P(═O)(R^{aa})₂, —P(═O)(OR^{cc})₂, —OP(═O)(R^{aa})₂, —OP(═O)(OR^{cc})₂, —P(═O)(N(R^{bb})₂)₂, —OP(═O)(N(R^{bb})₂)₂, —NR^{bb}P(═O)(R^{aa})₂, —NR^{bb}P(═O)(OR^{cc})₂, —NR^{bb}P(═O)(N(R^{bb})₂)₂, —P(R^{cc})₂, —P(OR^{cc})₂, —P(R^{cc})₃X, —P(OR^{cc})₃⁺X⁻, —P(R^{cc})₄, —P(OR^{cc})₄, —OP(R^{cc})₂, —OP(R^{cc})₃⁺X⁻, —OP(OR^{cc})₂, —OP(OR^{cc})₃⁺X⁻, —OP(R^{cc})₄, —OP(OR^{cc})₄, —B(R^{aa})₂, —B(OR^{cc})₂, —BR^{aa}(OR^{cc}), C₁₋₂₀ alkyl, C₁₋₂₀ perhaloalkyl, C₁₋₂₀ alkenyl, C₁₋₂₀ alkynyl, heteroC₁₋₂₀ alkyl, heteroC₁₋₂₀ alkenyl, heteroC₁₋₂₀ alkynyl, C₃₋₁₀ carbocyclyl, 3-14 membered heterocyclyl, C₆₋₁₄ aryl, and 5-14 membered heteroaryl, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 Rad groups; wherein X⁻ is a counterion;
or two geminal hydrogens on a carbon atom are replaced with the group ═O, ═S, ═NN(R^{bb})₂, ═NNR^{bb}C(═O)R^{aa}, ═NNR^{bb}C(═O)OR^{aa}, ═NNR^{bb}S(═O)₂R^{aa}, ═NR^{bb}, or ═NOR^{cc};
wherein:
   each instance of R^{aa} is, independently, selected from C₁₋₂₀ alkyl, C₁₋₂₀ perhaloalkyl, C₁₋₂₀ alkenyl, C₁₋₂₀ alkynyl, heteroC₁₋₂₀ alkyl, heteroC₁₋₂₀alkenyl, heteroC₁₋₂₀alkynyl, C₃₋₁₀ carbocyclyl, 3-14 membered heterocyclyl, C₆₋₁₄ aryl, and 5-14 membered heteroaryl, or two R^{aa} groups are joined to form a 3-14 membered heterocyclyl or 5-14 membered heteroaryl ring, wherein each of the alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{dd} groups;
   each instance of R^{bb} is, independently, selected from hydrogen, —OH, —OR^{aa}, —N(R^{cc})₂, —CN, —C(═O)R^{aa}, —C(═O)N(R^{cc})₂, —CO₂R^{aa}, —SO₂R^{aa}, —C(═NR^{cc})OR^{aa}, —C(═NR^{cc})N(R^{cc})₂, —SO₂N(R^{cc})₂, —SO₂R^{cc}, —SO₂OR^{cc}, —SOR^{aa}, —C(═S)N(R^{cc})₂, —C(═O)SR^{cc}, —C(═S)SR^{cc}, —P(═O)(R^{aa})₂, —P(═O)(OR^{cc})₂, —P(═O)(N(R^{cc})₂)₂, C₁₋₂₀ alkyl, C₁₋₂₀ perhaloalkyl, C₁₋₂₀ alkenyl, C₁₋₂₀ alkynyl, heteroC₁₋₂₀alkyl, heteroC₁₋₂₀alkenyl, heteroC₁₋₂₀alkynyl, C₃₋₁₀ carbocyclyl, 3-14 membered heterocyclyl, C₆₋₁₄ aryl, and 5-14 membered heteroaryl, or two R^{bb} groups are joined to form a 3-14 membered heterocyclyl or 5-14 membered heteroaryl ring, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{dd} groups;
   each instance of R^{cc} is, independently, selected from hydrogen, C₁₋₂₀ alkyl, C₁₋₂₀ perhaloalkyl, C₁₋₂₀ alkenyl, C₁₋₂₀ alkynyl, heteroC₁₋₂₀ alkyl, heteroC₁₋₂₀ alkenyl, heteroC₁₋₂₀ alkynyl, C₃₋₁₀ carbocyclyl, 3-14 membered heterocyclyl, C₆₋₁₄ aryl, and 5-14 membered heteroaryl, or two R^{cc} groups are joined to form a 3-14 membered heterocyclyl or 5-14 membered heteroaryl ring, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{dd} groups;
   each instance of R^{dd} is, independently, selected from halogen, —CN, —NO₂, —N₃, —SO₂H, —SO₃H, —OH, —OR^{ee}, —ON(R^{ff})₂, —N(R^{ff})₂, —N(R^{ff})₃⁺X⁻, —N(OR^{ee})R^{ff}, —SH, —SR^{ee}, —SSR^{ee}, —C(═O)R^{ee}, —CO₂H, —CO₂R^{ee}, —OC(═O)R^{ee}, —OCO₂R^{ee}, —C(═O)N(R^{ff})₂, —OC(═O)N(R^{ff})₂, —NR^{ff}C(═O)R^{ee}, —NR^{ff}CO₂R^{ee}, —NR^{ff}C(═O)N(R^{ff})₂, —C(═NR^{ff})OR^{ee}, —OC(═NR^{ff})R^{ee}, —OC(═NR^{ff})OR^{ee}, —C(═NR^{ff})N(R^{ff})₂, —OC(═NR^{ff})N(R^{ff})₂, —NR^{ff}C(═NR^{ff})N(R^{ff})₂, —NR^{ff}SO₂R^{ee}, —SO₂N(R^{ff})₂, —SO₂R^{ee}, —SO₂OR^{ee}, —OSO₂R^{ee}, —S(═O)R^{ee}, —Si(R^{ee})₃, —OSi(R^{ee})₃, —C(═S)N(R^{ff})₂, —C(═O)SR^{ee}, —C(═S)SR^{ee}, —SC(═S)SR^{ee}, —P(═O)(OR^{ee})₂, —P(═O)(R^{ee})₂—OP(═O)(R^{ee})₂, —OP(═O)(OR^{ee})₂, C₁₋₁₀ alkyl, C₁₋₁₀ perhaloalkyl, C₁₋₁₀ alkenyl, C₁₋₁₀ alkynyl, heteroC₁₋₁₀alkyl, heteroC₁₋₁₀alkenyl, heteroC₁₋₁₀alkynyl, C₃₋₁₀ carbocyclyl, 3-10 membered heterocyclyl, C₆₋₁₀ aryl, and 5-10 membered heteroaryl, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{gg} groups, or two geminal R^{dd} substituents are joined to form ═O or ═S; wherein X⁻ is a counterion;
   each instance of R^{ee} is, independently, selected from C₁₋₁₀ alkyl, C₁₋₁₀ perhaloalkyl, C₁₋₁₀ alkenyl, C₁₋₁₀ alkynyl, heteroC₁₋₁₀ alkyl, heteroC₁₋₁₀ alkenyl, heteroC₁₋₁₀ alkynyl, C₃₋₁₀ carbocyclyl, C₆₋₁₀ aryl, 3-10 membered heterocyclyl, and 3-10 membered heteroaryl, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{gg} groups;
   each instance of R^{f} is, independently, selected from hydrogen, C₁₋₁₀ alkyl, C₁₋₁₀ perhaloalkyl, C₁₋₁₀ alkenyl, C₁₋₁₀ alkynyl, heteroC₁₋₁₀ alkyl, heteroC₁₋₁₀ alkenyl, heteroC₁₋₁₀ alkynyl, C₃₋₁₀ carbocyclyl, 3-10 membered heterocyclyl, C₆₋₁₀ aryl, and 5-10 membered heteroaryl, or two R^{f} groups are joined to form a 3-10 membered heterocyclyl or 5-10 membered heteroaryl ring, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{gg} groups;
   each instance of R^{gg} is, independently, halogen, —CN, —NO₂, —N₃, —SO₂H, —SO₃H, —OH, —OC₁₋₆ alkyl, —ON(C₁₋₆ alkyl)₂, —N(C₁₋₆ alkyl)₂, —N(C₁₋₆ alkyl)₃⁺X⁻, —NH(C₁₋₆ alkyl)₂⁺X⁻, —NH₂(C₁₋₆ alkyl)⁺X⁻, —NH₃⁺X⁻, —N(OC₁₋₆ alkyl)(C₁₋₆ alkyl), —N(OH)(C₁₋₆ alkyl), —NH(OH), —SH, —SC₁₋₆ alkyl, —SS(C₁₋₆ alkyl), —C(═O)(C₁₋₆ alkyl), —CO₂H, —CO₂(C₁₋₆ alkyl), —OC(═O)(C₁₋₆ alkyl), —OCO₂(C₁₋₆ alkyl), —C(═O)NH₂, —C(═O)N(C₁₋₆ alkyl)₂, —OC(═O)NH(C₁₋₆ alkyl), —NHC(═O)(C₁₋₆ alkyl), —N(C₁₋₆ alkyl)C(═O)(C₁₋₆ alkyl), —NHCO₂(C₁₋₆ alkyl), —NHC(═O)N(C₁₋₆ alkyl)₂, —NHC(═O)NH(C₁₋₆ alkyl), —NHC(═O)NH₂, —C(═NH)O(C₁₋₆ alkyl), —OC(═NH)(C₁₋₆ alkyl), —OC(═NH)OC₁₋₆ alkyl, —C(═NH)N(C₁₋₆ alkyl)₂, —C(═NH)NH(C₁₋₆ alkyl), —C(═NH)NH₂, —OC(═NH)N(C₁₋₆ alkyl)₂, —OC(NH)NH(C₁₋₆ alkyl), —OC(NH)NH₂, —NHC(NH)N(C₁₋₆ alkyl)₂, —NHC(═NH)NH₂, —NHSO₂(C₁₋₆ alkyl), —SO₂N(C₁₋₆ alkyl)₂, —SO₂NH(C₁₋₆ alkyl), —SO₂NH₂, —SO₂C₁₋₆ alkyl, —SO₂OC₁₋₆ alkyl, —OSO₂C₁₋₆ alkyl, —SOC₁₋₆ alkyl, —Si(C₁₋₆ alkyl)₃, —OSi(C₁₋₆ alkyl)₃ —C(═S)N(C₁₋₆ alkyl)₂, C(═S)NH(C₁₋₆ alkyl), C(═S)NH₂, —C(═O)S(C₁₋₆ alkyl), —C(═S)SC₁₋₆ alkyl, —SC(═S)SC₁₋₆ alkyl, —P(═O)(OC₁₋₆ alkyl)₂, —P(═O)(C₁₋₆ alkyl)₂, —OP(═O)(C₁₋₆ alkyl)₂, —OP(═O)(OC₁₋₆ alkyl)₂, C₁₋₁₀ alkyl, C₁₋₁₀ perhaloalkyl, C₁₋₁₀ alkenyl, C₁₋₁₀ alkynyl, heteroC₁₋₁₀ alkyl, heteroC₁₋₁₀ alkenyl, heteroC₁₋₁₀ alkynyl, C₃₋₁₀ carbocyclyl, C₆₋₁₀ aryl, 3-10 membered heterocyclyl, or 5-10 membered heteroaryl;
or two geminal R^{gg} substituents can be joined to form ═O or ═S; and
   each X⁻ is a counterion.

In certain embodiments, each carbon atom substituent is independently halogen, substituted (e.g., substituted with one or more halogen) or unsubstituted C₁₋₆ alkyl, —OR^{aa}, —SR^{aa}, —N(R^{bb})₂, —CN, —SCN, —NO₂, —C(═O)R^{aa}, —CO₂R^{aa}, —C(═O)N(R^{bb})₂, —OC(═O)R^{aa}, —OCO₂R^{aa}, —OC(═O)N(R^{bb})₂, —NR^{bb}C(═O)R^{aa}, —NR^{bb}CO₂R^{aa}, or —NR^{bb}C(═O)N(R^{bb})₂. In certain embodiments, each carbon atom substituent is independently halogen, substituted (e.g., substituted with one or more halogen) or unsubstituted C₁₋₁₀ alkyl, —OR^{aa}, —SR^{aa}, —N(R^{bb})₂, —CN, —SCN, —NO₂, —C(═O)R^{aa}, —CO₂R^{aa}, —C(═O)N(R^{bb})₂, —OC(═O)R^{aa}, —OCO₂R^{aa}, —OC(═O)N(R^{bb})₂, —NR^{bb}C(═O)R^{aa}, —NR^{bb}CO₂R^{aa}, or —NR^{bb}C(═O)N(R^{bb})₂, wherein R^{aa} is hydrogen, substituted (e.g., substituted with one or more halogen) or unsubstituted C₁₋₁₀ alkyl, an oxygen protecting group (e.g., silyl, TBDPS, TBDMS, TIPS, TES, TMS, MOM, THP, t-Bu, Bn, allyl, acetyl, pivaloyl, or benzoyl) when attached to an oxygen atom, or a sulfur protecting group (e.g., acetamidomethyl, t-Bu, 3-nitro-2-pyridine sulfenyl, 2-pyridine-sulfenyl, or triphenylmethyl) when attached to a sulfur atom; and each R^{bb} is independently hydrogen, substituted (e.g., substituted with one or more halogen) or unsubstituted C₁₋₁₀ alkyl, or a nitrogen protecting group (e.g., Bn, Boc, Cbz, Fmoc, trifluoroacetyl, triphenylmethyl, acetyl, or Ts). In certain embodiments, each carbon atom substituent is independently halogen, substituted (e.g., substituted with one or more halogen) or unsubstituted C₁₋₆ alkyl, —OR^{aa}, —SR^{a}, —N(R^{bb})₂, —CN, —SCN, or —NO₂. In certain embodiments, each carbon atom substituent is independently halogen, substituted (e.g., substituted with one or more halogen moieties) or unsubstituted C₁₋₁₀ alkyl, —OR^{aa}, —SR^{aa}, —N(R^{bb})₂, —CN, —SCN, or —NO₂, wherein R^{aa} is hydrogen, substituted (e.g., substituted with one or more halogen) or unsubstituted C₁₋₁₀ alkyl, an oxygen protecting group (e.g., silyl, TBDPS, TBDMS, TIPS, TES, TMS, MOM, THP, t-Bu, Bn, allyl, acetyl, pivaloyl, or benzoyl) when attached to an oxygen atom, or a sulfur protecting group (e.g., acetamidomethyl, t-Bu, 3-nitro-2-pyridine sulfenyl, 2-pyridine-sulfenyl, or triphenylmethyl) when attached to a sulfur atom; and each R^{bb} is independently hydrogen, substituted (e.g., substituted with one or more halogen) or unsubstituted C₁₋₁₀ alkyl, or a nitrogen protecting group (e.g., Bn, Boc, Cbz, Fmoc, trifluoroacetyl, triphenylmethyl, acetyl, or Ts).

In certain embodiments, the molecular weight of a carbon atom substituent is lower than 250, lower than 200, lower than 150, lower than 100, or lower than 50 g/mol. In certain embodiments, a carbon atom substituent consists of carbon, hydrogen, fluorine, chlorine, bromine, iodine, oxygen, sulfur, nitrogen, and/or silicon atoms. In certain embodiments, a carbon atom substituent consists of carbon, hydrogen, fluorine, chlorine, bromine, iodine, oxygen, sulfur, and/or nitrogen atoms. In certain embodiments, a carbon atom substituent consists of carbon, hydrogen, fluorine, chlorine, bromine, and/or iodine atoms. In certain embodiments, a carbon atom substituent consists of carbon, hydrogen, fluorine, and/or chlorine atoms.

The term “halo” or “halogen” refers to fluorine (fluoro, —F), chlorine (chloro, —Cl), bromine (bromo, —Br), or iodine (iodo, —I).

The term “hydroxyl” or “hydroxy” refers to the group —OH. The term “substituted hydroxyl” or “substituted hydroxyl,” by extension, refers to a hydroxyl group wherein the oxygen atom directly attached to the parent molecule is substituted with a group other than hydrogen, and includes groups selected from —OR^{aa}, —ON(R^{bb})₂, —OC(═O)SR^{aa}, —OC(═O)R^{aa}, —OCO₂R^{aa}, —OC(═O)N(R^{bb})₂, —OC(═NR^{bb})R^{aa}, —OC(═NR^{bb})OR^{aa}, —OC(═NR^{bb})N(R^{bb})₂, —OS(═O)R^{aa}, —OSO₂R^{aa}, —OSi(R^{aa})₃, —OP(R^{cc})₂, —OP(R^{cc})₃⁺X⁻, —OP(OR^{cc})₂, —OP(OR^{cc})₃⁺X⁻, —OP(═O)(R^{aa}), —OP(═O)(OR^{cc})₂, and —OP(═O)(N(R^{bb}))₂, wherein X⁻, R^{aa}, R^{bb}, and R^{cc} are as defined herein.

The term “thiol” or “thio” refers to the group —SH. The term “substituted thiol” or “substituted thio,” by extension, refers to a thiol group wherein the sulfur atom directly attached to the parent molecule is substituted with a group other than hydrogen, and includes groups selected from —SR^{aa}, —S═SR^{cc}, —SC(═S)SR^{aa}, —SC(═S)OR^{aa}, —SC(═S) N(R^{bb})₂, —SC(═O)SR^{aa}, —SC(═O)OR^{aa}, —SC(═O)N(R^{bb})₂, and —SC(═O)R^{aa}, wherein R^{aa} and R^{cc} are as defined herein.

The term “amino” refers to the group —NH₂. The term “substituted amino,” by extension, refers to a monosubstituted amino, a disubstituted amino, or a trisubstituted amino. In certain embodiments, the “substituted amino” is a monosubstituted amino or a disubstituted amino group.

The term “monosubstituted amino” refers to an amino group wherein the nitrogen atom directly attached to the parent molecule is substituted with one hydrogen and one group other than hydrogen, and includes groups selected from —NH(R^{bb}), —NHC(═O)R^{aa}, —NHCO₂R^{aa}, —NHC(═O)N(R^{bb})₂, —NHC(═NR^{bb})N(R^{bb})₂, —NHSO₂R^{aa}, —NHP(═O)(OR^{cc})₂, and —NHP(═O)(N(R^{bb})₂)₂, wherein R^{aa}, R^{bb} and R^{cc} are as defined herein, and wherein R^{bb} of the group —NH(R^{bb}) is not hydrogen.

The term “disubstituted amino” refers to an amino group wherein the nitrogen atom directly attached to the parent molecule is substituted with two groups other than hydrogen, and includes groups selected from —N(R^{bb})₂, —NR^{bb} C(═O)R^{aa}, NR^{b}CO₂R^{aa}, —NR^{bb}C(═O)N(R^{bb})₂, —NR^{bb}C(═NR^{bb})N(R^{bb})₂, —NR^{bb}SO₂R^{aa}, —NR^{bb}P(═O)(OR^{cc})₂, and —NR^{bb}P(═O)(N(R^{bb})₂)₂, wherein R^{aa}, R^{bb}, and R^{cc} are as defined herein, with the proviso that the nitrogen atom directly attached to the parent molecule is not substituted with hydrogen.

The term “sulfonyl” refers to a group selected from —SO₂N(R^{bb})₂, —SO₂R^{aa}, and —SO₂OR^{aa}, wherein R^{aa} and R^{bb} are as defined herein.

The term “sulfinyl” refers to the group —S(═O)R^{aa}, wherein R^{aa} is as defined herein.

The term “acyl” refers to a group having the general formula —C(═O)R^{aa}, —C(═O)OR^{aa}, —C(═O)—O—C(═O)R^{aa}, —C(═O)SR^{aa}, —C(═O)N(R^{bb})₂, —C(═S)R^{aa}, —C(═S)N(R^{bb})₂, and —C(═S)S(R^{aa}), —C(═NR^{bb})R^{aa}, —C(═NR^{bb})OR^{aa}, —C(═NR^{bb})SR^{aa}, and —C(═NR^{bb})N(R^{bb})₂, wherein R^{aa} and R^{bb} are as defined herein. Exemplary acyl groups include aldehydes (—CHO), carboxylic acids (—CO₂H), ketones, acyl halides, esters, amides, imines, carbonates, carbamates, and ureas.

The term “carbonyl” refers to a group wherein the carbon directly attached to the parent molecule is sp² hybridized, and is substituted with an oxygen, nitrogen or sulfur atom, e.g., a group selected from ketones (—C(═O)R^{aa}), carboxylic acids (—CO₂H), aldehydes (—CHO), esters (—CO₂R^{aa}, —C(═O)SR^{aa}, —C(═S)SR^{aa}), amides (—C(═O)N(R^{bb})₂, —C(═O)NR^{bb}SO₂R^{aa}, —C(═S)N(R^{bb})₂), and imines (—C(═NR^{bb})R^{aa}, —C(═NR^{bb})OR^{aa}), —C(═NR^{bb})N(R^{bb})₂), wherein R^{aa} and R^{bb} are as defined herein.

Nitrogen atoms can be substituted or unsubstituted as valency permits, and include primary, secondary, tertiary, and quaternary nitrogen atoms. Exemplary nitrogen atom substituents include hydrogen, —OH, —OR^{aa}, —N(R^{cc})₂, —CN, —C(═O)R^{aa}, —C(═O)N(R^{cc})₂, —CO₂R^{aa}, —SO₂R^{aa}, —C(═NR^{bb})R^{aa}, —C(═NR^{cc})OR^{aa}, —C(═NRC)N(R^{cc})₂, —SO₂N(R^{cc})₂, —SO₂R^{cc}, —SO₂OR^{cc}, —SOR^{aa}, —C(═S)N(R^{cc})₂, —C(═O)SR^{cc}, —C(═S)SR^{cc}, —P(═O)(OR^{cc})₂, —P(═O)(R^{aa})₂, —P(═O)(N(R^{cc})₂)₂, C₁₋₂₀ alkyl, C₁₋₂₀ perhaloalkyl, C₁₋₂₀ alkenyl, C₁₋₂₀ alkynyl, hetero C₁₋₂₀ alkyl, hetero C₁₋₂₀ alkenyl, hetero C₁₋₂₀ alkynyl, C₃₋₁₀ carbocyclyl, 3-14 membered heterocyclyl, C₆₋₁₄ aryl, and 5-14 membered heteroaryl, or two R^{cc} groups attached to an N atom are joined to form a 3-14 membered heterocyclyl or 5-14 membered heteroaryl ring, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{dd} groups, and wherein R^{aa}, R^{bb}, R^{cc} and R^{dd} are as defined above.

In certain embodiments, each nitrogen atom substituent is independently substituted (e.g., substituted with one or more halogen) or unsubstituted C₁₋₆ alkyl, —C(═O)R^{aa}, —CO₂R^{aa}, —C(═O)N(R^{bb})₂, or a nitrogen protecting group. In certain embodiments, each nitrogen atom substituent is independently substituted (e.g., substituted with one or more halogen) or unsubstituted C₁₋₁₀ alkyl, —C(═O)R^{aa}, —CO₂R^{aa}, —C(═O)N(R^{bb})₂, or a nitrogen protecting group, wherein R^{aa} is hydrogen, substituted (e.g., substituted with one or more halogen) or unsubstituted C₁₋₁₀ alkyl, or an oxygen protecting group when attached to an oxygen atom; and each R^{bb} is independently hydrogen, substituted (e.g., substituted with one or more halogen) or unsubstituted C₁₋₁₀ alkyl, or a nitrogen protecting group. In certain embodiments, each nitrogen atom substituent is independently substituted (e.g., substituted with one or more halogen) or unsubstituted C₁₋₆ alkyl or a nitrogen protecting group.

In certain embodiments, the substituent present on the nitrogen atom is a nitrogen protecting group (also referred to herein as an “amino protecting group”). Nitrogen protecting groups include —OH, —OR^{aa}, —N(R^{cc})₂, —C(═O)R^{aa}, —CO₂R^{aa}, —C(═O)N(R^{cc})₂, —SO₂R^{aa}, —C(═NR^{cc})R^{aa}, —C(═NR^{cc})OR^{aa}, —C(═NR^{cc})N(R^{cc})₂, —SO₂N(R^{cc})₂, —SO₂R^{cc}, —SO₂OR^{cc}, —SOR^{aa}, —C(═S)N(R^{cc})₂, —C(═O)SR^{cc}, —C(═S)SR^{cc}, C₁₋₁₀ alkyl (e.g., aralkyl, heteroaralkyl), C₁₋₂₀ alkenyl, C₁₋₂₀ alkynyl, hetero C₁₋₂₀ alkyl, hetero C₁₋₂₀ alkenyl, hetero C₁₋₂₀ alkynyl, C₃₋₁₀ carbocyclyl, 3-14 membered heterocyclyl, C₆₋₁₄ aryl, and 5-14 membered heteroaryl groups, wherein each alkyl, alkenyl, alkynyl, heteroalkyl, heteroalkenyl, heteroalkynyl, carbocyclyl, heterocyclyl, aralkyl, aryl, and heteroaryl is independently substituted with 0, 1, 2, 3, 4, or 5 R^{dd} groups, and wherein R^{aa}, R^{bb}, R^{cc} and R^{dd} are as defined herein. Nitrogen protecting groups are well known in the art and include those described in detail in Protecting Groups in Organic Synthesis, T. W. Greene and P. G. M. Wuts, 3^{rd} edition, John Wiley & Sons, 1999, incorporated herein by reference. In certain embodiments, the nitrogen protecting group does not form a urea with the nitrogen to which it is attached. In certain embodiments, the nitrogen protecting group is not —C(═O)N(R^{cc})₂.

For example, in certain embodiments, at least one nitrogen protecting group is an amide group (e.g., a moiety that include the nitrogen atom to which the nitrogen protecting groups (e.g., —C(═O)R^{aa}) is directly attached). In certain such embodiments, each nitrogen protecting group, together with the nitrogen atom to which the nitrogen protecting group is attached, is independently selected from the group consisting of formamide, acetamide, chloroacetamide, trichloroacetamide, trifluoroacetamide, phenylacetamide, 3-phenylpropanamide, picolinamide, 3-pyridylcarboxamide, N-benzoylphenylalanyl derivatives, benzamide, p-phenylbenzamide, o-nitophenylacetamide, o-nitrophenoxyacetamide, acetoacetamide, (N′-dithiobenzyloxyacylamino)acetamide, 3-(p-hydroxyphenyl)propanamide, 3-(o-nitrophenyl)propanamide, 2-methyl-2-(o-nitrophenoxy)propanamide, 2-methyl-2-(o-phenylazophenoxy)propanamide, 4-chlorobutanamide, 3-methyl-3-nitrobutanamide, o-nitrocinnamide, N-acetylmethionine derivatives, o-nitrobenzamide, and o-(benzoyloxymethyl)benzamide.

In certain embodiments, at least one nitrogen protecting group is a carbamate group (e.g., a moiety that include the nitrogen atom to which the nitrogen protecting groups (e.g., —C(═O)OR^{aa}) is directly attached). In certain such embodiments, each nitrogen protecting group, together with the nitrogen atom to which the nitrogen protecting group is attached, is independently selected from the group consisting of methyl carbamate, ethyl carbamate, 9-fluorenylmethyl carbamate (Fmoc), 9-(2-sulfo)fluorenylmethyl carbamate, 9-(2,7-dibromo)fluoroenylmethyl carbamate, 2,7-di-t-butyl-[9-(10,10-dioxo-10,10,10,10-tetrahydrothioxanthyl)]methyl carbamate (DBD-Tmoc), 4-methoxyphenacyl carbamate (Phenoc), 2,2,2-trichloroethyl carbamate (Troc), 2-trimethylsilylethyl carbamate (Teoc), 2-phenylethyl carbamate (hZ), 1-(1-adamantyl)-1-methylethyl carbamate (Adpoc), 1,1-dimethyl-2-haloethyl carbamate, 1,1-dimethyl-2,2-dibromoethyl carbamate (DB-t-BOC), 1,1-dimethyl-2,2,2-trichloroethyl carbamate (TCBOC), 1-methyl-1-(4-biphenylyl)ethyl carbamate (Bpoc), 1-(3,5-di-t-butylphenyl)-1-methylethyl carbamate (t-Bumeoc), 2-(2′- and 4′-pyridyl)ethyl carbamate (Pyoc), 2-(N,N-dicyclohexylcarboxamido)ethyl carbamate, t-butyl carbamate (BOC or Boc), 1-adamantyl carbamate (Adoc), vinyl carbamate (Voc), allyl carbamate (Alloc), 1-isopropylallyl carbamate (Ipaoc), cinnamyl carbamate (Coc), 4-nitrocinnamyl carbamate (Noc), 8-quinolyl carbamate, N-hydroxypiperidinyl carbamate, alkyldithio carbamate, benzyl carbamate (Cbz), p-methoxybenzyl carbamate (Moz), p-nitrobenzyl carbamate, p-bromobenzyl carbamate, p-chlorobenzyl carbamate, 2,4-dichlorobenzyl carbamate, 4-methylsulfinylbenzyl carbamate (Msz), 9-anthrylmethyl carbamate, diphenylmethyl carbamate, 2-methylthioethyl carbamate, 2-methylsulfonylethyl carbamate, 2-(p-toluenesulfonyl)ethyl carbamate, [2-(1,3-dithianyl)]methyl carbamate (Dmoc), 4-methylthiophenyl carbamate (Mtpc), 2,4-dimethylthiophenyl carbamate (Bmpc), 2-phosphonioethyl carbamate (Peoc), 2-triphenylphosphonioisopropyl carbamate (Ppoc), 1,1-dimethyl-2-cyanoethyl carbamate, m-chloro-p-acyloxybenzyl carbamate, p-(dihydroxyboryl)benzyl carbamate, 5-benzisoxazolylmethyl carbamate, 2-(trifluoromethyl)-6-chromonylmethyl carbamate (Teroc), m-nitrophenyl carbamate, 3,5-dimethoxybenzyl carbamate, o-nitrobenzyl carbamate, 3,4-dimethoxy-6-nitrobenzyl carbamate, phenyl(o-nitrophenyl)methyl carbamate, t-amyl carbamate, S-benzyl thiocarbamate, p-cyanobenzyl carbamate, cyclobutyl carbamate, cyclohexyl carbamate, cyclopentyl carbamate, cyclopropylmethyl carbamate, p-decyloxybenzyl carbamate, 2,2-dimethoxyacylvinyl carbamate, o-(N,N-dimethylcarboxamido)benzyl carbamate, 1,1-dimethyl-3-(N,N-dimethylcarboxamido)propyl carbamate, 1,1-dimethylpropynyl carbamate, di(2-pyridyl)methyl carbamate, 2-furanylmethyl carbamate, 2-iodoethyl carbamate, isoborynl carbamate, isobutyl carbamate, isonicotinyl carbamate, p-(p′-methoxyphenylazo)benzyl carbamate, 1-methylcyclobutyl carbamate, 1-methylcyclohexyl carbamate, 1-methyl-1-cyclopropylmethyl carbamate, 1-methyl-1-(3,5-dimethoxyphenyl)ethyl carbamate, 1-methyl-1-(p-phenylazophenyl)ethyl carbamate, 1-methyl-1-phenylethyl carbamate, 1-methyl-1-(4-pyridyl)ethyl carbamate, phenyl carbamate, p-(phenylazo)benzyl carbamate, 2,4,6-tri-t-butylphenyl carbamate, 4-(trimethylammonium)benzyl carbamate, and 2,4,6-trimethylbenzyl carbamate.

In certain embodiments, at least one nitrogen protecting group is a sulfonamide group (e.g., a moiety that include the nitrogen atom to which the nitrogen protecting groups (e.g., —S(═O)₂R^{aa}) is directly attached). In certain such embodiments, each nitrogen protecting group, together with the nitrogen atom to which the nitrogen protecting group is attached, is independently selected from the group consisting of p-toluenesulfonamide (Ts), benzenesulfonamide, 2,3,6-trimethyl-4-methoxybenzenesulfonamide (Mtr), 2,4,6-trimethoxybenzenesulfonamide (Mtb), 2,6-dimethyl-4-methoxybenzenesulfonamide (Pme), 2,3,5,6-tetramethyl-4-methoxybenzenesulfonamide (Mte), 4-methoxybenzenesulfonamide (Mbs), 2,4,6-trimethylbenzenesulfonamide (Mts), 2,6-dimethoxy-4-methylbenzenesulfonamide (iMds), 2,2,5,7,8-pentamethylchroman-6-sulfonamide (Pmc), methanesulfonamide (Ms), β-trimethylsilylethanesulfonamide (SES), 9-anthracenesulfonamide, 4-(4′,8′-dimethoxynaphthylmethyl)benzenesulfonamide (DNMBS), benzylsulfonamide, trifluoromethylsulfonamide, and phenacylsulfonamide.

In certain embodiments, each nitrogen protecting group, together with the nitrogen atom to which the nitrogen protecting group is attached, is independently selected from the group consisting of phenothiazinyl-(10)-acyl derivatives, N′-p-toluenesulfonylaminoacyl derivatives, N′-phenylaminothioacyl derivatives, N-benzoylphenylalanyl derivatives, N-acetylmethionine derivatives, 4,5-diphenyl-3-oxazolin-2-one, N-phthalimide, N-dithiasuccinimide (Dts), N-2,3-diphenylmaleimide, N-2,5-dimethylpyrrole, N-1,1,4,4-tetramethyldisilylazacyclopentane adduct (STABASE), 5-substituted 1,3-dimethyl-1,3,5-triazacyclohexan-2-one, 5-substituted 1,3-dibenzyl-1,3,5-triazacyclohexan-2-one, 1-substituted 3,5-dinitro-4-pyridone, N-methylamine, N-allylamine, N-[2-(trimethylsilyl)ethoxy]methylamine (SEM), N-3-acetoxypropylamine, N-(1-isopropyl-4-nitro-2-oxo-3-pyroolin-3-yl)amine, quaternary ammonium salts, N-benzylamine, N-di(4-methoxyphenyl)methylamine, N-5-dibenzosuberylamine, N-triphenylmethylamine (Tr), N-[(4-methoxyphenyl)diphenylmethyl]amine (MMTr), N-9-phenylfluorenylamine (PhF), N-2,7-dichloro-9-fluorenylmethyleneamine, N-ferrocenylmethylamino (Fcm), N-2-picolylamino N′-oxide, N-1,1-dimethylthiomethyleneamine, N-benzylideneamine, N-p-methoxybenzylideneamine, N-diphenylmethyleneamine, N-[(2-pyridyl)mesityl]methyleneamine, N—(N′,N′-dimethylaminomethylene)amine, N-p-nitrobenzylideneamine, N-salicylideneamine, N-5-chlorosalicylideneamine, N-(5-chloro-2-hydroxyphenyl)phenylmethyleneamine, N-cyclohexylideneamine, N-(5,5-dimethyl-3-oxo-1-cyclohexenyl)amine, N-borane derivatives, N-diphenylborinic acid derivatives, N-[phenyl(pentaacylchromium- or tungsten)acyl]amine, N-copper chelate, N-zinc chelate, N-nitroamine, N-nitrosoamine, amine N-oxide, diphenylphosphinamide (Dpp), dimethylthiophosphinamide (Mpt), diphenylthiophosphinamide (Ppt), dialkyl phosphoramidates, dibenzyl phosphoramidate, diphenyl phosphoramidate, benzenesulfenamide, o-nitrobenzenesulfenamide (Nps), 2,4-dinitrobenzenesulfenamide, pentachlorobenzenesulfenamide, 2-nitro-4-methoxybenzenesulfenamide, triphenylmethylsulfenamide, and 3-nitropyridinesulfenamide (Npys). In some embodiments, two instances of a nitrogen protecting group together with the nitrogen atoms to which the nitrogen protecting groups are attached are N,N′-isopropylidenediamine.

In certain embodiments, at least one nitrogen protecting group is Bn, Boc, Cbz, Fmoc, trifluoroacetyl, triphenylmethyl, acetyl, or Ts.

In certain embodiments, each oxygen atom substituent is independently substituted (e.g., substituted with one or more halogen) or unsubstituted C₁₋₁₀ alkyl, —C(═O)R^{aa}, —CO₂R^{aa}, —C(═O)N(R^{bb})₂, or an oxygen protecting group. In certain embodiments, each oxygen atom substituents is independently substituted (e.g., substituted with one or more halogen) or unsubstituted C₁₋₆ alkyl, —C(═O)R^{aa}, —CO₂R^{aa}, —C(═O)N(R^{bb})₂, or an oxygen protecting group, wherein R^{aa} is hydrogen, substituted (e.g., substituted with one or more halogen) or unsubstituted C₁₋₁₀ alkyl, or an oxygen protecting group when attached to an oxygen atom; and each R^{bb} is independently hydrogen, substituted (e.g., substituted with one or more halogen) or unsubstituted C₁₋₁₀ alkyl, or a nitrogen protecting group. In certain embodiments, each oxygen atom substituent is independently substituted (e.g., substituted with one or more halogen) or unsubstituted C₁₋₆ alkyl or an oxygen protecting group.

In certain embodiments, the substituent present on an oxygen atom is an oxygen protecting group (also referred to herein as an “hydroxyl protecting group”). Oxygen protecting groups include —R^{aa}, —N(R^{bb})₂, —C(═O)SR^{aa}, —C(═O)R^{aa}, —CO₂R^{aa}, —C(═O)N(R^{bb})₂, —C(═NR^{bb})R^{aa}, —C(═NR^{bb})OR^{aa}, —C(═NR^{bb})N(R^{bb})₂, —S(═O)R^{aa}, —SO₂R^{aa}, —Si(R^{aa})₃, —P(R^{cc})₂, —P(R^{cc})₃⁺X⁻, —P(OR^{cc})₂, —P(OR^{cc})₃⁺X⁻, —P(═O)(R^{aa})₂, —P(═O)(OR^{cc})₂, and —P(═O)(N(R^{bb})₂)₂, wherein X⁻, R^{aa}, R^{bb}, and R^{cc} are as defined herein. Oxygen protecting groups are well known in the art and include those described in detail in Protecting Groups in Organic Synthesis, T. W. Greene and P. G. M. Wuts, 3^{rd} edition, John Wiley & Sons, 1999, incorporated herein by reference.

In certain embodiments, each oxygen protecting group, together with the oxygen atom to which the oxygen protecting group is attached, is selected from the group consisting of methoxy, methoxylmethyl (MOM), methylthiomethyl (MTM), t-butylthiomethyl, (phenyldimethylsilyl)methoxymethyl (SMOM), benzyloxymethyl (BOM), p-methoxybenzyloxymethyl (PMBM), (4-methoxyphenoxy)methyl (p-AOM), guaiacolmethyl (GUM), t-butoxymethyl, 4-pentenyloxymethyl (POM), siloxymethyl, 2-methoxyethoxymethyl (MEM), 2,2,2-trichloroethoxymethyl, bis(2-chloroethoxy)methyl, 2-(trimethylsilyl)ethoxymethyl (SEMOR), tetrahydropyranyl (THP), 3-bromotetrahydropyranyl, tetrahydrothiopyranyl, 1-methoxycyclohexyl, 4-methoxytetrahydropyranyl (MTHP), 4-methoxytetrahydrothiopyranyl, 4-methoxytetrahydrothiopyranyl S,S-dioxide, 1-[(2-chloro-4-methyl)phenyl]-4-methoxypiperidin-4-yl (CTMP), 1,4-dioxan-2-yl, tetrahydrofuranyl, tetrahydrothiofuranyl, 2,3,3a,4,5,6,7,7a-octahydro-7,8,8-trimethyl-4,7-methanobenzofuran-2-yl, 1-ethoxyethyl, 1-(2-chloroethoxy)ethyl, 1-methyl-1-methoxyethyl, 1-methyl-1-benzyloxyethyl, 1-methyl-1-benzyloxy-2-fluoroethyl, 2,2,2-trichloroethyl, 2-trimethylsilylethyl, 2-(phenylselenyl)ethyl, t-butyl, allyl, p-chlorophenyl, p-methoxyphenyl, 2,4-dinitrophenyl, benzyl (Bn), p-methoxybenzyl (PMB), 3,4-dimethoxybenzyl, o-nitrobenzyl, p-nitrobenzyl, p-halobenzyl, 2,6-dichlorobenzyl, p-cyanobenzyl, p-phenylbenzyl, 2-picolyl, 4-picolyl, 3-methyl-2-picolyl N-oxido, diphenylmethyl, p,p′-dinitrobenzhydryl, 5-dibenzosuberyl, triphenylmethyl, a-naphthyldiphenylmethyl, p-methoxyphenyldiphenylmethyl, di(p-methoxyphenyl)phenylmethyl, tri(p-methoxyphenyl)methyl, 4-(4′-bromophenacyloxyphenyl)diphenylmethyl, 4,4′,4″-tris(4,5-dichlorophthalimidophenyl)methyl, 4,4′,4″-tris(levulinoyloxyphenyl)methyl, 4,4′,4″-tris(benzoyloxyphenyl)methyl, 4,4′-Dimethoxy-3′″-[N-(imidazolylmethyl)]trityl Ether (IDTr-OR), 4,4′-Dimethoxy-3′″-[N-(imidazolylethyl)carbamoyl]trityl Ether (IETr-OR), 1,1-bis(4-methoxyphenyl)-1′-pyrenylmethyl, 9-anthryl, 9-(9-phenyl)xanthenyl, 9-(9-phenyl-10-oxo)anthryl, 1,3-benzodithiolan-2-yl, benzisothiazolyl S,S-dioxido, trimethylsilyl (TMS), triethylsilyl (TES), triisopropylsilyl (TIPS), dimethylisopropylsilyl (IPDMS), diethylisopropylsilyl (DEIPS), dimethylthexylsilyl, t-butyldimethylsilyl (TBDMS), t-butyldiphenylsilyl (TBDPS), tribenzylsilyl, tri-p-xylylsilyl, triphenylsilyl, diphenylmethylsilyl (DPMS), t-butylmethoxyphenylsilyl (TBMPS), formate, benzoylformate, acetate, chloroacetate, dichloroacetate, trichloroacetate, trifluoroacetate, methoxyacetate, triphenylmethoxyacetate, phenoxyacetate, p-chlorophenoxyacetate, 3-phenylpropionate, 4-oxopentanoate (levulinate), 4,4-(ethylenedithio)pentanoate (levulinoyldithioacetal), pivaloate, adamantoate, crotonate, 4-methoxycrotonate, benzoate, p-phenylbenzoate, 2,4,6-trimethylbenzoate (mesitoate), methyl carbonate, 9-fluorenylmethyl carbonate (Fmoc), ethyl carbonate, 2,2,2-trichloroethyl carbonate (Troc), 2-(trimethylsilyl)ethyl carbonate (TMSEC), 2-(phenylsulfonyl) ethyl carbonate (Psec), 2-(triphenylphosphonio) ethyl carbonate (Peoc), isobutyl carbonate, vinyl carbonate, allyl carbonate, t-butyl carbonate (BOC or Boc), p-nitrophenyl carbonate, benzyl carbonate, p-methoxybenzyl carbonate, 3,4-dimethoxybenzyl carbonate, o-nitrobenzyl carbonate, p-nitrobenzyl carbonate, S-benzyl thiocarbonate, 4-ethoxy-1-napththyl carbonate, methyl dithiocarbonate, 2-iodobenzoate, 4-azidobutyrate, 4-nitro-4-methylpentanoate, o-(dibromomethyl)benzoate, 2-formylbenzenesulfonate, 2-(methylthiomethoxy)ethyl carbonate (MTMEC-OR), 4-(methylthiomethoxy)butyrate, 2-(methylthiomethoxymethyl)benzoate, 2,6-dichloro-4-methylphenoxyacetate, 2,6-dichloro-4-(1,1,3,3-tetramethylbutyl)phenoxyacetate, 2,4-bis(1,1-dimethylpropyl)phenoxyacetate, chlorodiphenylacetate, isobutyrate, monosuccinoate, (E)-2-methyl-2-butenoate, o-(methoxyacyl)benzoate, a-naphthoate, nitrate, alkyl N,N,N′,N′-tetramethylphosphorodiamidate, alkyl N-phenylcarbamate, borate, dimethylphosphinothioyl, alkyl 2,4-dinitrophenylsulfenate, sulfate, methanesulfonate (mesylate), benzylsulfonate, and tosylate (Ts).

In certain embodiments, at least one oxygen protecting group is silyl, TBDPS, TBDMS, TIPS, TES, TMS, MOM, THP, t-Bu, Bn, allyl, acetyl, pivaloyl, or benzoyl.

In certain embodiments, each sulfur atom substituent is independently substituted (e.g., substituted with one or more halogen) or unsubstituted C₁₋₁₀ alkyl, —C(═O)R^{aa}, —CO₂R^{aa}, —C(═O)N(R^{bb})₂, or a sulfur protecting group. In certain embodiments, each sulfur atom substituent is independently substituted (e.g., substituted with one or more halogen) or unsubstituted C₁₋₁₀ alkyl, —C(═O)R^{aa}, —CO₂R^{aa}, —C(═O)N(R^{bb})₂, or a sulfur protecting group, wherein R^{aa} is hydrogen, substituted (e.g., substituted with one or more halogen) or unsubstituted C₁₋₁₀ alkyl, or an oxygen protecting group when attached to an oxygen atom; and each R^{bb} is independently hydrogen, substituted (e.g., substituted with one or more halogen) or unsubstituted C₁₋₁₀ alkyl, or a nitrogen protecting group. In certain embodiments, each sulfur atom substituent is independently substituted (e.g., substituted with one or more halogen) or unsubstituted C₁₋₆ alkyl or a sulfur protecting group.

In certain embodiments, the substituent present on a sulfur atom is a sulfur protecting group (also referred to as a “thiol protecting group”). In some embodiments, each sulfur protecting group is selected from the group consisting of —R^{aa}, —N(R^{bb})₂, —C(═O)SR^{aa}, —C(═O)R^{aa}, —CO₂R^{aa}, —C(═O)N(R^{bb})₂, —C(═NR^{bb})R^{aa}, —C(═NR^{bb})OR^{aa}, —C(═NR^{bb})N(R^{bb})₂, —S(═O)R^{aa}, —SO₂R^{aa}, —Si(R^{aa})₃, —P(R^{cc})₂, —P(R^{cc})₃⁺X, —P(OR^{cc})₂, —P(OR^{cc})₃X, —P(═O)(R^{aa})₂, —P(═O)(OR^{cc})₂, and —P(═O)(N(R^{bb}) 2)₂, wherein R^{aa}, R^{bb}, and R^{cc} are as defined herein. Sulfur protecting groups are well known in the art and include those described in detail in Protecting Groups in Organic Synthesis, T. W. Greene and P. G. M. Wuts, 3^{rd} edition, John Wiley & Sons, 1999, incorporated herein by reference.

In certain embodiments, the molecular weight of a substituent is lower than 250, lower than 200, lower than 150, lower than 100, or lower than 50 g/mol. In certain embodiments, a substituent consists of carbon, hydrogen, fluorine, chlorine, bromine, iodine, oxygen, sulfur, nitrogen, and/or silicon atoms. In certain embodiments, a substituent consists of carbon, hydrogen, fluorine, chlorine, bromine, iodine, oxygen, sulfur, and/or nitrogen atoms. In certain embodiments, a substituent consists of carbon, hydrogen, fluorine, chlorine, bromine, and/or iodine atoms. In certain embodiments, a substituent consists of carbon, hydrogen, fluorine, and/or chlorine atoms. In certain embodiments, a substituent comprises 0, 1, 2, or 3 hydrogen bond donors. In certain embodiments, a substituent comprises 0, 1, 2, or 3 hydrogen bond acceptors.

Use of the phrase “at least one instance” refers to 1, 2, 3, 4, or more instances, but also encompasses a range, e.g., for example, from 1 to 4, from 1 to 3, from 1 to 2, from 2 to 4, from 2 to 3, or from 3 to 4 instances, inclusive.

A “non-hydrogen group” refers to any group that is defined for a particular variable that is not hydrogen.

The disclosure is not intended to be limited in any manner by the above exemplary listing of substituents. Additional terms may be defined in other sections of this disclosure.

### Other Definitions

The following definitions are more general terms used throughout the present application.

As used herein, the term “salt” refers to any and all salts, and encompasses pharmaceutically acceptable salts. Salts include ionic compounds that result from the neutralization reaction of an acid and a base. A salt is composed of one or more cations (positively charged ions) and one or more anions (negative ions) so that the salt is electrically neutral (without a net charge). Salts of the compounds of this invention include those derived from inorganic and organic acids and bases. Examples of acid addition salts are salts of an amino group formed with inorganic acids, such as hydrochloric acid, hydrobromic acid, phosphoric acid, sulfuric acid, and perchloric acid, or with organic acids, such as acetic acid, oxalic acid, maleic acid, tartaric acid, citric acid, succinic acid, or malonic acid or by using other methods known in the art such as ion exchange. Other salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate, hippurate, and the like. Salts derived from appropriate bases include alkali metal, alkaline earth metal, ammonium and N⁺(C₁₋₄ alkyl)₄ salts. Representative alkali or alkaline earth metal salts include sodium, lithium, potassium, calcium, magnesium, and the like. Further salts include ammonium, quaternary ammonium, and amine cations formed using counterions such as halide, hydroxide, carboxylate, sulfate, phosphate, nitrate, lower alkyl sulfonate, and aryl sulfonate.

The term “pharmaceutically acceptable salt” refers to those salts which are, within the scope of sound medical judgment, suitable for use in contact with the tissues of humans and lower animals without undue toxicity, irritation, allergic response, and the like, and are commensurate with a reasonable benefit/risk ratio. Pharmaceutically acceptable salts are well known in the art. For example, Berge et al. describe pharmaceutically acceptable salts in detail in J. Pharmaceutical Sciences, 1977, 66, 1-19, incorporated herein by reference. Pharmaceutically acceptable salts of the compounds of this invention include those derived from suitable inorganic and organic acids and bases. Examples of pharmaceutically acceptable, nontoxic acid addition salts are salts of an amino group formed with inorganic acids, such as hydrochloric acid, hydrobromic acid, phosphoric acid, sulfuric acid, and perchloric acid or with organic acids, such as acetic acid, oxalic acid, maleic acid, tartaric acid, citric acid, succinic acid, or malonic acid or by using other methods known in the art such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate salts, and the like. Salts derived from appropriate bases include alkali metal, alkaline earth metal, ammonium, and N⁺(C₁₋₄ alkyl)₄⁻ salts. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, lower alkyl sulfonate, and aryl sulfonate.

The term “solvate” refers to forms of the compound, or a salt thereof, that are associated with a solvent, usually by a solvolysis reaction. This physical association may include hydrogen bonding. Conventional solvents include water, methanol, ethanol, acetic acid, DMSO, THF, diethyl ether, and the like. The compounds described herein may be prepared, e.g., in crystalline form, and may be solvated. Suitable solvates include pharmaceutically acceptable solvates and further include both stoichiometric solvates and non-stoichiometric solvates. In certain instances, the solvate will be capable of isolation, for example, when one or more solvent molecules are incorporated in the crystal lattice of a crystalline solid. “Solvate” encompasses both solution-phase and isolatable solvates. Representative solvates include hydrates, ethanolates, and methanolates. The term “stoichiometric solvate” refers to a solvate, which comprises a compound (e.g., a compound disclosed herein) and a solvent, wherein the solvent molecules are an integral part of the crystal lattice, in which they interact strongly with the compound and each other. The removal of the solvent molecules will cause instability of the crystal network, which subsequently collapses into an amorphous phase or recrystallizes as a new crystalline form with reduced solvent content.

The term “non-stoichiometric solvate” refers to a solvate, which comprises a compound (e.g., a compound disclosed herein) and a solvent, wherein the solvent content may vary without major changes in the crystal structure. The amount of solvent in the crystal lattice only depends on the partial pressure of solvent in the surrounding atmosphere. In the fully solvated state, non-stoichiometric solvates may, but not necessarily have to, show an integer molar ratio of solvent to the compound. During drying of a non-stoichiometric solvate, a portion of the solvent may be removed without significantly disturbing the crystal network, and the resulting solvate can subsequently be resolvated to give the initial crystalline form. Unlike stoichiometric solvates, the desolvation and resolvation of non-stoichiometric solvates is not accompanied by a phase transition, and all solvation states represent the same crystal form.

The term “hydrate” refers to a compound that is associated with water. Typically, the number of the water molecules contained in a hydrate of a compound is in a definite ratio to the number of the compound molecules in the hydrate. Therefore, a hydrate of a compound may be represented, for example, by the general formula R·x H₂O, wherein R is the compound, and x is a number greater than 0. A given compound may form more than one type of hydrate, including, e.g., monohydrates (x is 1), lower hydrates (x is a number greater than 0 and smaller than 1, e.g., hemihydrates (R·0.5H₂O)), and polyhydrates (x is a number greater than 1, e.g., dihydrates (R·2H₂O) and hexahydrates (R·6H₂O)).

The term “tautomers” or “tautomeric” refers to two or more interconvertible compounds resulting from at least one formal migration of a hydrogen atom and at least one change in valency (e.g., a single bond to a double bond, a triple bond to a single bond, or vice versa). The exact ratio of the tautomers depends on several factors, including temperature, solvent, and pH. Tautomerizations (i.e., the reaction providing a tautomeric pair) may catalyzed by acid or base. Exemplary tautomerizations include keto-to-enol, amide-to-imide, lactam-to-lactim, enamine-to-imine, and enamine-to-(a different enamine) tautomerizations.

It is also to be understood that compounds that have the same molecular formula but differ in the nature or sequence of bonding of their atoms or the arrangement of their atoms in space are termed “isomers”. Isomers that differ in the arrangement of their atoms in space are termed “stereoisomers”.

Stereoisomers that are not mirror images of one another are termed “diastereomers” and those that are non-superimposable mirror images of each other are termed “enantiomers”. When a compound has an asymmetric center, for example, it is bonded to four different groups, a pair of enantiomers is possible. An enantiomer can be characterized by the absolute configuration of its asymmetric center and is described by the R- and S-sequencing rules of Cahn and Prelog, or by the manner in which the molecule rotates the plane of polarized light and designated as dextrorotatory or levorotatory (i.e., as (+) or (−)-isomers respectively). A chiral compound can exist as either individual enantiomer or as a mixture thereof. A mixture containing equal proportions of the enantiomers is called a “racemic mixture”.

The term “co-crystal” refers to a crystalline structure comprising at least two different components (e.g., a compound disclosed herein and an acid), wherein each of the components is independently an atom, ion, or molecule. In certain embodiments, none of the components is a solvent. In certain embodiments, at least one of the components is a solvent. A co-crystal of a compound disclosed herein and an acid is different from a salt formed from a compound disclosed herein and the acid. In the salt, a compound disclosed herein is complexed with the acid in a way that proton transfer (e.g., a complete proton transfer) from the acid to a compound disclosed herein easily occurs at room temperature. In the co-crystal, however, a compound disclosed herein is complexed with the acid in a way that proton transfer from the acid to a compound disclosed herein does not easily occur at room temperature. In certain embodiments, in the co-crystal, there is no proton transfer from the acid to a compound disclosed herein. In certain embodiments, in the co-crystal, there is partial proton transfer from the acid to a compound disclosed herein. Co-crystals may be useful to improve the properties (e.g., solubility, stability, and ease of formulation) of a compound disclosed herein.

The term “polymorph” refers to a crystalline form of a compound (or a salt, hydrate, or solvate thereof). All polymorphs have the same elemental composition. Different crystalline forms usually have different X-ray diffraction patterns, infrared spectra, melting points, density, hardness, crystal shape, optical and electrical properties, stability, and solubility. Recrystallization solvent, rate of crystallization, storage temperature, and other factors may cause one crystal form to dominate. Various polymorphs of a compound can be prepared by crystallization under different conditions.

The term “prodrugs” refers to compounds that have cleavable groups and become by solvolysis or under physiological conditions the compounds described herein, which are pharmaceutically active in vivo. Such examples include, but are not limited to, choline ester derivatives and the like, N-alkylmorpholine esters and the like. Other derivatives of the compounds described herein have activity in both their acid and acid derivative forms, but in the acid sensitive form often offer advantages of solubility, tissue compatibility, or delayed release in the mammalian organism (see, Bundgard, H., Design of Prodrugs, pp. 7-9, 21-24, Elsevier, Amsterdam 1985). Prodrugs include acid derivatives well known to practitioners of the art, such as, for example, esters prepared by reaction of the parent acid with a suitable alcohol, or amides prepared by reaction of the parent acid compound with a substituted or unsubstituted amine, or acid anhydrides, or mixed anhydrides. Simple aliphatic or aromatic esters, amides, and anhydrides derived from acidic groups pendant on the compounds described herein are particular prodrugs. In some cases it is desirable to prepare double ester type prodrugs such as (acyloxy)alkyl esters or ((alkoxycarbonyl)oxy)alkylesters. C₁-C₈ alkyl, C₂-C₈ alkenyl, C₂-C₈ alkynyl, aryl, C₇-C₁₂ substituted aryl, and C₇-C₁₂ arylalkyl esters of the compounds described herein may be preferred.

The terms “composition” and “formulation” are used interchangeably.

A “subject” to which administration is contemplated refers to a human (i.e., male or female of any age group, e.g., pediatric subject (e.g., infant, child, or adolescent) or adult subject (e.g., young adult, middle-aged adult, or senior adult)) or non-human animal. In certain embodiments, the non-human animal is a mammal (e.g., primate (e.g., cynomolgus monkey or rhesus monkey), commercially relevant mammal (e.g., cattle, pig, horse, sheep, goat, cat, or dog), or bird (e.g., commercially relevant bird, such as chicken, duck, goose, or turkey)). In certain embodiments, the non-human animal is a fish, reptile, or amphibian. The non-human animal may be a male or female at any stage of development. The non-human animal may be a transgenic animal or genetically engineered animal. The term “patient” refers to a human subject in need of treatment of a disease.

The term “biological sample” refers to any sample including tissue samples (such as tissue sections and needle biopsies of a tissue); cell samples (e.g., cytological smears (such as Pap or blood smears) or samples of cells obtained by microdissection); samples of whole organisms (such as samples of yeasts or bacteria); or cell fractions, fragments or organelles (such as obtained by lysing cells and separating the components thereof by centrifugation or otherwise). Other examples of biological samples include blood, serum, urine, semen, fecal matter, cerebrospinal fluid, interstitial fluid, mucous, tears, sweat, pus, biopsied tissue (e.g., obtained by a surgical biopsy or needle biopsy), nipple aspirates, milk, vaginal fluid, saliva, swabs (such as buccal swabs), or any material containing biomolecules that is derived from a first biological sample.

The term “administer,” “administering,” or “administration” refers to implanting, absorbing, ingesting, injecting, inhaling, or otherwise introducing a compound described herein, or a composition thereof, in or on a subject.

The terms “treatment,” “treat,” and “treating” refer to reversing, alleviating, delaying the onset of, or inhibiting the progress of a disease described herein. In some embodiments, treatment may be administered after one or more signs or symptoms of the disease have developed or have been observed. In other embodiments, treatment may be administered in the absence of signs or symptoms of the disease. For example, treatment may be administered to a susceptible subject prior to the onset of symptoms (e.g., in light of a history of symptoms and/or in light of exposure to a pathogen). Treatment may also be continued after symptoms have resolved, for example, to delay or prevent recurrence.

The terms “condition,” “disease,” and “disorder” are used interchangeably.

An “effective amount” of a compound described herein refers to an amount sufficient to elicit the desired biological response. An effective amount of a compound described herein may vary depending on such factors as the desired biological endpoint, severity of side effects, disease, or disorder, the identity, pharmacokinetics, and pharmacodynamics of the particular compound, the condition being treated, the mode, route, and desired or required frequency of administration, the species, age and health or general condition of the subject. In certain embodiments, an effective amount is a therapeutically effective amount. In certain embodiments, an effective amount is a prophylactic treatment. In certain embodiments, an effective amount is the amount of a compound described herein in a single dose. In certain embodiments, an effective amount is the combined amounts of a compound described herein in multiple doses. In certain embodiments, the desired dosage is delivered three times a day, two times a day, once a day, every other day, every third day, every week, every two weeks, every three weeks, or every four weeks. In certain embodiments, the desired dosage is delivered using multiple administrations (e.g., two, three, four, five, six, seven, eight, nine, ten, eleven, twelve, thirteen, fourteen, or more administrations).

In certain embodiments, an effective amount of a compound for administration one or more times a day to a 70 kg adult human comprises about 0.0001 mg to about 3000 mg, about 0.0001 mg to about 2000 mg, about 0.0001 mg to about 1000 mg, about 0.001 mg to about 1000 mg, about 0.01 mg to about 1000 mg, about 0.1 mg to about 1000 mg, about 1 mg to about 1000 mg, about 1 mg to about 100 mg, about 10 mg to about 1000 mg, or about 100 mg to about 1000 mg, of a compound per unit dosage form.

In certain embodiments, the compounds of the invention may be administered orally or parenterally at dosage levels sufficient to deliver from about 0.001 mg/kg to about 100 mg/kg, from about 0.01 mg/kg to about 50 mg/kg, preferably from about 0.1 mg/kg to about 40 mg/kg, preferably from about 0.5 mg/kg to about 30 mg/kg, from about 0.01 mg/kg to about 10 mg/kg, from about 0.1 mg/kg to about 10 mg/kg, and more preferably from about 1 mg/kg to about 25 mg/kg, of subject body weight per day, one or more times a day, to obtain the desired therapeutic effect.

It will be appreciated that dose ranges as described herein provide guidance for the administration of provided pharmaceutical compositions to an adult. The amount to be administered to, for example, a child or an adolescent can be determined by a medical practitioner or person skilled in the art and can be lower or the same as that administered to an adult.

A “therapeutically effective amount” of a compound described herein is an amount sufficient to provide a therapeutic benefit in the treatment of a condition or to delay or minimize one or more symptoms associated with the condition. A therapeutically effective amount of a compound means an amount of therapeutic agent, alone or in combination with other therapies, which provides a therapeutic benefit in the treatment of the condition. The term “therapeutically effective amount” can encompass an amount that improves overall therapy, reduces or avoids symptoms, signs, or causes of the condition, and/or enhances the therapeutic efficacy of another therapeutic agent. In certain embodiments, a therapeutically effective amount is an amount sufficient for inhibiting kinase activity in a subject. In certain embodiments, a therapeutically effective amount is an amount sufficient for inhibiting DDR2 activity in a subject. In certain embodiments, a therapeutically effective amount is an amount sufficient for inhibiting DDR1 activity in a subject. In certain embodiments, a therapeutically effective amount is an amount sufficient for inhibiting p38 MAPK (e.g., p38-alpha) activity in a subject. In certain embodiments, a therapeutically effective amount is an amount sufficient for treating inflammatory disease, joint disease, proliferative disease, fibrosis, or pain in a subject. In certain embodiments, a therapeutically effective amount is an amount sufficient for inhibiting kinase activity and treating inflammatory disease, joint disease, proliferative disease, fibrosis, or pain in a subject. In certain embodiments, a therapeutically effective amount is an amount sufficient for inhibiting DDR2 activity and treating inflammatory disease, joint disease, proliferative disease, fibrosis, or pain in a subject. In certain embodiments, a therapeutically effective amount is an amount sufficient for inhibiting DDR1 activity and treating inflammatory disease, joint disease, proliferative disease, fibrosis, or pain in a subject. In certain embodiments, a therapeutically effective amount is an amount sufficient for inhibiting p38 MAPK (e.g., p38-alpha) activity and treating inflammatory disease, joint disease, proliferative disease, fibrosis, or pain in a subject.

A “prophylactically effective amount” of a compound described herein is an amount sufficient to prevent a condition, or one or more symptoms associated with the condition or prevent its recurrence. A prophylactically effective amount of a compound means an amount of a therapeutic agent, alone or in combination with other agents, which provides a prophylactic benefit in the prevention of the condition. The term “prophylactically effective amount” can encompass an amount that improves overall prophylaxis or enhances the prophylactic efficacy of another prophylactic agent. In certain embodiments, a prophylactically effective amount is an amount sufficient for inhibiting kinase activity in a subject. In certain embodiments, a prophylactically effective amount is an amount sufficient for inhibiting DDR2 activity in a subject. In certain embodiments, a prophylactically effective amount is an amount sufficient for inhibiting DDR1 activity in a subject. In certain embodiments, a prophylactically effective amount is an amount sufficient for inhibiting p38 MAPK (e.g., p38-alpha) activity in a subject. In certain embodiments, a prophylactically effective amount is an amount sufficient for preventing an inflammatory disease, joint disease, proliferative disease, fibrosis, or pain in a subject. In certain embodiments, a prophylactically effective amount is an amount sufficient for inhibiting kinase activity and preventing an inflammatory disease, joint disease, proliferative disease, fibrosis, or pain in a subject. In certain embodiments, a prophylactically effective amount is an amount sufficient for inhibiting DDR2 activity and preventing an inflammatory disease, joint disease, proliferative disease, fibrosis, or pain in a subject. In certain embodiments, a prophylactically effective amount is an amount sufficient for inhibiting DDR1 activity and preventing an inflammatory disease, joint disease, proliferative disease, fibrosis, or pain in a subject. In certain embodiments, a prophylactically effective amount is an amount sufficient for inhibiting p38 MAPK (e.g., p38-alpha) activity and preventing an inflammatory disease, joint disease, proliferative disease, fibrosis, or pain in a subject.

The term “prevent,” “preventing,” or “prevention” refers to a prophylactic treatment of a subject who is not and was not with a disease but is at risk of developing the disease or who was with a disease, is not with the disease, but is at risk of regression of the disease. In certain embodiments, the subject is at a higher risk of developing the disease or at a higher risk of regression of the disease than an average healthy member of a population.

A “proliferative disease” refers to a disease that occurs due to abnormal growth or extension by the multiplication of cells (Walker, Cambridge Dictionary of Biology; Cambridge University Press: Cambridge, UK, 1990). A proliferative disease may be associated with: 1) the pathological proliferation of normally quiescent cells; 2) the pathological migration of cells from their normal location (e.g., metastasis of neoplastic cells); 3) the pathological expression of proteolytic enzymes such as the matrix metalloproteinases (e.g., collagenases, gelatinases, and elastases); or 4) the pathological angiogenesis as in proliferative retinopathy and tumor metastasis. Exemplary proliferative diseases include cancers (i.e., “malignant neoplasms”), benign neoplasms, angiogenesis, inflammatory diseases, and autoimmune diseases.

The term “angiogenesis” refers to the physiological process through which new blood vessels form from pre-existing vessels. Angiogenesis is distinct from vasculogenesis, which is the de novo formation of endothelial cells from mesoderm cell precursors. The first vessels in a developing embryo form through vasculogenesis, after which angiogenesis is responsible for most blood vessel growth during normal or abnormal development. Angiogenesis is a vital process in growth and development, as well as in wound healing and in the formation of granulation tissue. However, angiogenesis is also a fundamental step in the transition of tumors from a benign state to a malignant one, leading to the use of angiogenesis inhibitors in the treatment of cancer. Angiogenesis may be chemically stimulated by angiogenic proteins, such as growth factors (e.g., VEGF). “Pathological angiogenesis” refers to abnormal (e.g., excessive or insufficient) angiogenesis that amounts to and/or is associated with a disease.

The terms “neoplasm” and “tumor” are used herein interchangeably and refer to an abnormal mass of tissue wherein the growth of the mass surpasses and is not coordinated with the growth of a normal tissue. A neoplasm or tumor may be “benign” or “malignant,” depending on the following characteristics: degree of cellular differentiation (including morphology and functionality), rate of growth, local invasion, and metastasis. A “benign neoplasm” is generally well differentiated, has characteristically slower growth than a malignant neoplasm, and remains localized to the site of origin. In addition, a benign neoplasm does not have the capacity to infiltrate, invade, or metastasize to distant sites. Exemplary benign neoplasms include, but are not limited to, lipoma, chondroma, adenomas, acrochordon, senile angiomas, seborrheic keratoses, lentigos, and sebaceous hyperplasias. In some cases, certain “benign” tumors may later give rise to malignant neoplasms, which may result from additional genetic changes in a subpopulation of the tumor's neoplastic cells, and these tumors are referred to as “pre-malignant neoplasms.” An exemplary pre-malignant neoplasm is a teratoma. In contrast, a “malignant neoplasm” is generally poorly differentiated (anaplasia) and has characteristically rapid growth accompanied by progressive infiltration, invasion, and destruction of the surrounding tissue. Furthermore, a malignant neoplasm generally has the capacity to metastasize to distant sites. The term “metastasis,” “metastatic,” or “metastasize” refers to the spread or migration of cancerous cells from a primary or original tumor to another organ or tissue and is typically identifiable by the presence of a “secondary tumor” or “secondary cell mass” of the tissue type of the primary or original tumor and not of that of the organ or tissue in which the secondary (metastatic) tumor is located. For example, a prostate cancer that has migrated to bone is said to be metastasized prostate cancer and includes cancerous prostate cancer cells growing in bone tissue.

The term “cancer” refers to a class of diseases characterized by the development of abnormal cells that proliferate uncontrollably and have the ability to infiltrate and destroy normal body tissues. See e.g., Stedman's Medical Dictionary, 25th ed.; Hensyl ed.; Williams & Wilkins: Philadelphia, 1990. Exemplary cancers include, but are not limited to, acoustic neuroma; adenocarcinoma; adrenal gland cancer; anal cancer; angiosarcoma (e.g., lymphangiosarcoma, lymphangioendotheliosarcoma, hemangiosarcoma); appendix cancer; benign monoclonal gammopathy; biliary cancer (e.g., cholangiocarcinoma); bladder cancer; breast cancer (e.g., adenocarcinoma of the breast, papillary carcinoma of the breast, mammary cancer, medullary carcinoma of the breast); brain cancer (e.g., meningioma, glioblastomas, glioma (e.g., astrocytoma, oligodendroglioma), medulloblastoma); bronchus cancer; carcinoid tumor; cervical cancer (e.g., cervical adenocarcinoma); choriocarcinoma; chordoma; craniopharyngioma; colorectal cancer (e.g., colon cancer, rectal cancer, colorectal adenocarcinoma); connective tissue cancer; epithelial carcinoma; ependymoma; endotheliosarcoma (e.g., Kaposi's sarcoma, multiple idiopathic hemorrhagic sarcoma); endometrial cancer (e.g., uterine cancer, uterine sarcoma); esophageal cancer (e.g., adenocarcinoma of the esophagus, Barrett's adenocarcinoma); Ewing's sarcoma; ocular cancer (e.g., intraocular melanoma, retinoblastoma); familiar hypereosinophilia; gall bladder cancer; gastric cancer (e.g., stomach adenocarcinoma); gastrointestinal stromal tumor (GIST); germ cell cancer; head and neck cancer (e.g., head and neck squamous cell carcinoma, oral cancer (e.g., oral squamous cell carcinoma), throat cancer (e.g., laryngeal cancer, pharyngeal cancer, nasopharyngeal cancer, oropharyngeal cancer)); hematopoietic cancers (e.g., leukemia such as acute lymphocytic leukemia (ALL) (e.g., B-cell ALL, T-cell ALL), acute myelocytic leukemia (AML) (e.g., B-cell AML, T-cell AML), chronic myelocytic leukemia (CML) (e.g., B-cell CML, T-cell CML), and chronic lymphocytic leukemia (CLL) (e.g., B-cell CLL, T-cell CLL)); lymphoma such as Hodgkin lymphoma (HL) (e.g., B-cell HL, T-cell HL) and non-Hodgkin lymphoma (NHL) (e.g., B-cell NHL such as diffuse large cell lymphoma (DLCL) (e.g., diffuse large B-cell lymphoma), follicular lymphoma, chronic lymphocytic leukemia/small lymphocytic lymphoma (CLL/SLL), mantle cell lymphoma (MCL), marginal zone B-cell lymphomas (e.g., mucosa-associated lymphoid tissue (MALT) lymphomas, nodal marginal zone B-cell lymphoma, splenic marginal zone B-cell lymphoma), primary mediastinal B-cell lymphoma, Burkitt lymphoma, lymphoplasmacytic lymphoma (i.e., Waldenstrom's macroglobulinemia), hairy cell leukemia (HCL), immunoblastic large cell lymphoma, precursor B-lymphoblastic lymphoma and primary central nervous system (CNS) lymphoma; and T-cell NHL such as precursor T-lymphoblastic lymphoma/leukemia, peripheral T-cell lymphoma (PTCL) (e.g., cutaneous T-cell lymphoma (CTCL) (e.g., mycosis fungoides, Sezary syndrome), angioimmunoblastic T-cell lymphoma, extranodal natural killer T-cell lymphoma, enteropathy type T-cell lymphoma, subcutaneous panniculitis-like T-cell lymphoma, and anaplastic large cell lymphoma); a mixture of one or more leukemia/lymphoma as described above; and multiple myeloma (MM)), heavy chain disease (e.g., alpha chain disease, gamma chain disease, mu chain disease); hemangioblastoma; hypopharynx cancer; inflammatory myofibroblastic tumors; immunocytic amyloidosis; kidney cancer (e.g., nephroblastoma a.k.a. Wilms' tumor, renal cell carcinoma); liver cancer (e.g., hepatocellular cancer (HCC), malignant hepatoma); lung cancer (e.g., bronchogenic carcinoma, small cell lung cancer (SCLC), non-small cell lung cancer (NSCLC), adenocarcinoma of the lung); leiomyosarcoma (LMS); mastocytosis (e.g., systemic mastocytosis); muscle cancer; myelodysplastic syndrome (MDS); mesothelioma; myeloproliferative disorder (MPD) (e.g., polycythemia vera (PV), essential thrombocytosis (ET), agnogenic myeloid metaplasia (AMM) a.k.a. myelofibrosis (MF), chronic idiopathic myelofibrosis, chronic myelocytic leukemia (CML), chronic neutrophilic leukemia (CNL), hypereosinophilic syndrome (HES)); neuroblastoma; neurofibroma (e.g., neurofibromatosis (NF) type 1 or type 2, schwannomatosis); neuroendocrine cancer (e.g., gastroenteropancreatic neuroendoctrine tumor (GEP-NET), carcinoid tumor); osteosarcoma (e.g., bone cancer); ovarian cancer (e.g., cystadenocarcinoma, ovarian embryonal carcinoma, ovarian adenocarcinoma); papillary adenocarcinoma; pancreatic cancer (e.g., pancreatic andenocarcinoma, intraductal papillary mucinous neoplasm (IPMN), Islet cell tumors); penile cancer (e.g., Paget's disease of the penis and scrotum); pinealoma; primitive neuroectodermal tumor (PNT); plasma cell neoplasia; paraneoplastic syndromes; intraepithelial neoplasms; prostate cancer (e.g., prostate adenocarcinoma); rectal cancer; rhabdomyosarcoma; salivary gland cancer; skin cancer (e.g., squamous cell carcinoma (SCC), keratoacanthoma (KA), melanoma, basal cell carcinoma (BCC)); small bowel cancer (e.g., appendix cancer); soft tissue sarcoma (e.g., malignant fibrous histiocytoma (MFH), liposarcoma, malignant peripheral nerve sheath tumor (MPNST), chondrosarcoma, fibrosarcoma, myxosarcoma); sebaceous gland carcinoma; small intestine cancer; sweat gland carcinoma; synovioma; testicular cancer (e.g., seminoma, testicular embryonal carcinoma); thyroid cancer (e.g., papillary carcinoma of the thyroid, papillary thyroid carcinoma (PTC), medullary thyroid cancer); urethral cancer; vaginal cancer; and vulvar cancer (e.g., Paget's disease of the vulva).

The terms “inflammatory disease” and “inflammatory condition” are used interchangeably herein, and refer to a disease or condition caused by, resulting from, or resulting in inflammation. Inflammatory diseases and conditions include those diseases, disorders or conditions that are characterized by signs of pain (dolor, from the generation of noxious substances and the stimulation of nerves), heat (calor, from vasodilatation), redness (rubor, from vasodilatation and increased blood flow), swelling (tumor, from excessive inflow or restricted outflow of fluid), and/or loss of function (functio laesa, which can be partial or complete, temporary or permanent. Inflammation takes on many forms and includes, but is not limited to, acute, adhesive, atrophic, catarrhal, chronic, cirrhotic, diffuse, disseminated, exudative, fibrinous, fibrosing, focal, granulomatous, hyperplastic, hypertrophic, interstitial, metastatic, necrotic, obliterative, parenchymatous, plastic, productive, proliferous, pseudomembranous, purulent, sclerosing, seroplastic, serous, simple, specific, subacute, suppurative, toxic, traumatic, and/or ulcerative inflammation. The term “inflammatory disease” may also refer to a dysregulated inflammatory reaction that causes an exaggerated response by macrophages, granulocytes, and/or T-lymphocytes leading to abnormal tissue damage and/or cell death. An inflammatory disease can be either an acute or chronic inflammatory condition and can result from infections or non-infectious causes. Inflammatory diseases include, without limitation, atherosclerosis, arteriosclerosis, autoimmune disorders, multiple sclerosis, systemic lupus erythematosus, polymyalgia rheumatica (PMR), gouty arthritis, degenerative arthritis, tendonitis, bursitis, psoriasis, cystic fibrosis, arthrosteitis, rheumatoid arthritis, inflammatory arthritis, Sjogren's syndrome, giant cell arteritis, progressive systemic sclerosis (scleroderma), ankylosing spondylitis, polymyositis, dermatomyositis, pemphigus, pemphigoid, diabetes (e.g., Type I), myasthenia gravis, Hashimoto's thyroiditis, Graves' disease, Goodpasture's disease, mixed connective tissue disease, sclerosing cholangitis, inflammatory bowel disease, Crohn's disease, ulcerative colitis, pernicious anemia, inflammatory dermatoses, usual interstitial pneumonitis (UIP), asbestosis, silicosis, bronchiectasis, berylliosis, talcosis, pneumoconiosis, sarcoidosis, desquamative interstitial pneumonia, lymphoid interstitial pneumonia, giant cell interstitial pneumonia, cellular interstitial pneumonia, extrinsic allergic alveolitis, Wegener's granulomatosis and related forms of angiitis (temporal arteritis and polyarteritis nodosa), inflammatory dermatoses, hepatitis, delayed-type hypersensitivity reactions (e.g., poison ivy dermatitis), pneumonia, respiratory tract inflammation, Adult Respiratory Distress Syndrome (ARDS), encephalitis, immediate hypersensitivity reactions, asthma, hayfever, allergies, acute anaphylaxis, rheumatic fever, glomerulonephritis, pyelonephritis, cellulitis, cystitis, chronic cholecystitis, ischemia (ischemic injury), reperfusion injury, allograft rejection, host-versus-graft rejection, appendicitis, arteritis, blepharitis, bronchiolitis, bronchitis, cervicitis, cholangitis, chorioamnionitis, conjunctivitis, dacryoadenitis, dermatomyositis, endocarditis, endometritis, enteritis, enterocolitis, epicondylitis, epididymitis, fasciitis, fibrositis, gastritis, gastroenteritis, gingivitis, ileitis, iritis, laryngitis, myelitis, myocarditis, nephritis, omphalitis, oophoritis, orchitis, osteitis, otitis, pancreatitis, parotitis, pericarditis, pharyngitis, pleuritis, phlebitis, pneumonitis, proctitis, prostatitis, rhinitis, salpingitis, sinusitis, stomatitis, synovitis, testitis, tonsillitis, urethritis, urocystitis, uveitis, vaginitis, vasculitis, vulvitis, vulvovaginitis, angitis, chronic bronchitis, osteomyelitis, optic neuritis, temporal arteritis, transverse myelitis, necrotizing fasciitis, and necrotizing enterocolitis. An ocular inflammatory disease includes, but is not limited to, post-surgical inflammation.

Additional exemplary inflammatory conditions include, but are not limited to, inflammation associated with acne, anemia (e.g., aplastic anemia, haemolytic autoimmune anaemia), asthma, arteritis (e.g., polyarteritis, temporal arteritis, periarteritis nodosa, Takayasu's arteritis), arthritis (e.g., crystalline arthritis, osteoarthritis, psoriatic arthritis, gouty arthritis, reactive arthritis, rheumatoid arthritis and Reiter's arthritis), ankylosing spondylitis, amylosis, amyotrophic lateral sclerosis, autoimmune diseases, allergies or allergic reactions, atherosclerosis, bronchitis, bursitis, chronic prostatitis, conjunctivitis, Chagas disease, chronic obstructive pulmonary disease, cermatomyositis, diverticulitis, diabetes (e.g., type I diabetes mellitus, Type II diabetes mellitus), a skin condition (e.g., psoriasis, eczema, burns, dermatitis, pruritus (itch)), endometriosis, Guillain-Barre syndrome, infection, ischaemic heart disease, Kawasaki disease, glomerulonephritis, gingivitis, hypersensitivity, headaches (e.g., migraine headaches, tension headaches), ileus (e.g., postoperative ileus and ileus during sepsis), idiopathic thrombocytopenic purpura, interstitial cystitis (painful bladder syndrome), gastrointestinal disorder (e.g., selected from peptic ulcers, regional enteritis, diverticulitis, gastrointestinal bleeding, eosinophilic gastrointestinal disorders (e.g., eosinophilic esophagitis, eosinophilic gastritis, eosinophilic gastroenteritis, eosinophilic colitis), gastritis, diarrhea, gastroesophageal reflux disease (GORD, or its synonym GERD), inflammatory bowel disease (IBD) (e.g., Crohn's disease, ulcerative colitis, collagenous colitis, lymphocytic colitis, ischaemic colitis, diversion colitis, Behcet's syndrome, indeterminate colitis) and inflammatory bowel syndrome (IBS)), lupus, multiple sclerosis, morphea, myeasthenia gravis, myocardial ischemia, nephrotic syndrome, pemphigus vulgaris, pernicious aneaemia, peptic ulcers, polymyositis, primary biliary cirrhosis, neuroinflammation associated with brain disorders (e.g., Parkinson's disease, Huntington's disease, and Alzheimer's disease), prostatitis, chronic inflammation associated with cranial radiation injury, pelvic inflammatory disease, reperfusion injury, regional enteritis, rheumatic fever, systemic lupus erythematosus, schleroderma, scierodoma, sarcoidosis, spondyloarthopathies, Sjogren's syndrome, thyroiditis, transplantation rejection, tendonitis, trauma or injury (e.g., frostbite, chemical irritants, toxins, scarring, burns, physical injury), vasculitis, vitiligo and Wegener's granulomatosis. In certain embodiments, the inflammatory disorder is selected from arthritis (e.g., rheumatoid arthritis), inflammatory bowel disease, inflammatory bowel syndrome, asthma, psoriasis, endometriosis, interstitial cystitis and prostatistis. In certain embodiments, the inflammatory condition is an acute inflammatory condition (e.g., for example, inflammation resulting from infection). In certain embodiments, the inflammatory condition is a chronic inflammatory condition (e.g., conditions resulting from asthma, arthritis and inflammatory bowel disease). The compounds may also be useful in treating inflammation associated with trauma and non-inflammatory myalgia. The compounds disclosed herein may also be useful in treating inflammation associated with cancer.

Immune disorders, such as auto-immune disorders, include, but are not limited to, arthritis (including rheumatoid arthritis, spondyloarthopathies, gouty arthritis, degenerative joint diseases such as osteoarthritis, systemic lupus erythematosus, Sjogren's syndrome, ankylosing spondylitis, undifferentiated spondylitis, Behcet's disease, haemolytic autoimmune anaemias, multiple sclerosis, amyotrophic lateral sclerosis, amylosis, acute painful shoulder, psoriatic, and juvenile arthritis), asthma, atherosclerosis, osteoporosis, bronchitis, tendonitis, bursitis, skin condition (e.g., psoriasis, eczema, burns, dermatitis, pruritus (itch)), enuresis, eosinophilic disease, gastrointestinal disorder (e.g., selected from peptic ulcers, regional enteritis, diverticulitis, gastrointestinal bleeding, eosinophilic gastrointestinal disorders (e.g., eosinophilic esophagitis, eosinophilic gastritis, eosinophilic gastroenteritis, eosinophilic colitis), gastritis, diarrhea, gastroesophageal reflux disease (GORD, or its synonym GERD), inflammatory bowel disease (IBD) (e.g., Crohn's disease, ulcerative colitis, collagenous colitis, lymphocytic colitis, ischaemic colitis, diversion colitis, Behcet's syndrome, indeterminate colitis) and inflammatory bowel syndrome (IBS)), and disorders ameliorated by a gastroprokinetic agent (e.g., ileus, postoperative ileus and ileus during sepsis; gastroesophageal reflux disease (GORD, or its synonym GERD); eosinophilic esophagitis, gastroparesis such as diabetic gastroparesis; food intolerances and food allergies and other functional bowel disorders, such as non-ulcerative dyspepsia (NUD) and non-cardiac chest pain (NCCP, including costo-chondritis)).

In certain embodiments, the inflammatory disorder and/or the immune disorder is a gastrointestinal disorder. In some embodiments, the gastrointestinal disorder is selected from gastrointestinal disorder (e.g., selected from peptic ulcers, regional enteritis, diverticulitis, gastrointestinal bleeding, eosinophilic gastrointestinal disorders (e.g., eosinophilic esophagitis, eosinophilic gastritis, eosinophilic gastroenteritis, eosinophilic colitis), gastritis, diarrhea, gastroesophageal reflux disease (GORD, or its synonym GERD), inflammatory bowel disease (IBD) (e.g., Crohn's disease, ulcerative colitis, collagenous colitis, lymphocytic colitis, ischaemic colitis, diversion colitis, Behcet's syndrome, indeterminate colitis) and inflammatory bowel syndrome (IBS)). In certain embodiments, the gastrointestinal disorder is inflammatory bowel disease (IBD).

In certain embodiments, the inflammatory condition and/or immune disorder is a skin condition. In some embodiments, the skin condition is pruritus (itch), psoriasis, eczema, burns or dermatitis. In certain embodiments, the skin condition is psoriasis. In certain embodiments, the skin condition is pruritis.

The term “fibrosis” as used herein refers to the formation or development of excess fibrous connective tissue in an organ or tissue. Fibrosis may result from injury or inflammation or interference with blood supply. Fibrosis may be due to pathological conditions or diseases, physical trauma (e.g., traumatic fibrosis), radiation damage, or exposure to chemotherapeutic compounds. Fibrosis may also be a consequence of the normal healing response leading to a scar, an abnormal, reactive process, or without known or understood causation.

The term “pain” includes, but is not limited to, neuropathic pain (e.g., peripheral neuropathic pain), central pain, deafferentiation pain, chronic pain (e.g., chronic nociceptive pain, and other forms of chronic pain such as post-operative pain, e.g., pain arising after hip, knee, or other replacement surgery), pre-operative pain, stimulus of nociceptive receptors (nociceptive pain), acute pain (e.g., phantom and transient acute pain), noninflammatory pain, inflammatory pain, pain associated with cancer, wound pain, burn pain, postoperative pain, pain associated with medical procedures, pain resulting from pruritus, painful bladder syndrome, pain associated with premenstrual dysphoric disorder and/or premenstrual syndrome, pain associated with chronic fatigue syndrome, pain associated with pre-term labor, pain associated with withdrawal symptoms from drug addiction, joint pain, arthritic pain (e.g., pain associated with crystalline arthritis, osteoarthritis, psoriatic arthritis, gouty arthritis, reactive arthritis, rheumatoid arthritis or Reiter's arthritis), lumbosacral pain, musculo-skeletal pain, headache, migraine, muscle ache, lower back pain, neck pain, toothache, dental/maxillofacial pain, visceral pain and the like. As described herein, pain can comprise mixtures of various types of pain provided above and herein (e.g., nociceptive pain, inflammatory pain, neuropathic pain, etc.). In some embodiments, a particular pain can dominate. In other embodiments, the pain comprises two or more types of pains without one dominating. A skilled clinician can determine the dosage to achieve a therapeutically effective amount for a particular subject based on the pain.

In certain embodiments, the pain is inflammatory pain. In certain embodiments, the pain (e.g., inflammatory pain) is associated with an inflammatory condition and/or an immune disorder.

In certain embodiments, the pain is non-inflammatory pain. The types of non-inflammatory pain include, without limitation, peripheral neuropathic pain (e.g., pain caused by a lesion or dysfunction in the peripheral nervous system), central pain (e.g., pain caused by a lesion or dysfunction of the central nervous system), deafferentation pain (e.g., pain due to loss of sensory input to the central nervous system), chronic nociceptive pain (e.g., certain types of cancer pain), noxious stimulus of nociceptive receptors (e.g., pain felt in response to tissue damage or impending tissue damage), phantom pain (e.g., pain felt in a part of the body that no longer exists, such as a limb that has been amputated), pain felt by psychiatric subjects (e.g., pain where no physical cause may exist), and wandering pain (e.g., wherein the pain repeatedly changes location in the body).

In addition, there is pain associated with normally non-painful sensations such as “pins and needles” (paraesthesias and dysesthesias), increased sensitivity to touch (hyperesthesia), painful sensation following innocuous stimulation (dynamic, static or thermal allodynia), increased sensitivity to noxious stimuli (thermal, cold, mechanical hyperalgesia), continuing pain sensation after removal of the stimulation (hyperpathia) or an absence of or deficit in selective sensory pathways (hypoalgesia).

The term “joint disease” as used herein refers to an abnormal condition of the joint, especially due to injury, trauma, degeneration, inflammation, infection, or autoimmune causes. Joint diseases include, but are not limited to, rheumatoid arthritis (RA), ankylosing spondylitis (AS), juvenile idiopathic arthritis (JIA), gout, infectious arthritis, dry arthritis, and osteoarthritis (OA), calcium pyrophosphate dihydrate deposition disease, hydroxyapatite crystal deposition disease, or cancers (e.g., chondrosarcoma, osteosarcoma, fibrosarcoma, and multiple myeloma), tendonitis, bursitis, fractures and cartilage or bone damage. In some embodiments, joint disease refers to deterioration or destruction of articular cartilage tissue surrounding the joint.

The term “joint” as used herein refers to an anatomical structure where two or more bones meet, including the ligaments that connect the bones to one another, the tendons that attach muscles to the bones, the joint capsule, bursae, and synovium. Non-limiting types of joints include fibrous joints, cartilaginous, and synovial joints. Joints that can be treated with the methods provided herein include fixed, hinge, pivot, or ball-and-socket joints. Examples of joints include, but are not limited to, the knee, wrist, ankle, hip, shoulder, elbow, neck, spine, vertebral disc joints, finger joints, and toe joints.

The term “intraarticular” as used herein means located inside, occurring internally, or applied by injection into a joint. In some embodiments, the term “intraarticular” refers to the space confined by the articular capsule, which may or may not contain synovial fluid.

The term “intraarticular injection” refers to an injection into a joint.

As used herein the term “inhibit” or “inhibition” in the context of enzymes (e.g., receptor tyrosine kinases or mitogen-activated protein kinases), for example, in the context of DDR1, DDR2, and/or p38 MAPK (e.g., p38-alpha) refers to a reduction in the activity of the enzyme (e.g., the receptor tyrosine kinase or mitogen-activated protein kinase). In some embodiments, the term refers to a reduction of the level of enzyme activity, e.g., DDR1, DDR2, and/or p38 MAPK (e.g., p38-alpha) activity, to a level that is statistically significantly lower than an initial level, which may, for example, be a baseline level of enzyme activity. In some embodiments, the term refers to a reduction of the level of enzyme activity, e.g., DDR1, DDR2, and/or p38 MAPK (e.g., p38-alpha) activity, to a level that is less than 75%, less than 50%, less than 40%, less than 30%, less than 25%, less than 20%, less than 10%, less than 9%, less than 8%, less than 7%, less than 6%, less than 5%, less than 4%, less than 3%, less than 2%, less than 1%, less than 0.5%, less than 0.1%, less than 0.01%, less than 0.001%, or less than 0.0001% of an initial level, which may, for example, be a baseline level of enzyme activity.

Other than in the examples, or where otherwise indicated, all numbers expressing quantities of ingredients or reaction conditions used herein should be understood as modified in all instances by the term “about.” “About” and “approximately” shall generally mean an acceptable degree of error for the quantity measured given the nature or precision of the measurements. Exemplary degrees of error are within 20 percent (%), typically, within 10%, or more typically, within 5%, 4%, 3%, 2%, or 1% of a given value or range of values.

Unless otherwise required by context, singular terms shall include pluralities, and plural terms shall include the singular.

### DETAILED DESCRIPTION OF CERTAIN EMBODIMENTS

Provided herein are compounds (e.g., compounds of Formulae (I) and (II)), and pharmaceutically acceptable salts, solvates, hydrates, polymorphs, co-crystals, tautomers, stereoisomers, isotopically labeled derivatives, and prodrugs thereof, and pharmaceutical compositions and kits thereof. The compounds provided herein are DDR1, DDR2, and/or p38 MAPK (e.g., p38-alpha) inhibitors and can therefore be used to treat and/or prevent associated diseases and conditions in a subject. Also provided herein are methods of treating and/or preventing a disease or condition (e.g., an inflammatory disease, joint disease, proliferative disease, fibrosis, or pain) in a subject comprising administering an effective amount of a compound or composition provided herein to the subject. The compound or composition may be administered as a monotherapy or in combination with another therapy, as described herein. Other uses of the compounds and pharmaceutical compositions provided herein include methods of inhibiting DDR1, DDR2, and/or p38 MAPK (e.g., p38-alpha) activity, preventing cartilage degradation, or inhibiting cell migration (e.g., in a subject or biological sample).

### Compounds

Provided herein are compounds of Formula (I):

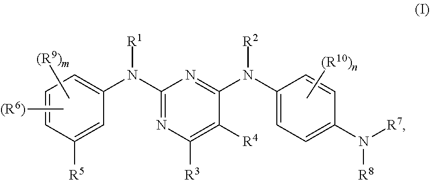

and pharmaceutically acceptable salts, solvates, hydrates, polymorphs, co-crystals, tautomers, stereoisomers, isotopically labeled derivatives, and prodrugs thereof, wherein:
R¹ and R² are each independently —H, optionally substituted aliphatic, optionally substituted acyl, or a nitrogen protecting group;
R³ and R⁴ are each independently —H, halogen, —OR^{a3}, —N(R^{a3})₂, —CN, —SCN, —NO₂, —N₃, —SR^{a3}, optionally substituted aliphatic, optionally substituted heteroaliphatic, or optionally substituted acyl;
R⁵ is —S(═O)₂N(R^{a5})₂, —S(═O)₂OR^{a5}, —S(═O)₂R^{a5}, —S(═O)₂Cl, or —SR^{a5};
R⁶ is —OR^{a6}, —N(R^{a6})₂, —CN, —SCN, —NO₂, —N₃, —SR^{a6}, optionally substituted heteroaliphatic, optionally substituted acyl, optionally substituted sulfonyl, or optionally substituted sulfinyl;
R⁷ and R⁸ are each independently —H, unsubstituted aliphatic, unsubstituted heteroaliphatic, —C(═O)R^{a7}, —C(═O)OR^{a7}, or a nitrogen protecting group; optionally wherein R⁷ and R⁸ are joined together with the intervening atoms to form optionally substituted heterocyclyl or optionally substituted heteroaryl;
each instance of R⁹ and R¹⁰ is independently —H, —OR^{a9}, —C(═O)R^{a9}, —OC(═O)R^{a9}, —C(═O)OR^{a9}, —N(R^{a9})₂, —N(C═O)R^{a9}, —(C═O)N(R^{a9})₂, —CN, —SCN, —NO₂, —N₃, —SR^{a9}, halogen, optionally substituted aliphatic, optionally substituted heteroaliphatic, optionally substituted sulfonyl, or optionally substituted sulfinyl;
each instance of R^{a3}, R^{a5}, R^{a6}, and R^{a9} is independently —H, optionally substituted aliphatic, optionally substituted heteroaliphatic, optionally substituted aryl, optionally substituted heteroaryl, optionally substituted carbocyclyl, optionally substituted heterocyclyl, optionally substituted acyl, optionally substituted sulfonyl, or optionally substituted sulfinyl, an oxygen protecting group when attached to an oxygen atom, a nitrogen protecting group when attached to a nitrogen atom, or a sulfur protecting group when attached to a sulfur atom; optionally wherein any two instances of R^{a3}, R^{a5}, R^{a6}, or R^{a9} attached to the same nitrogen atom are joined together with the intervening atoms to form optionally substituted heterocyclyl or optionally substituted heteroaryl;
each instance of R^{a7} is independently —H, or unsubstituted aliphatic;
m is 0, 1, 2, or 3; and
n is 0, 1, 2, 3, or 4.

In certain embodiments, a compound of Formula (I) is not one of the following formulae:

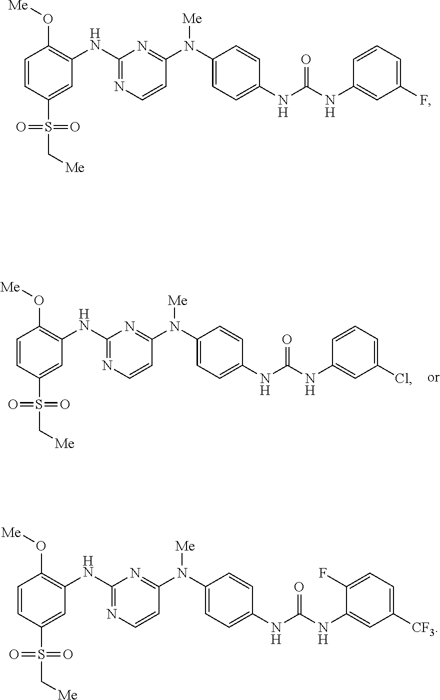 [SMILES: CCS(=O)(=O)c1ccc(OC)c(Nc2nccc(N(C)c3ccc(NC(=O)Nc4cccc(F)c4)cc3)n2)c1 ; CCS(=O)(=O)c1ccc(OC)c(Nc2nccc(N(C)c3ccc(NC(=O)Nc4cccc(Cl)c4)cc3)n2)c1 ; "or" ; CCS(=O)(=O)c1ccc(OC)c(Nc2nccc(N(C)c3ccc(NC(=O)Nc4cc(C(F)(F)F)ccc4F)cc3)n2)c1]

In certain embodiments, a compound of Formula (I) is of Formula (I′):

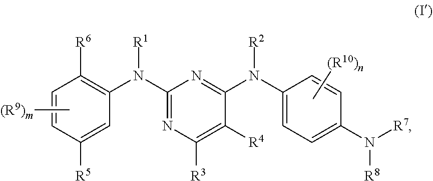

or a pharmaceutically acceptable salt, solvate, hydrate, polymorph, co-crystal, tautomer, stereoisomer, isotopically labeled derivative, or prodrug thereof.

In certain embodiments, a compound of Formula (I) is of Formula (Ia):

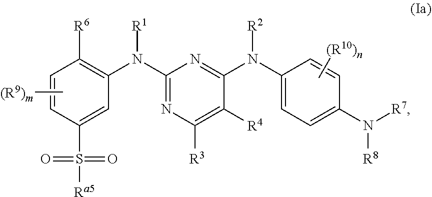

or a pharmaceutically acceptable salt, solvate, hydrate, polymorph, co-crystal, tautomer, stereoisomer, isotopically labeled derivative, or prodrug thereof.

In certain embodiments, a compound of Formula (I) is of Formula (Ib):

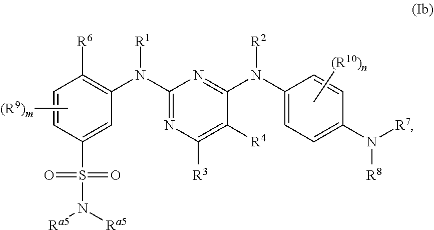

or a pharmaceutically acceptable salt, solvate, hydrate, polymorph, co-crystal, tautomer, stereoisomer, isotopically labeled derivative, or prodrug thereof.

In certain embodiments, a compound of Formula (I) is of Formula (Ic):

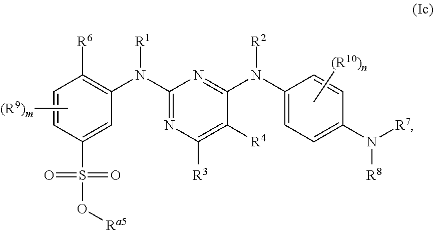

or a pharmaceutically acceptable salt, solvate, hydrate, polymorph, co-crystal, tautomer, stereoisomer, isotopically labeled derivative, or prodrug thereof.

In certain embodiments, a compound of Formula (I) is of Formula (Id):

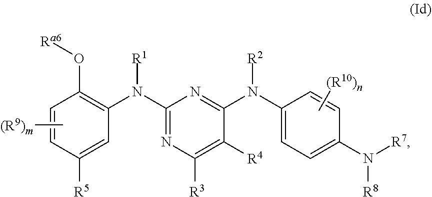

or a pharmaceutically acceptable salt, solvate, hydrate, polymorph, co-crystal, tautomer, stereoisomer, isotopically labeled derivative, or prodrug thereof.

In certain embodiments, a compound of Formula (I) is of Formula (Ie):

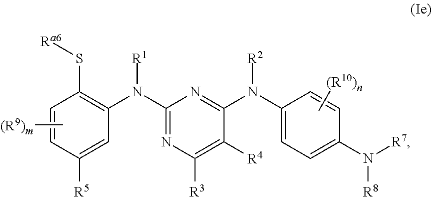

or a pharmaceutically acceptable salt, solvate, hydrate, polymorph, co-crystal, tautomer, stereoisomer, isotopically labeled derivative, or prodrug thereof.

In certain embodiments, a compound of Formula (I) is of Formula (If):

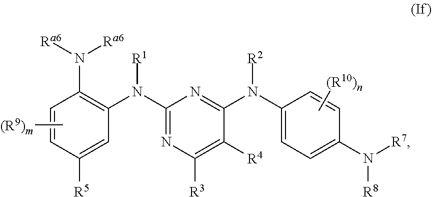

or a pharmaceutically acceptable salt, solvate, hydrate, polymorph, co-crystal, tautomer, stereoisomer, isotopically labeled derivative, or prodrug thereof.

In certain embodiments, a compound of Formula (I) is of Formula (Ij):

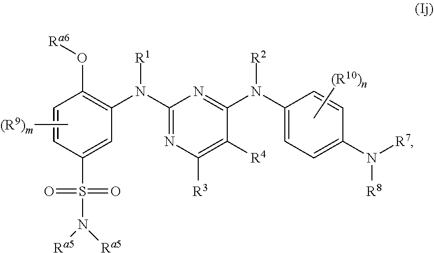

or a pharmaceutically acceptable salt, solvate, hydrate, polymorph, co-crystal, tautomer, stereoisomer, isotopically labeled derivative, or prodrug thereof.

In certain embodiments, a compound of Formula (I) is of Formula (Ik):

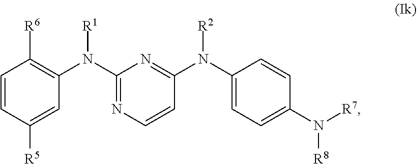

or a pharmaceutically acceptable salt, solvate, hydrate, polymorph, co-crystal, tautomer, stereoisomer, isotopically labeled derivative, or prodrug thereof.

In certain embodiments, a compound of Formula (I) is of Formula (Ii):

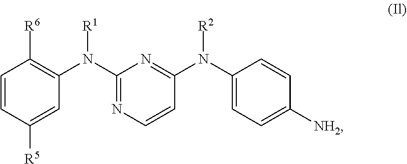

or a pharmaceutically acceptable salt, solvate, hydrate, polymorph, co-crystal, tautomer, stereoisomer, isotopically labeled derivative, or prodrug thereof.

In certain embodiments, a compound of Formula (I) is of Formula (Im):

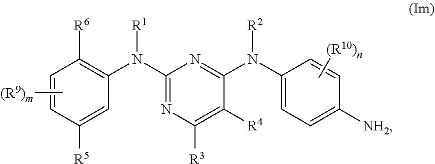

or a pharmaceutically acceptable salt, solvate, hydrate, polymorph, co-crystal, tautomer, stereoisomer, isotopically labeled derivative, or prodrug thereof.

In certain embodiments, a compound of Formula (I) is of Formula (In):

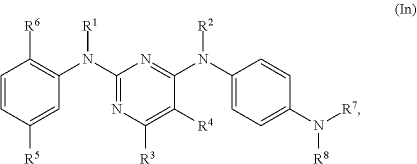

or a pharmaceutically acceptable salt, solvate, hydrate, polymorph, co-crystal, tautomer, stereoisomer, isotopically labeled derivative, or prodrug thereof.

In certain embodiments, a compound of Formula (I) is of Formula (I″):

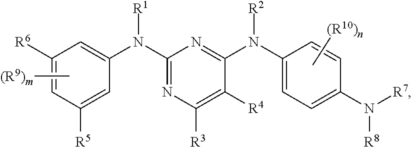

or a pharmaceutically acceptable salt, solvate, hydrate, polymorph, co-crystal, tautomer, stereoisomer, isotopically labeled derivative, or prodrug thereof.

In certain embodiments, a compound of Formula (I) is of Formula (Ig):

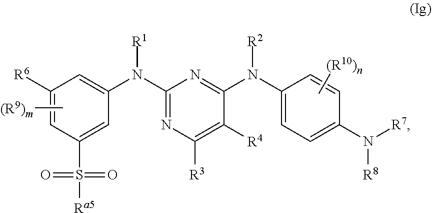

or a pharmaceutically acceptable salt, solvate, hydrate, polymorph, co-crystal, tautomer, stereoisomer, isotopically labeled derivative, or prodrug thereof.

In certain embodiments, a compound of Formula (I) is of Formula (Ih):

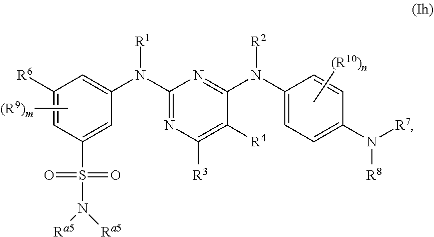

or a pharmaceutically acceptable salt, solvate, hydrate, polymorph, co-crystal, tautomer, stereoisomer, isotopically labeled derivative, or prodrug thereof.

In certain embodiments, a compound of Formula (I) is of Formula (Ii):

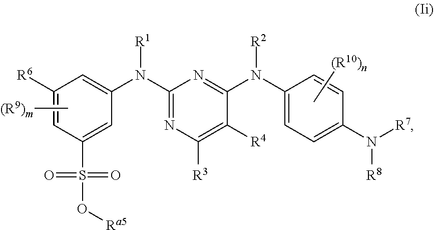

or a pharmaceutically acceptable salt, solvate, hydrate, polymorph, co-crystal, tautomer, stereoisomer, isotopically labeled derivative, or prodrug thereof.

In certain embodiments, a compound of Formula (I) is of Formula (Io):

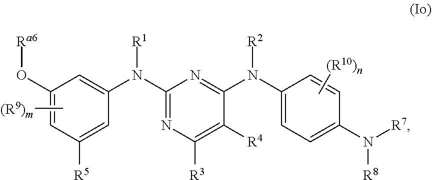

or a pharmaceutically acceptable salt, solvate, hydrate, polymorph, co-crystal, tautomer, stereoisomer, isotopically labeled derivative, or prodrug thereof.

In certain embodiments, a compound of Formula (I) is of Formula (Ip):

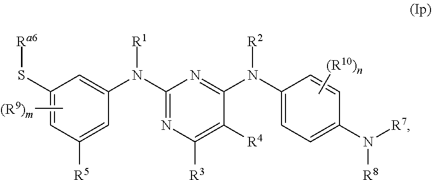

or a pharmaceutically acceptable salt, solvate, hydrate, polymorph, co-crystal, tautomer, stereoisomer, isotopically labeled derivative, or prodrug thereof.

In certain embodiments, a compound of Formula (I) is of Formula (Iq):

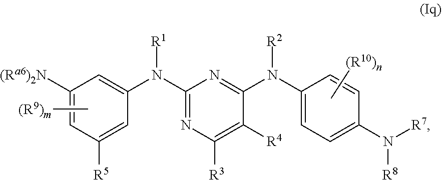

or a pharmaceutically acceptable salt, solvate, hydrate, polymorph, co-crystal, tautomer, stereoisomer, isotopically labeled derivative, or prodrug thereof.

In certain embodiments, a compound of Formula (I) is of Formula (Ir):

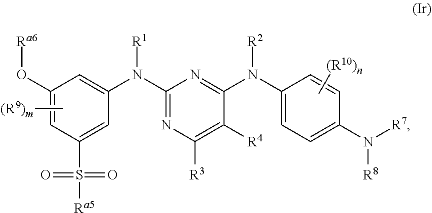

or a pharmaceutically acceptable salt, solvate, hydrate, polymorph, co-crystal, tautomer, stereoisomer, isotopically labeled derivative, or prodrug thereof.

In certain embodiments, a compound of Formula (I) is of Formula (Is):

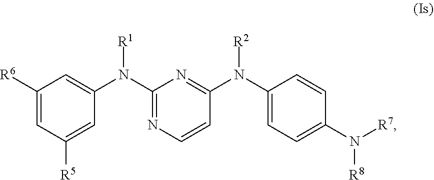

or a pharmaceutically acceptable salt, solvate, hydrate, polymorph, co-crystal, tautomer, stereoisomer, isotopically labeled derivative, or prodrug thereof.

In certain embodiments, a compound of Formula (I) is of Formula (It):

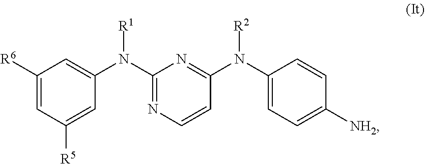

or a pharmaceutically acceptable salt, solvate, hydrate, polymorph, co-crystal, tautomer, stereoisomer, isotopically labeled derivative, or prodrug thereof.

In certain embodiments, a compound of Formula (I) is of Formula (Iu):

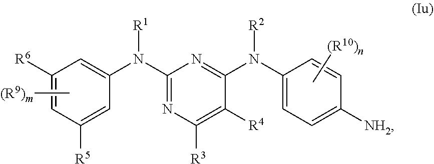

or a pharmaceutically acceptable salt, solvate, hydrate, polymorph, co-crystal, tautomer, stereoisomer, isotopically labeled derivative, or prodrug thereof.

In certain embodiments, a compound of Formula (I) is of Formula (Iv):

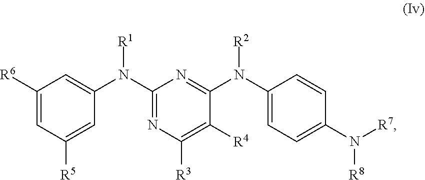

or a pharmaceutically acceptable salt, solvate, hydrate, polymorph, co-crystal, tautomer, stereoisomer, isotopically labeled derivative, or prodrug thereof.

In certain embodiments, a compound of Formula (I) is of Formula (I′″):

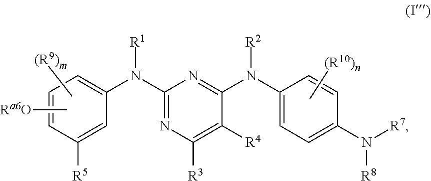

or a pharmaceutically acceptable salt, solvate, hydrate, polymorph, co-crystal, tautomer, stereoisomer, isotopically labeled derivative, or prodrug thereof.

In certain embodiments, a compound of Formula (I) is of Formula (Iw):

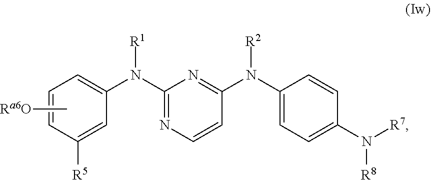

or a pharmaceutically acceptable salt, solvate, hydrate, polymorph, co-crystal, tautomer, stereoisomer, isotopically labeled derivative, or prodrug thereof.

In certain embodiments, a compound of Formula (I) is of Formula (Ix):

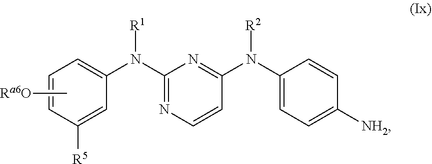

or a pharmaceutically acceptable salt, solvate, hydrate, polymorph, co-crystal, tautomer, stereoisomer, isotopically labeled derivative, or prodrug thereof.

In certain embodiments, a compound of Formula (I) is of Formula (Iy):

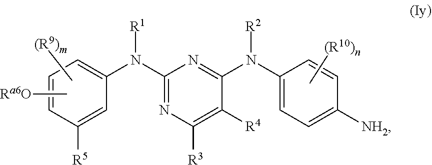

or a pharmaceutically acceptable salt, solvate, hydrate, polymorph, co-crystal, tautomer, stereoisomer, isotopically labeled derivative, or prodrug thereof.

In certain embodiments, a compound of Formula (I) is of Formula (Iz).

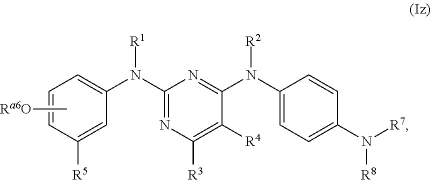

or a pharmaceutically acceptable salt, solvate, hydrate, polymorph, co-crystal, tautomer, stereoisomer, isotopically labeled derivative, or prodrug thereof.

The following chemical group definitions and embodiments apply to compounds of Formula (I) and all subgenera thereof provided herein.

As defined herein, R¹ is —H, optionally substituted aliphatic, optionally substituted acyl, or a nitrogen protecting group. In certain embodiments, R¹ is —H. In some embodiments, R¹ is substituted aliphatic. In certain embodiments, R¹ is unsubstituted aliphatic. In some embodiments, R¹ is substituted alkyl. In certain embodiments, R¹ is unsubstituted alkyl. In certain embodiments, R¹ is substituted C₁₋₆ alkyl. In some embodiments, R¹ is unsubstituted C₁₋₆ alkyl. In certain embodiments, R¹ is C₁ alkyl. In some embodiments, R¹ is C₂ alkyl. In certain embodiments, R¹ is C₃ alkyl. In some embodiments, R¹ is C₄ alkyl. In certain embodiments, R¹ is C₅ alkyl. In some embodiments, R¹ is C₆ alkyl. In certain embodiments, R¹ is methyl. In some embodiments, R¹ is ethyl. In certain embodiments, R¹ is n-propyl. In some embodiments, R¹ is isopropyl. In certain embodiments, R¹ is n-butyl. In some embodiments, R¹ is sec-butyl. In certain embodiments, R¹ is isobutyl. In some embodiments, R¹ is tert-butyl. In some embodiments, R¹ is substituted alkenyl. In certain embodiments, R¹ is unsubstituted alkenyl. In certain embodiments, R¹ is substituted C₁₋₆ alkenyl. In some embodiments, R¹ is unsubstituted C₁₋₆ alkenyl. In some embodiments, R¹ is substituted alkynyl. In certain embodiments, R¹ is unsubstituted alkynyl. In certain embodiments, R¹ is substituted C₁₋₆ alkynyl. In some embodiments, R¹ is unsubstituted C₁₋₆ alkynyl. In certain embodiments, R¹ is unsubstituted acyl. In some embodiments, R¹ is substituted acyl. In certain embodiments, R¹ is a nitrogen protecting group. In some embodiments, R¹ is Bn, Boc, Cbz, Fmoc, trifluoroacetyl, triphenylmethyl, acetyl, or Ts.

As defined herein, R² is —H, optionally substituted aliphatic, optionally substituted acyl, or a nitrogen protecting group. In certain embodiments, R² is —H. In some embodiments, R² is substituted aliphatic. In certain embodiments, R² is unsubstituted aliphatic. In some embodiments, R² is substituted alkyl. In certain embodiments, R² is unsubstituted alkyl. In certain embodiments, R² is optionally substituted C₁₋₆ alkyl. In certain embodiments, R² is substituted C₁₋₆ alkyl. In some embodiments, R² is unsubstituted C₁₋₆ alkyl. In certain embodiments, R² is C₁ alkyl. In some embodiments, R² is C₂ alkyl. In certain embodiments, R² is C₃ alkyl. In some embodiments, R² is C₄ alkyl. In certain embodiments, R² is C₅ alkyl. In some embodiments, R² is C₆ alkyl. In certain embodiments, R² is methyl. In some embodiments, R² is ethyl. In certain embodiments, R² is n-propyl. In some embodiments, R² is isopropyl. In certain embodiments, R² is n-butyl. In some embodiments, R² is sec-butyl. In certain embodiments, R² is isobutyl. In some embodiments, R² is tert-butyl. In some embodiments, R² is substituted alkenyl. In certain embodiments, R² is unsubstituted alkenyl. In certain embodiments, R² is substituted C₁₋₆ alkenyl. In some embodiments, R² is unsubstituted C₁₋₆ alkenyl. In some embodiments, R² is substituted alkynyl. In certain embodiments, R² is unsubstituted alkynyl. In certain embodiments, R² is substituted C₁₋₆ alkynyl. In some embodiments, R² is unsubstituted C₁₋₆ alkynyl. In certain embodiments, R² is unsubstituted acyl. In some embodiments, R² is substituted acyl. In certain embodiments, R² is a nitrogen protecting group. In some embodiments, R² is Bn, Boc, Cbz, Fmoc, trifluoroacetyl, triphenylmethyl, acetyl, or Ts. In some embodiments, R² is —H or optionally substituted C₁₋₆ alkyl. In some embodiments, R² is —H or -Me.

In certain embodiments, R¹ is —H, and R² is optionally substituted aliphatic. In some embodiments, R¹ is —H, and R² is unsubstituted alkyl. In certain embodiments, R¹ is —H, and R² is unsubstituted C₁₋₆ alkyl. In some embodiments, R¹ is —H, and R² is methyl. In some embodiments, R¹ is —H, and R² is ethyl. In some embodiments, R¹ is —H, and R² is n-propyl. In certain embodiments, R¹ is —H, and R² is n-butyl. In some embodiments, R¹ is —H, and R² is a nitrogen protecting group.

In certain embodiments, R² is —H, and R¹ is optionally substituted aliphatic. In some embodiments, R² is —H, and R¹ is unsubstituted alkyl. In certain embodiments, R² is —H, and R¹ is unsubstituted C₁₋₆ alkyl. In some embodiments, R² is —H, and R¹ is methyl. In some embodiments, R² is —H, and R¹ is ethyl. In some embodiments, R² is —H, and R¹ is n-propyl. In certain embodiments, R² is —H, and R¹ is n-butyl. In some embodiments, R² is —H, and R¹ is a nitrogen protecting group.

In certain embodiments, R¹ and R² are both —H. In some embodiments, R¹ and R² are both -Me. In certain embodiments, R¹ and R² are each independently nitrogen protecting groups.

As defined herein, R³ is —H, halogen, —OR^{a3}, —N(R^{a3})₂, —CN, —SCN, —NO₂, —N₃, —SR^{a3}, optionally substituted aliphatic, optionally substituted heteroaliphatic, or optionally substituted acyl. In some embodiments, R³ is —H, —OR^{a3}, —CN, —SCN, —NO₂, —N₃, —SR^{a3}, optionally substituted aliphatic, or optionally substituted acyl. In certain embodiments, R³ is —H. In some embodiments, R³ is halogen. In certain embodiments, R³ is —F. In some embodiments, R³ is —Cl. In certain embodiments, R³ is —Br. In some embodiments, R³ is —I. In certain embodiments, R³ is —OR^{a3}. In some embodiments, R³ is —N(R^{a3})₂. In certain embodiments, R³ is —CN. In some embodiments, R³ is —SCN. In certain embodiments, R³ is —NO₂. In some embodiments, R³ is —N₃. In certain embodiments, R³ is —SR^{a3}. In certain embodiments, R³ is optionally substituted aliphatic. In some embodiments, R³ is substituted aliphatic. In certain embodiments, R³ is unsubstituted aliphatic. In some embodiments, R³ is substituted alkyl. In certain embodiments, R³ is haloalkyl. In some embodiments, R³ is unsubstituted alkyl. In certain embodiments, R³ is substituted C₁₋₆ alkyl. In some embodiments, R³ is C₁₋₆ haloalkyl. In some embodiments, R³ is unsubstituted C₁₋₆ alkyl. In some embodiments, R³ is substituted alkenyl. In certain embodiments, R³ is unsubstituted alkenyl. In certain embodiments, R³ is substituted C₁₋₆ alkenyl. In some embodiments, R³ is unsubstituted C₁₋₆ alkenyl. In some embodiments, R³ is substituted alkynyl. In certain embodiments, R³ is unsubstituted alkynyl. In certain embodiments, R³ is substituted C₁₋₆ alkynyl. In some embodiments, R³ is unsubstituted C₁₋₆ alkynyl. In certain embodiments, R³ is optionally substituted heteroaliphatic. In some embodiments, R³ is substituted heteroaliphatic. In certain embodiments, R³ is unsubstituted heteroaliphatic. In some embodiments, R³ is substituted heteroalkyl. In certain embodiments, R³ is unsubstituted heteroalkyl. In certain embodiments, R³ is substituted C₁₋₆ heteroalkyl. In some embodiments, R³ is unsubstituted C₁₋₆ heteroalkyl. In some embodiments, R³ is substituted heteroalkenyl. In certain embodiments, R³ is unsubstituted heteroalkenyl. In certain embodiments, R³ is substituted C₁₋₆ heteroalkenyl. In some embodiments, R³ is unsubstituted C₁₋₆ heteroalkenyl. In some embodiments, R³ is substituted heteroalkynyl. In certain embodiments, R³ is unsubstituted heteroalkynyl. In certain embodiments, R³ is substituted C₁₋₆ heteroalkynyl. In some embodiments, R³ is unsubstituted C₁₋₆ heteroalkynyl. In certain embodiments, R³ is optionally substituted acyl. In some embodiments, R³ is unsubstituted acyl. In certain embodiments, R³ is substituted acyl. In some embodiments, R³ is not —Cl. In certain embodiments, R³ is not —NMe₃.

As defined herein, each instance of R^{a3} is independently —H, optionally substituted aliphatic, optionally substituted heteroaliphatic, optionally substituted aryl, optionally substituted heteroaryl, optionally substituted carbocyclyl, optionally substituted heterocyclyl, optionally substituted acyl, optionally substituted sulfonyl, or optionally substituted sulfinyl, an oxygen protecting group when attached to an oxygen atom, a nitrogen protecting group when attached to a nitrogen atom, or a sulfur protecting group when attached to a sulfur atom; optionally wherein any two instances of R^{a3} attached to the same nitrogen atom are joined together with the intervening atoms to form optionally substituted heterocyclyl or optionally substituted heteroaryl. In some embodiments, R^{a3} is independently —H, optionally substituted aliphatic, optionally substituted heteroaliphatic, optionally substituted aryl, optionally substituted heteroaryl, optionally substituted carbocyclyl, optionally substituted heterocyclyl, optionally substituted acyl, optionally substituted sulfonyl, or optionally substituted sulfinyl, an oxygen protecting group when attached to an oxygen atom, a nitrogen protecting group when attached to a nitrogen atom, or a sulfur protecting group when attached to a sulfur atom. In certain embodiments, R^{a3} is independently —H, optionally substituted aliphatic, an oxygen protecting group when attached to an oxygen atom, a nitrogen protecting group when attached to a nitrogen atom, or a sulfur protecting group when attached to a sulfur atom. In some embodiments, R^{a3} is independently —H, or C₁₋₆ aliphatic, an oxygen protecting group when attached to an oxygen atom, a nitrogen protecting group when attached to a nitrogen atom, or a sulfur protecting group when attached to a sulfur atom.

As defined herein, R⁴ is —H, halogen, —OR^{a3}, —N(R^{a3})₂, —CN, —SCN, —NO₂, —N₃, —SR^{a5}, optionally substituted aliphatic, optionally substituted heteroaliphatic, or optionally substituted acyl. In some embodiments, R⁴ is —H, —OR^{a3}, —N(R^{a3})₂, —SCN, —NO₂, —N₃, —SR^{a5}, unsubstituted aliphatic, optionally substituted heteroaliphatic, or optionally substituted acyl. In certain embodiments, R⁴ is —H. In some embodiments, R⁴ is halogen. In certain embodiments, R⁴ is —F. In certain embodiments, R⁴ is —Cl. In some embodiments, R⁴ is —Br. In certain embodiments, R⁴ is —I. In certain embodiments, R⁴ is —OR^{a3}. In some embodiments, R⁴ is —N(R^{a3})₂. In certain embodiments, R⁴ is —CN. In some embodiments, R⁴ is —SCN. In certain embodiments, R⁴ is —NO₂. In some embodiments, R⁴ is —N₃. In certain embodiments, R⁴ is —SR^{a3}. In certain embodiments, R⁴ is optionally substituted aliphatic. In some embodiments, R⁴ is substituted aliphatic. In certain embodiments, R⁴ is unsubstituted aliphatic. In some embodiments, R⁴ is substituted alkyl. In certain embodiments, R⁴ is haloalkyl. In some embodiments, R⁴ is unsubstituted alkyl. In certain embodiments, R⁴ is substituted C₁₋₆ alkyl. In some embodiments, R⁴ is C₁₋₆ haloalkyl. In certain embodiments, R⁴ is unsubstituted C₁₋₆ alkyl. In some embodiments, R⁴ is substituted alkenyl. In certain embodiments, R⁴ is unsubstituted alkenyl. In certain embodiments, R⁴ is substituted C₁₋₆ alkenyl. In some embodiments, R⁴ is unsubstituted C₁₋₆ alkenyl. In some embodiments, R⁴ is substituted alkynyl. In certain embodiments, R⁴ is unsubstituted alkynyl. In certain embodiments, R⁴ is substituted C₁₋₆ alkynyl. In some embodiments, R⁴ is unsubstituted C₁₋₆ alkynyl. In certain embodiments, R⁴ is optionally substituted heteroaliphatic. In some embodiments, R⁴ is substituted heteroaliphatic. In certain embodiments, R⁴ is unsubstituted heteroaliphatic. In some embodiments, R⁴ is substituted heteroalkyl. In certain embodiments, R⁴ is unsubstituted heteroalkyl. In certain embodiments, R⁴ is substituted C₁₋₆ heteroalkyl. In some embodiments, R⁴ is unsubstituted C₁₋₆ heteroalkyl. In some embodiments, R⁴ is substituted heteroalkenyl. In certain embodiments, R⁴ is unsubstituted heteroalkenyl. In certain embodiments, R⁴ is substituted C₁₋₆ heteroalkenyl. In some embodiments, R⁴ is unsubstituted C₁₋₆ heteroalkenyl. In some embodiments, R⁴ is substituted heteroalkynyl. In certain embodiments, R⁴ is unsubstituted heteroalkynyl. In certain embodiments, R⁴ is substituted C₁₋₆ heteroalkynyl. In some embodiments, R⁴ is unsubstituted C₁₋₆ heteroalkynyl. In certain embodiments, R⁴ is optionally substituted acyl. In some embodiments, R⁴ is unsubstituted acyl. In certain embodiments, R⁴ is substituted acyl. In some embodiments, R⁴ is not halogen. In certain embodiments, R⁴ is not —F. In some embodiments, R⁴ is not —Cl. In some embodiments, R⁴ is not haloalkyl. In certain embodiments, R⁴ is not —CF₃.

In certain embodiments, R³ and R⁴ are both —H. In some embodiments, R³ is —H, and R⁴ is —H, halogen, —OR^{a3}, —N(R^{a3})₂, —CN, —SCN, —NO₂, —N₃, —SR^{a3}, optionally substituted aliphatic, optionally substituted heteroaliphatic, or optionally substituted acyl. In certain embodiments, R⁴ is —H, and R³ is —H, halogen, —OR^{a3}, —N(R^{a3})₂, —CN, —SCN, —NO₂, —N₃, —SR^{a3}, optionally substituted aliphatic, optionally substituted heteroaliphatic, or optionally substituted acyl.

As defined herein, R⁵ is —S(═O)₂N(R^{a5})₂, —S(═O)₂OR^{a5}, —S(═O)₂R^{a5}, —S(═O)₂Cl, or —SR^{a5}. In some embodiments, R⁵ is —S(═O)₂N(R^{a5})₂, —S(═O)₂OR^{a5}, or —S(═O)₂R^{a5}. In certain embodiments, R⁵ is —S(═O)₂N(R^{a5})₂. In some embodiments, R⁵ is —S(═O)₂N(R^{a5})₂, wherein R^{a5} is C₁₋₆ aliphatic. In certain embodiments, R⁵ is —S(═O)₂NH(R^{a5}), wherein R^{a5} is C₁₋₆ aliphatic. In some embodiments, R⁵ is —S(═O)₂NH₂. In certain embodiments, R⁵ is —S(═O)₂OR^{a5}. In some embodiments, R⁵ is —S(═O)₂OR^{a5}, wherein R^{a5} is C₁₋₆ aliphatic. In certain embodiments, R⁵ is —S(═O)₂OH. In certain embodiments, R⁵ is —S(═O)₂R^{a5}. In some embodiments, R⁵ is —S(═O)₂R^{a5}, wherein R^{a5} is C₁₋₆ aliphatic. In certain embodiments, R⁵ is —S(═O)₂Cl. In some embodiments, R⁵ is —SR^{a5}. In certain embodiments, R⁵ is —SH. In some embodiments, R⁵ is —SR^{a5}, wherein R^{a5} is C₁₋₆ aliphatic. In certain embodiments, R⁵ is —SR^{a5}, wherein R^{a5} is a sulfur protecting group.

As defined herein, each instance of R^{a5} is independently —H, optionally substituted aliphatic, optionally substituted heteroaliphatic, optionally substituted aryl, optionally substituted heteroaryl, optionally substituted carbocyclyl, optionally substituted heterocyclyl, optionally substituted acyl, optionally substituted sulfonyl, or optionally substituted sulfinyl, an oxygen protecting group when attached to an oxygen atom, a nitrogen protecting group when attached to a nitrogen atom, or a sulfur protecting group when attached to a sulfur atom; optionally wherein any two instances of R^{a5} attached to the same nitrogen atom are joined together with the intervening atoms to form optionally substituted heterocyclyl or optionally substituted heteroaryl. In some embodiments, each instance of R^{a5} is independently —H, optionally substituted aliphatic, optionally substituted heteroaliphatic, optionally substituted aryl, optionally substituted heteroaryl, optionally substituted carbocyclyl, optionally substituted heterocyclyl, optionally substituted acyl, optionally substituted sulfonyl, or optionally substituted sulfinyl, an oxygen protecting group when attached to an oxygen atom, a nitrogen protecting group when attached to a nitrogen atom, or a sulfur protecting group when attached to a sulfur atom. In certain embodiments, each instance of R^{a5} is independently —H, optionally substituted aliphatic, an oxygen protecting group when attached to an oxygen atom, a nitrogen protecting group when attached to a nitrogen atom, or a sulfur protecting group when attached to a sulfur atom. In some embodiments, each instance of R^{a5} is independently —H, or C₁₋₆ aliphatic, an oxygen protecting group when attached to an oxygen atom, a nitrogen protecting group when attached to a nitrogen atom, or a sulfur protecting group when attached to a sulfur atom. In certain embodiments, each instance of R^{a5} is independently —H, or C₁₋₆ aliphatic.

As defined herein, R⁶ is —OR^{a6}, —N(R^{a6})₂, —CN, —SCN, —NO₂, —N₃, —SR^{a6}, optionally substituted heteroaliphatic, optionally substituted acyl, optionally substituted sulfonyl, or optionally substituted sulfinyl. In some embodiments, R⁶ is —OR^{a6}, —N(R^{a6})₂, or —SR^{a6}. In certain embodiments, R⁶ is —OR^{a6}. In some embodiments, R⁶ is —OH. In certain embodiments, R⁶ is —OMe. In some embodiments, R⁶ is —OEt. In certain embodiments, R⁶ is —OR^{a6}, wherein R^{a6} is an oxygen protecting group. In some embodiments, R⁶ is —N(R^{a6})₂. In certain embodiments, R⁶ is —NH₂. In some embodiments, R⁶ is —N(R^{a6})₂, wherein R^{a6} is a nitrogen protecting group. In certain embodiments, R⁶ is —CN. In some embodiments, R⁶ is —SCN. In certain embodiments, R⁶ is —NO₂. In some embodiments, R⁶ is —N₃. In certain embodiments, R⁶ is —SR^{a6}. In some embodiments, R⁶ is —SH. In certain embodiments, R⁶ is —SR^{a6}, wherein R^{a6} is a sulfur protecting group. In certain embodiments, R⁶ is optionally substituted heteroaliphatic. In certain embodiments, R⁶ is optionally substituted heteroaliphatic. In some embodiments, R⁶ is substituted heteroaliphatic. In certain embodiments, R⁶ is unsubstituted heteroaliphatic. In some embodiments, R⁶ is substituted heteroalkyl. In certain embodiments, R⁶ is unsubstituted heteroalkyl. In certain embodiments, R⁶ is substituted C₁₋₆ heteroalkyl. In some embodiments, R⁶ is unsubstituted C₁₋₆ heteroalkyl. In some embodiments, R⁶ is substituted heteroalkenyl. In certain embodiments, R⁶ is unsubstituted heteroalkenyl. In certain embodiments, R⁶ is substituted C₁₋₆ heteroalkenyl. In some embodiments, R⁶ is unsubstituted C₁₋₆ heteroalkenyl. In some embodiments, R⁶ is substituted heteroalkynyl. In certain embodiments, R⁶ is unsubstituted heteroalkynyl. In certain embodiments, R⁶ is substituted C₁₋₆ heteroalkynyl. In some embodiments, R⁶ is unsubstituted C₁₋₆ heteroalkynyl. In certain embodiments, R⁶ is optionally substituted acyl. In some embodiments, R⁶ is unsubstituted acyl. In certain embodiments, R⁶ is substituted acyl. In some embodiments, R⁶ is optionally substituted sulfonyl. In certain embodiments, R⁶ is unsubstituted sulfonyl. In some embodiments, R⁶ is substituted sulfonyl. In certain embodiments, R⁶ is optionally substituted sulfinyl. In some embodiments, R⁶ is unsubstituted sulfinyl. In certain embodiments, R⁶ is unsubstituted sulfinyl.

As defined herein, each instance of R^{a6} is independently —H, optionally substituted aliphatic, optionally substituted heteroaliphatic, optionally substituted aryl, optionally substituted heteroaryl, optionally substituted carbocyclyl, optionally substituted heterocyclyl, optionally substituted acyl, optionally substituted sulfonyl, or optionally substituted sulfinyl, an oxygen protecting group when attached to an oxygen atom, a nitrogen protecting group when attached to a nitrogen atom, or a sulfur protecting group when attached to a sulfur atom; optionally wherein any two instances of R^{a6} attached to the same nitrogen atom are joined together with the intervening atoms to form optionally substituted heterocyclyl or optionally substituted heteroaryl. In some embodiments, each instance of R^{a6} is independently —H, optionally substituted aliphatic, optionally substituted heteroaliphatic, optionally substituted aryl, optionally substituted heteroaryl, optionally substituted carbocyclyl, optionally substituted heterocyclyl, optionally substituted acyl, optionally substituted sulfonyl, or optionally substituted sulfinyl, an oxygen protecting group when attached to an oxygen atom, a nitrogen protecting group when attached to a nitrogen atom, or a sulfur protecting group when attached to a sulfur atom. In certain embodiments, each instance of R^{a6} is independently —H, optionally substituted aliphatic, an oxygen protecting group when attached to an oxygen atom, a nitrogen protecting group when attached to a nitrogen atom, or a sulfur protecting group when attached to a sulfur atom. In some embodiments, each instance of R^{a6} is independently —H, or C₁₋₆ aliphatic, an oxygen protecting group when attached to an oxygen atom, a nitrogen protecting group when attached to a nitrogen atom, or a sulfur protecting group when attached to a sulfur atom.

In some embodiments, R⁵ is —S(═O)₂N(R^{a5})₂, —S(═O)₂OR^{a5}, —S(═O)₂R^{a5}, —S(═O)₂Cl, or —SR^{a5}, and R⁶ is —OR^{a6}, —N(R^{a6})₂, or —SR^{a6}. In certain embodiments, R⁵ is —S(═O)₂N(R^{a5})₂, —S(═O)₂OR^{a5}, —S(═O)₂R^{a5}, —S(═O)₂Cl, or —SR^{a5}, and R⁶ is —OR^{a6}. In some embodiments, R⁵ is —S(═O)₂N(R^{a5})₂, —S(═O)₂OR^{a5}, or —S(═O)₂R^{a5}, and R⁶ is —OR^{a6}, —N(R^{a6})₂, or —SR^{a6}. In certain embodiments, R⁵ is —S(═O)₂N(R^{a5})₂, —S(═O)₂OR^{a5}, or —S(═O)₂R^{a5}, and R⁶ is —OR^{a6} In some embodiments, R⁵ is —S(═O)₂N(R^{a5})₂, and R⁶ is —OR^{a6}, —N(R^{a6})₂, —CN, —SCN, —NO₂, —N₃, —SR^{a6}, optionally substituted heteroaliphatic, optionally substituted acyl, optionally substituted sulfonyl, or optionally substituted sulfinyl. In certain embodiments, R⁵ is —S(═O)₂N(R^{a5})₂, and R⁶ is —OR^{a6}, —N(R^{a6})₂, or —SR^{a6}. In some embodiments, R⁵ is —S(═O)₂N(R^{a5})₂, and R⁶ is —OR^{a6}.

As defined herein, R⁷ is —H, unsubstituted aliphatic, unsubstituted heteroaliphatic, —C(═O)R^{a7}, —C(═O)OR^{a7}, or a nitrogen protecting group; optionally wherein R⁷ and R⁸ are joined together with the intervening atoms to form optionally substituted heterocyclyl or optionally substituted heteroaryl. In some embodiments, R⁷ is —H, unsubstituted aliphatic, unsubstituted heteroaliphatic, —C(═O)R^{a7}, —C(═O)OR^{a7}, or a nitrogen protecting group. In certain embodiments, R⁷ is —H, unsubstituted aliphatic, unsubstituted heteroaliphatic, —C(═O)R^{a7}, or —C(═O)OR^{a7}. In some embodiments, R⁷ is —H or unsubstituted aliphatic. In certain embodiments, R⁷ is —H. In some embodiments, R⁷ is unsubstituted aliphatic. In some embodiments, R⁷ is unsubstituted alkyl. In certain embodiments, R⁷ is unsubstituted C₁₋₆ alkyl. In some embodiments, R⁷ is unsubstituted alkenyl. In certain embodiments, R⁷ is unsubstituted C₁₋₆ alkenyl. In some embodiments, R⁷ is unsubstituted alkynyl. In certain embodiments, R⁷ is unsubstituted C₁₋₆ alkynyl. In some embodiments, R⁷ is unsubstituted heteroaliphatic. In certain embodiments, R⁷ is unsubstituted heteroalkyl. In some embodiments, R⁷ is unsubstituted C₁₋₆ heteroalkyl. In certain embodiments, R⁷ is unsubstituted heteroalkenyl. In some embodiments, R⁷ is unsubstituted C₁₋₆ heteroalkenyl. In certain embodiments, R⁷ is unsubstituted heteroalkynyl. In some embodiments, R⁷ is unsubstituted C₁₋₆ heteroalkynyl. In certain embodiments, R⁷ is —C(═O)R^{a7}. In some embodiments, R⁷ is —C(═O)OR^{a7}. In certain embodiments, R⁷ is a nitrogen protecting group. In some embodiments, the nitrogen protecting group does not form a urea with the nitrogen to which it is attached. In certain embodiments, R⁷ is Bn, Boc, Cbz, Fmoc, trifluoroacetyl, triphenylmethyl, acetyl, or Ts. In some embodiments, R⁷ is not —C(═O)N(R^{a7})₂.

As defined herein, R⁸ is —H, unsubstituted aliphatic, unsubstituted heteroaliphatic, —C(═O)R^{a7}, —C(═O)OR^{a7}, or a nitrogen protecting group; optionally wherein R⁷ and R⁸ are joined together with the intervening atoms to form optionally substituted heterocyclyl or optionally substituted heteroaryl. In some embodiments, R⁸ is —H, unsubstituted aliphatic, unsubstituted heteroaliphatic, —C(═O)R^{a7}, —C(═O)OR^{a7}, or a nitrogen protecting group. In certain embodiments, R⁸ is —H, unsubstituted aliphatic, unsubstituted heteroaliphatic, —C(═O)R^{a7}, or —C(═O)OR^{a7}. In some embodiments, R⁸ is —H or unsubstituted aliphatic. In certain embodiments, R⁸ is —H. In some embodiments, R⁸ is unsubstituted aliphatic. In some embodiments, R⁸ is unsubstituted alkyl. In certain embodiments, R⁸ is unsubstituted C₁₋₆ alkyl. In some embodiments, R⁸ is unsubstituted alkenyl. In certain embodiments, R⁸ is unsubstituted C₁₋₆ alkenyl. In some embodiments, R⁸ is unsubstituted alkynyl. In certain embodiments, R⁸ is unsubstituted C₁₋₆ alkynyl. In some embodiments, R⁸ is unsubstituted heteroaliphatic. In certain embodiments, R⁸ is unsubstituted heteroalkyl. In some embodiments, R⁸ is unsubstituted C₁₋₆ heteroalkyl. In certain embodiments, R⁸ is unsubstituted heteroalkenyl. In some embodiments, R⁸ is unsubstituted C₁₋₆ heteroalkenyl. In certain embodiments, R⁸ is unsubstituted heteroalkynyl. In some embodiments, R⁸ is unsubstituted C₁₋₆ heteroalkynyl. In certain embodiments, R⁸ is —C(═O)R^{a7}. In some embodiments, R⁸ is —C(═O)OR^{a7}. In certain embodiments, R⁸ is a nitrogen protecting group. In some embodiments, the nitrogen protecting group does not form a urea with the nitrogen to which it is attached. In certain embodiments, R⁸ is Bn, Boc, Cbz, Fmoc, trifluoroacetyl, triphenylmethyl, acetyl, or Ts. In some embodiments, R⁸ is not —C(═O)N(R^{a7})₂.

In certain embodiments, R⁷ and R⁸ are joined together with the intervening atoms to form optionally substituted heterocyclyl or optionally substituted heteroaryl. In some embodiments, R⁷ and R⁸ are joined together with the intervening atoms to form optionally substituted heterocyclyl. In certain embodiments, R⁷ and R⁸ are joined together with the intervening atoms to form optionally substituted heteroaryl.

In certain embodiments, R⁷ and R⁸ are each independently —H, optionally substituted C₁₋₆ alkyl, or a nitrogen protecting group. In some embodiments, R⁷ and R⁸ are both —H. In certain embodiments, R⁷ and R⁸ are both optionally substituted C₁₋₆ alkyl. In some embodiments, R⁷ and R⁸ are both nitrogen protecting groups. In certain embodiments, R⁷ is —H, and R⁸ is —H, optionally substituted C₁₋₆ alkyl, or a nitrogen protecting group. In some embodiments, R⁸ is —H, and R⁷ is —H, optionally substituted C₁₋₆ alkyl, or a nitrogen protecting group.

As defined herein, each instance of R^{a7} is independently —H, or unsubstituted aliphatic. In certain embodiments, R^{a7} is —H. In some embodiments, R^{a7} is unsubstituted aliphatic. In certain embodiments, R^{a7} is unsubstituted C₁₋₆ aliphatic. In some embodiments, R^{a7} is —H, or unsubstituted C₁₋₆ aliphatic.

As defined herein, each instance of R⁹ is independently —H, —OR^{a9}, —C(═O)R^{a9}, —OC(═O)R^{a9}, —C(═O)OR^{a9}, —N(R^{a9})₂, —N(C═O)R^{a9}, —(C═O)N(R^{a9})₂, —CN, —SCN, —NO₂, —N₃, —SR^{a9}, halogen, optionally substituted aliphatic, optionally substituted heteroaliphatic, optionally substituted sulfonyl, or optionally substitute d sulfinyl. In some embodiments, R⁹ is —H, —OR^{a9}, —C(═O)R^{a9}, —C(═O)OR^{a9}, —N(R^{a9})₂, —N(C═O)R^{a9}, —(C═O)N(R^{a9})₂, —NO₂, halogen, optionally substituted aliphatic, or optionally substituted heteroaliphatic. In certain embodiments, R⁹ is —H. In some embodiments, R⁹ is —OR^{a9}. In certain embodiments, R⁹ is —C(═O)R^{a9}. In some embodiments, R⁹ is —OC(═O)R^{a9}. In certain embodiments, R⁹ is —C(═O)OR^{a9}. In some embodiments, R⁹ is —N(R^{a9})₂. In certain embodiments, R⁹ is —N(C═O)R^{a9}. In some embodiments, R⁹ is —(C═O)N(R^{a9})₂. In certain embodiments, R⁹ is —CN. In some embodiments, R⁹ is —SCN. In certain embodiments, R⁹ is —NO₂. In some embodiments, R⁹ is —N₃. In certain embodiments, R⁹ is —SR^{a9}. In some embodiments, R⁹ is halogen. In certain embodiments, R⁹ is —F. In some embodiments, R⁹ is —Cl. In certain embodiments, R⁹ is —Br. In some embodiments, R⁹ is —I. In certain embodiments, R⁹ is optionally substituted aliphatic. In some embodiments, R⁹ is unsubstituted aliphatic. In certain embodiments, R⁹ is substituted aliphatic. In some embodiments, R⁹ is unsubstituted C₁₋₆ aliphatic. In certain embodiments, R⁹ is substituted C₁₋₆ aliphatic. In some embodiments, R⁹ is optionally substituted heteroaliphatic. In certain embodiments, R⁹ is unsubstituted heteroaliphatic. In some embodiments, R⁹ is substituted heteroaliphatic. In certain embodiments, R⁹ is unsubstituted C₁₋₆ heteroaliphatic. In some embodiments, R⁹ is substituted C₁₋₆ heteroaliphatic. In certain embodiments, R⁹ is optionally substituted sulfonyl. In some embodiments, R⁹ is unsubstituted sulfonyl. In certain embodiments, R⁹ is substituted sulfonyl. In certain embodiments, R⁹ is optionally substituted sulfinyl. In some embodiments, R⁹ is unsubstituted sulfinyl. In certain embodiments, R⁹ is substituted sulfinyl.

As defined herein, each instance of R¹⁰ is independently —H, —OR^{a9}, —C(═O)R^{a9}, —OC(═O)R^{a9}, —C(═O)OR^{a9}, —N(R^{a9})₂, —N(C═O)R^{a9}, —(C═O)N(R^{a9})₂, —CN, —SCN, —NO₂, —N₃, —SR^{a9}, halogen, optionally substituted aliphatic, optionally substituted heteroaliphatic, optionally substituted sulfonyl, or optionally substituted sulfinyl. In some embodiments, R¹⁰ is —H, —OR^{a9}, —C(═O)R^{a9}, —C(═O)OR^{a9}, —N(R^{a9})₂, —N(C═O)R^{a9}, —(C═O)N(R^{a9})₂, —NO₂, halogen, optionally substituted aliphatic, or optionally substituted heteroaliphatic. In certain embodiments, R¹⁰ is —H. In some embodiments, R¹⁰ is —OR^{a9}. In certain embodiments, R¹⁰ is —C(═O)R^{a9}. In some embodiments, R¹⁰ is —OC(═O)R^{a9}. In certain embodiments, R¹⁰ is —C(═O)OR^{a9}. In some embodiments, R¹⁰ is —N(R^{a9})₂. In certain embodiments, R¹⁰ is —N(C═O)R^{a9}. In some embodiments, R¹⁰ is —(C═O)N(R^{a9})₂. In certain embodiments, R¹⁰ is —CN. In some embodiments, R¹⁰ is —SCN. In certain embodiments, R¹⁰ is —NO₂. In some embodiments, R¹⁰ is —N₃. In certain embodiments, R¹⁰ is —SR^{a9}. In some embodiments, R¹⁰ is halogen. In certain embodiments, R¹⁰ is —F. In some embodiments, R¹⁰ is —Cl. In certain embodiments, R¹⁰ is —Br. In some embodiments, R¹⁰ is —I. In certain embodiments, R¹⁰ is optionally substituted aliphatic. In some embodiments, R¹⁰ is unsubstituted aliphatic. In certain embodiments, R¹⁰ is substituted aliphatic. In some embodiments, R¹⁰ is unsubstituted C₁₋₆ aliphatic. In certain embodiments, R¹⁰ is substituted C₁₋₆ aliphatic. In some embodiments, R¹⁰ is optionally substituted heteroaliphatic. In certain embodiments, R¹⁰ is unsubstituted heteroaliphatic. In some embodiments, R¹⁰ is substituted heteroaliphatic. In certain embodiments, R¹⁰ is unsubstituted C₁₋₆ heteroaliphatic. In some embodiments, R¹⁰ is substituted C₁₋₆ heteroaliphatic. In certain embodiments, R¹⁰ is optionally substituted sulfonyl. In some embodiments, R¹⁰ is unsubstituted sulfonyl. In certain embodiments, R¹⁰ is substituted sulfonyl. In certain embodiments, R¹⁰ is optionally substituted sulfinyl. In some embodiments, R¹⁰ is unsubstituted sulfinyl. In certain embodiments, R¹⁰ is substituted sulfinyl.

As defined herein, each instance of R^{a9} is independently —H, optionally substituted aliphatic, optionally substituted heteroaliphatic, optionally substituted aryl, optionally substituted heteroaryl, optionally substituted carbocyclyl, optionally substituted heterocyclyl, optionally substituted acyl, optionally substituted sulfonyl, or optionally substituted sulfinyl, an oxygen protecting group when attached to an oxygen atom, a nitrogen protecting group when attached to a nitrogen atom, or a sulfur protecting group when attached to a sulfur atom; optionally wherein any two instances of R^{a9} attached to the same nitrogen atom are joined together with the intervening atoms to form optionally substituted heterocyclyl or optionally substituted heteroaryl. In some embodiments, each instance of R^{a9} is independently —H, optionally substituted aliphatic, optionally substituted heteroaliphatic, optionally substituted aryl, optionally substituted heteroaryl, optionally substituted carbocyclyl, optionally substituted heterocyclyl, optionally substituted acyl, optionally substituted sulfonyl, or optionally substituted sulfinyl, an oxygen protecting group when attached to an oxygen atom, a nitrogen protecting group when attached to a nitrogen atom, or a sulfur protecting group when attached to a sulfur atom. In certain embodiments, each instance of R^{a9} is independently —H, optionally substituted aliphatic, an oxygen protecting group when attached to an oxygen atom, a nitrogen protecting group when attached to a nitrogen atom, or a sulfur protecting group when attached to a sulfur atom. In some embodiments, each instance of R^{a9} is independently —H, or C₁₋₆ aliphatic, an oxygen protecting group when attached to an oxygen atom, a nitrogen protecting group when attached to a nitrogen atom, or a sulfur protecting group when attached to a sulfur atom.

In some embodiments, R⁵ is —S(═O)₂N(R^{a5})₂, —S(═O)₂OR^{a5}, —S(═O)₂R^{a5}, —S(═O)₂Cl, or —SR^{a5}, R⁶ is —OR^{a6}, —N(R^{a6})₂, or —SR^{a6}, and each instance of R⁹ is —H. In certain embodiments, R⁵ is —S(═O)₂N(R^{a5})₂, —S(═O)₂OR^{a5}, —S(═O)₂R^{a5}, —S(═O)₂Cl, or —SR^{a5}, R⁶ is —OR^{a6}, and each instance of R⁹ is —H. In some embodiments, R⁵ is —S(═O)₂N(R^{a5})₂, —S(═O)₂OR^{a5}, or —S(═O)₂R^{a5}, R⁶ is —OR^{a6}, —N(R^{a6})₂, or —SR^{a6}, and each instance of R⁹ is —H. In certain embodiments, R⁵ is —S(═O)₂N(R^{a5})₂, —S(═O)₂OR^{a5}, or —S(═O)₂R^{a5}, R⁶ is —OR^{a6}, and each instance of R⁹ is —H. In some embodiments, R⁵ is —S(═O)₂N(R^{a5})₂, R⁶ is —OR^{a6}, —N(R^{a6})₂, —CN, —SCN, —NO₂, —N₃, —SR^{a6}, optionally substituted heteroaliphatic, optionally substituted acyl, optionally substituted sulfonyl, or optionally substituted sulfinyl, and each instance of R⁹ is —H. In certain embodiments, R⁵ is —S(═O)₂N(R^{a5})₂, R⁶ is —OR^{a6}, —N(R^{a6})₂, or —SR^{a6}, and each instance of R⁹ is —H. In some embodiments, R⁵ is —S(═O)₂N(R^{a5})₂, R⁶ is —OR^{a6}, and each instance of R⁹ is —H.

In certain embodiments, R⁷ and R⁸ are each independently —H, optionally substituted C₁₋₆ alkyl, or a nitrogen protecting group, and each instance of R¹⁰ is —H. In some embodiments, R⁷ and R⁸ are both —H, and each instance of R¹⁰ is —H. In certain embodiments, R⁷ and R⁸ are both optionally substituted C₁₋₆ alkyl, and each instance of R¹⁰ is —H. In some embodiments, R⁷ and R⁸ are both nitrogen protecting groups, and each instance of R¹⁰ is —H. In certain embodiments, R⁷ is —H, and R⁸ is —H, optionally substituted C₁₋₆ alkyl, or a nitrogen protecting group, and each instance of R¹⁰ is —H. In some embodiments, R⁸ is —H, and R⁷ is —H, optionally substituted C₁₋₆ alkyl, or a nitrogen protecting group, and each instance of R¹⁰ is —H.

As defined herein, m is 0, 1, 2, or 3. In certain embodiments, m is 0. In some embodiments, m is 1. In certain embodiments, m is 2. In some embodiments, m is 3.

As defined herein, n is 0, 1, 2, 3, or 4. In certain embodiments, n is 0. In some embodiments, n is 1. In certain embodiments, n is 2. In some embodiments, n is 3. In certain embodiments, n is 4.

In certain embodiments, m is 0, and n is 0. In some embodiments, m is 0, and n is 1. In certain embodiments, m is 0, and n is 2. In some embodiments, m is 0, and n is 3. In certain embodiments, m is 0, and n is 4. In some embodiments, m is 1, and n is 0. In certain embodiments, m is 1, and n is 1. In some embodiments, m is 1, and n is 2. In certain embodiments, m is 1, and n is 3. In some embodiments, m is 1, and n is 4. In certain embodiments, m is 2, and n is 0. In some embodiments, m is 2, and n is 1. In certain embodiments, m is 2, and n is 2. In some embodiments, m is 2, and n is 3. In certain embodiments, m is 2, and n is 4. In some embodiments, m is 3, and n is 0. In certain embodiments, m is 3, and n is 1. In some embodiments, m is 3, and n is 2. In certain embodiments, m is 3, and n is 3. In some embodiments, m is 3, and n is 4.

In certain embodiments, a compound of Formula (I) is the following:

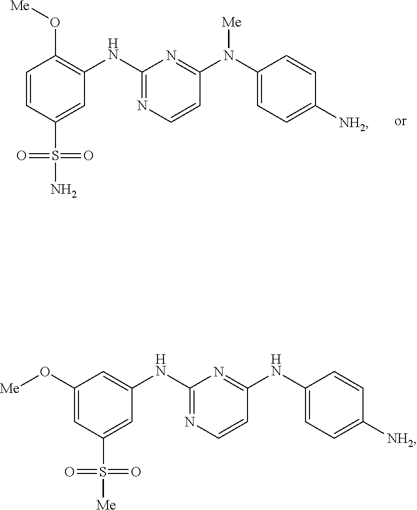 [SMILES: COc1ccc(S(N)(=O)=O)cc1Nc1nccc(N(C)c2ccc(N)cc2)n1 ; "or" ; COc1cc(Nc2nccc(Nc3ccc(N)cc3)n2)cc(S(C)(=O)=O)c1]

or a pharmaceutically acceptable salt, solvate, hydrate, polymorph, co-crystal, tautomer, stereoisomer, isotopically labeled derivative, or prodrug thereof.

Also provided herein are compounds of Formula (II):

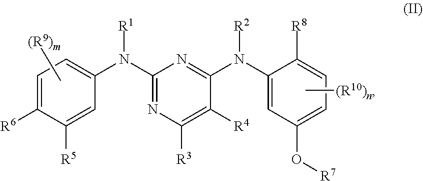

and pharmaceutically acceptable salts, solvates, hydrates, polymorphs, co-crystals, tautomers, stereoisomers, isotopically labeled derivatives, and prodrugs thereof, wherein:
R¹ and R² are each independently —H, optionally substituted aliphatic, optionally substituted acyl, or a nitrogen protecting group;
R³ is —H, halogen, —OR^{a3}, —N(R^{a3})₂, —CN, —SCN, —NO₂, —N₃, —SR^{a3}, optionally substituted aliphatic, optionally substituted heteroaliphatic, or optionally substituted acyl;
R⁴ is —H, —Br, —OR^{a4}, —N(R^{a4})₂, —CN, —SCN, —N₃, —SR^{a4}, branched or unbranched aliphatic, optionally substituted heteroaliphatic, or optionally substituted acyl;
R⁵ and R⁶ are each independently —H, —S(═O)₂N(R^{a5})₂, —S(═O)₂OR^{a5}, —S(═O)₂R^{a5}, —S(═O)₂Cl, —SR^{a5}, —C(═O)N(R^{a6})₂, —C(═O)OR^{a6}, or —C(═O)R^{a6}; provided that R⁵ and R⁶ are not both —H;
R⁷ is —H, optionally substituted aliphatic, optionally substituted acyl, or an oxygen protecting group;
R⁸ is —N(R^{a8})₂, —CN, —SCN, —NO₂, —N₃, —SR^{a8}, halogen, optionally substituted aliphatic, optionally substituted acyl, optionally substituted sulfonyl, or optionally substituted sulfinyl;
each instance of R⁹ is independently —H, —OR^{a9}, —C(═O)R^{a9}, —C(═O)OR^{a9}, —OC(═O)R^{a9}, —N(R^{a9})₂, —N(C═O)R^{a9}, —(C═O)N(R^{a9})₂, —CN, —SCN, —NO₂, —N₃, —SR^{a9}, halogen, optionally substituted aliphatic, optionally substituted heteroaliphatic, optionally substituted sulfonyl, or optionally substituted sulfinyl;
each instance of R¹⁰ is independently —H, —C(═O)R^{a10}, —C(═O)OR^{a10}, —OC(═O)R^{a10}, —N(C═O)R^{a10}, —(C═O)N(R^{a10})₂, —CN, —SCN, —NO₂, —N₃, —SR^{a10}, halogen, optionally substituted aliphatic, optionally substituted heteroaliphatic, optionally substituted sulfonyl, or optionally substituted sulfinyl;
each instance of R^{a5}, R^{a4}, R^{a5}, R^{a6}, R^{a8}, R^{a9}, and R^{a10} is independently —H, optionally substituted aliphatic, optionally substituted heteroaliphatic, optionally substituted aryl, optionally substituted heteroaryl, optionally substituted carbocyclyl, optionally substituted heterocyclyl, optionally substituted acyl, optionally substituted sulfonyl, or optionally substituted sulfinyl, an oxygen protecting group when attached to an oxygen atom, a nitrogen protecting group when attached to a nitrogen atom, or a sulfur protecting group when attached to a sulfur atom; optionally wherein any two instances of R^{a3}, R^{a4}, R^{a5}, R^{a6}, R^{a8}, R^{a9}, or R^{a10} attached to the same nitrogen atom are joined together with the intervening atoms to form optionally substituted heterocyclyl or optionally substituted heteroaryl;
m is 0, 1, 2, or 3; and
n is 0, 1, 2, or 3.

In certain embodiments, a compound of Formula (II) is not one of the following formulae:

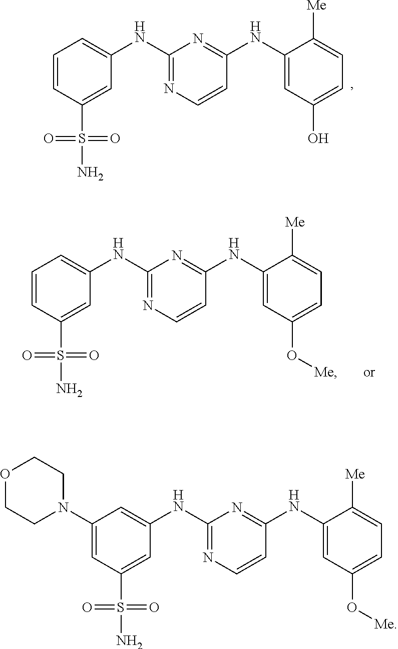 [SMILES: Cc1ccc(O)cc1Nc1ccnc(Nc2cccc(S(N)(=O)=O)c2)n1 ; COc1ccc(C)c(Nc2ccnc(Nc3cccc(S(N)(=O)=O)c3)n2)c1 ; COc1ccc(C)c(Nc2ccnc(Nc3cc(N4CCOCC4)cc(S(N)(=O)=O)c3)n2)c1]

In certain embodiments, a compound of Formula (II) is of Formula (IIa):

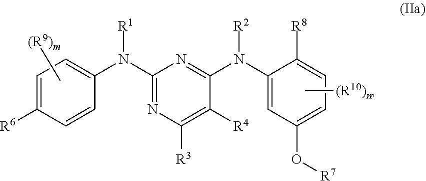

or a pharmaceutically acceptable salt, solvate, hydrate, polymorph, co-crystal, tautomer, stereoisomer, isotopically labeled derivative, or prodrug thereof.

In certain embodiments, a compound of Formula (II) is of Formula (IIb):

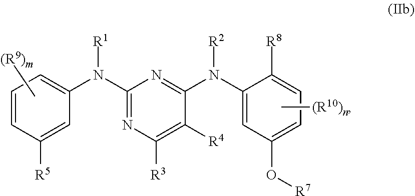

or a pharmaceutically acceptable salt, solvate, hydrate, polymorph, co-crystal, tautomer, stereoisomer, isotopically labeled derivative, or prodrug thereof.

In certain embodiments, a compound of Formula (II) is of Formula (IIa-i):

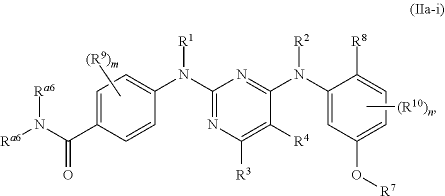

or a pharmaceutically acceptable salt, solvate, hydrate, polymorph, co-crystal, tautomer, stereoisomer, isotopically labeled derivative, or prodrug thereof.

In certain embodiments, a compound of Formula (II) is of Formula (IIa-ii):

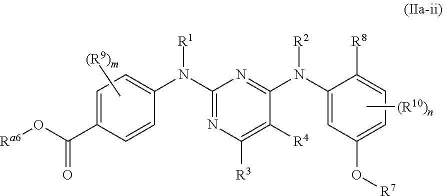

or a pharmaceutically acceptable salt, solvate, hydrate, polymorph, co-crystal, tautomer, stereoisomer, isotopically labeled derivative, or prodrug thereof.

In certain embodiments, a compound of Formula (II) is of Formula (IIa-iii):

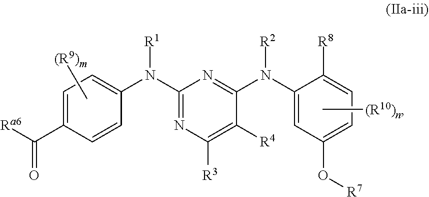

or a pharmaceutically acceptable salt, solvate, hydrate, polymorph, co-crystal, tautomer, stereoisomer, isotopically labeled derivative, or prodrug thereof.

In certain embodiments, a compound of Formula (II) is of Formula (IIIa-iv):

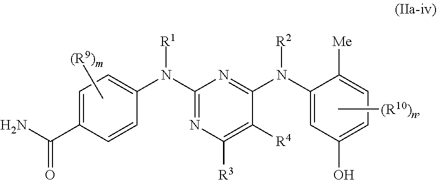

or a pharmaceutically acceptable salt, solvate, hydrate, polymorph, co-crystal, tautomer, stereoisomer, isotopically labeled derivative, or prodrug thereof.

In certain embodiments, a compound of Formula (II) is of Formula (IIIa-v):

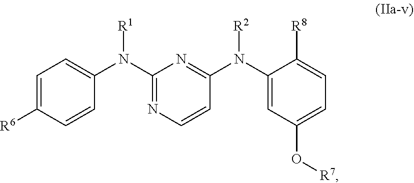

or a pharmaceutically acceptable salt, solvate, hydrate, polymorph, co-crystal, tautomer, stereoisomer, isotopically labeled derivative, or prodrug thereof.

In certain embodiments, a compound of Formula (II) is of Formula (IIIa-vi):

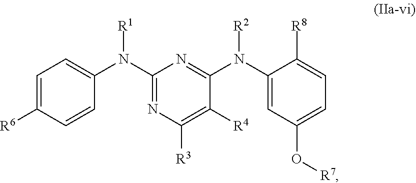

or a pharmaceutically acceptable salt, solvate, hydrate, polymorph, co-crystal, tautomer, stereoisomer, isotopically labeled derivative, or prodrug thereof.

In certain embodiments, a compound of Formula (II) is of Formula (IIb-i):

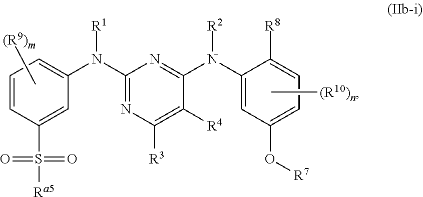

or a pharmaceutically acceptable salt, solvate, hydrate, polymorph, co-crystal, tautomer, stereoisomer, isotopically labeled derivative, or prodrug thereof.

In certain embodiments, a compound of Formula (II) is of Formula (IIb-ii):

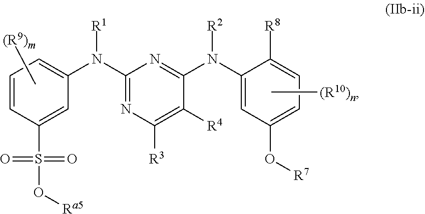

or a pharmaceutically acceptable salt, solvate, hydrate, polymorph, co-crystal, tautomer, stereoisomer, isotopically labeled derivative, or prodrug thereof.

In certain embodiments, a compound of Formula (II) is of Formula (IIb-iii):

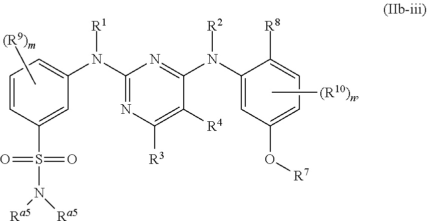

or a pharmaceutically acceptable salt, solvate, hydrate, polymorph, co-crystal, tautomer, stereoisomer, isotopically labeled derivative, or prodrug thereof.

In certain embodiments, a compound of Formula (II) is of Formula (IIb-iv):

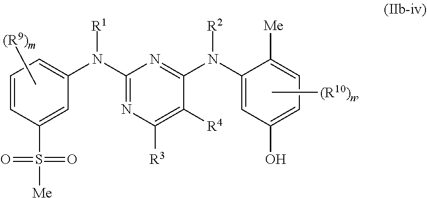

or a pharmaceutically acceptable salt, solvate, hydrate, polymorph, co-crystal, tautomer, stereoisomer, isotopically labeled derivative, or prodrug thereof.

In certain embodiments, a compound of Formula (II) is of Formula (IIb-v):

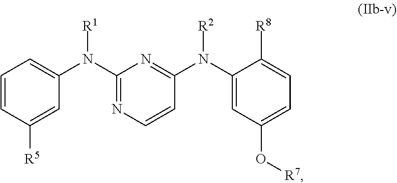

or a pharmaceutically acceptable salt, solvate, hydrate, polymorph, co-crystal, tautomer, stereoisomer, isotopically labeled derivative, or prodrug thereof.

In certain embodiments, a compound of Formula (II) is of Formula (IIb-vi):

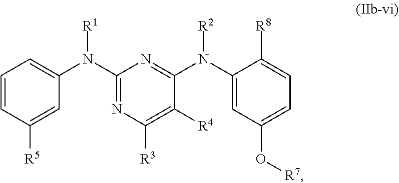

or a pharmaceutically acceptable salt, solvate, hydrate, polymorph, co-crystal, tautomer, stereoisomer, isotopically labeled derivative, or prodrug thereof.

The following chemical group definitions and embodiments apply to compounds of Formula (II) and all subgenera thereof provided herein.

As defined herein, R¹ is —H, optionally substituted aliphatic, optionally substituted acyl, or a nitrogen protecting group. In certain embodiments, R¹ is —H. In some embodiments, R¹ is substituted aliphatic. In certain embodiments, R¹ is unsubstituted aliphatic. In some embodiments, R¹ is substituted alkyl. In certain embodiments, R¹ is unsubstituted alkyl. In certain embodiments, R¹ is substituted C₁₋₆ alkyl. In some embodiments, R¹ is unsubstituted C₁₋₆ alkyl. In certain embodiments, R¹ is C₁ alkyl. In some embodiments, R¹ is C₂ alkyl. In certain embodiments, R¹ is C₃ alkyl. In some embodiments, R¹ is C₄ alkyl. In certain embodiments, R¹ is C₅ alkyl. In some embodiments, R¹ is C₆ alkyl. In certain embodiments, R¹ is methyl. In some embodiments, R¹ is ethyl. In certain embodiments, R¹ is n-propyl. In some embodiments, R¹ is isopropyl. In certain embodiments, R¹ is n-butyl. In some embodiments, R¹ is sec-butyl. In certain embodiments, R¹ is isobutyl. In some embodiments, R¹ is tert-butyl. In some embodiments, R¹ is substituted alkenyl. In certain embodiments, R¹ is unsubstituted alkenyl. In certain embodiments, R¹ is substituted C₁₋₆ alkenyl. In some embodiments, R¹ is unsubstituted C₁₋₆ alkenyl. In some embodiments, R¹ is substituted alkynyl. In certain embodiments, R¹ is unsubstituted alkynyl. In certain embodiments, R¹ is substituted C₁₋₆ alkynyl. In some embodiments, R¹ is unsubstituted C₁₋₆ alkynyl. In certain embodiments, R¹ is unsubstituted acyl. In some embodiments, R¹ is substituted acyl. In certain embodiments, R¹ is a nitrogen protecting group. In some embodiments, R¹ is Bn, Boc, Cbz, Fmoc, trifluoroacetyl, triphenylmethyl, acetyl, or Ts.

As defined herein, R² is —H, optionally substituted aliphatic, optionally substituted acyl, or a nitrogen protecting group. In certain embodiments, R² is —H. In some embodiments, R² is substituted aliphatic. In certain embodiments, R² is unsubstituted aliphatic. In some embodiments, R² is substituted alkyl. In certain embodiments, R² is unsubstituted alkyl. In certain embodiments, R² is substituted C₁₋₆ alkyl. In some embodiments, R² is unsubstituted C₁₋₆ alkyl. In certain embodiments, R² is C₁ alkyl. In some embodiments, R² is C₂ alkyl. In certain embodiments, R² is C₃ alkyl. In some embodiments, R² is C₄ alkyl. In certain embodiments, R² is C₅ alkyl. In some embodiments, R² is C₆ alkyl. In certain embodiments, R² is methyl. In some embodiments, R² is ethyl. In certain embodiments, R² is n-propyl. In some embodiments, R² is isopropyl. In certain embodiments, R² is n-butyl. In some embodiments, R² is sec-butyl. In certain embodiments, R² is isobutyl. In some embodiments, R² is tert-butyl. In some embodiments, R² is substituted alkenyl. In certain embodiments, R² is unsubstituted alkenyl. In certain embodiments, R² is substituted C₁₋₆ alkenyl. In some embodiments, R² is unsubstituted C₁₋₆ alkenyl. In some embodiments, R² is substituted alkynyl. In certain embodiments, R² is unsubstituted alkynyl. In certain embodiments, R² is substituted C₁₋₆ alkynyl. In some embodiments, R² is unsubstituted C₁₋₆ alkynyl. In certain embodiments, R² is unsubstituted acyl. In some embodiments, R² is substituted acyl. In certain embodiments, R² is a nitrogen protecting group. In some embodiments, R² is Bn, Boc, Cbz, Fmoc, trifluoroacetyl, triphenylmethyl, acetyl, or Ts.

In certain embodiments, R¹ is —H, and R² is optionally substituted aliphatic. In some embodiments, R¹ is —H, and R² is unsubstituted alkyl. In certain embodiments, R¹ is —H, and R² is unsubstituted C₁₋₆ alkyl. In some embodiments, R¹ is —H, and R² is methyl. In some embodiments, R¹ is —H, and R² is ethyl. In some embodiments, R¹ is —H, and R² is n-propyl. In certain embodiments, R¹ is —H, and R² is n-butyl.

In certain embodiments, R² is —H, and R¹ is optionally substituted aliphatic. In some embodiments, R² is —H, and R¹ is unsubstituted alkyl. In certain embodiments, R² is —H, and R¹ is unsubstituted C₁₋₆ alkyl. In some embodiments, R² is —H, and R¹ is methyl. In some embodiments, R² is —H, and R¹ is ethyl. In some embodiments, R² is —H, and R¹ is n-propyl. In certain embodiments, R² is —H, and R¹ is n-butyl.

In certain embodiments, R¹ and R² are both —H. In some embodiments, R¹ and R² are both -Me. In certain embodiments, R¹ and R² are each independently nitrogen protecting groups.

As defined herein, R³ is —H, halogen, —OR^{a3}, —N(R^{a3})₂, —CN, —SCN, —NO₂, —N₃, —SR^{a5}, optionally substituted aliphatic, optionally substituted heteroaliphatic, or optionally substituted acyl. In some embodiments, R³ is —H, —OR^{a3}, —N(R^{a3})₂, —CN, —SCN, —NO₂, —N₃, or —SR^{a3}. In certain embodiments, R³ is —H. In some embodiments, R³ is halogen. In certain embodiments, R³ is —F. In some embodiments, R³ is —Cl. In certain embodiments, R³ is —Br. In some embodiments, R³ is —I. In certain embodiments, R³ is —OR^{a3}. In some embodiments, R³ is —N(R^{a3})₂. In certain embodiments, R³ is —CN. In some embodiments, R³ is —SCN. In certain embodiments, R³ is —NO₂. In some embodiments, R³ is —N₃. In certain embodiments, R³ is —SR^{a3}. In certain embodiments, R³ is optionally substituted aliphatic. In some embodiments, R³ is substituted aliphatic. In certain embodiments, R³ is unsubstituted aliphatic. In some embodiments, R³ is substituted alkyl. In certain embodiments, R³ is haloalkyl. In some embodiments, R³ is unsubstituted alkyl. In certain embodiments, R³ is substituted C₁₋₆ alkyl. In some embodiments, R³ is C₁₋₆ haloalkyl. In some embodiments, R³ is unsubstituted C₁₋₆ alkyl. In some embodiments, R³ is substituted alkenyl. In certain embodiments, R³ is unsubstituted alkenyl. In certain embodiments, R³ is substituted C₁₋₆ alkenyl. In some embodiments, R³ is unsubstituted C₁₋₆ alkenyl. In some embodiments, R³ is substituted alkynyl. In certain embodiments, R³ is unsubstituted alkynyl. In certain embodiments, R³ is substituted C₁₋₆ alkynyl. In some embodiments, R³ is unsubstituted C₁₋₆ alkynyl. In certain embodiments, R³ is optionally substituted heteroaliphatic. In some embodiments, R³ is substituted heteroaliphatic. In certain embodiments, R³ is unsubstituted heteroaliphatic. In some embodiments, R³ is substituted heteroalkyl. In certain embodiments, R³ is unsubstituted heteroalkyl. In certain embodiments, R³ is substituted C₁₋₆ heteroalkyl. In some embodiments, R³ is unsubstituted C₁₋₆ heteroalkyl. In some embodiments, R³ is substituted heteroalkenyl. In certain embodiments, R³ is unsubstituted heteroalkenyl. In certain embodiments, R³ is substituted C₁₋₆ heteroalkenyl. In some embodiments, R³ is unsubstituted C₁₋₆ heteroalkenyl. In some embodiments, R³ is substituted heteroalkynyl. In certain embodiments, R³ is unsubstituted heteroalkynyl. In certain embodiments, R³ is substituted C₁₋₆ heteroalkynyl. In some embodiments, R³ is unsubstituted C₁₋₆ heteroalkynyl. In certain embodiments, R³ is optionally substituted acyl. In some embodiments, R³ is unsubstituted acyl. In certain embodiments, R³ is substituted acyl. In some embodiments, R³ is not -Me. In certain embodiments, R³ is not —C(═O)(substituted aryl).

As defined herein, each instance of R^{a3} is independently —H, optionally substituted aliphatic, optionally substituted heteroaliphatic, optionally substituted aryl, optionally substituted heteroaryl, optionally substituted carbocyclyl, optionally substituted heterocyclyl, optionally substituted acyl, optionally substituted sulfonyl, or optionally substituted sulfinyl, an oxygen protecting group when attached to an oxygen atom, a nitrogen protecting group when attached to a nitrogen atom, or a sulfur protecting group when attached to a sulfur atom; optionally wherein any two instances of R^{a3} attached to the same nitrogen atom are joined together with the intervening atoms to form optionally substituted heterocyclyl or optionally substituted heteroaryl. In some embodiments, each instance of R^{a3} is independently —H, optionally substituted aliphatic, optionally substituted heteroaliphatic, optionally substituted aryl, optionally substituted heteroaryl, optionally substituted carbocyclyl, optionally substituted heterocyclyl, optionally substituted acyl, optionally substituted sulfonyl, or optionally substituted sulfinyl, an oxygen protecting group when attached to an oxygen atom, a nitrogen protecting group when attached to a nitrogen atom, or a sulfur protecting group when attached to a sulfur atom. In certain embodiments, each instance of R^{a3} is independently —H, optionally substituted aliphatic, an oxygen protecting group when attached to an oxygen atom, a nitrogen protecting group when attached to a nitrogen atom, or a sulfur protecting group when attached to a sulfur atom. In some embodiments, each instance of R^{a3} is independently —H, or C₁₋₆ aliphatic, an oxygen protecting group when attached to an oxygen atom, a nitrogen protecting group when attached to a nitrogen atom, or a sulfur protecting group when attached to a sulfur atom.

As defined herein, R⁴ is —H, —Br, —OR^{a4}, —N(R^{a4})₂, —CN, —SCN, —N₃, —SR^{a4}, branched or unbranched aliphatic, optionally substituted heteroaliphatic, or optionally substituted acyl. In certain embodiments, R⁴ is —H, —OR^{a4}, —N(R^{a4})₂, —CN, —SCN, —N₃, —SR^{a4}, branched or unbranched aliphatic, optionally substituted heteroaliphatic, or optionally substituted acyl. In some embodiments, R⁴ is —H, —OR^{a4}, —N(R^{a4})₂, —CN, —SCN, —N₃, —SR^{a4}, optionally substituted heteroaliphatic, or optionally substituted acyl. In certain embodiments, R⁴ is —H or branched or unbranched C₂₋₂₀ aliphatic. In some embodiments, R⁴ is —H or C₂₋₂₀ branched or unbranched alkyl. In certain embodiments, R⁴ is —H. In some embodiments, R⁴ is —Br. In certain embodiments, R⁴ is —OR^{a4}. In some embodiments, R⁴ is —N(R^{a4})₂. In certain embodiments, R⁴ is —CN. In some embodiments, R⁴ is —SCN. In some embodiments, R⁴ is —N₃. In certain embodiments, R⁴ is —SR^{a4}. In certain embodiments, R⁴ is branched or unbranched aliphatic. In some embodiments, R⁴ is branched aliphatic. In certain embodiments, R⁴ is unbranched aliphatic. In some embodiments, R⁴ is branched alkyl. In certain embodiments, R⁴ is unbranched alkyl. In certain embodiments, R⁴ is branched C₁₋₆ alkyl. In some embodiments, R⁴ is unbranched C₁₋₈ alkyl. In some embodiments, R⁴ is branched alkenyl. In certain embodiments, R⁴ is unbranched alkenyl. In certain embodiments, R⁴ is branched C₁₋₆ alkenyl. In some embodiments, R⁴ is unbranched C₁₋₆ alkenyl. In some embodiments, R⁴ is branched alkynyl. In certain embodiments, R⁴ is unbranched alkynyl. In certain embodiments, R⁴ is branched C₁₋₆ alkynyl. In some embodiments, R⁴ is unbranched C₁₋₆ alkynyl. In certain embodiments, R⁴ is optionally substituted heteroaliphatic. In some embodiments, R⁴ is substituted heteroaliphatic. In certain embodiments, R⁴ is unsubstituted heteroaliphatic. In some embodiments, R⁴ is substituted heteroalkyl. In certain embodiments, R⁴ is unsubstituted heteroalkyl. In certain embodiments, R⁴ is substituted C₁₋₆ heteroalkyl. In some embodiments, R⁴ is unsubstituted C₁₋₆ heteroalkyl. In some embodiments, R⁴ is substituted heteroalkenyl. In certain embodiments, R⁴ is unsubstituted heteroalkenyl. In certain embodiments, R⁴ is substituted C₁₋₆ heteroalkenyl. In some embodiments, R⁴ is unsubstituted C₁₋₆ heteroalkenyl. In some embodiments, R⁴ is substituted heteroalkynyl. In certain embodiments, R⁴ is unsubstituted heteroalkynyl. In certain embodiments, R⁴ is substituted C₁₋₆ heteroalkynyl. In some embodiments, R⁴ is unsubstituted C₁₋₆ heteroalkynyl. In certain embodiments, R⁴ is optionally substituted acyl. In some embodiments, R⁴ is unsubstituted acyl. In certain embodiments, R⁴ is substituted acyl.

As defined herein, each instance of R^{a4} is independently —H, optionally substituted aliphatic, optionally substituted heteroaliphatic, optionally substituted aryl, optionally substituted heteroaryl, optionally substituted carbocyclyl, optionally substituted heterocyclyl, optionally substituted acyl, optionally substituted sulfonyl, or optionally substituted sulfinyl, an oxygen protecting group when attached to an oxygen atom, a nitrogen protecting group when attached to a nitrogen atom, or a sulfur protecting group when attached to a sulfur atom; optionally wherein any two instances of R^{a4} attached to the same nitrogen atom are joined together with the intervening atoms to form optionally substituted heterocyclyl or optionally substituted heteroaryl. In some embodiments, each instance of R^{a4} is independently —H, optionally substituted aliphatic, optionally substituted heteroaliphatic, optionally substituted aryl, optionally substituted heteroaryl, optionally substituted carbocyclyl, optionally substituted heterocyclyl, optionally substituted acyl, optionally substituted sulfonyl, or optionally substituted sulfinyl, an oxygen protecting group when attached to an oxygen atom, a nitrogen protecting group when attached to a nitrogen atom, or a sulfur protecting group when attached to a sulfur atom. In certain embodiments, each instance of R^{a4} is independently —H, optionally substituted aliphatic, an oxygen protecting group when attached to an oxygen atom, a nitrogen protecting group when attached to a nitrogen atom, or a sulfur protecting group when attached to a sulfur atom. In some embodiments, each instance of R^{a4} is independently —H, or C₁₋₆ aliphatic, an oxygen protecting group when attached to an oxygen atom, a nitrogen protecting group when attached to a nitrogen atom, or a sulfur protecting group when attached to a sulfur atom.

In certain embodiments, R³ and R⁴ are both —H. In some embodiments, R³ is —H, and R⁴ is —H, halogen, —OR^{a4}, —N(R^{a4})₂, —CN, —SCN, —NO₂, —N₃, —SR^{a4}, optionally substituted aliphatic, optionally substituted heteroaliphatic, or optionally substituted acyl. In certain embodiments, R⁴ is —H, and R³ is —H, halogen, —OR^{a3}, —N(R^{a3})₂, —CN, —SCN, —NO₂, —N₃, —SR^{a3}, optionally substituted aliphatic, optionally substituted heteroaliphatic, or optionally substituted acyl.

As defined herein, R⁵ is —H, —S(═O)₂N(R^{a5})₂, —S(═O)₂OR^{a5}, —S(═O)₂R^{a5}, —S(═O)₂Cl, —SR^{a5}, —C(═O)N(R^{a6})₂, —C(═O)OR^{a6}, or —C(═O)R^{a6}; provided that R⁵ and R⁶ are not both —H. In certain embodiments, R⁵ is —H, —S(═O)₂N(R^{a5})₂, —S(═O)₂OR^{a5}, —S(═O)₂R^{a5}, —S(═O)₂Cl, —SR^{a5}, —C(═O)N(R^{a6})₂, —C(═O)OR^{a6}, or —C(═O)R^{a6}. In some embodiments, R⁵ is —H, —S(═O)₂N(R^{a5})₂, —S(═O)₂OR^{a5}, —S(═O)₂R^{a5}, —S(═O)₂Cl, or —SR^{a5}. In certain embodiments, R⁵ is —H, —S(═O)₂OR^{a5}, —S(═O)₂R^{a5}, —S(═O)₂Cl, or —SR^{a5}. In some embodiments, R⁵ is —H. In certain embodiments, R⁵ is —S(═O)₂N(R^{a5})₂. In some embodiments, R⁵ is —S(═O)₂NH₂. In certain embodiments, R⁵ is —S(═O)₂NH(R^{a5}), wherein R^{a5} is C₁₋₆ aliphatic. In some embodiments, R⁵ is —S(═O)₂N(R^{a5})₂, wherein R^{a5} is C₁₋₆ aliphatic. In certain embodiments, R⁵ is —S(═O)₂OR^{a5}. In some embodiments, R⁵ is —S(═O)₂OH. In certain embodiments, R⁵ is —S(═O)₂OR^{a5}, wherein R^{a5} is C₁₋₆ aliphatic. In some embodiments, R⁵ is —S(═O)₂R^{a5}. In certain embodiments, R⁵ is —S(═O)₂R^{a5}, wherein R^{a5} is C₁₋₆ aliphatic. In some embodiments, R⁵ is —S(═O)₂Cl. In certain embodiments, R⁵ is —SR^{a5}. In some embodiments, R⁵ is —SH. In certain embodiments, R⁵ is —SR^{a5}, wherein R^{a5} is C₁₋₆ aliphatic. In some embodiments, R⁵ is —SR^{a5}, wherein R^{a5} is a sulfur protecting group.

As defined herein, each instance of R^{a5} is independently —H, optionally substituted aliphatic, optionally substituted heteroaliphatic, optionally substituted aryl, optionally substituted heteroaryl, optionally substituted carbocyclyl, optionally substituted heterocyclyl, optionally substituted acyl, optionally substituted sulfonyl, or optionally substituted sulfinyl, an oxygen protecting group when attached to an oxygen atom, a nitrogen protecting group when attached to a nitrogen atom, or a sulfur protecting group when attached to a sulfur atom; optionally wherein any two instances of R^{a5} attached to the same nitrogen atom are joined together with the intervening atoms to form optionally substituted heterocyclyl or optionally substituted heteroaryl. In some embodiments, each instance of R^{a5} is independently —H, optionally substituted aliphatic, optionally substituted heteroaliphatic, optionally substituted aryl, optionally substituted heteroaryl, optionally substituted carbocyclyl, optionally substituted heterocyclyl, optionally substituted acyl, optionally substituted sulfonyl, or optionally substituted sulfinyl, an oxygen protecting group when attached to an oxygen atom, a nitrogen protecting group when attached to a nitrogen atom, or a sulfur protecting group when attached to a sulfur atom. In certain embodiments, each instance of R^{a5} is independently —H, optionally substituted aliphatic, an oxygen protecting group when attached to an oxygen atom, a nitrogen protecting group when attached to a nitrogen atom, or a sulfur protecting group when attached to a sulfur atom. In some embodiments, each instance of R^{a} is independently —H, or C₁₋₆ aliphatic, an oxygen protecting group when attached to an oxygen atom, a nitrogen protecting group when attached to a nitrogen atom, or a sulfur protecting group when attached to a sulfur atom.

As defined herein, R⁶ is —H, —S(═O)₂N(R^{a5})₂, —S(═O)₂OR^{a5}, —S(═O)₂R^{a5}, —S(═O)₂Cl, —SR^{a5}, —C(═O)N(R^{a6})₂, —C(═O)OR^{a6}, or —C(═O)R^{a6}; provided that R⁵ and R⁶ are not both —H. In certain embodiments, R⁶ is —H, —S(═O)₂N(R^{a5})₂, —S(═O)₂OR^{a5}, —S(═O)₂R^{a5}, —S(═O)₂Cl, —SR^{a5}, —C(═O)N(R^{a6})₂, —C(═O)OR^{a6}, or —C(═O)R^{a6}. In some embodiments, R⁶ is —H, —C(═O)N(R^{a6})₂, —C(═O)OR^{a6}, or —C(═O)R^{a6}. In certain embodiments, R⁶ is —H, —C(═O)N(R^{a6})₂, or —C(═O)OR^{a6}. In some embodiments, R⁶ is —H, —C(═O)N(R^{a6})₂, or —C(═O)R^{a6}. In certain embodiments, R⁶ is —H or —C(═O)N(R^{a6})₂. In some embodiments, R⁶ is —H. In certain embodiments, R⁶ is —C(═O)N(R^{a6})₂. In some embodiments, R⁶ is —C(═O)OR^{a6}. In certain embodiments, R⁶ is —C(═O)R^{a6}. In some embodiments, R⁶ is —C(═O)NH₂. In certain embodiments, R⁶ is —C(═O)NH(R^{a6}), wherein R^{a6} is C₁₋₆ aliphatic. In some embodiments, R⁶ is —C(═O)N(R^{a6})₂, wherein R^{a6} is C₁₋₆ aliphatic. In certain embodiments, R⁶ is —C(═O)OH. In some embodiments, R⁶ is —C(═O)OH, wherein R^{a6} is C₁₋₆ aliphatic. In certain embodiments, R⁶ is —C(═O)H. In some embodiments, R⁶ is —C(═O)R^{a6}, wherein R^{a6} is C₁₋₆ aliphatic.

As defined herein, each instance of R^{a6} is independently —H, optionally substituted aliphatic, optionally substituted heteroaliphatic, optionally substituted aryl, optionally substituted heteroaryl, optionally substituted carbocyclyl, optionally substituted heterocyclyl, optionally substituted acyl, optionally substituted sulfonyl, or optionally substituted sulfinyl, an oxygen protecting group when attached to an oxygen atom, a nitrogen protecting group when attached to a nitrogen atom, or a sulfur protecting group when attached to a sulfur atom; optionally wherein any two instances of R^{a6} attached to the same nitrogen atom are joined together with the intervening atoms to form optionally substituted heterocyclyl or optionally substituted heteroaryl. In some embodiments, each instance of R^{a6} is independently —H, optionally substituted aliphatic, optionally substituted heteroaliphatic, optionally substituted aryl, optionally substituted heteroaryl, optionally substituted carbocyclyl, optionally substituted heterocyclyl, optionally substituted acyl, optionally substituted sulfonyl, or optionally substituted sulfinyl, an oxygen protecting group when attached to an oxygen atom, a nitrogen protecting group when attached to a nitrogen atom, or a sulfur protecting group when attached to a sulfur atom. In certain embodiments, each instance of R^{a6} is independently —H, optionally substituted aliphatic, an oxygen protecting group when attached to an oxygen atom, a nitrogen protecting group when attached to a nitrogen atom, or a sulfur protecting group when attached to a sulfur atom. In some embodiments, each instance of R^{a6} is independently —H, or C₁₋₆ aliphatic, an oxygen protecting group when attached to an oxygen atom, or a nitrogen protecting group when attached to a nitrogen atom, or a sulfur protecting group when attached to a sulfur atom.

In certain embodiments, R⁵ is —S(═O)₂N(R^{a5})₂, —S(═O)₂OR^{a5}, —S(═O)₂R^{a5}, —S(═O)₂Cl, —SR^{a5}, —C(═O)N(R^{a6})₂, —C(═O)OR^{a6}, or —C(═O)R^{a6}, and R⁶ is —H. In some embodiments, R⁵ is —H, and R⁶ is —S(═O)₂N(R^{a5})₂, —S(═O)₂OR^{a5}, —S(═O)₂R^{a5}, —S(═O)₂Cl, —SR^{a5}, —C(═O)N(R^{a6})₂, —C(═O)OR^{a6}, or —C(═O)R^{a6}. In certain embodiments, R⁵ is —S(═O)₂N(R^{a5})₂, —S(═O)₂OR^{a5}, —S(═O)₂R^{a5}, —S(═O)₂Cl, or —SR^{a5}, and R⁶ is —H. In some embodiments, R⁵ is —S(═O)₂OR^{a5}, —S(═O)₂R^{a5}, —S(═O)₂Cl, or —SR^{a5}, and R⁶ is —H. In certain embodiments, R⁵ is —S(═O)₂R^{a5}, and R⁶ is —H. In some embodiments, R⁵ is —H, and R⁶ is —C(═O)N(R^{a6})₂, —C(═O)OR^{a6}, or —C(═O)R^{a6}. In certain embodiments, R⁵ is —H, and R⁶ is —C(═O)N(R^{a6})₂ or —C(═O)OR^{a6}. In some embodiments, R⁵ is —H, and R⁶ is —C(═O)N(R^{a6})₂ or —C(═O)R^{a6}. In certain embodiments, R⁵ is —H, and R⁶ is —C(═O)N(R^{a6})₂.

As defined herein, R⁷ is —H, optionally substituted aliphatic, optionally substituted acyl, or an oxygen protecting group. In some embodiments, R⁷ is —H, optionally substituted C₁₋₆ alkyl, or an oxygen protecting group. In certain embodiments, R⁷ is —H. In some embodiments, R⁷ is optionally substituted aliphatic. In certain embodiments, R⁷ is substituted aliphatic. In some embodiments, R⁷ is unsubstituted aliphatic. In certain embodiments, R⁷ is substituted alkyl. In some embodiments, R⁷ is unsubstituted alkyl. In certain embodiments, R⁷ is substituted C₁₋₆ alkyl. In some embodiments, R⁷ is unsubstituted C₁₋₆ alkyl. In certain embodiments, R⁷ is substituted alkenyl. In some embodiments, R⁷ is unsubstituted alkenyl. In certain embodiments, R⁷ is substituted C₁₋₆ alkenyl. In some embodiments, R⁷ is unsubstituted C₁₋₆ alkenyl. In certain embodiments, R⁷ is substituted alkynyl. In some embodiments, R⁷ is unsubstituted alkynyl. In certain embodiments, R⁷ is substituted C₁₋₆ alkynyl. In some embodiments, R⁷ is unsubstituted C₁₋₆ alkynyl. In certain embodiments, R⁷ is optionally substituted acyl. In some embodiments, R⁷ is unsubstituted acyl. In certain embodiments, R⁷ is substituted acyl. In some embodiments, R⁷ is an oxygen protecting group. In certain embodiments, R⁷ is silyl, TBDPS, TBDMS, TIPS, TES, TMS, MOM, THP, t-Bu, Bn, allyl, acetyl, pivaloyl, or benzoyl.

As defined herein, R⁸ is —N(R^{a8})₂, —CN, —SCN, —NO₂, —N₃, —SR^{a8}, halogen, optionally substituted aliphatic, optionally substituted acyl, optionally substituted sulfonyl, or optionally substituted sulfinyl. In some embodiments, R⁸ is —N(R^{a8})₂, —SR^{a8}, halogen, optionally substituted aliphatic, or optionally substituted heteroaliphatic. In some embodiments, R⁸ is —N(R^{a8})₂. In certain embodiments, R⁸ is —CN. In some embodiments, R⁸ is —SCN. In certain embodiments, R⁸ is —NO₂. In some embodiments, R⁸ is —N₃. In certain embodiments, R⁸ is —SR^{a8}. In some embodiments, R⁸ is halogen. In certain embodiments, R⁸ is —F. In some embodiments, R⁸ is —Cl. In certain embodiments, R⁸ is —Br. In some embodiments, R⁸ is —I. In certain embodiments, R⁸ is optionally substituted aliphatic. In some embodiments, R⁸ is substituted aliphatic. In certain embodiments, R⁸ is unsubstituted aliphatic. In some embodiments, R⁸ is substituted alkyl. In certain embodiments, R⁸ is unsubstituted alkyl. In certain embodiments, R⁸ is optionally substituted C₁₋₆ alkyl. In some embodiments, R⁸ is substituted C₁₋₆ alkyl. In certain embodiments, R⁸ is unsubstituted C₁₋₆ alkyl. In certain embodiments, R⁸ is C₁ alkyl. In some embodiments, R⁸ is C₂ alkyl. In certain embodiments, R⁸ is C₃ alkyl. In some embodiments, R⁸ is C₄ alkyl. In certain embodiments, R⁸ is C₅ alkyl. In some embodiments, R⁸ is C₆ alkyl. In certain embodiments, R⁸ is methyl. In some embodiments, R⁸ is ethyl. In certain embodiments, R⁸ is n-propyl. In some embodiments, R⁸ is isopropyl. In certain embodiments, R⁸ is n-butyl. In some embodiments, R⁸ is sec-butyl. In certain embodiments, R⁸ is isobutyl. In some embodiments, R⁸ is tert-butyl. In some embodiments, R⁸ is substituted alkenyl. In certain embodiments, R⁸ is unsubstituted alkenyl. In certain embodiments, R⁸ is substituted C₁₋₆ alkenyl. In some embodiments, R⁸ is unsubstituted C₁₋₆ alkenyl. In some embodiments, R⁸ is substituted alkynyl. In certain embodiments, R⁸ is unsubstituted alkynyl. In certain embodiments, R⁸ is substituted C₁₋₆ alkynyl. In some embodiments, R⁸ is unsubstituted C₁₋₆ alkynyl. In certain embodiments, R⁸ is optionally substituted acyl. In some embodiments, R⁸ is unsubstituted acyl. In certain embodiments, R⁸ is substituted acyl. In certain embodiments, R⁸ is optionally substituted sulfonyl. In some embodiments, R⁸ is unsubstituted sulfonyl. In certain embodiments, R⁸ is substituted sulfonyl. In certain embodiments, R⁸ is or optionally substituted sulfinyl. In some embodiments, R⁸ is unsubstituted sulfinyl. In certain embodiments, R⁸ is substituted sulfinyl.

As defined herein, each instance of R^{a8} is independently —H, optionally substituted aliphatic, optionally substituted heteroaliphatic, optionally substituted aryl, optionally substituted heteroaryl, optionally substituted carbocyclyl, optionally substituted heterocyclyl, optionally substituted acyl, optionally substituted sulfonyl, or optionally substituted sulfinyl, an oxygen protecting group when attached to an oxygen atom, a nitrogen protecting group when attached to a nitrogen atom, or a sulfur protecting group when attached to a sulfur atom; optionally wherein any two instances of R^{a8} attached to the same nitrogen atom are joined together with the intervening atoms to form optionally substituted heterocyclyl or optionally substituted heteroaryl. In some embodiments, each instance of R^{a8} is independently —H, optionally substituted aliphatic, optionally substituted heteroaliphatic, optionally substituted aryl, optionally substituted heteroaryl, optionally substituted carbocyclyl, optionally substituted heterocyclyl, optionally substituted acyl, optionally substituted sulfonyl, or optionally substituted sulfinyl, an oxygen protecting group when attached to an oxygen atom, a nitrogen protecting group when attached to a nitrogen atom, or a sulfur protecting group when attached to a sulfur atom. In certain embodiments, each instance of R^{a8} is independently —H, optionally substituted aliphatic, an oxygen protecting group when attached to an oxygen atom, a nitrogen protecting group when attached to a nitrogen atom, or a sulfur protecting group when attached to a sulfur atom. In some embodiments, each instance of R^{a8} is independently —H, or C₁₋₆ aliphatic, an oxygen protecting group when attached to an oxygen atom, a nitrogen protecting group when attached to a nitrogen atom, or a sulfur protecting group when attached to a sulfur atom.

In certain embodiments, R⁷ is —H, and R⁸ is optionally substituted C₁₋₆ alkyl. In some embodiments, R⁷ is —H, and R⁸ is C₁ alkyl. In certain embodiments, R⁷ is —H, and R⁸ is C₂ alkyl. In some embodiments, R⁷ is —H, and R⁸ is C₃ alkyl. In certain embodiments, R⁷ is —H, and R⁸ is C₄ alkyl. In some embodiments, R⁷ is —H, and R⁸ is C₅ alkyl. In certain embodiments, R⁷ is —H, and R⁸ is C₆ alkyl. In some embodiments, R⁷ is —H, and R⁸ is methyl. In certain embodiments, R⁷ is —H, and R⁸ is ethyl.

In certain embodiments, R⁷ and R⁸ are each independently optionally substituted C₁₋₆ alkyl. In some embodiments, R⁷ is unsubstituted C₁₋₆ alkyl, and R⁸ is substituted C₁₋₆ alkyl. In certain embodiments, R⁷ is substituted C₁₋₆ alkyl, and R⁸ is unsubstituted C₁₋₆ alkyl. In some embodiments, R⁷ and R⁸ are each independently unsubstituted C₁₋₆ alkyl. In certain embodiments, R⁷ and R⁸ are each independently substituted C₁₋₆ alkyl.

In certain embodiments, R⁵ is —S(═O)₂R^{a5}, and R⁸ is optionally substituted C₁₋₆ alkyl. In some embodiments, R⁵ is —S(═O)₂R^{a5}, and R⁸ is substituted C₁₋₆ alkyl. In certain embodiments, R⁵ is —S(═O)₂R^{a5}, and R⁸ is unsubstituted C₁₋₆ alkyl. In some embodiments, R⁵ is —S(═O)₂NH₂, and R⁸ is not -Me. In certain embodiments, R⁵ is —S(═O)₂NH₂, and R⁸ is not unsubstituted C₁₋₆ alkyl. In some embodiments, R⁵ is —S(═O)₂NH₂, and R⁸ is not —NH₂.

In certain embodiments, R⁶ is —C(═O)N(R^{a6})₂, and R⁸ is optionally substituted C₁₋₆ alkyl. In some embodiments, R⁶ is —C(═O)N(R^{a6})₂, and R⁸ is substituted C₁₋₆ alkyl. In certain embodiments, R⁶ is —C(═O)N(R^{a6})₂, and R⁸ is unsubstituted C₁₋₆ alkyl.

As defined herein, each instance of R⁹ is independently —H, —OR^{a9}, —C(═O)R^{a9}, —C(═O)OR^{a9}, —OC(═O)R^{a9}, —N(R^{a9})₂, —N(C═O)R^{a9}, —(C═O)N(R^{a9})₂, —CN, —SCN, —NO₂, —N₃, —SR^{a9}, halogen, optionally substituted aliphatic, optionally substituted heteroaliphatic, optionally substituted sulfonyl, or optionally substituted sulfinyl. In some embodiments, R⁹ is —H, —OR^{a9}, —C(═O)R^{a9}, —C(═O)OR^{a9}, —OC(═O)R^{a9}, —N(R^{a9})₂, —N(C═O)R^{a9}, —(C═O)N(R^{a9})₂, —CN, —NO₂, —SR^{a9}, halogen, optionally substituted aliphatic, or optionally substituted heteroaliphatic. In some embodiments, each instance of R⁹ is independently —H, —OR^{a9}, —C(═O)R^{a9}, —C(═O)OR^{a9}, —OC(═O)R^{a9}, —N(C═O)R^{a9}, —(C═O)N(R^{a9})₂, —CN, —SCN, —NO₂, —N₃, —SR^{a9}, halogen, optionally substituted aliphatic, optionally substituted sulfonyl, or optionally substituted sulfinyl. In some embodiments, R⁹ is —H, —OR^{a9}, —C(═O)R^{a9}, —C(═O)OR^{a9}, —OC(═O)R^{a9}, —N(R^{a9})₂, —N(C═O)R^{a9}, —(C═O)N(R^{a9})₂, —CN, —NO₂, —SR^{a9}, halogen, optionally substituted aliphatic, or optionally substituted heteroaliphatic. In certain embodiments, R⁹ is —H. In some embodiments, R⁹ is —OR^{a9}. In certain embodiments, R⁹ is —C(═O)R^{a9}. In some embodiments, R⁹ is —OC(═O)R^{a9}. In certain embodiments, R⁹ is —C(═O)OR^{a9}. In some embodiments, R⁹ is —N(R^{a9})₂. In certain embodiments, R⁹ is —N(C═O)R^{a9}. In some embodiments, R⁹ is —(C═O)N(R^{a9})₂. In certain embodiments, R⁹ is —CN. In some embodiments, R⁹ is —SCN. In certain embodiments, R⁹ is —NO₂. In some embodiments, R⁹ is —N₃. In certain embodiments, R⁹ is —SR^{a9}. In some embodiments, R⁹ is halogen. In certain embodiments, R⁹ is —F. In some embodiments, R⁹ is —Cl. In certain embodiments, R⁹ is —Br. In some embodiments, R⁹ is —I. In certain embodiments, R⁹ is optionally substituted aliphatic. In some embodiments, R⁹ is unsubstituted aliphatic. In certain embodiments, R⁹ is substituted aliphatic. In some embodiments, R⁹ is unsubstituted C₁₋₆ aliphatic. In certain embodiments, R⁹ is substituted C₁₋₆ aliphatic. In some embodiments, R⁹ is optionally substituted heteroaliphatic. In certain embodiments, R⁹ is unsubstituted heteroaliphatic. In some embodiments, R⁹ is substituted heteroaliphatic. In certain embodiments, R⁹ is unsubstituted C₁₋₆ heteroaliphatic. In some embodiments, R⁹ is substituted C₁₋₆ heteroaliphatic. In certain embodiments, R⁹ is optionally substituted sulfonyl. In some embodiments, R⁹ is unsubstituted sulfonyl. In certain embodiments, R⁹ is substituted sulfonyl. In certain embodiments, R⁹ is optionally substituted sulfinyl. In some embodiments, R⁹ is unsubstituted sulfinyl. In certain embodiments, R⁹ is substituted sulfinyl.

As defined herein, each instance of R^{a9} is independently —H, optionally substituted aliphatic, optionally substituted heteroaliphatic, optionally substituted aryl, optionally substituted heteroaryl, optionally substituted carbocyclyl, optionally substituted heterocyclyl, optionally substituted acyl, optionally substituted sulfonyl, or optionally substituted sulfinyl, an oxygen protecting group when attached to an oxygen atom, a nitrogen protecting group when attached to a nitrogen atom, or a sulfur protecting group when attached to a sulfur atom; optionally wherein any two instances of R^{a9} attached to the same nitrogen atom are joined together with the intervening atoms to form optionally substituted heterocyclyl or optionally substituted heteroaryl. In some embodiments, each instance of R^{a9} is independently —H, optionally substituted aliphatic, optionally substituted heteroaliphatic, optionally substituted aryl, optionally substituted heteroaryl, optionally substituted carbocyclyl, optionally substituted heterocyclyl, optionally substituted acyl, optionally substituted sulfonyl, or optionally substituted sulfinyl, an oxygen protecting group when attached to an oxygen atom, a nitrogen protecting group when attached to a nitrogen atom, or a sulfur protecting group when attached to a sulfur atom. In certain embodiments, each instance of R^{a9} is independently —H, optionally substituted aliphatic, optionally substituted aryl, optionally substituted heteroaryl, optionally substituted carbocyclyl, optionally substituted acyl, optionally substituted sulfonyl, or optionally substituted sulfinyl, an oxygen protecting group when attached to an oxygen atom, a nitrogen protecting group when attached to a nitrogen atom, or a sulfur protecting group when attached to a sulfur atom. In certain embodiments, each instance of R^{a9} is independently —H, optionally substituted aliphatic, an oxygen protecting group when attached to an oxygen atom, a nitrogen protecting group when attached to a nitrogen atom, or a sulfur protecting group when attached to a sulfur atom. In some embodiments, each instance of R^{a9} is independently —H, or C₁₋₆ aliphatic, an oxygen protecting group when attached to an oxygen atom, a nitrogen protecting group when attached to a nitrogen atom, or a sulfur protecting group when attached to a sulfur atom.

In certain embodiments, R⁵ is —S(═O)₂N(R^{a5})₂, —S(═O)₂OR^{a5}, —S(═O)₂R^{a5}, —S(═O)₂Cl, —SR^{a5}, —C(═O)N(R^{a6})₂, —C(═O)OR^{a6}, or —C(═O)R^{a6}, R⁶ is —H, and each instance of R⁹ is —H. In some embodiments, R⁵ is —H, R⁶ is —S(═O)₂N(R^{a5})₂, —S(═O)₂OR^{a5}, —S(═O)₂R^{a5}, —S(═O)₂Cl, —SR^{a5}, —C(═O)N(R^{a6})₂, —C(═O)OR^{a6}, or —C(═O)R^{a6}, and each instance of R⁹ is —H. In certain embodiments, R⁵ is —S(═O)₂N(R^{a5})₂, —S(═O)₂OR^{a5}, —S(═O)₂R^{a5}, —S(═O)₂Cl, or —SR^{a5}, R⁶ is —H, and each instance of R⁹ is —H. In some embodiments, R⁵ is —S(═O)₂OR^{al}, —S(═O)₂R^{a5}, —S(═O)₂Cl, or —SR^{a5}, R⁶ is —H, and each instance of R⁹ is —H. In certain embodiments, R⁵ is —S(═O)₂R^{a5}, R⁶ is —H, and each instance of R⁹ is —H. In some embodiments, R⁵ is —H, R⁶ is —C(═O)N(R^{a6})₂, —C(═O)OR^{a6}, or —C(═O)R^{a6}, and each instance of R⁹ is —H. In certain embodiments, R⁵ is —H, R⁶ is —C(═O)N(R^{a6})₂ or —C(═O)OR^{a6}, and each instance of R⁹ is —H. In some embodiments, R⁵ is —H, R⁶ is —C(═O)N(R^{a6})₂ or —C(═O)R^{a6}, and each instance of R⁹ is —H. In certain embodiments, R⁵ is —H, R⁶ is —C(═O)N(R^{a6})₂, and each instance of R⁹ is —H.

As defined herein, each instance of R¹⁰ is independently —H, —C(═O)R^{a10}, —C(═O)OR^{a10}, —OC(═O)R^{a10}, —N(C═O)R^{a10}, —(C═O)N(R^{a10})₂, —CN, —SCN, —NO₂, —N₃, —SR^{a10}, halogen, optionally substituted aliphatic, optionally substituted heteroaliphatic, optionally substituted sulfonyl, or optionally substituted sulfinyl. In certain embodiments, R¹⁰ is —H, —C(═O)R^{a10}, —C(═O)OR^{a10}, —OC(═O)R^{a10}, —N(C═O)R^{a10}, —(C═O)N(R^{a10})₂—CN, —NO₂, —SR^{a10}, halogen, optionally substituted aliphatic, or optionally substituted heteroaliphatic. In some embodiments, R¹⁰ is —H, —C(═O)R^{a10}, —C(═O)OR^{a10}, —OC(═O)R^{a10}, —N(C═O)R^{a10}, —(C═O)N(R^{a10})₂—CN, —NO₂, —SR^{a10}, halogen, or optionally substituted aliphatic. In certain embodiments, R¹⁰ is —H. In certain embodiments, R¹⁰ is —C(═O)R^{a10}. In some embodiments, R¹⁰ is —OC(═O)R^{a10}. In certain embodiments, R¹⁰ is —C(═O)OR^{a10}. In certain embodiments, R¹⁰ is —N(C═O)R^{a10}. In some embodiments, R¹⁰ is —(C═O)N(R^{a10})₂. In certain embodiments, R¹⁰ is —CN. In some embodiments, R¹⁰ is —SCN. In certain embodiments, R¹⁰ is —NO₂. In some embodiments, R¹⁰ is —N₃. In certain embodiments, R¹⁰ is —SR^{a10}. In some embodiments, R¹⁰ is halogen. In certain embodiments, R¹⁰ is —F. In some embodiments, R¹⁰ is —Cl. In certain embodiments, R¹⁰ is —Br. In some embodiments, R¹⁰ is —I. In certain embodiments, R¹⁰ is optionally substituted aliphatic. In some embodiments, R¹⁰ is unsubstituted aliphatic. In certain embodiments, R¹⁰ is substituted aliphatic. In some embodiments, R¹⁰ is unsubstituted C₁₋₆ aliphatic. In certain embodiments, R¹⁰ is substituted C₁₋₆ aliphatic. In some embodiments, R¹⁰ is optionally substituted heteroaliphatic. In certain embodiments, R¹⁰ is unsubstituted heteroaliphatic. In some embodiments, R¹⁰ is substituted heteroaliphatic. In certain embodiments, R¹⁰ is unsubstituted C₁₋₆ heteroaliphatic. In some embodiments, R¹⁰ is substituted C₁₋₆ heteroaliphatic. In certain embodiments, R¹⁰ is optionally substituted sulfonyl. In some embodiments, R¹⁰ is unsubstituted sulfonyl. In certain embodiments, R¹⁰ is substituted sulfonyl. In certain embodiments, R¹⁰ is optionally substituted sulfinyl. In some embodiments, R¹⁰ is unsubstituted sulfinyl. In certain embodiments, R¹⁰ is substituted sulfinyl.

In some embodiments, R⁸ is —N(R^{a8})₂, —CN, —SCN, —NO₂, —N₃, —SR^{a8}, halogen, optionally substituted aliphatic, optionally substituted acyl, optionally substituted sulfonyl, or optionally substituted sulfinyl, and each instance of R¹⁰ is —H. In some embodiments, R⁸ is optionally substituted aliphatic, and each instance of R¹⁰ is —H. In certain embodiments, R⁸ is optionally substituted C₁₋₆ alkyl, and each instance of R¹⁰ is —H.

In some embodiments, R⁷ and R⁸ are each independently optionally substituted C₁₋₆ alkyl, and each instance of R¹⁰ is —H. In certain embodiments, R⁷ and R⁸ are each independently unsubstituted C₁₋₆ alkyl, and each instance of R¹⁰ is —H. In some embodiments, R⁷ is —H, R⁸ is optionally substituted C₁₋₆ alkyl, and each instance of R¹⁰ is —H. In certain embodiments, R⁷ is —H, R⁸ is unsubstituted C₁₋₆ alkyl, and each instance of R¹⁰ is —H.

As defined herein, each instance of R^{a10} is independently —H, optionally substituted aliphatic, optionally substituted heteroaliphatic, optionally substituted aryl, optionally substituted heteroaryl, optionally substituted carbocyclyl, optionally substituted heterocyclyl, optionally substituted acyl, optionally substituted sulfonyl, or optionally substituted sulfinyl, an oxygen protecting group when attached to an oxygen atom, a nitrogen protecting group when attached to a nitrogen atom, or a sulfur protecting group when attached to a sulfur atom; optionally wherein any two instances of R^{a10} attached to the same nitrogen atom are joined together with the intervening atoms to form optionally substituted heterocyclyl or optionally substituted heteroaryl. In some embodiments, each instance of R^{a10} is independently —H, optionally substituted aliphatic, optionally substituted heteroaliphatic, optionally substituted aryl, optionally substituted heteroaryl, optionally substituted carbocyclyl, optionally substituted heterocyclyl, optionally substituted acyl, optionally substituted sulfonyl, or optionally substituted sulfinyl, an oxygen protecting group when attached to an oxygen atom, a nitrogen protecting group when attached to a nitrogen atom, or a sulfur protecting group when attached to a sulfur atom. In certain embodiments, each instance of R^{a10} is independently —H, optionally substituted aliphatic, an oxygen protecting group when attached to an oxygen atom, a nitrogen protecting group when attached to a nitrogen atom, or a sulfur protecting group when attached to a sulfur atom. In some embodiments, each instance of R^{a10} is independently —H, or C₁₋₆ aliphatic, an oxygen protecting group when attached to an oxygen atom, a nitrogen protecting group when attached to a nitrogen atom, or a sulfur protecting group when attached to a sulfur atom.

As defined herein, m is 0, 1, 2, or 3, inclusive. In certain embodiments, m is 0. In some embodiments, m is 1. In some embodiments, m is 2. In certain embodiments, m is 3.

As defined herein, n is 0, 1, 2, or 3, inclusive. In certain embodiments, n is 0. In some embodiments, n is 1. In certain embodiments, n is 2. In some embodiments, n is 3.

In certain embodiments, m is 0, and n is 0. In some embodiments, m is 0, and n is 1. In certain embodiments, m is 0, and n is 2. In some embodiments, m is 0, and n is 3. In certain embodiments, m is 1, and n is 0. In some embodiments, m is 1, and n is 1. In certain embodiments, m is 1, and n is 2. In some embodiments, m is 1, and n is 3. In certain embodiments, m is 2, and n is 0. In some embodiments, m is 2, and n is 1. In certain embodiments, m is 2, and n is 2. In some embodiments, m is 2, and n is 3. In certain embodiments, m is 3, and n is 0. In some embodiments, m is 3, and n is 1. In certain embodiments, m is 3, and n is 2. In some embodiments, m is 3, and n is 3.

In certain embodiments, a compound of Formula (II) is one of the following:

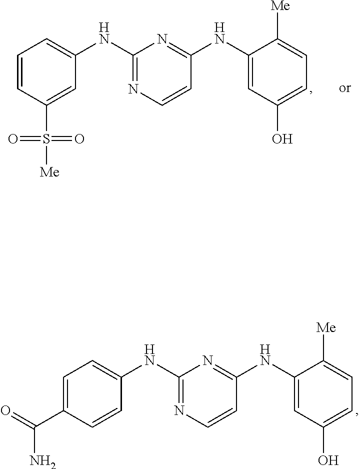 [SMILES: Cc1ccc(O)cc1Nc1ccnc(Nc2cccc(S(C)(=O)=O)c2)n1 ; Cc1ccc(O)cc1Nc1ccnc(Nc2ccc(C(N)=O)cc2)n1]

or a pharmaceutically acceptable salt, solvate, hydrate, polymorph, co-crystal, tautomer, stereoisomer, isotopically labeled derivative, or prodrug thereof.

### Pharmaceutical Compositions, Kits, and Administration

The present disclosure provides pharmaceutical compositions comprising a compound described herein (e.g., a compound of Formula (I) or (II)), or a pharmaceutically acceptable salt, solvate, hydrate, polymorph, co-crystal, tautomer, stereoisomer, isotopically labeled derivative, or prodrug thereof, and optionally a pharmaceutically acceptable carrier and/or excipient. In certain embodiments, the pharmaceutical composition described herein comprises a compound of Formula (I) or (II), or a pharmaceutically acceptable salt, solvate, hydrate, polymorph, co-crystal, tautomer, stereoisomer, isotopically labeled derivative, or prodrug thereof, and a pharmaceutically acceptable carrier and/or excipient.

In certain embodiments, the compound described herein, or a pharmaceutically acceptable salt, solvate, hydrate, polymorph, co-crystal, tautomer, stereoisomer, isotopically labeled derivative, or prodrug thereof, is provided in an effective amount in the pharmaceutical composition. In certain embodiments, the effective amount is a therapeutically or prophylactically effective amount. In certain embodiments, the effective amount is an amount effective for treating a disease or condition (e.g., an inflammatory disease, joint disease, proliferative disease, fibrosis, or pain) in a subject in need thereof. In certain embodiments, the effective amount is an amount effective for preventing a disease or condition (e.g., an inflammatory disease, joint disease, proliferative disease, fibrosis, or pain) in a subject in need thereof. In certain embodiments, the effective amount is an amount effective for reducing the risk of developing a disease or condition (e.g., an inflammatory disease, joint disease, proliferative disease, fibrosis, or pain) in a subject in need thereof.

Pharmaceutical compositions described herein can be prepared by any method known in the art of pharmacology. In general, such preparatory methods include bringing the compound described herein (i.e., the “active ingredient”) into association with a carrier or excipient, and/or one or more other accessory ingredients, and then, if necessary and/or desirable, shaping, and/or packaging the product into a desired single- or multi-dose unit.

Pharmaceutical compositions can be prepared, packaged, and/or sold in bulk, as a single unit dose, and/or as a plurality of single unit doses. A “unit dose” is a discrete amount of the pharmaceutical composition comprising a predetermined amount of the active ingredient. The amount of the active ingredient is generally equal to the dosage of the active ingredient which would be administered to a subject and/or a convenient fraction of such a dosage, such as one-half or one-third of such a dosage.

Relative amounts of the compound of the disclosure, pharmaceutically acceptable excipient, agent, and/or any additional ingredients in a composition described herein will vary, depending upon the identity, size, and/or condition of the subject treated and further depending upon the route by which the composition is to be administered. The composition may comprise between 0.1% and 100% (w/w) agent.

Pharmaceutically acceptable excipients used in manufacture of provided pharmaceutical compositions include inert diluents, dispersing and/or granulating agents, surface active agents and/or emulsifiers, disintegrating agents, binding agents, preservatives, buffering agents, lubricating agents, and/or oils. Excipients and accessory ingredients, such as cocoa butter and suppository waxes, coloring agents, coating agents, sweetening, flavoring, and perfuming agents, may also be present in the composition.

Exemplary diluents include calcium carbonate, sodium carbonate, calcium phosphate, dicalcium phosphate, calcium sulfate, calcium hydrogen phosphate, sodium phosphate lactose, sucrose, cellulose, microcrystalline cellulose, kaolin, mannitol, sorbitol, inositol, sodium chloride, dry starch, cornstarch, powdered sugar, and mixtures thereof.

Exemplary granulating and/or dispersing agents include potato starch, corn starch, tapioca starch, sodium starch glycolate, clays, alginic acid, guar gum, citrus pulp, agar, bentonite, cellulose, and wood products, natural sponge, cation-exchange resins, calcium carbonate, silicates, sodium carbonate, cross-linked poly(vinyl-pyrrolidone) (crospovidone), sodium carboxymethyl starch (sodium starch glycolate), carboxymethyl cellulose, cross-linked sodium carboxymethyl cellulose (croscarmellose), methylcellulose, pregelatinized starch (starch 1500), microcrystalline starch, water insoluble starch, calcium carboxymethyl cellulose, magnesium aluminum silicate (Veegum), sodium lauryl sulfate, quaternary ammonium compounds, and mixtures thereof.

Exemplary surface active agents and/or emulsifiers include natural emulsifiers (e.g., acacia, agar, alginic acid, sodium alginate, tragacanth, chondrux, cholesterol, xanthan, pectin, gelatin, egg yolk, casein, wool fat, cholesterol, wax, and lecithin), colloidal clays (e.g., bentonite (aluminum silicate) and Veegum (magnesium aluminum silicate)), long chain amino acid derivatives, high molecular weight alcohols (e.g., stearyl alcohol, cetyl alcohol, oleyl alcohol, triacetin monostearate, ethylene glycol distearate, glyceryl monostearate, and propylene glycol monostearate, polyvinyl alcohol), carbomers (e.g., carboxy polymethylene, polyacrylic acid, acrylic acid polymer, and carboxyvinyl polymer), carrageenan, cellulosic derivatives (e.g., carboxymethylcellulose sodium, powdered cellulose, hydroxymethyl cellulose, hydroxypropyl cellulose, hydroxypropyl methylcellulose, methylcellulose), sorbitan fatty acid esters (e.g., polyoxyethylene sorbitan monolaurate (Tween® 20), polyoxyethylene sorbitan (Tween® 60), polyoxyethylene sorbitan monooleate (Tween® 80), sorbitan monopalmitate (Span® 40), sorbitan monostearate (Span® 60), sorbitan tristearate (Span® 65), glyceryl monooleate, sorbitan monooleate (Span® 80), polyoxyethylene esters (e.g., polyoxyethylene monostearate (Myrj® 45), polyoxyethylene hydrogenated castor oil, polyethoxylated castor oil, polyoxymethylene stearate, and Solutol®), sucrose fatty acid esters, polyethylene glycol fatty acid esters (e.g., Cremophor®), polyoxyethylene ethers, (e.g., polyoxyethylene lauryl ether (Brij® 30)), poly(vinyl-pyrrolidone), diethylene glycol monolaurate, triethanolamine oleate, sodium oleate, potassium oleate, ethyl oleate, oleic acid, ethyl laurate, sodium lauryl sulfate, Pluronic® F-68, poloxamer P-188, cetrimonium bromide, cetylpyridinium chloride, benzalkonium chloride, docusate sodium, and/or mixtures thereof.

Exemplary binding agents include starch (e.g., cornstarch and starch paste), gelatin, sugars (e.g., sucrose, glucose, dextrose, dextrin, molasses, lactose, lactitol, mannitol, etc.), natural and synthetic gums (e.g., acacia, sodium alginate, extract of Irish moss, panwar gum, ghatti gum, mucilage of isapol husks, carboxymethylcellulose, methylcellulose, ethylcellulose, hydroxyethylcellulose, hydroxypropyl cellulose, hydroxypropyl methylcellulose, microcrystalline cellulose, cellulose acetate, poly(vinyl-pyrrolidone), magnesium aluminum silicate (Veegum®), and larch arabogalactan), alginates, polyethylene oxide, polyethylene glycol, inorganic calcium salts, silicic acid, polymethacrylates, waxes, water, alcohol, and/or mixtures thereof.

Exemplary preservatives include antioxidants, chelating agents, antimicrobial preservatives, antifungal preservatives, antiprotozoan preservatives, alcohol preservatives, acidic preservatives, and other preservatives. In certain embodiments, the preservative is an antioxidant. In other embodiments, the preservative is a chelating agent.

Exemplary antioxidants include alpha tocopherol, ascorbic acid, ascorbyl palmitate, butylated hydroxyanisole, butylated hydroxytoluene, monothioglycerol, potassium metabisulfite, propionic acid, propyl gallate, sodium ascorbate, sodium bisulfite, sodium metabisulfite, and sodium sulfite.

Exemplary chelating agents include ethylenediaminetetraacetic acid (EDTA) and salts and hydrates thereof (e.g., sodium edetate, disodium edetate, trisodium edetate, calcium disodium edetate, dipotassium edetate, and the like), citric acid and salts and hydrates thereof (e.g., citric acid monohydrate), fumaric acid and salts and hydrates thereof, malic acid and salts and hydrates thereof, phosphoric acid and salts and hydrates thereof, and tartaric acid and salts and hydrates thereof. Exemplary antimicrobial preservatives include benzalkonium chloride, benzethonium chloride, benzyl alcohol, bronopol, cetrimide, cetylpyridinium chloride, chlorhexidine, chlorobutanol, chlorocresol, chloroxylenol, cresol, ethyl alcohol, glycerin, hexetidine, imidurea, phenol, phenoxyethanol, phenylethyl alcohol, phenylmercuric nitrate, propylene glycol, and thimerosal.

Exemplary antifungal preservatives include butyl paraben, methyl paraben, ethyl paraben, propyl paraben, benzoic acid, hydroxybenzoic acid, potassium benzoate, potassium sorbate, sodium benzoate, sodium propionate, and sorbic acid.

Exemplary alcohol preservatives include ethanol, polyethylene glycol, phenol, phenolic compounds, bisphenol, chlorobutanol, hydroxybenzoate, and phenylethyl alcohol.

Exemplary acidic preservatives include vitamin A, vitamin C, vitamin E, beta-carotene, citric acid, acetic acid, dehydroacetic acid, ascorbic acid, sorbic acid, and phytic acid.

Other preservatives include tocopherol, tocopherol acetate, deteroxime mesylate, cetrimide, butylated hydroxyanisol (BHA), butylated hydroxytoluened (BHT), ethylenediamine, sodium lauryl sulfate (SLS), sodium lauryl ether sulfate (SLES), sodium bisulfite, sodium metabisulfite, potassium sulfite, potassium metabisulfite, Glydant® Plus, Phenonip®, methylparaben, Germall® 115, Germaben® II, Neolone®, Kathon®, and Euxyl®.

Exemplary buffering agents include citrate buffer solutions, acetate buffer solutions, phosphate buffer solutions, ammonium chloride, calcium carbonate, calcium chloride, calcium citrate, calcium glubionate, calcium gluceptate, calcium gluconate, D-gluconic acid, calcium glycerophosphate, calcium lactate, propanoic acid, calcium levulinate, pentanoic acid, dibasic calcium phosphate, phosphoric acid, tribasic calcium phosphate, calcium hydroxide phosphate, potassium acetate, potassium chloride, potassium gluconate, potassium mixtures, dibasic potassium phosphate, monobasic potassium phosphate, potassium phosphate mixtures, sodium acetate, sodium bicarbonate, sodium chloride, sodium citrate, sodium lactate, dibasic sodium phosphate, monobasic sodium phosphate, sodium phosphate mixtures, tromethamine, magnesium hydroxide, aluminum hydroxide, alginic acid, pyrogen-free water, isotonic saline, Ringer's solution, ethyl alcohol, and mixtures thereof.

Exemplary lubricating agents include magnesium stearate, calcium stearate, stearic acid, silica, talc, malt, glyceryl behanate, hydrogenated vegetable oils, polyethylene glycol, sodium benzoate, sodium acetate, sodium chloride, leucine, magnesium lauryl sulfate, sodium lauryl sulfate, and mixtures thereof.

Exemplary natural oils include almond, apricot kernel, avocado, babassu, bergamot, black current seed, borage, cade, camomile, canola, caraway, carnauba, castor, cinnamon, cocoa butter, coconut, cod liver, coffee, corn, cotton seed, emu, eucalyptus, evening primrose, fish, flaxseed, geraniol, gourd, grape seed, hazel nut, hyssop, isopropyl myristate, jojoba, kukui nut, lavandin, lavender, lemon, Litsea cubeba, macademia nut, mallow, mango seed, meadowfoam seed, mink, nutmeg, olive, orange, orange roughy, palm, palm kernel, peach kernel, peanut, poppy seed, pumpkin seed, rapeseed, rice bran, rosemary, safflower, sandalwood, sasquana, savoury, sea buckthorn, sesame, shea butter, silicone, soybean, sunflower, tea tree, thistle, tsubaki, vetiver, walnut, and wheat germ oils. Exemplary synthetic oils include, but are not limited to, butyl stearate, caprylic triglyceride, capric triglyceride, cyclomethicone, diethyl sebacate, dimethicone 360, isopropyl myristate, mineral oil, octyldodecanol, oleyl alcohol, silicone oil, and mixtures thereof.

Liquid dosage forms for oral and parenteral administration include pharmaceutically acceptable emulsions, microemulsions, solutions, suspensions, syrups and elixirs. In addition to the active ingredients, the liquid dosage forms may comprise inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (e.g., cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, the oral compositions can include adjuvants such as wetting agents, emulsifying and suspending agents, sweetening, flavoring, and perfuming agents. In certain embodiments for parenteral administration, the conjugates described herein are mixed with solubilizing agents such as Cremophor®, alcohols, oils, modified oils, glycols, polysorbates, cyclodextrins, polymers, and mixtures thereof.

Injectable preparations, for example, sterile injectable aqueous or oleaginous suspensions can be formulated according to the known art using suitable dispersing or wetting agents and suspending agents. The sterile injectable preparation can be a sterile injectable solution, suspension, or emulsion in a nontoxic parenterally acceptable diluent or solvent, for example, as a solution in 1,3-butanediol. Among the acceptable vehicles and solvents that can be employed are water, Ringer's solution, U.S.P., and isotonic sodium chloride solution. In addition, sterile, fixed oils are conventionally employed as a solvent or suspending medium. For this purpose any bland fixed oil can be employed including synthetic mono- or di-glycerides. In addition, fatty acids such as oleic acid are used in the preparation of injectables.

The injectable formulations can be sterilized, for example, by filtration through a bacterial-retaining filter, or by incorporating sterilizing agents in the form of sterile solid compositions which can be dissolved or dispersed in sterile water or other sterile injectable medium prior to use.

In order to prolong the effect of a drug, it is often desirable to slow the absorption of the drug from subcutaneous or intramuscular injection. This can be accomplished by the use of a liquid suspension of crystalline or amorphous material with poor water solubility. The rate of absorption of the drug then depends upon its rate of dissolution, which, in turn, may depend upon crystal size and crystalline form. Alternatively, delayed absorption of a parenterally administered drug form may be accomplished by dissolving or suspending the drug in an oil vehicle.

Compositions for rectal or vaginal administration are typically suppositories which can be prepared by mixing the conjugates described herein with suitable non-irritating excipients or carriers such as cocoa butter, polyethylene glycol, or a suppository wax which are solid at ambient temperature but liquid at body temperature and therefore melt in the rectum or vaginal cavity and release the active ingredient.

Solid dosage forms for oral administration include capsules, tablets, pills, powders, and granules. In such solid dosage forms, the active ingredient is mixed with at least one inert, pharmaceutically acceptable excipient or carrier such as sodium citrate or dicalcium phosphate and/or (a) fillers or extenders such as starches, lactose, sucrose, glucose, mannitol, and silicic acid, (b) binders such as, for example, carboxymethylcellulose, alginates, gelatin, polyvinylpyrrolidinone, sucrose, and acacia, (c) humectants such as glycerol, (d) disintegrating agents such as agar, calcium carbonate, potato or tapioca starch, alginic acid, certain silicates, and sodium carbonate, (e) solution retarding agents such as paraffin, (f) absorption accelerators such as quaternary ammonium compounds, (g) wetting agents such as, for example, cetyl alcohol and glycerol monostearate, (h) absorbents such as kaolin and bentonite clay, and (i) lubricants such as talc, calcium stearate, magnesium stearate, solid polyethylene glycols, sodium lauryl sulfate, and mixtures thereof. In the case of capsules, tablets, and pills, the dosage form may include a buffering agent.

Solid compositions of a similar type can be employed as fillers in soft and hard-filled gelatin capsules using such excipients as lactose or milk sugar as well as high molecular weight polyethylene glycols and the like. The solid dosage forms of tablets, dragees, capsules, pills, and granules can be prepared with coatings and shells such as enteric coatings and other coatings well known in the art of pharmacology. They may optionally comprise opacifying agents and can be of a composition that they release the active ingredient(s) only, or preferentially, in a certain part of the intestinal tract, optionally, in a delayed manner. Examples of encapsulating compositions which can be used include polymeric substances and waxes. Solid compositions of a similar type can be employed as fillers in soft and hard-filled gelatin capsules using such excipients as lactose or milk sugar as well as high molecular weight polethylene glycols and the like.

The active ingredient can be in a micro-encapsulated form with one or more excipients as noted above. The solid dosage forms of tablets, dragees, capsules, pills, and granules can be prepared with coatings and shells such as enteric coatings, release controlling coatings, and other coatings well known in the pharmaceutical formulating art. In such solid dosage forms the active ingredient can be admixed with at least one inert diluent such as sucrose, lactose, or starch. Such dosage forms may comprise, as is normal practice, additional substances other than inert diluents, e.g., tableting lubricants and other tableting aids such a magnesium stearate and microcrystalline cellulose. In the case of capsules, tablets and pills, the dosage forms may comprise buffering agents. They may optionally comprise opacifying agents and can be of a composition that they release the active ingredient(s) only, or preferentially, in a certain part of the intestinal tract, optionally, in a delayed manner. Examples of encapsulating agents which can be used include polymeric substances and waxes.

Dosage forms for topical and/or transdermal administration of a compound described herein may include ointments, pastes, creams, lotions, gels, powders, solutions, sprays, inhalants, and/or patches. Generally, the active ingredient is admixed under sterile conditions with a pharmaceutically acceptable carrier or excipient and/or any needed preservatives and/or buffers as can be required. Additionally, the present disclosure contemplates the use of transdermal patches, which often have the added advantage of providing controlled delivery of an active ingredient to the body. Such dosage forms can be prepared, for example, by dissolving and/or dispensing the active ingredient in the proper medium. Alternatively or additionally, the rate can be controlled by either providing a rate controlling membrane and/or by dispersing the active ingredient in a polymer matrix and/or gel.

Formulations suitable for topical administration include, but are not limited to, liquid and/or semi-liquid preparations such as liniments, lotions, oil-in-water and/or water-in-oil emulsions such as creams, ointments, and/or pastes, and/or solutions and/or suspensions. Topically administrable formulations may, for example, comprise from about 1% to about 10% (w/w) active ingredient, although the concentration of the active ingredient can be as high as the solubility limit of the active ingredient in the solvent. Formulations for topical administration may further comprise one or more of the additional ingredients described herein.

Suitable devices for use in delivering injectable pharmaceutical compositions described herein include short needle devices. Injectable compositions can be administered by devices which limit the effective penetration length of a needle into the skin. Alternatively or additionally, conventional syringes can be used in the classical mantoux method of administration. Jet injection devices which deliver liquid formulations via a liquid jet injector and/or via a needle. Ballistic powder/particle delivery devices which use compressed gas to accelerate the compound in powder form are suitable.

A pharmaceutical composition described herein can be prepared, packaged, and/or sold in a formulation suitable for pulmonary administration via the buccal cavity. Such a formulation may comprise dry particles which comprise the active ingredient and which have a diameter in the range from about 0.5 to about 7 nanometers, or from about 1 to about 6 nanometers. Such compositions are conveniently in the form of dry powders for administration using a device comprising a dry powder reservoir to which a stream of propellant can be directed to disperse the powder and/or using a self-propelling solvent/powder dispensing container such as a device comprising the active ingredient dissolved and/or suspended in a low-boiling propellant in a sealed container. Such powders comprise particles wherein at least 98% of the particles by weight have a diameter greater than 0.5 nanometers and at least 95% of the particles by number have a diameter less than 7 nanometers. Alternatively, at least 95% of the particles by weight have a diameter greater than 1 nanometer and at least 90% of the particles by number have a diameter less than 6 nanometers. Dry powder compositions may include a solid fine powder diluent such as sugar and are conveniently provided in a unit dose form.

Low boiling propellants generally include liquid propellants having a boiling point of below 65° F. at atmospheric pressure. Generally, the propellant may constitute 50 to 99.9% (w/w) of the composition, and the active ingredient may constitute 0.1 to 20% (w/w) of the composition. The propellant may further comprise additional ingredients such as a liquid non-ionic and/or solid anionic surfactant and/or a solid diluent (which may have a particle size of the same order as particles comprising the active ingredient).

Pharmaceutical compositions described herein formulated for pulmonary delivery may provide the active ingredient in the form of droplets of a solution and/or suspension. Such formulations can be prepared, packaged, and/or sold as aqueous and/or dilute alcoholic solutions and/or suspensions, optionally sterile, comprising the active ingredient, and may conveniently be administered using any nebulization and/or atomization device. Such formulations may further comprise one or more additional ingredients including, but not limited to, a flavoring agent such as saccharin sodium, a volatile oil, a buffering agent, a surface active agent, and/or a preservative such as methylhydroxybenzoate. The droplets provided by this route of administration may have an average diameter in the range from about 0.1 to about 200 nanometers.

Formulations described herein as being useful for pulmonary delivery are useful for intranasal delivery of a pharmaceutical composition described herein. Another formulation suitable for intranasal administration is a coarse powder comprising the active ingredient and having an average particle from about 0.2 to 500 micrometers. Such a formulation is administered by rapid inhalation through the nasal passage from a container of the powder held close to the nares.

Formulations for nasal administration may, for example, comprise from about as little as 0.1% (w/w) to as much as 100% (w/w) of the active ingredient, and may comprise one or more of the additional ingredients described herein. A pharmaceutical composition described herein can be prepared, packaged, and/or sold in a formulation for buccal administration. Such formulations may, for example, be in the form of tablets and/or lozenges made using conventional methods, and may contain, for example, 0.1 to 20% (w/w) active ingredient, the balance comprising an orally dissolvable and/or degradable composition and, optionally, one or more of the additional ingredients described herein. Alternately, formulations for buccal administration may comprise a powder and/or an aerosolized and/or atomized solution and/or suspension comprising the active ingredient. Such powdered, aerosolized, and/or aerosolized formulations, when dispersed, may have an average particle and/or droplet size in the range from about 0.1 to about 200 nanometers, and may further comprise one or more of the additional ingredients described herein.

A pharmaceutical composition described herein can be prepared, packaged, and/or sold in a formulation for ophthalmic administration. Such formulations may, for example, be in the form of eye drops including, for example, a 0.1-1.0% (w/w) solution and/or suspension of the active ingredient in an aqueous or oily liquid carrier or excipient. Such drops may further comprise buffering agents, salts, and/or one or more other of the additional ingredients described herein. Other ophthalmically-administrable formulations which are useful include those which comprise the active ingredient in microcrystalline form and/or in a liposomal preparation. Ear drops and/or eye drops are also contemplated as being within the scope of this disclosure.

Although the descriptions of pharmaceutical compositions provided herein are principally directed to pharmaceutical compositions which are suitable for administration to humans, it will be understood by the skilled artisan that such compositions are generally suitable for administration to animals of all sorts. Modification of pharmaceutical compositions suitable for administration to humans in order to render the compositions suitable for administration to various animals is well understood, and the ordinarily skilled veterinary pharmacologist can design and/or perform such modification with ordinary experimentation.

Compounds provided herein are typically formulated in dosage unit form for ease of administration and uniformity of dosage. It will be understood, however, that the total daily usage of the compositions described herein will be decided by a physician within the scope of sound medical judgment. The specific therapeutically effective dose level for any particular subject or organism will depend upon a variety of factors including the disease being treated and the severity of the disorder; the activity of the specific active ingredient employed; the specific composition employed; the age, body weight, general health, sex, and diet of the subject; the time of administration, route of administration, and rate of excretion of the specific active ingredient employed; the duration of the treatment; drugs used in combination or coincidental with the specific active ingredient employed; and like factors well known in the medical arts.

The compounds and compositions provided herein can be administered by any route, including enteral (e.g., oral), parenteral, intravenous, intramuscular, intraarticular, intra-arterial, intramedullary, intrathecal, subcutaneous, intraventricular, transdermal, interdermal, rectal, intravaginal, intraperitoneal, topical (as by powders, ointments, creams, and/or drops), mucosal, nasal, bucal, sublingual; by intratracheal instillation, bronchial instillation, and/or inhalation; and/or as an oral spray, nasal spray, and/or aerosol. Specifically, contemplated routes are intraarticular administration, oral administration, intravenous administration (e.g., systemic intravenous injection), regional administration via blood and/or lymph supply, and/or direct administration to an affected site. In general, the most appropriate route of administration will depend upon a variety of factors including the nature of the agent (e.g., its stability in the environment of the gastrointestinal tract), and/or the condition of the subject (e.g., whether the subject is able to tolerate oral administration).

In certain embodiments, the compound or pharmaceutical composition described herein is suitable for intraarticular administration. In some embodiments the compound or pharmaceutical composition described herein is suitable for intraarticular injection. In certain embodiments, the intraarticular injection comprises direct injection into the closed cavity of a joint. In some embodiments, the joint is a knee joint, wrist joint, ankle joint, hip joint, shoulder joint, elbow joint, neck joint, spine joint, vertebral disc joints, finger joints, and toe joints.

The exact amount of a compound of the disclosure required to achieve an effective amount will vary from subject to subject, depending, for example, on species, age, and general condition of a subject, severity of the side effects or disorder, identity of the particular compound of the disclosure, mode of administration, and the like. An effective amount may be included in a single dose (e.g., single oral dose) or multiple doses (e.g., multiple oral doses). In certain embodiments, when multiple doses are administered to a subject or applied to a biological sample, tissue, or cell, any two doses of the multiple doses include different or substantially the same amounts of an agent described herein.

In certain embodiments, when multiple doses are administered to a subject or applied to a tissue or cell, the frequency of administering the multiple doses to the subject or applying the multiple doses to the tissue or cell may be, in non-limiting examples, three doses a day, two doses a day, one dose a day, one dose every other day, one dose every third day, one dose every week, one dose every two weeks, one dose every three weeks, or one dose every four weeks, or even slow dose controlled delivery over a selected period of time using a drug delivery device. In certain embodiments, when multiple doses are administered to a subject or applied to a biological sample, tissue, or cell, the duration between the first dose and last dose of the multiple doses is one day, two days, four days, one week, two weeks, three weeks, one month, two months, three months, four months, six months, nine months, one year, two years, three years, four years, five years, seven years, ten years, fifteen years, twenty years, or the lifetime of the subject, tissue, or cell. In certain embodiments, the duration between the first dose and last dose of the multiple doses is three months, six months, or one year. In certain embodiments, the duration between the first dose and last dose of the multiple doses is the lifetime of the subject, tissue, or cell. In certain embodiments, a dose (e.g., a single dose, or any dose of multiple doses) described herein includes independently between 0.1 pg and 1 pg, between 0.001 mg and 0.01 mg, between 0.01 mg and 0.1 mg, between 0.1 mg and 1 mg, between 1 mg and 3 mg, between 3 mg and 10 mg, between 10 mg and 30 mg, between 30 mg and 100 mg, between 100 mg and 300 mg, between 300 mg and 1,000 mg, or between 1 g and 10 g, inclusive, of a compound described herein. In certain embodiments, a dose described herein includes independently between 1 mg and 3 mg, inclusive, of a compound described herein. In certain embodiments, a dose described herein includes independently between 3 mg and 10 mg, inclusive, of a compound described herein. In certain embodiments, a dose described herein includes independently between 10 mg and 30 mg, inclusive, of a compound described herein. In certain embodiments, a dose described herein includes independently between 30 mg and 100 mg, inclusive, of a compound described herein.

Dose ranges as described herein provide guidance for the administration of provided compounds or compositions to an adult. The amount to be administered to, for example, a child or an adolescent can be determined by a medical practitioner or person skilled in the art and can be lower or the same as that administered to an adult. In certain embodiments, a dose described herein is a dose to an adult human whose body weight is 70 kg.

A compound or composition, as described herein, can be administered in combination with one or more additional pharmaceutical agents (e.g., therapeutically and/or prophylactically active agents). The compounds or compositions can be administered in combination with additional pharmaceutical agents that improve their activity (e.g., potency and/or efficacy), improve bioavailability, improve safety, reduce drug resistance, reduce and/or modify metabolism, inhibit excretion, and/or modify distribution in a subject or cell. It will also be appreciated that the therapy employed may achieve a desired effect for the same disorder, and/or it may achieve different effects. In certain embodiments, a pharmaceutical composition described herein including a compound described herein and an additional pharmaceutical agent shows a synergistic effect that is absent in a pharmaceutical composition including one of the compound and the additional pharmaceutical agent, but not both. In some embodiments, the additional pharmaceutical agent achieves a desired effect for the same disorder. In some embodiments, the additional pharmaceutical agent achieves different effects.

The compound or composition can be administered concurrently with, prior to, or subsequent to one or more additional pharmaceutical agents, which may be useful as, e.g., combination therapies. Pharmaceutical agents include therapeutically active agents. Pharmaceutical agents also include prophylactically active agents. Pharmaceutical agents include small organic molecules such as drug compounds (e.g., compounds approved for human or veterinary use by the U.S. Food and Drug Administration as provided in the Code of Federal Regulations (CFR)), peptides, proteins, carbohydrates, monosaccharides, oligosaccharides, polysaccharides, nucleoproteins, mucoproteins, lipoproteins, synthetic polypeptides or proteins, small molecules linked to proteins, glycoproteins, steroids, nucleic acids, DNAs, RNAs, nucleotides, nucleosides, oligonucleotides, antisense oligonucleotides, lipids, hormones, vitamins, and cells. In certain embodiments, the additional pharmaceutical agent is a pharmaceutical agent useful for treating and/or preventing a disease or condition (e.g., an inflammatory disease, joint disease, proliferative disease, fibrosis, or pain).

Each additional pharmaceutical agent may be administered at a dose and/or on a time schedule determined for that pharmaceutical agent. The additional pharmaceutical agents may also be administered together with each other and/or with the compound or composition described herein in a single dose or composition or administered separately in different doses or compositions. The particular combination to employ in a regimen will take into account compatibility of the compound described herein with the additional pharmaceutical agent(s) and/or the desired therapeutic and/or prophylactic effect to be achieved. In general, it is expected that the additional pharmaceutical agent(s) in combination be utilized at levels that do not exceed the levels at which they are utilized individually. In some embodiments, the levels utilized in combination will be lower than those utilized individually.

The additional pharmaceutical agents include, but are not limited to, anti-proliferative agents, anti-cancer agents, anti-angiogenesis agents, steroidal or non-steroidal anti-inflammatory agents, immunosuppressants, anti-bacterial agents, anti-viral agents, cardiovascular agents, cholesterol-lowering agents, anti-diabetic agents, anti-allergic agents, contraceptive agents, pain-relieving agents, anesthetics, anti-coagulants, inhibitors of an enzyme, steroidal agents, steroidal or antihistamine, antigens, vaccines, antibodies, decongestant, sedatives, opioids, analgesics, anti-pyretics, hormones, and prostaglandins. In certain embodiments, the additional pharmaceutical agent is an anti-proliferative agent. In certain embodiments, the additional pharmaceutical agent is an anti-cancer agent.

Anti-cancer agents encompass biotherapeutic anti-cancer agents as well as chemotherapeutic agents. Exemplary biotherapeutic anti-cancer agents include, but are not limited to, interferons, cytokines (e.g., tumor necrosis factor, interferon α, interferon γ), vaccines, hematopoietic growth factors, monoclonal serotherapy, immunostimulants and/or immunodulatory agents (e.g., IL-1, 2, 4, 6, or 12), immune cell growth factors (e.g., GM-CSF) and antibodies (e.g., HERCEPTIN (trastuzumab), T-DM1, AVASTIN (bevacizumab), ERBITUX (cetuximab), VECTIBIX (panitumumab), RITUXAN (rituximab), BEXXAR (tositumomab)).

Exemplary chemotherapeutic agents include, but are not limited to, anti-estrogens (e.g., tamoxifen, raloxifene, and megestrol), LHRH agonists (e.g., goscrclin and leuprolide), anti-androgens (e.g., flutamide and bicalutamide), photodynamic therapies (e.g., vertoporfin (BPD-MA), phthalocyanine, photosensitizer Pc4, and demethoxy-hypocrellin A (2BA-2-DMHA)), nitrogen mustards (e.g., cyclophosphamide, ifosfamide, trofosfamide, chlorambucil, estramustine, and melphalan), nitrosoureas (e.g., carmustine (BCNU) and lomustine (CCNU)), alkylsulphonates (e.g., busulfan and treosulfan), triazenes (e.g., dacarbazine, temozolomide), platinum containing compounds (e.g., cisplatin, carboplatin, oxaliplatin), vinca alkaloids (e.g., vincristine, vinblastine, vindesine, and vinorelbine), taxoids (e.g., paclitaxel or a paclitaxel equivalent such as nanoparticle albumin-bound paclitaxel (ABRAXANE), docosahexaenoic acid bound-paclitaxel (DHA-paclitaxel, Taxoprexin), polyglutamate bound-paclitaxel (PG-paclitaxel, paclitaxel poliglumex, CT-2103, XYOTAX), the tumor-activated prodrug (TAP) ANG1005 (Angiopep-2 bound to three molecules of paclitaxel), paclitaxel-EC-1 (paclitaxel bound to the erbB2-recognizing peptide EC-1), and glucose-conjugated paclitaxel, e.g., 2′-paclitaxel methyl 2-glucopyranosyl succinate; docetaxel, taxol), epipodophyllins (e.g., etoposide, etoposide phosphate, teniposide, topotecan, 9-aminocamptothecin, camptoirinotecan, irinotecan, crisnatol, mytomycin C), anti-metabolites, DI-FR inhibitors (e.g., methotrexate, dichloromethotrexate, trimetrexate, edatrexate), IMP dehydrogenase inhibitors (e.g., mycophenolic acid, tiazofurin, ribavirin, and EICAR), ribonuclotide reductase inhibitors (e.g., hydroxyurea and deferoxamine), uracil analogs (e.g., 5-fluorouracil (5-FU), floxuridine, doxifluridine, ratitrexed, tegafur-uracil, capecitabine), cytosine analogs (e.g., cytarabine (ara C), cytosine arabinoside, and fludarabine), purine analogs (e.g., mercaptopurine and Thioguanine), Vitamin D3 analogs (e.g., EB 1089, CB 1093, and KH 1060), isoprenylation inhibitors (e.g., lovastatin), dopaminergic neurotoxins (e.g., 1-methyl-4-phenylpyridinium ion), cell cycle inhibitors (e.g., staurosporine), actinomycin (e.g., actinomycin D, dactinomycin), bleomycin (e.g., bleomycin A2, bleomycin B2, peplomycin), anthracycline (e.g., daunorubicin, doxorubicin, pegylated liposomal doxorubicin, idarubicin, epirubicin, pirarubicin, zorubicin, mitoxantrone), MDR inhibitors (e.g., verapamil), Ca²⁺ ATPase inhibitors (e.g., thapsigargin), imatinib, thalidomide, lenalidomide, tyrosine kinase inhibitors (e.g., axitinib (AG013736), bosutinib (SKI-606), cediranib (RECENTIN™, AZD2171), dasatinib (SPRYCEL®, BMS-354825), erlotinib (TARCEVA®), gefitinib (IRESSA®), imatinib (Gleevec®, CGP57148B, STI-571), lapatinib (TYKERB®, TYVERB®), lestaurtinib (CEP-701), neratinib (HKI-272), nilotinib (TASIGNA®), semaxanib (semaxinib, SU5416), sunitinib (SUTENT®, SU11248), toceranib (PALLADIA®), vandetanib (ZACTIMA®, ZD6474), vatalanib (PTK787, PTK/ZK), trastuzumab (HERCEPTIN®), bevacizumab (AVASTIN®), rituximab (RITUXAN®), cetuximab (ERBITUX®), panitumumab (VECTIBIX®), ranibizumab (Lucentis®), nilotinib (TASIGNA®), sorafenib (NEXAVAR®), everolimus (AFINITOR®), alemtuzumab (CAMPATH®), gemtuzumab ozogamicin (MYLOTARG®), temsirolimus (TORISEL®), ENMD-2076, PCI-32765, AC220, dovitinib lactate (TK1258, CHIR-258), BIBW 2992 (TOVOK™), SGX523, PF-04217903, PF-02341066, PF-299804, BMS-777607, ABT-869, MP470, BIBF 1120 (VARGATEF®), AP24534, JNJ-26483327, MGCD265, DCC-2036, BMS-690154, CEP-11981, tivozanib (AV-951), OSI-930, MM-121, XL-184, XL-647, and/or XL228), proteasome inhibitors (e.g., bortezomib (VELCADE)), mTOR inhibitors (e.g., rapamycin, temsirolimus (CCI-779), everolimus (RAD-001), ridaforolimus, AP23573 (Ariad), AZD8055 (AstraZeneca), BEZ235 (Novartis), BGT226 (Norvartis), XL765 (Sanofi Aventis), PF-4691502 (Pfizer), GDC0980 (Genetech), SF1126 (Semafoe) and OSI-027 (OSI)), oblimersen, gemcitabine, carminomycin, leucovorin, pemetrexed, cyclophosphamide, dacarbazine, procarbizine, prednisolone, dexamethasone, campathecin, plicamycin, asparaginase, aminopterin, methopterin, porfiromycin, melphalan, leurosidine, leurosine, chlorambucil, trabectedin, procarbazine, discodermolide, carminomycin, aminopterin, and hexamethyl melamine.

In certain embodiments, the compounds described herein or pharmaceutical compositions can be administered in combination with an anti-cancer therapy including, but not limited to, surgery, radiation therapy, transplantation (e.g., stem cell transplantation, bone marrow transplantation), immunotherapy, and chemotherapy.

In certain embodiments, the additional pharmaceutical agent is an anti-inflammatory agent. In certain embodiments, the additional pharmaceutical agent is a non-steroidal anti-inflammatory drug (NSAID). In certain embodiments, the additional pharmaceutical agent is an analgesic or painkiller.

Also encompassed by the disclosure are kits (e.g., pharmaceutical packs). The kits provided may comprise a pharmaceutical composition or compound provided herein and a container (e.g., a vial, ampule, bottle, syringe, and/or dispenser package, or other suitable container). In some embodiments, provided kits may optionally further include a second container comprising a pharmaceutical excipient for dilution or suspension of a pharmaceutical composition or compound provided herein. In some embodiments, the pharmaceutical composition or compound provided in the first container and the second container are combined to form one unit dosage form. A kit described herein may include one or more additional pharmaceutical agents described herein as a separate composition.

Thus, in one aspect, provided are kits including a first container comprising a compound or pharmaceutical composition described herein. In certain embodiments, the kits are useful for treating a disease or condition (e.g., an inflammatory disease, joint disease, proliferative disease, fibrosis, or pain) in a subject in need thereof. In certain embodiments, the kits are useful for preventing a disease or condition (e.g., an inflammatory disease, joint disease, proliferative disease, fibrosis, or pain) in a subject in need thereof. In certain embodiments, the kits are useful for reducing the risk of developing a disease (e.g., an inflammatory disease, joint disease, proliferative disease, fibrosis, or pain) in a subject in need thereof.

In certain embodiments, a kit described herein further includes instructions for using the kit. A kit described herein may also include information as required by a regulatory agency such as the U.S. Food and Drug Administration (FDA). In certain embodiments, the information included in the kits is prescribing information. In certain embodiments, the kits and instructions provide for treating a disease or condition (e.g., an inflammatory disease, joint disease, proliferative disease, fibrosis, or pain) in a subject in need thereof. In certain embodiments, the kits and instructions provide for preventing a disease or condition (e.g., an inflammatory disease, joint disease, proliferative disease, fibrosis, or pain) in a subject in need thereof. In certain embodiments, the kits and instructions provide for reducing the risk of developing a disease (e.g., an inflammatory disease, joint disease, proliferative disease, fibrosis, or pain) in a subject in need thereof. A kit described herein may include one or more additional pharmaceutical agents described herein as a separate composition.

### Methods of Treatment and Uses

Provided herein are methods of using the compounds provided herein (e.g., compounds of Formulae (I) and (II)), and pharmaceutically acceptable salts, solvates, hydrates, polymorphs, co-crystals, tautomers, stereoisomers, isotopically labeled derivatives, and prodrugs thereof, and pharmaceutical compositions thereof.

Provided herein are methods of treating and/or preventing a disease or condition in a subject, the methods comprising administering to the subject a compound of Formula (I) or (II), or a pharmaceutically acceptable salt, solvate, hydrate, polymorph, co-crystal, tautomer, stereoisomer, isotopically labeled derivative, or prodrug thereof, or a pharmaceutical composition thereof. In certain embodiments, the disease or condition is an inflammatory disease, joint disease, proliferative disease (e.g., cancer), fibrosis, or pain.

Also provided herein are uses of the compounds of Formulae (I) and (II), and pharmaceutically acceptable salts, solvates, hydrates, polymorphs, co-crystals, tautomers, stereoisomers, isotopically labeled derivatives, and prodrugs thereof, and pharmaceutical composition thereof, for the preparation of a medicament for treating and/or preventing a disease or condition in a subject. In certain embodiments, the disease or condition is an inflammatory disease, joint disease, proliferative disease (e.g., cancer), fibrosis, or pain.

Also provided herein are compounds of Formulae (I) and (II), and pharmaceutically acceptable salts, solvates, hydrates, polymorphs, co-crystals, tautomers, stereoisomers, isotopically labeled derivatives, and prodrugs thereof, and pharmaceutical composition thereof, for use in treating and/or preventing a disease or condition in a subject. In certain embodiments, the disease or condition is an inflammatory disease, joint disease, proliferative disease (e.g., cancer), fibrosis, or pain.

In certain embodiments, the disease is an inflammatory disease. In some embodiments, the inflammatory disease is arthritis. In certain embodiments, the inflammatory disease is rheumatoid arthritis. In some embodiments, the inflammatory disease is osteoarthritis. In certain embodiments, the inflammatory disease is spondyloarthropathies. In some embodiments, the inflammatory disease is gouty arthritis. In certain embodiments, the inflammatory disease is systemic lupus erythematosus. In some embodiments, the inflammatory disease juvenile arthritis.

In certain embodiments, the disease is a joint disease. In some embodiments, the joint disease is traumatic cartilage injury.

In certain embodiments, the disease is a proliferative disease. In some embodiments, the proliferative disease is cancer. In certain embodiments, the cancer is ovarian cancer. In some embodiments, the cancer is lung cancer. In certain embodiments, the cancer is head and neck cancer. In some embodiments, the cancer is pancreatic cancer. Other non-limiting examples of cancer are provided herein.

In certain embodiments, the disease is fibrosis. In some embodiments, the fibrosis is hepatic cirrhosis. In some embodiments, the fibrosis is pulmonary fibrosis. In certain embodiments, the fibrosis is cystic fibrosis. In some embodiments, the fibrosis is liver fibrosis. In certain embodiments, the fibrosis is renal fibrosis. In some embodiments, the fibrosis is skin fibrosis. In some embodiments, the fibrosis is scleroderma. In certain embodiments, the fibrosis is atrial fibrosis. In some embodiments, the fibrosis is endomyocardial fibrosis. In certain embodiments, the fibrosis is arthrofibrosis. In some embodiments, the fibrosis is intestinal fibrosis (e.g., Crohn's disease). In certain embodiments, the fibrosis is myelofibrosis. In some embodiments, the fibrosis is skin fibrosis.

In certain embodiments, the condition is pain. In some embodiments, the pain is associated with allodynia. In certain embodiments, the pain is associated with hyperalgesia. In some embodiments, the pain is joint pain.

In certain embodiments, the compound of Formula (I) or (II), or a pharmaceutically acceptable salt, solvate, hydrate, polymorph, co-crystal, tautomer, stereoisomer, isotopically labeled derivative, or prodrugs thereof, or a pharmaceutical composition thereof, is administered intraarticularly. In some embodiments, the compound of Formula (I) or (II), or a pharmaceutically acceptable salt, solvate, hydrate, polymorph, co-crystal, tautomer, stereoisomer, isotopically labeled derivative, or prodrugs thereof, or a pharmaceutical composition thereof, is administered by intraarticular injection.

Also provided herein are methods of inhibiting DDR2 activity in a subject, the methods comprising administering to the subject a compound of Formula (I) or (II), or a pharmaceutically acceptable salt, solvate, hydrate, polymorph, co-crystal, tautomer, stereoisomer, isotopically labeled derivative, or prodrug thereof, or a pharmaceutical composition thereof.

Also provided herein are methods of inhibiting DDR1 activity in a subject, the methods comprising administering to the subject a compound of Formula (I) or (II), or a pharmaceutically acceptable salt, solvate, hydrate, polymorph, co-crystal, tautomer, stereoisomer, isotopically labeled derivative, or prodrug thereof, or a pharmaceutical composition thereof.

Also provided herein are methods of inhibiting p38 MAPK activity in a subject, the methods comprising administering to the subject a compound of Formula (I) or (II), or a pharmaceutically acceptable salt, solvate, hydrate, polymorph, co-crystal, tautomer, stereoisomer, isotopically labeled derivative, or prodrug thereof, or a pharmaceutical composition thereof. In some embodiments, the p38 MAPK is p38-alpha.

Also provided herein are methods of preventing articular cartilage degradation in a subject, the methods comprising administering to the subject a compound of Formula (I) or (II), or a pharmaceutically acceptable salt, solvate, hydrate, polymorph, co-crystal, tautomer, stereoisomer, isotopically labeled derivative, or prodrug thereof, or a pharmaceutical composition thereof.

Also provided herein are methods of inhibiting cell migration in a subject, the methods comprising administering to the subject a compound of Formula (I) or (II), or a pharmaceutically acceptable salt, solvate, hydrate, polymorph, co-crystal, tautomer, stereoisomer, isotopically labeled derivative, or prodrug thereof, or a pharmaceutical composition thereof. In some embodiments, the cell expresses DDR2. In some embodiments, the cell expresses DDR1. In some embodiments, the cell expresses p38 MAPK. In some embodiments, the cell expresses p38-alpha.

Also provided herein are uses of the compounds of Formulae (I) and (II), and pharmaceutically acceptable salts, solvates, hydrates, polymorphs, co-crystals, tautomers, stereoisomers, isotopically labeled derivatives, and prodrugs thereof, and pharmaceutical composition thereof, for the preparation of a medicament for inhibiting DDR2 activity in a subject.

Also provided herein are uses of the compounds of Formulae (I) and (II), and pharmaceutically acceptable salts, solvates, hydrates, polymorphs, co-crystals, tautomers, stereoisomers, isotopically labeled derivatives, and prodrugs thereof, and pharmaceutical composition thereof, for the preparation of a medicament for inhibiting DDR1 activity in a subject.

Also provided herein are uses of the compounds of Formulae (I) and (II), and pharmaceutically acceptable salts, solvates, hydrates, polymorphs, co-crystals, tautomers, stereoisomers, isotopically labeled derivatives, and prodrugs thereof, and pharmaceutical composition thereof, for the preparation of a medicament for inhibiting p38 MAPK activity in a subject. In some embodiments, the p38 MAPK is p38-alpha.

Also provided herein are uses of the compounds of Formulae (I) and (II), and pharmaceutically acceptable salts, solvates, hydrates, polymorphs, co-crystals, tautomers, stereoisomers, isotopically labeled derivatives, and prodrugs thereof, and pharmaceutical composition thereof, for the preparation of a medicament for preventing articular cartilage degradation in a subject.

Also provided herein are uses of the compounds of Formulae (I) and (II), and pharmaceutically acceptable salts, solvates, hydrates, polymorphs, co-crystals, tautomers, stereoisomers, isotopically labeled derivatives, and prodrugs thereof, and pharmaceutical composition thereof, for the preparation of a medicament for inhibiting cell migration in a subject. In some embodiments, the cell expresses DDR2. In some embodiments, the cell expresses DDR1. In some embodiments, the cell expresses p38 MAPK. In some embodiments, the cell expresses p38-alpha.

Also provided herein are compounds of Formulae (I) and (II), and pharmaceutically acceptable salts, solvates, hydrates, polymorphs, co-crystals, tautomers, stereoisomers, isotopically labeled derivatives, and prodrugs thereof, and pharmaceutical composition thereof, for use in inhibiting DDR2 activity in a subject.

Also provided herein are compounds of Formulae (I) and (II), and pharmaceutically acceptable salts, solvates, hydrates, polymorphs, co-crystals, tautomers, stereoisomers, isotopically labeled derivatives, and prodrugs thereof, and pharmaceutical composition thereof, for use in inhibiting DDR1 activity in a subject.

Also provided herein are compounds of Formulae (I) and (II), and pharmaceutically acceptable salts, solvates, hydrates, polymorphs, co-crystals, tautomers, stereoisomers, isotopically labeled derivatives, and prodrugs thereof, and pharmaceutical composition thereof, for use in inhibiting p38 MAPK activity in a subject. In some embodiments, the p38 MAPK is p38-alpha.

Also provided herein are compounds of Formulae (I) and (II), and pharmaceutically acceptable salts, solvates, hydrates, polymorphs, co-crystals, tautomers, stereoisomers, isotopically labeled derivatives, and prodrugs thereof, and pharmaceutical composition thereof, for use in preventing articular cartilage damage in a subject.

Also provided herein are compounds of Formulae (I) and (II), and pharmaceutically acceptable salts, solvates, hydrates, polymorphs, co-crystals, tautomers, stereoisomers, isotopically labeled derivatives, and prodrugs thereof, and pharmaceutical composition thereof, for use in inhibiting cell migration in a subject. In some embodiments, the cell expresses DDR2. In some embodiments, the cell expresses DDR1. In some embodiments, the cell expresses p38 MAPK. In some embodiments, the cell expresses p38-alpha.

### Additional Methods

The present disclosure also provides methods comprising contacting a cell, tissue, or biological sample with an effective amount of a compound provided herein (e.g., a compound of Formula (I) or (II)), or a pharmaceutically acceptable salt, solvate, hydrate, polymorph, co-crystal, tautomer, stereoisomer, isotopically labeled derivative, or prodrug thereof, or a pharmaceutical composition thereof. In certain embodiments, these methods inhibit DDR2 activity. In certain embodiments, these methods inhibit DDR1 activity. In certain embodiments, these methods inhibit p38 MAPK activity. In certain embodiments, these methods inhibit p38-alpha activity. In some embodiments, these methods prevent cartilage degradation. In certain embodiments, these methods inhibit cell migration. In some embodiments, the cell expresses DDR2. In some embodiments, the cell expresses DDR1. In some embodiments, the cell expresses p38 MAPK. In some embodiments, the cell expresses p38-alpha.

Also provided herein are methods for inhibiting DDR2 activity (e.g., in vivo or in vitro) comprising contacting a DDR2 protein with a compound of Formula (I) or (II), or a pharmaceutically acceptable salt, stereoisomer, tautomer, isotopically labeled derivative, solvate, hydrate, polymorph, co-crystal, or prodrug thereof, or a pharmaceutical composition thereof. Also provided herein are methods for inhibiting DDR1 activity (e.g., in vivo or in vitro) comprising contacting a DDR1 protein with a compound of Formula (I) or (II), or a pharmaceutically acceptable salt, stereoisomer, tautomer, isotopically labeled derivative, solvate, hydrate, polymorph, co-crystal, or prodrug thereof, or a pharmaceutical composition thereof. Also provided herein are methods for inhibiting p38 MAPK activity (e.g., in vivo or in vitro) comprising contacting a p38 MAPK protein with a compound of Formula (I) or (II), or a pharmaceutically acceptable salt, stereoisomer, tautomer, isotopically labeled derivative, solvate, hydrate, polymorph, co-crystal, or prodrug thereof, or a pharmaceutical composition thereof. In some embodiments, the p38 MAPK is p38-alpha. In certain embodiments, the inhibiting is in vivo. In certain embodiments, the inhibiting is in vitro.

Also provided herein are compounds of Formulae (I) and (II), and pharmaceutically acceptable salts, stereoisomers, tautomers, isotopically labeled derivatives, solvates, hydrates, polymorphs, co-crystals, and prodrugs thereof, and pharmaceutical compositions thereof, for use in inhibiting DDR2 activity (e.g., in vivo or in vitro). Also provided herein are compounds of Formulae (I) and (II), and pharmaceutically acceptable salts, stereoisomers, tautomers, isotopically labeled derivatives, solvates, hydrates, polymorphs, co-crystals, and prodrugs thereof, and pharmaceutical compositions thereof, for use in inhibiting DDR1 activity (e.g., in vivo or in vitro). Also provided herein are compounds of Formulae (I) and (II), and pharmaceutically acceptable salts, stereoisomers, tautomers, isotopically labeled derivatives, solvates, hydrates, polymorphs, co-crystals, and prodrugs thereof, and pharmaceutical compositions thereof, for use in inhibiting p38 MAPK activity (e.g., in vivo or in vitro). In some embodiments, the p38 MAPK is p38-alpha.

Also provided herein are uses of compounds of Formulae (I) and (II), and pharmaceutically acceptable salts, stereoisomers, tautomers, isotopically labeled derivatives, solvates, hydrates, polymorphs, co-crystals, and prodrugs thereof, and pharmaceutical compositions thereof, for the manufacture of a medicament for inhibiting DDR2 activity in vivo. Also provided herein are uses of compounds of Formulae (I) and (II), and pharmaceutically acceptable salts, stereoisomers, tautomers, isotopically labeled derivatives, solvates, hydrates, polymorphs, co-crystals, and prodrugs thereof, and pharmaceutical compositions thereof, for the manufacture of a medicament for inhibiting DDR1 activity in vivo. Also provided herein are uses of compounds of Formulae (I) and (II), and pharmaceutically acceptable salts, stereoisomers, tautomers, isotopically labeled derivatives, solvates, hydrates, polymorphs, co-crystals, and prodrugs thereof, and pharmaceutical compositions thereof, for the manufacture of a medicament for inhibiting p38 MAPK activity in vivo. In some embodiments, the p38 MAPK is p38-alpha.

In certain embodiments, in vivo methods provided herein comprise administering to a subject an effective amount of a compound of Formula (I) or (II), or a pharmaceutically acceptable salt, stereoisomer, tautomer, isotopically labeled derivative, solvate, hydrate, polymorph, co-crystal, or prodrug thereof, or a pharmaceutical composition thereof.

In certain embodiments, in vitro methods provided herein can be carried out, e.g., in an assay, cell culture, or biological sample. In certain embodiments, methods for inhibiting DDR2 activity in vitro comprise contacting a DDR2 protein with a compound of Formula (I) or (II), or a pharmaceutically acceptable salt, stereoisomer, tautomer, isotopically labeled derivative, solvate, hydrate, polymorph, co-crystal, or prodrug thereof, or a pharmaceutical composition thereof. In certain embodiments, methods for inhibiting DDR1 activity in vitro comprise contacting a DDR1 protein with a compound of Formula (I) or (II), or a pharmaceutically acceptable salt, stereoisomer, tautomer, isotopically labeled derivative, solvate, hydrate, polymorph, co-crystal, or prodrug thereof, or a pharmaceutical composition thereof. In certain embodiments, methods for inhibiting p38 MAPK activity in vitro comprise contacting a p38 MAPK protein with a compound of Formula (I) or (II), or a pharmaceutically acceptable salt, stereoisomer, tautomer, isotopically labeled derivative, solvate, hydrate, polymorph, co-crystal, or prodrug thereof, or a pharmaceutical composition thereof. In some embodiments, the p38 MAPK is p38-alpha. As will be evident to one of skill in the art, the term protein may be used to refer to a single protein as well as a plurality of proteins.

In other embodiments, methods for inhibiting DDR2 activity in a biological sample comprise contacting the biological sample with a compound of Formula (I) or (II), or a pharmaceutically acceptable salt, stereoisomer, tautomer, isotopically labeled derivative, solvate, hydrate, polymorph, co-crystal, or prodrug thereof, or a pharmaceutical composition thereof. In other embodiments, methods for inhibiting DDR1 activity in a biological sample comprise contacting the biological sample with a compound of Formula (I) or (II), or a pharmaceutically acceptable salt, stereoisomer, tautomer, isotopically labeled derivative, solvate, hydrate, polymorph, co-crystal, or prodrug thereof, or a pharmaceutical composition thereof. In other embodiments, methods for inhibiting p38 MAPK activity in a biological sample comprise contacting the biological sample with a compound of Formula (I) or (II), or a pharmaceutically acceptable salt, stereoisomer, tautomer, isotopically labeled derivative, solvate, hydrate, polymorph, co-crystal, or prodrug thereof, or a pharmaceutical composition thereof. In some embodiments, the p38 MAPK is p38-alpha.

In other embodiments, methods for inhibiting DDR2 activity in a cell comprise contacting the cell with a compound of Formula (I) or (II), or a pharmaceutically acceptable salt, stereoisomer, tautomer, isotopically labeled derivative, solvate, hydrate, polymorph, co-crystal, or prodrug thereof, or a pharmaceutical composition thereof. In certain embodiments, the cell expresses DDR2. In other embodiments, methods for inhibiting DDR1 activity in a cell comprise contacting the cell with a compound of Formula (I) or (II), or a pharmaceutically acceptable salt, stereoisomer, tautomer, isotopically labeled derivative, solvate, hydrate, polymorph, co-crystal, or prodrug thereof, or a pharmaceutical composition thereof. In certain embodiments, the cell expresses DDR1. In other embodiments, methods for inhibiting p38 MAPK activity in a cell comprise contacting the cell with a compound of Formula (I) or (II), or a pharmaceutically acceptable salt, stereoisomer, tautomer, isotopically labeled derivative, solvate, hydrate, polymorph, co-crystal, or prodrug thereof, or a pharmaceutical composition thereof. In certain embodiments, the cell expresses p38 MAPK. In some embodiments, the cell expresses p38-alpha.

In some embodiments, any of the compounds or compositions described herein are contacted with a cell in vivo, e.g., in an organism. In some embodiments, any of the compounds or compositions described herein are contacted with a cell in vitro, e.g., in cell culture. In some embodiments, any of the compounds or compositions described herein are contacted with a cell ex vivo, meaning the cell is removed from an organism prior to the contacting. As will be evident to one of skill in the art, the term cell may be used to refer to a single cell as well as a population of cells. In some embodiments, the populations cells are contacted with any of the compounds described herein to regenerate or differentiate one or more cells in the population of cells. In some embodiments, the populations cells are contacted with any of the compounds described herein for use in personalized medicine, for example for diagnostic and/or therapeutic purposes.

In general, any cells known in the art may be used in the methods and uses described herein. In certain embodiments, the cell is of a cell line. In some embodiments, the cell is obtained from an organism, such as a subject. In some embodiments, the cell expresses DDR2. In some embodiments, the cell expresses DDR1. In some embodiments, the cell expresses p38 MAPK. In some embodiments, the cell expresses p38-alpha.

In some embodiments, the methods further comprise measuring or assessing the level of one or more properties of the cell. In some embodiments, the level of one or more properties of the cell is assessed following contacting the cell with any of the compounds or compositions described herein. In some embodiments, the level of one or more properties following contacting the cell with any of the compounds or compositions described herein is compared to the level of one or more properties in a reference sample or prior to contacting the cell with the compounds or composition. In some embodiments, the contacting the cell with any of the compounds or compositions described herein increases one or more properties of the cell. In some aspects, the methods described herein may be used to determine whether a cell is susceptible to treatment with the compounds or compositions described herein. In some embodiments, if the level of one or more properties is increased following contacting the cell with any of the compounds or compositions described herein, the cell is determined to be susceptible to treatment with the compound or composition. In some embodiments, if the level of one or more properties is increased following contacting the cell with any of the compounds or compositions described herein, the compound or composition is determined to be a candidate for a disease or disorder associated with the cell.

### EXAMPLES

In order that the present disclosure may be more fully understood, the following examples are set forth. The synthetic and biological examples described in this application are offered to illustrate the compounds, pharmaceutical compositions, and methods provided herein and are not to be construed in any way as limiting in their scope.

### Synthesis and Characterization of Compounds

### General Procedure for Preparation of Compound B

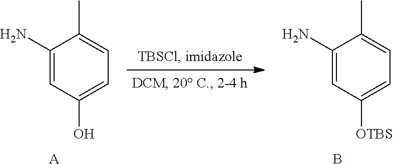

To a solution of 3-amino-4-methyl-phenol (A, 20 g, 162.40 mmol, 1 eq) in DCM (200 mL) was added TBSCl (29.37 g, 194.88 mmol, 23.88 mL, 1.2 eq) and imidazole (13.27 g, 194.88 mmol, 1.2 eq). The mixture was stirred at 20° C. for 2 h. TLC indicated reactant A was consumed completely and one new spot formed. The reaction mixture was diluted with H₂O (200 mL) and extracted with DCM (100 mL). The organic layer was dried over Na₂SO₄, filtered and the filtrate was concentrated under reduced pressure to give a residue. The residue was purified by column chromatography (SiO₂, Petroleum ether/Ethyl acetate=1/0 to 10/1). 5-[tert-butyl(dimethyl)silyl]oxy-2-methyl-aniline (B, 35 g, 147.42 mmol, 90.78% yield, 100% purity) was obtained as colorless oil. LCMS: Retention time 0.806 min, M+H⁺=238.2.

### General Procedure for Preparation of Compound D

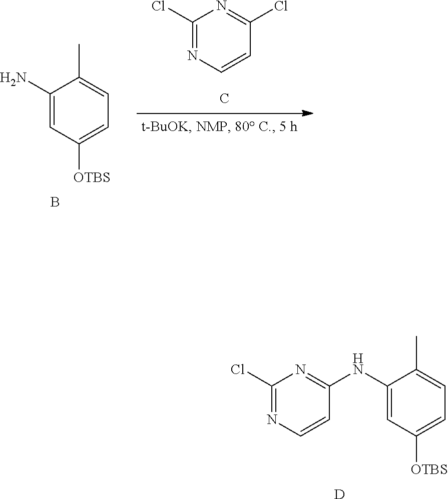

To a solution of 5-[tert-butyl(dimethyl)silyl]oxy-2-methyl-aniline (B, 30 g, 126.36 mmol, 1 eq) and 2,4-dichloropyrimidine (C, 28.24 g, 189.54 mmol, 1.5 eq) in NMP (300 mL) was added t-BuOK (21.27 g, 189.54 mmol, 1.5 eq). The mixture was stirred at 80° C. for 5 h. TLC indicated compound B was consumed completely. The reaction mixture was quenched by addition H₂O (300 mL), and then diluted with brine (300 mL) and extracted with EtOAc (200 mL*3). The combined organic layers were washed with brine (500 mL), dried over Na₂SO₄, filtered, and concentrated under reduced pressure to give a residue. The residue was purified by column chromatography (SiO₂, Petroleum ether/Ethyl acetate=20/1 to 5/1). N-[5-[tert-butyl(dimethyl)silyl]oxy-2-methyl-phenyl]-2-chloro-pyrimidin-4-amine (D, 6 g, 15.09 mmol, 11.94% yield, 88% purity) was obtained as a yellow solid. LCMS confirmed the identity of the product. NOE showed the analyzed structure conforms to the target structure. LCMS: Retention time 0.951 min, M+H⁺=350.2.

### Synthesis and Characterization of Compound 1

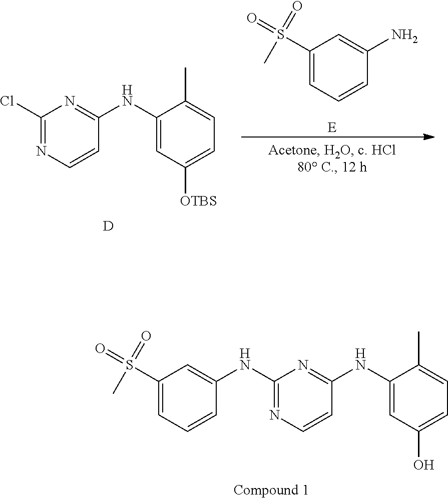

General procedure for preparation of Compound 1. A mixture of N-[5-[tert-butyl(dimethyl)silyl]oxy-2-methyl-phenyl]-2-chloro-pyrimidin-4-amine (D, 4 g, 11.43 mmol, 1 eq) and 3-methylsulfonylaniline (E, 2.94 g, 17.15 mmol, 1.5 eq) in acetone (60 mL), H₂O (40 mL) and HCl solution (12 M, 952.57 μL, 1 eq) was stirred at 80° C. for 12 h under N₂ atmosphere. TLC indicated compound 3 was consumed completely and one new spot formed. The reaction mixture was quenched by addition sat. NaHCO₃. aq. (200 mL), and then diluted with H₂O (100 ML), extracted with EtOAc (100 mL). The organic layer was washed with brine (100 mL), dried over Na₂SO₄, filtered, and concentrated under reduced pressure to give a residue. The residue was purified by column chromatography (SiO₂, Petroleum ether/Ethyl acetate=10/1 to 1/1). 4-methyl-3-[[2-(3-methylsulfonylanilino) pyrimidin-4-yl]amino]phenol (Compound 1, 1.3 g, 3.46 mmol, 30.30% yield, 98.7% purity) was obtained as a white solid. LCMS: Retention time 1.744 min, M+H⁺=371.0. ¹H NMR (400 MHz, DMSO) δ ppm 9.44 (s, 1H), 9.25 (s, 1H), 8.71 (s, 1H), 8.19-8.16 (m, 2H), 8.00 (d, J=5.6 Hz, 1H), 7.42-7.37 (m, 2H), 7.05 (d, J=8.4 Hz, 1H), 6.81 (d, J=2.4 Hz, 1H), 6.57 (dd, J1=2.4 Hz, J2=8 Hz, 1H), 6.10 (d, J=6 Hz, 1H), 3.13 (s, 3H), 2.08 (s, 3H).

### Synthesis and Characterization of Compound 2

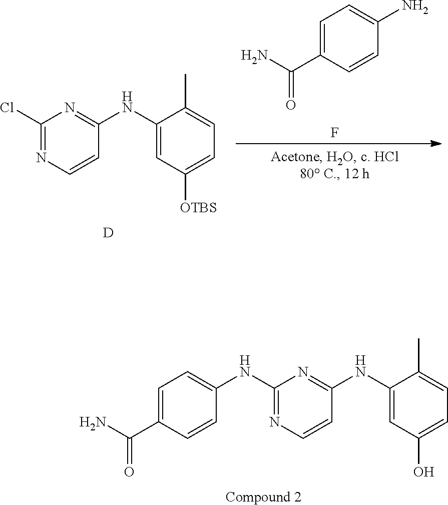

A mixture of N-[5-[tert-butyl(dimethyl)silyl]oxy-2-methyl-phenyl]-2-chloro-pyrimidin-4-amine (D, 4 g, 11.43 mmol, 1 eq) and 4-aminobenzamide (F, 1.87 g, 13.72 mmol, 1.2 eq) in acetone (60 mL), H₂O (40 mL) and HCl solution (12 M, 952.50 μL, 1 eq) was degassed and purged with N₂ for 3 times, and then the mixture was stirred at 80° C. for 12 h under N₂ atmosphere. TLC indicated compound D was consumed completely, and one new spot formed. The reaction mixture was quenched by addition sat. NaHCO₃. aq. (100 mL), and then diluted with H₂O (100 mL) and extracted with EtOAc (100 mL). The organic layer was washed with brine (100 mL), dried over Na₂SO₄, filtered, and concentrated under reduced pressure to give a residue. The residue was purified by column chromatography (SiO2, Petroleum ether/Ethyl acetate=10/1 to 1/1). 4-[[4-(5-hydroxy-2-methyl-anilino)pyrimidin-2-yl]amino]benzamide (Compound 2, 1.2 g, 3.49 mmol, 30.52% yield, 97.5% purity) was obtained as a yellow solid. LCMS: Retention time 1.603 min, M+H⁺=336.0. ¹H NMR (400 MHz, DMSO) δ ppm 9.33 (s, 1H), 9.26 (s, 1H), 8.73 (s, 1H), 7.98 (d, J=5.6 1H), 7.76 (d, J=8.8 Hz, 3H), 7.68 (d, J=8.8 Hz, 2H), 7.12 (s, 1H), 7.06 (d, J=8.4 Hz, 1H), 6.82 (d, J=2 Hz, 1H), 6.60 (dd, J=8.0 Hz, J=5.6 Hz 1H), 6.10 (t, J=9.2 Hz, 1H), 2.09 (s, 3H).

### Synthesis and Characterization of Compound 3

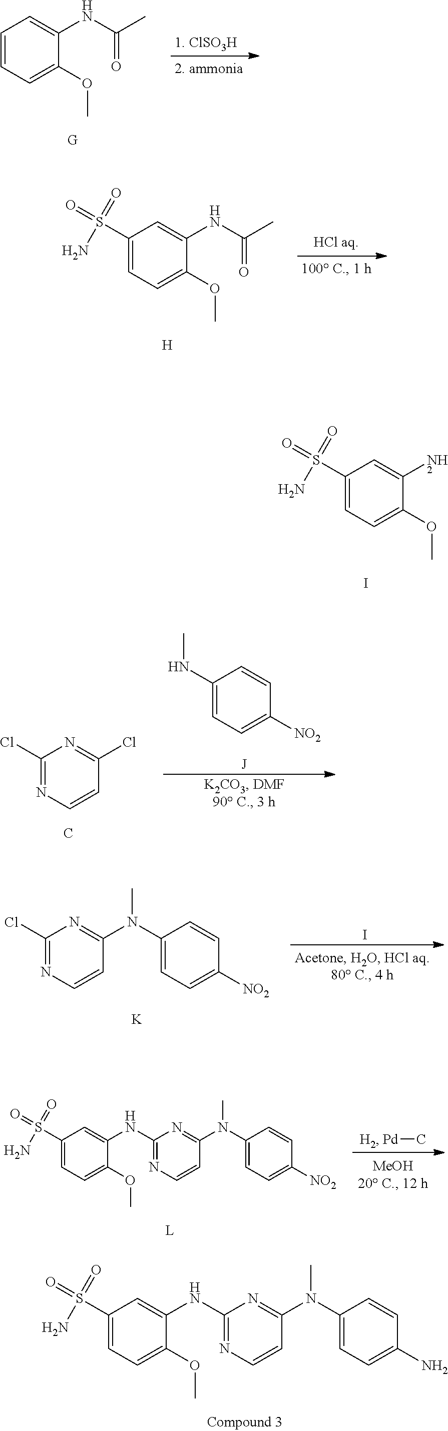

General procedure for preparation of Compound H. A solution of N-(2-methoxyphenyl)acetamide (G, 1 g, 6.05 mmol, 1 eq) in sulfurochloridic acid (2.12 g, 18.16 mmol, 1.21 mL, 3 eq) was degassed and purged with N₂ for 3 times, and then the mixture was stirred at 20° C. for 1 h. The mixture was poured into NH₃·H₂O (7.07 g, 60.54 mmol, 7.77 mL, 30% purity, 10 eq) and stirred for 1 h. The mixture was concentrated in vacuum to yield N-(2-methoxy-5-sulfamoyl-phenyl)acetamide (H, 1.5 g, crude) as a white solid which was used directly in next step.

General procedure for preparation of Compound I. A solution of N-(2-methoxy-5-sulfamoyl-phenyl)acetamide (H, 1 g, 4.09 mmol, 1 eq) in HCl (12 M, 10 mL, 29.31 eq) was stirred at 100° C. for 1 h. The reaction mixture was concentrated under reduced pressure to yield 3-amino-4-methoxy-benzenesulfonamide (I, 1 g, crude) as a white solid which was used directly in next step. ¹H NMR (400 MHz, MeOD) δ ppm 7.56 (s, 1H), 7.49-7.41 (m, 1H), 7.29 (s, 1H), 7.21˜7.15 (m, 4H), 3.9 (s, 3H).

General procedure for preparation of Compound K. To a mixture of 2,4-dichloropyrimidine (C, 14 g, 93.97 mmol, 1 eq) and N-methyl-4-nitro-aniline (J, 14.30 g, 93.97 mmol, 1 eq) in DMF (150 mL) was added K₂CO₃ (25.98 g, 187.95 mmol, 2 eq) in one portion at 25° C. under N₂. The mixture was stirred at 90° C. for 3 hours. The mixture was cooled to 25° C. and poured into ice-water (w/w=1/1) (100 mL) and stirred for 10 min. The aqueous phase was extracted with ethyl acetate (100 mL*2). The combined organic phase was washed with brine (200 mL), dried with anhydrous Na₂SO₄, filtered, and concentrated in vacuum. The residue was purified by silica gel chromatography (column height: 250 mm, diameter: 100 mm, 100-200 mesh silica gel, Petroleum ether/Ethyl acetate=12/1, 1/1) to afford 2-chloro-N-methyl-N-(4-nitrophenyl)pyrimidin-4-amine (K, 7 g, 26.45 mmol, 28.14% yield) as a yellow solid. LCMS: Retention time 0.859 min, M+H⁺=265.0.

General procedure for preparation of Compound L. To a mixture of 2-chloro-N-methyl-N-(4-nitrophenyl)pyrimidin-4-amine (K, 6 g, 22.58 mmol, 1.0 eq), 3-amino-4-methoxy-benzenesulfonamide (I, 4.57 g, 22.58 mmol, 1.00 eq) in H₂O (5 mL) and acetone (2.5 mL) was added HCl (12 M, 1.20 mL). The mixture was stirred at 80° C. for 4 h under N₂ atmosphere. The reaction mixture was extracted with EtOAc (50 mL*2). The combined organic layers were concentrated under reduced pressure to give a residue. The residue was purified by silica gel chromatography (column height: 250 mm, diameter: 100 mm, 100-200 mesh silica gel, Petroleum ether/Ethyl acetate=12/1, 1/1) to afford 4-methoxy-3-[[4-(N-methyl-4-nitro-anilino)pyrimidin-2-yl]amino]benzenesulfonamide (L, 3 g, 6.97 mmol, 30.86% yield) as a yellow solid. The structure was confirmed by 2D NMR.

General procedure for preparation of Compound 3. To a solution of 4-methoxy-3-[[4-(N-methyl-4-nitro-anilino)pyrimidin-2-yl]amino]benzenesulfonamide (L, 3 g, 6.97 mmol, 1 eq) in MeOH (80 mL) was added Pd/C (0.5 g, 929.29 umol, 10% purity) under N₂. The suspension was degassed under vacuum and purged with H₂ several times. The mixture was stirred under H₂ (15 Psi) at 20° C. for 12 hours. The reaction mixture was filtered, and the filtrate was concentrated. The crude product was purified by silica gel chromatography eluted with Petroleum ether/Ethyl acetate=0:1 to give 3-[[4-(4-amino-N-methyl-anilino)pyrimidin-2-yl]amino]-4-methoxy-benzenesulfonamide (Compound 3, 1.3 g, 3.17 mmol, 45.41% yield, 97.5% purity). LCMS: Retention time 1.578 min, M+H⁺=401.0. ¹H NMR (400 MHz, d6-DMSO) δ ppm 9.68 (brs, 1H), 8.75 (brs, 1H), 7.87 (d, J=7.6 Hz, 1H), 7.64 (dd, J1=2 Hz, J2=2.4 Hz, 1H), 7.33-7.27 (m, 3H), 7.03 (d, J=8.4 Hz, 2H), 6.69 (d, J=8.8 Hz, 2H), 5.86 (d, J=7.2 Hz, 1H), 3.97 (s, 3H), 3.45 (s, 3H).

### Synthesis and Characterization of Compound 4

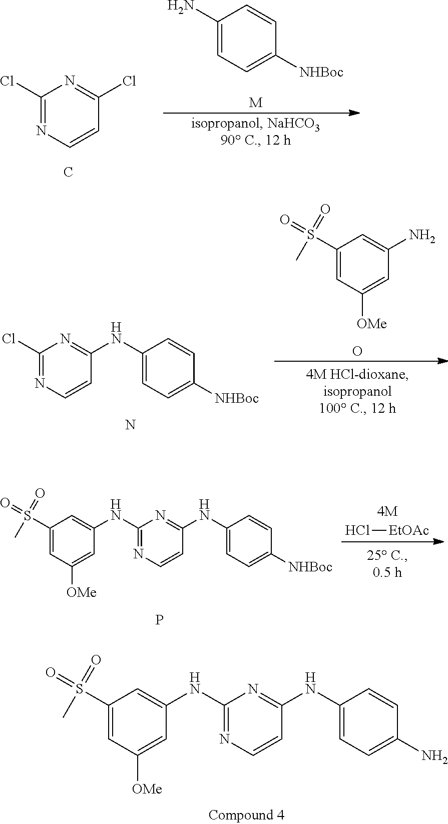

General procedure for preparation of tert-butyl (4-((2-chloropyrimidin-4-yl)amino)phenyl)carbamate. To a suspension of tert-butyl (4-aminophenyl)carbamate (M, 5 g, 24.01 mmol) and 2,4-dichloropyrimidine (C, 4.65 g, 31.21 mmol) in isopropanol (17.76 mL) was added sodium hydrogen carbonate (6.05 g, 72.03 mmol, 2.80 mL). The mixture was stirred at 90° C. for 12 h under N₂ atmosphere. The mixture was filtered to remove the solid. The filtrate was concentrated to give a residue. To the residue was added DCM (120 mL) and the suspension was stirred at 25° C. for 30 min. The precipitated solid was collected by filtration and dried to obtain tert-butyl (4-((2-chloropyrimidin-4-yl)amino)phenyl)carbamate (N, 5.5 g, 17.15 mmol, 71.42% yield) as a yellow solid. ¹H NMR: (400 MHz, DMSO-d₆) δ=9.87 (s, 1H), 9.32 (br s, 1H), 8.09 (d, J=6 Hz, 1H), 7.48-7.37 (m, 4H), 6.66 (d, J=6 Hz, 1H), 1.47 (s, 9H).

Tert-butyl (4-((2-((3-methoxy-5-(methylsulfonyl)phenyl)amino)pyrimidin-4-yl)amino)-phenyl)carbamate. To a suspension of tert-butyl (4-((2-chloropyrimidin-4-yl)amino)phenyl)carbamate (N, 2.77 g, 8.64 mmol) and 3-methoxy-5-(methylsulfonyl)aniline (0, 2.00 g, 9.93 mmol) in isopropanol (150 mL) was added HCl/dioxane (4 M, 798.77 L). The mixture was stirred at 100° C. for 12 h under N₂ atmosphere. The mixture was filtered to obtain a filter cake. The filter cake was dissolved in 500 mL of mix solvent (THF: Ethyl acetate=1:4). Then the solution was basified with 2M aq. sodium carbonate solution to pH=8. The mixture was extracted with ethyl acetate (300 mL*2). The combined organic phase was washed with brine (500 mL), dried with anhydrous Na₂SO₄, filtered, and concentrated in vacuum. The residue was purified by flash silica gel chromatography (silica flash column, eluent of 20˜80% Ethyl acetate/Petroleum ether gradient) to obtain tert-butyl (4-((2-((3-methoxy-5-(methylsulfonyl)phenyl)amino)pyrimidin-4-yl)amino)phenyl)carbamate (P, 2.2 g, 4.51 mmol, 52.26% yield, 99.61% purity) as a yellow solid.

N4-(4-aminophenyl)-N2-(3-methoxy-5-(methylsulfonyl)phenyl)pyrimidine-2,4-diamine. A suspension of tert-butyl (4-((2-((3-methoxy-5-(methylsulfonyl)phenyl)amino)pyrimidin-4-yl)amino)phenyl)carbamate (P, 2.20 g, 4.53 mmol) in HCl-EtOAc (4 M, 60 mL) was stirred at 25° C. for 0.5 h. The mixture was basified with 2M aq. sodium carbonate solution to pH 8. The mixture was extracted with ethyl acetate (80 mL*2). The combined organic phase was washed with brine (100 mL), dried with anhydrous Na₂SO₄, filtered, and concentrated in vacuum to obtain N4-(4-aminophenyl)-N2-(3-methoxy-5-(methylsulfonyl)phenyl)pyrimidine-2,4-diamine (Compound 4, 1.3 g, 3.28 mmol, 97.16% purity) as a yellow solid. HPLC: 97.16% purity. ¹H NMR: (400 MHz, DMSO-d₆) δ=9.38 (s, 1H), 8.93 (br s, 1H), 7.94 (d, J=6 Hz, 1H), 7.90-7.80 (m, 2H), 7.22 (br d, J=7.2 Hz, 2H), 6.95-6.91 (m, 1H), 6.61-6.53 (m, 2H), 6.11 (br d, J=6 Hz, 1H), 5.00 (br s, 2H), 3.78 (s, 3H), 3.16 (s, 3H). LCMS: 95.71% purity, MS+H⁺=386.1.

### Inhibition of DDR1 and DDR2

As shown in Table 1 below, compounds of Formulae (I) and (II) are effective DDR2 inhibitors.

DDR1 (h) was incubated with 8 mM MOPS pH 7.0, 0.2 mM EDTA, 250 μM KKKSPGEYVNIEFG (SEQ ID NO: 1), 10 mM magnesium acetate and [γ-33P]-ATP (specific activity and concentration as required). The reaction was initiated by the addition of the Mg/ATP mix. After incubation for 40 minutes at room temperature, the reaction was stopped by the addition of phosphoric acid to a concentration of 0.5%. An aliquot of the reaction was then spotted onto a filter and washed four times for 4 minutes in 0.425% phosphoric acid and once in methanol prior to drying and scintillation counting. Results are provided in the second column of Table 1.

DDR2 (h) was incubated with 8 mM MOPS pH 7.0, 0.2 mM EDTA, 250 μM KKSRGDYMTMQIG (SEQ TD NO: 2), 10 mM MnCl2, 10 mM magnesium acetate and [γ-33P]-ATP (specific activity and concentration as required). The reaction was initiated by the addition of the Mg/ATP mix. After incubation for 40 minutes at room temperature, the reaction was stopped by the addition of phosphoric acid to a concentration of 0.5%. An aliquot of the reaction was then spotted onto a filter and washed four times for 4 minutes in 0.425% phosphoric acid and once in methanol prior to drying and scintillation counting. Results are provided in the third column of Table 1.

TABLE 1
Inhibition of DDR1 and DDR2

| | DDR1 IC₅₀ | DDR2 IC₅₀ |
|---|---|---|
| Compound | (nM) | (nM) |
| 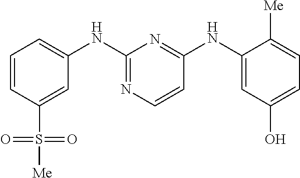 [SMILES: Cc1ccc(O)cc1Nc1ccnc(Nc2cccc(S(C)(=O)=O)c2)n1] | 13 | 260 |
| Compound 1 | | |
| 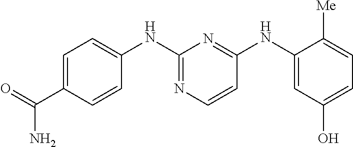 [SMILES: Cc1ccc(O)cc1Nc1ccnc(Nc2ccc(C(N)=O)cc2)n1] | 9 | 156 |
| Compound 2 | | |
| 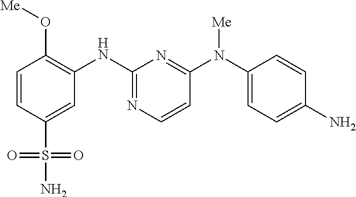 [SMILES: COc1ccc(S(N)(=O)=O)cc1Nc1nccc(N(C)c2ccc(N)cc2)n1] | 55 | >3,300 |
| Compound 3 | | |
| 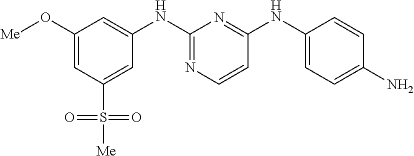 [SMILES: COc1cc(Nc2nccc(Nc3ccc(N)cc3)n2)cc(S(C)(=O)=O)c1] | 66 | 1574 |
| Compound 4 | | |

### Selectivity for DDR2

Selectivity profiling of Compounds 1-4 was performed against a diverse set of 97 kinases (90 WT kinase and 7 mutants, ScanEDGE, DiscoveRx) by 1 mM inhibitors.

Compounds that bind the kinase active site and directly (sterically) or indirectly (allosterically) prevent kinase binding to the immobilized ligand, will reduce the amount of kinase captured on the solid support. Conversely, compounds that do not bind the kinase have no effect on the amount of kinase captured on the solid support. Screening “hits” are identified by measuring the amount of kinase captured in test versus control samples by using a quantitative qPCR method that detects the associated DNA label. Dissociation constants (Kds) for test compound-kinase interactions can be calculated by measuring the amount of kinase captured on the solid support as a function of the test compound concentration.

Kinase-tagged T7 phage strains were grown in parallel in 24-well blocks in an E. coli host derived from the BL21 strain. E. coli were grown to log-phase and infected with T7 phage from a frozen stock (multiplicity of infection=0.4) and incubated with shaking at 32° C. until lysis (90-150 minutes). The lysates were centrifuged (6,000×g) and filtered (0.2 μm) to remove cell debris. The remaining kinases were produced in HEK-293 cells and subsequently tagged with DNA for qPCR detection. Streptavidin-coated magnetic beads were treated with biotinylated small molecule ligands for 30 minutes at room temperature to generate affinity resins for kinase assays. The liganded beads were blocked with excess biotin and washed with blocking buffer (SeaBlock (Pierce), 1% BSA, 0.05% Tween 20, 1 mM DTT) to remove unbound ligand and to reduce non-specific phage binding. Binding reactions were assembled by combining kinases, liganded affinity beads, and test compounds in 1x binding buffer (20% SeaBlock, 0.1π PBS, 0.05% Tween 20, 6 mM DTT). Test compounds were prepared as 40x stocks in 100% DMSO and directly diluted into the assay. All reactions were performed in polypropylene 384-well plates in a final volume of 0.02 mL. The assay plates were incubated at room temperature with shaking for 1 hour and the affinity beads were washed with wash buffer (1×PBS, 0.05% Tween 20). The beads were then re-suspended in elution buffer (1×PBS, 0.05% Tween 20, 0.5 M non-biotinylated affinity ligand) and incubated at room temperature with shaking for 30 minutes. The kinase concentration in the eluates was measured by qPCR.

The compounds were screened at 1000 nM, and results for primary screen binding interactions were determined as “% Ctrl”. % Ctrl=[(test compound signal−positive control signal)/(negative control signal−positive control signal)]*100. Negative control was 100% DMSO. Test compounds were Compounds 1-4. Positive control was a control compound.

Selectivity Score or S-score is a quantitative measure of compound selectivity for DDR2. It was calculated by dividing the number of kinases that compounds bind to by the total number of distinct kinases tested, excluding mutant variants. S=number of hits/number of assays. % Ctrl was used as a potency threshold. Selectivity score values of CS analogs provided in Table 2 reflect the number of kinases inhibited by >65% (S35), >90% (S10), or 99% (S1) by Compounds 1-4.

TABLE 2
Selectivity of Compounds 1-4 for DDR2

| Compound | Selectivity Score Type | # of Hits | Selectivity Score |
|---|---|---|---|
| Compound 1 | S(35) | 29 | 0.322 |
| | S(10) | 22 | 0.244 |
| | S(1) | 7 | 0.078 |
| Compound 2 | S(35) | 22 | 0.244 |
| | S(10) | 8 | 0.089 |
| | S(1) | 3 | 0.033 |
| Compound 3 | S(35) | 16 | 0.178 |
| | S(10) | 8 | 0.089 |
| | S(1) | 5 | 0.056 |
| Compound 4 | S(35) | 21 | 0.233 |
| | S(10) | 11 | 0.122 |
| | S(1) | 2 | 0.022 |

Binding Affinity of Compound 1 to p38-Alpha

Table 3 provides binding affinity scores for p38-alpha as a percent relative to DMSO control. Smaller numbers indicate stronger binding. Of note, the interaction of DDR2 with type II collagen activates the receptor and downstream signaling pathways p38 and ERK to induce expression of MMP13.⁴ Cytokine inhibition by blocking p38 MAPK pathway has been shown to attenuate pain and joint degeneration in a rat models of osteoarthritis.¹

TABLE 3
Binding Affinity of Compound 1 to p38-alpha

| Target | Compound 1 | Compound 2 | Compound 3 | Compound 4 |
|---|---|---|---|---|
| P38-alpha | 0.5 | 30 | 83 | 87 |

### Solubility, Stability, Permeability, and Pharmacokinetics

The solubility, stability, permeability, and pharmacokinetic properties of compounds of Formulae (I) and (II) are provided in Table 4.

TABLE 4

| Test | Compound 1 | | Compound 2 | | Compound 3 | | Compound 4 | |
|---|---|---|---|---|---|---|---|---|
| Solubility in ph 7.4 | 16.0 | μM | 770 | μM | 13.3 | μM | 213 | μM |
| phosphate buffer | | | | | | | | |
| Stability in HBSS | >120 min in buffer | | >120 min in buffer | | >120 min in buffer | | >120 min in buffer | |
| and plasma | >360 min (all | | >360 min (all | | >360 min (all | | >360 min (all | |
| | animals) | | animals) | | animals) | | animals) | |
| Caco-2 | Uni -: 26.2 Pₐₚₚ | | | | Very high | | Very high | |
| permeability | Bi-direc: 4.27 efflux | | | | unidirectional | | unidirectional | |
| | r. | | | | permeability | | permeability | |
| | Very high | | | | | | | |
| | unidirectional | | | | | | | |
| | permeability | | | | | | | |
| Liver S9 stability | Rapid clearance | | Rapid clearance | | Very stable | | Very stable | |
| (SD rat, beagle, | (20.0 min half life in | | | | | | | |
| human) | rat, <10 min half life | | | | | | | |
| | in human, dog) | | | | | | | |
| Stability in | >5 | days | >5 | days | >5 | days | >5 | days |
| simulated synovial | | | | | | | | |
| fluid | | | | | | | | |

| | |
|---|---|
| PK following IV | t_{1/2} (hr) 0.319 +/− |
| dosing (1 mg/kg) | 0.029 |
| in male SD rats | C₀ (ng/mL) 548 +/− |
| | 30 |
| | CL (mL/min/kg) 137 |
| | V_{SS} (L/kg) 2.11 +/− |
| | 0.15 |
| | AUCₗₐₛₜ (hr · ng/mL) 122 +/− |
| | 8 |

Compounds exhibited high clearance, rapid elimination, MRT 0.24 h, good characteristics for avoiding systemic exposure following IA injection

### Evaluation of DDR2 Inhibitor, Compound 3, on Articular Cartilage of Knee Joint in an Injurious Mouse Model of Osteoarthritis

Significant delay of the progressive process of articular cartilage degeneration, induced by DMM, in mouse knee joints was achieved by the treatment of compound 3.

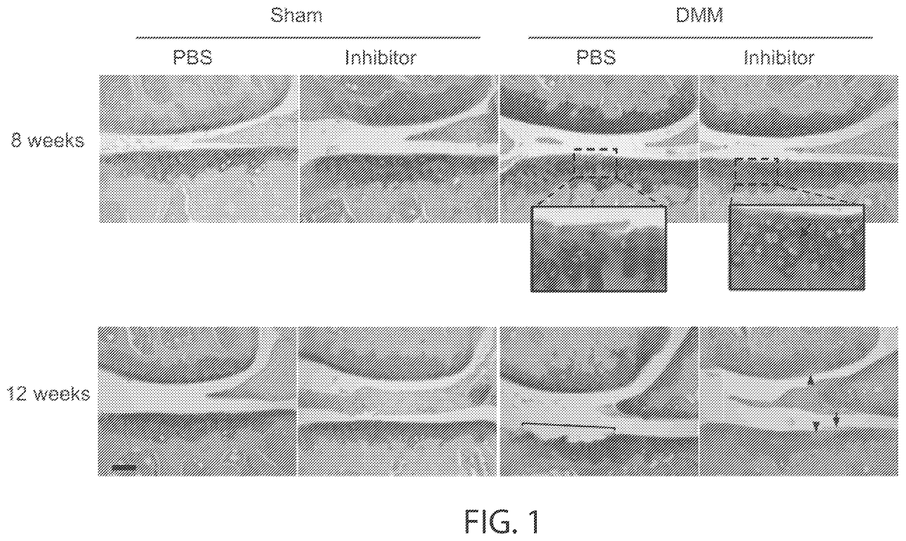

Based on a pilot experiment,¹ the sample size of minimum 6 is required by the power analysis to achieve the specified confidence interval (95%) with at least 50% reduction of the score in the treatment group. Mouse knee joints were subjected to DMM surgery. Four weeks following the surgery, mice were treated with compound 3. Significant disparity was found in the progression of articular cartilage degeneration in knee joints with or without the treatment of compound 3 (FIG. 1). The fibrillation was observed in mouse knee joints at 8 weeks following DMM surgery without the treatment of compound 3. However, with the treatment of the compound 3, only chondrocyte clusters were seen in mouse knee joints at weeks after DMM surgery. At the 12 weeks after DMM surgery, there was loss of the articular cartilage in knee joints of mice not treated with compound 3, whereas only localized proteoglycan degradation was seen in knee joints of mice treated with compound 3. The progressive process of the cartilage degeneration was dramatically delayed in the mice treated with compound 3.

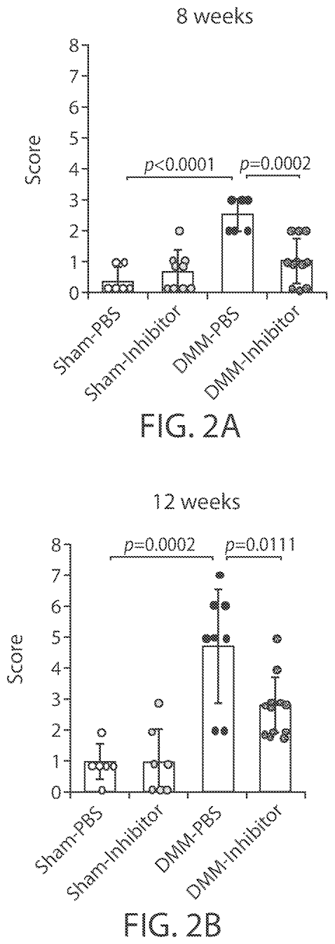

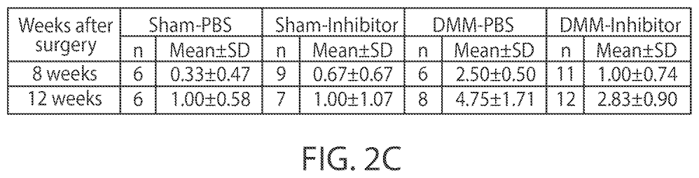

The condition of the articular cartilage was also evaluated by the scoring system.³ At 8 weeks after DMM surgery, the average scores for mice without and with treatment using compound 3 were 2.50 and 1.00, respectively. At 12 weeks following DMM surgery, the average scores were 4.75 for mice not treated with compound 3 and 2.83 for mice treated with compound 3. There were significant differences in the scores between the two groups at both time points (FIGS. 2A-C). In addition, osteophytes and other abnormal morphology were not observed in the subchondral bone and synovial tissue in mice with or without the treatment with compound 3.

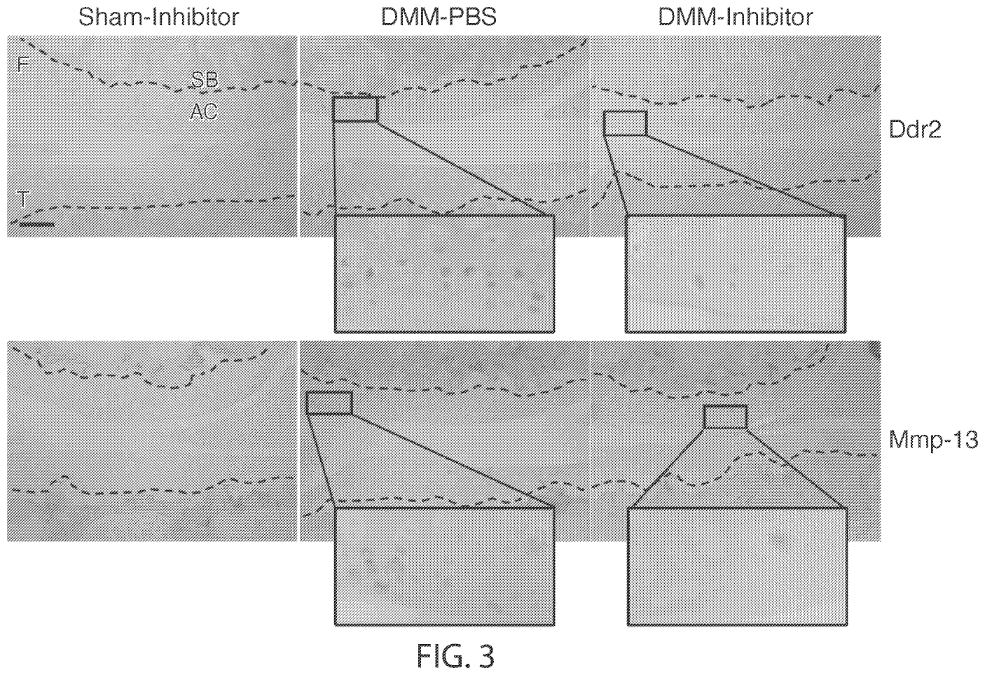

Inhibitory induction of Ddr2 and Mmp-13 in articular cartilage of knee joints was observed after treatment with the compound 3. The protein expressions of Ddr2 and Mmp-13 were examined in the articular cartilage of mouse knee joints at 8 weeks after DMM, with or without the treatment with compound 3. The localized brown-color staining cells (positive) of Ddr2 and Mmp-13 were observed in all of mice without the treatment with compound 3. However, there was a significant reduction in number of the positive cells of Ddr2 and Mmp-13 in mice with the treatment of the compound 3 (FIG. 3).

### REFERENCES

1. Brown, K. K. et al. P38 MAP kinase inhibitors as potential therapeutics for the treatment of joint degeneration and pain associated with osteoarthritis. J. Inflamm. 2008, 5, 22.
2. Manning, L. B. et al. Discoidin Domain Receptor 2 as a Potential Therapeutic Target for Development of Disease-Modifying Osteoarthritis Drugs. Am Jour. Pathol. 2016, 186, 3000-3010.
3. Glasson, S. S., et al. The OARSI histopathology initiative: recommendations for histological assessments of osteoarthritis in the mouse. Osteoarthr. Cartil. 2010, 18, S17-S23.
4. Xiao, L., et al. Targeting Discoidin Domain Receptor 2 for the Development of Disease-Modifying Osteoarthritis Drugs. Cartilage 2021, 13, 1285S-1291S.

### EQUIVALENTS AND SCOPE

In the claims articles such as “a,” “an,” and “the” may mean one or more than one unless indicated to the contrary or otherwise evident from the context. Claims or descriptions that include “or” between one or more members of a group are considered satisfied if one, more than one, or all of the group members are present in, employed in, or otherwise relevant to a given product or process unless indicated to the contrary or otherwise evident from the context. The invention includes embodiments in which exactly one member of the group is present in, employed in, or otherwise relevant to a given product or process. The invention includes embodiments in which more than one, or all of the group members are present in, employed in, or otherwise relevant to a given product or process.

Furthermore, the invention encompasses all variations, combinations, and permutations in which one or more limitations, elements, clauses, and descriptive terms from one or more of the listed claims is introduced into another claim. For example, any claim that is dependent on another claim can be modified to include one or more limitations found in any other claim that is dependent on the same base claim. Where elements are presented as lists, e.g., in Markush group format, each subgroup of the elements is also disclosed, and any element(s) can be removed from the group. It should it be understood that, in general, where the invention, or aspects of the invention, is/are referred to as comprising particular elements and/or features, certain embodiments of the invention or aspects of the invention consist, or consist essentially of, such elements and/or features. For purposes of simplicity, those embodiments have not been specifically set forth in haec verba herein. It is also noted that the terms “comprising” and “containing” are intended to be open and permits the inclusion of additional elements or steps. Where ranges are given, endpoints are included. Furthermore, unless otherwise indicated or otherwise evident from the context and understanding of one of ordinary skill in the art, values that are expressed as ranges can assume any specific value or sub-range within the stated ranges in different embodiments of the invention, to the tenth of the unit of the lower limit of the range, unless the context clearly dictates otherwise.

Those skilled in the art will recognize or be able to ascertain using no more than routine experimentation many equivalents to the specific embodiments described herein. The scope of the present embodiments described herein is not intended to be limited to the above Description, but rather is as set forth in the appended claims. Those of ordinary skill in the art will appreciate that various changes and modifications to this description may be made without departing from the spirit or scope of the present invention, as defined in the following claims.

What is claimed is:

1. A compound of Formula (I): 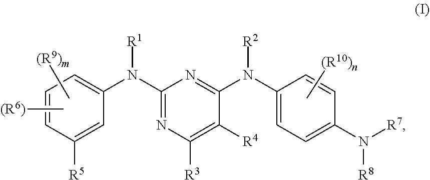
or a pharmaceutically acceptable salt, tautomer, or isotopically labeled derivative thereof, wherein:
R¹ and R² are each independently —H, optionally substituted aliphatic, optionally substituted acyl, or a nitrogen protecting group;
R³ and R⁴ are each independently —H, halogen, —OR^{a3}, —N(R^{a3})₂, —CN, —SCN, —NO₂, —N₃, —SR^{a3}, optionally substituted aliphatic, optionally substituted heteroaliphatic, or optionally substituted acyl;
R⁵ is —S(═O)₂N(R^{a5})₂, —S(═O)₂OR^{a5}, —S(═O)₂R^{a5}, —S(═O)₂Cl, or —SR^{a5};
R⁶ is —OR^{a6}, —N(R^{a6})₂, —CN, —SCN, —NO₂, —N₃, —SR^{a6}, optionally substituted heteroaliphatic, optionally substituted acyl, optionally substituted sulfonyl, or optionally substituted sulfinyl;
R⁷ and R⁸ are each independently —H, unsubstituted aliphatic, unsubstituted heteroaliphatic, —C(═O)R^{a7}, —C(═O)OR^{a7}, or a nitrogen protecting group; optionally wherein R⁷ and R⁸ are joined together with the intervening atoms to form optionally substituted heterocyclyl or optionally substituted heteroaryl;
each instance of R⁹ and R¹⁰ is independently —H, —OR^{a9}, —C(═O)R^{a9}, —OC(═O)R^{a9}, —C(═O)OR^{a9}, —N(R^{a9})₂, —N(C═O)R^{a9}, —(C═O)N(R^{a9})₂, —CN, —SCN, —NO₂, —N₃, —SR^{a9}, halogen, optionally substituted aliphatic, optionally substituted heteroaliphatic, optionally substituted sulfonyl, or optionally substituted sulfinyl;
each instance of R^{a3}, R^{a5}, R^{a6}, and R^{a9} is independently —H, optionally substituted aliphatic, optionally substituted heteroaliphatic, optionally substituted aryl, optionally substituted heteroaryl, optionally substituted carbocyclyl, optionally substituted heterocyclyl, optionally substituted acyl, optionally substituted sulfonyl, or optionally substituted sulfinyl, an oxygen protecting group when attached to an oxygen atom, a nitrogen protecting group when attached to a nitrogen atom, or a sulfur protecting group when attached to a sulfur atom; optionally wherein any two instances of R^{a3}, R^{a5}, R^{a6}, or R^{a9} attached to the same nitrogen atom are joined together with the intervening atoms to form optionally substituted heterocyclyl or optionally substituted heteroaryl;
each instance of R^{a7} is independently —H, or unsubstituted aliphatic;
m is 0, 1, 2, or 3; and
n is 0, 1, 2, 3, or 4.

2. The compound of claim 1, wherein the compound is of Formula (I′): 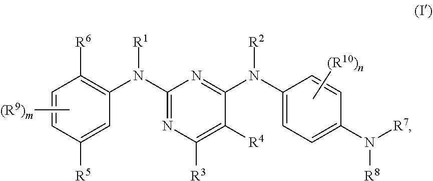
or a pharmaceutically acceptable salt, tautomer, or isotopically labeled derivative thereof.

3. The compound of claim 2, wherein the compound is of one of the following formulae: 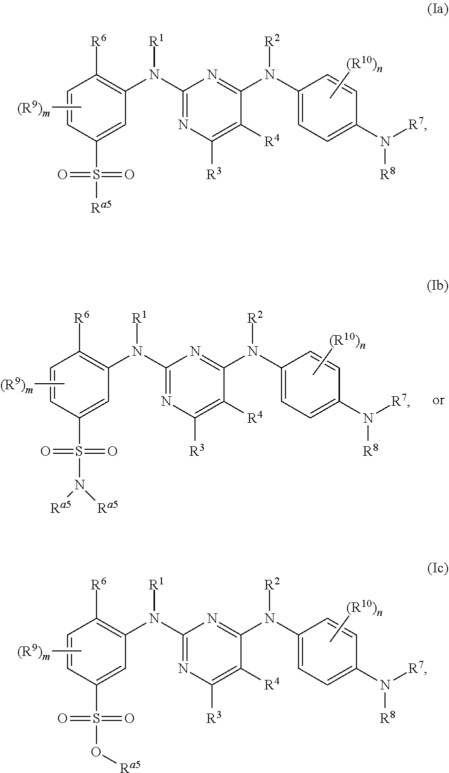
or a pharmaceutically acceptable salt, tautomer, or isotopically labeled derivative thereof.

4. The compound of claim 2, wherein the compound is of one of the following formulae: 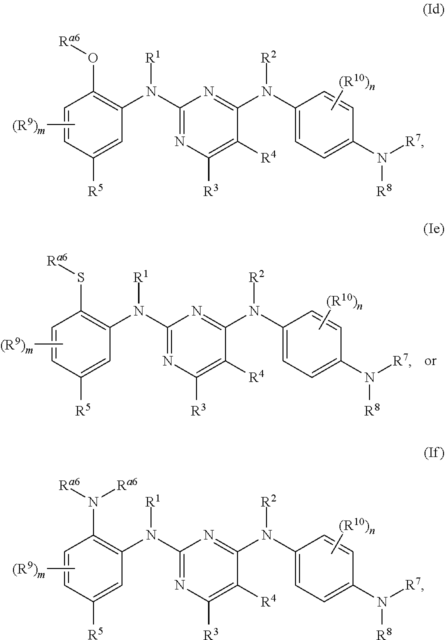
or a pharmaceutically acceptable salt, tautomer, or isotopically labeled derivative thereof.

5. The compound of claim 2, wherein the compound is of Formula (Ij): 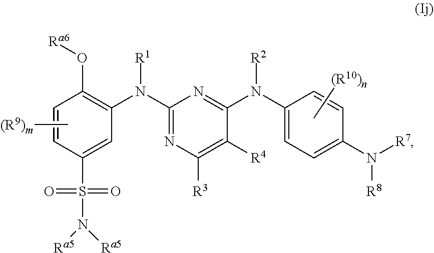
or a pharmaceutically acceptable salt, tautomer, or isotopically labeled derivative thereof.

6. The compound of claim 1, wherein the compound is of Formula (I″): 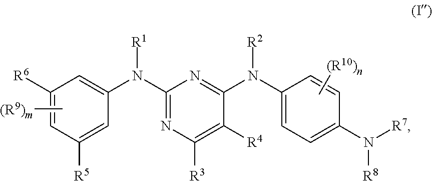
or a pharmaceutically acceptable salt, tautomer, or isotopically labeled derivative thereof.

7. The compound of claim 6, wherein the compound is of one of the following formulae: 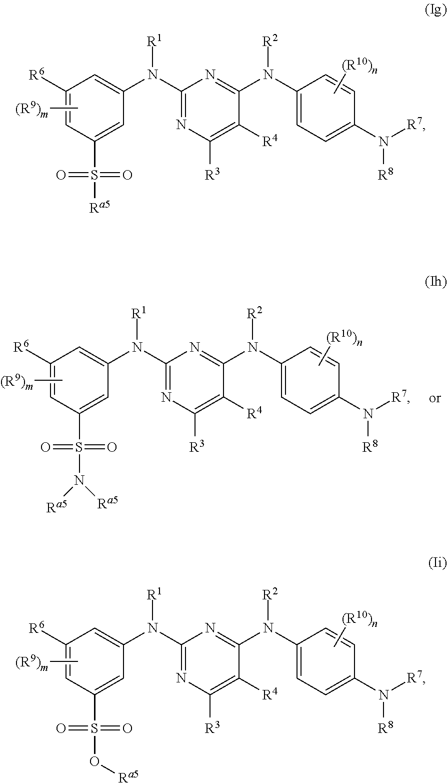
or a pharmaceutically acceptable salt, tautomer, or isotopically labeled derivative thereof.

8. The compound of claim 6, wherein the compound is of one of the following formulae: 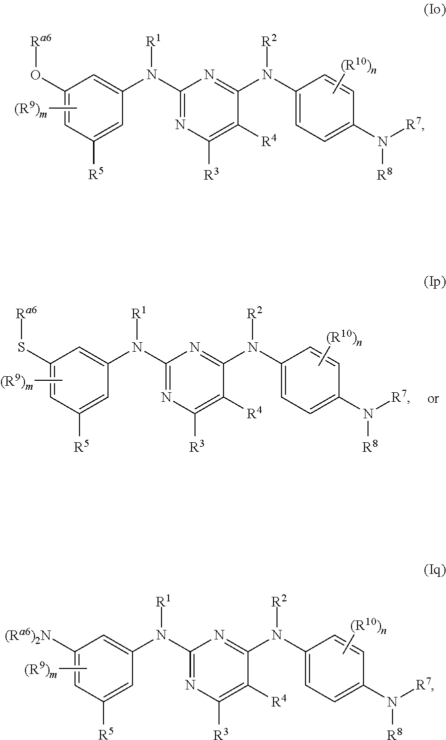
or a pharmaceutically acceptable salt, tautomer, or isotopically labeled derivative thereof.

9. The compound of claim 6, wherein the compound is of Formula (Ir): 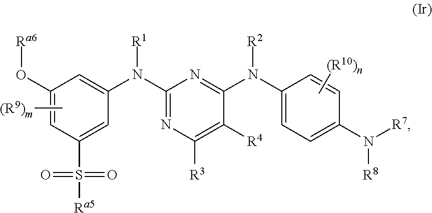
or a pharmaceutically acceptable salt, tautomer, or isotopically labeled derivative thereof.

10. The compound of claim 1, wherein the compound is of Formula (I′″): 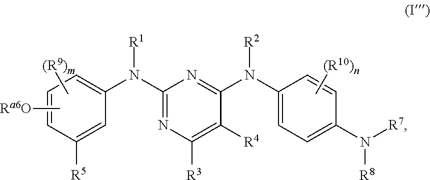
or a pharmaceutically acceptable salt, tautomer, or isotopically labeled derivative thereof.

11. The compound of claim 1-3, 6, or 7, or a pharmaceutically acceptable salt, tautomer, or isotopically labeled derivative thereof, wherein R⁶ is —OR^{a6}, —N(R^{a6})₂, or —SR^{a6}.

12. The compound of claim 1-3, 6, or 7, or a pharmaceutically acceptable salt, tautomer, or isotopically labeled derivative thereof, wherein R⁶ is —OR^{a6}.

13. The compound of claim 1-3, 6, or 7, or a pharmaceutically acceptable salt, tautomer, or isotopically labeled derivative thereof, wherein R⁶ is —OMe.

14. The compound of claim 1, 2, 4, 6, 8, or 10-13, or a pharmaceutically acceptable salt, tautomer, or isotopically labeled derivative thereof, wherein R⁵ is —S(═O)₂N(R^{a5})₂.

15. The compound of claim 1, 2, 4, 6, 8, or 10-13, or a pharmaceutically acceptable salt, tautomer, or isotopically labeled derivative thereof, wherein R⁵ is —S(═O)₂NH₂.

16. The compound of any one of the preceding claims, or a pharmaceutically acceptable salt, tautomer, or isotopically labeled derivative thereof, wherein R¹ is —H.

17. The compound of any one of the preceding claims, or a pharmaceutically acceptable salt, tautomer, or isotopically labeled derivative thereof, wherein R² is —H or -Me.

18. The compound of any one of the preceding claims, or a pharmaceutically acceptable salt, tautomer, or isotopically labeled derivative thereof, wherein R¹ is —H and R² is -Me.

19. The compound of any one of claims 1-15, or a pharmaceutically acceptable salt, tautomer, or isotopically labeled derivative thereof, wherein R¹ and is -Me and R² is —H.

20. The compound of any one of claims 1-15, or a pharmaceutically acceptable salt, tautomer, or isotopically labeled derivative thereof, wherein R¹ and R² are both -Me.

21. The compound of any one of claims 1-17, or a pharmaceutically acceptable salt, tautomer, or isotopically labeled derivative thereof, wherein R¹ and R² are both —H.

22. The compound of any one of the preceding claims, or a pharmaceutically acceptable salt, tautomer, or isotopically labeled derivative thereof, wherein R³ and R⁴ are both —H.

23. The compound of any one of the preceding claims, or a pharmaceutically acceptable salt, tautomer, or isotopically labeled derivative thereof, wherein R⁷ and R⁸ are each independently —H, optionally substituted C₁₋₆ alkyl, or a nitrogen protecting group.

24. The compound of any one of the preceding claims, or a pharmaceutically acceptable salt, tautomer, or isotopically labeled derivative thereof, wherein R⁷ and R⁸ are each —H.

25. The compound of any one of the preceding claims, or a pharmaceutically acceptable salt, tautomer, or isotopically labeled derivative thereof, wherein n and m are 0.

26. The compound of claim 1, having the structure: 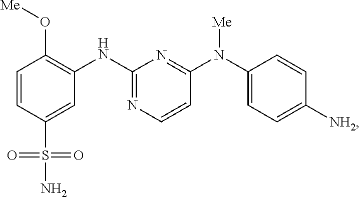 [SMILES: COc1ccc(S(N)(=O)=O)cc1Nc1nccc(N(C)c2ccc(N)cc2)n1]
or a pharmaceutically acceptable salt, tautomer, or isotopically labeled derivative thereof.

27. The compound of claim 1, having the structure: 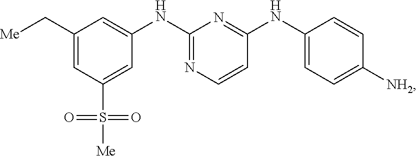 [SMILES: CCc1cc(Nc2nccc(Nc3ccc(N)cc3)n2)cc(S(C)(=O)=O)c1]
or a pharmaceutically acceptable salt, tautomer, or isotopically labeled derivative thereof.

28. A compound of Formula (II): 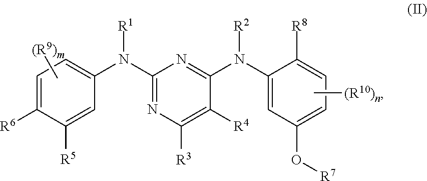
or a pharmaceutically acceptable salt, tautomer, or isotopically labeled derivative thereof, wherein:
R¹ and R² are each independently —H, optionally substituted aliphatic, optionally substituted acyl, or a nitrogen protecting group;
R³ is —H, halogen, —OR^{a3}, —N(R^{a3})₂, —CN, —SCN, —NO₂, —N₃, —SR^{a3}, optionally substituted aliphatic, optionally substituted heteroaliphatic, or optionally substituted acyl;
R⁴ is —H, —Br, —OR^{a4}, —N(R^{a4})₂, —CN, —SCN, —N₃, —SR^{a4}, branched or unbranched aliphatic, optionally substituted heteroaliphatic, or optionally substituted acyl;
R⁵ and R⁶ are each independently —H, —S(═O)₂N(R^{a5})₂, —S(═O)₂OR^{a5}, —S(═O)₂R^{a5}, —S(═O)₂Cl, —SR^{a5}, —C(═O)N(R^{a6})₂, —C(═O)OR^{a6}, or —C(═O)R^{a6};
provided that R⁵ and R⁶ are not both —H;
R⁷ is —H, optionally substituted aliphatic, optionally substituted acyl, or an oxygen protecting group;
R⁸ is —N(R^{a8})₂, —CN, —SCN, —NO₂, —N₃, —SR^{a5}, halogen, optionally substituted aliphatic, optionally substituted acyl, optionally substituted sulfonyl, or optionally substituted sulfinyl;
each instance of R⁹ is independently —H, —OR^{a9}, —C(═O)R^{a9}, —C(═O)OR^{a9}, —OC(═O)R^{a9}, —N(R^{a9})₂, —N(C═O)R^{a9}, —(C═O)N(R^{a9})₂, —CN, —SCN, —NO₂, —N₃, —SR^{a9}, halogen, optionally substituted aliphatic, optionally substituted heteroaliphatic, optionally substituted sulfonyl, or optionally substituted sulfinyl;
each instance of R¹⁰ is independently —H, —C(═O)R^{a10}, —C(═O)OR^{a10}, —OC(═O)R^{a10}, —N(C═O)R^{a10}, —(C═O)N(R^{a10})₂, —CN, —SCN, —NO₂, —N₃, —SR^{a10}, halogen, optionally substituted aliphatic, optionally substituted heteroaliphatic, optionally substituted sulfonyl, or optionally substituted sulfinyl;
each instance of R^{a3}, R^{a4}, R^{a5}, R^{a6}, R^{a8}, R^{a9}, and R^{a10} is independently —H, optionally substituted aliphatic, optionally substituted heteroaliphatic, optionally substituted aryl, optionally substituted heteroaryl, optionally substituted carbocyclyl, optionally substituted heterocyclyl, optionally substituted acyl, optionally substituted sulfonyl, or optionally substituted sulfinyl, an oxygen protecting group when attached to an oxygen atom, a nitrogen protecting group when attached to a nitrogen atom, or a sulfur protecting group when attached to a sulfur atom; optionally wherein any two instances of R^{a3}, R^{a4}, R^{a5}, R^{a6}, R^{a8}, R^{a9}, or R^{a10} attached to the same nitrogen atom are joined together with the intervening atoms to form optionally substituted heterocyclyl or optionally substituted heteroaryl;
m is 0, 1, 2, or 3;
n is 0, 1, 2, or 3; and
provided that the compound is not of the formula:

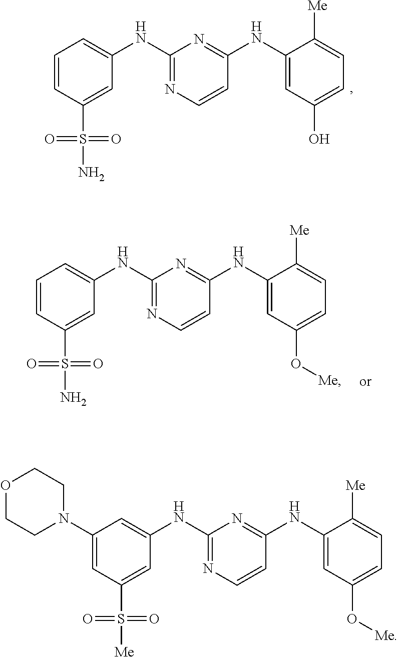 [SMILES: Cc1ccc(O)cc1Nc1ccnc(Nc2cccc(S(N)(=O)=O)c2)n1 ; COc1ccc(C)c(Nc2ccnc(Nc3cccc(S(N)(=O)=O)c3)n2)c1 ; COc1ccc(C)c(Nc2ccnc(Nc3cc(N4CCOCC4)cc(S(C)(=O)=O)c3)n2)c1]

29. The compound of claim 28, wherein the compound is of one of the following formulae: 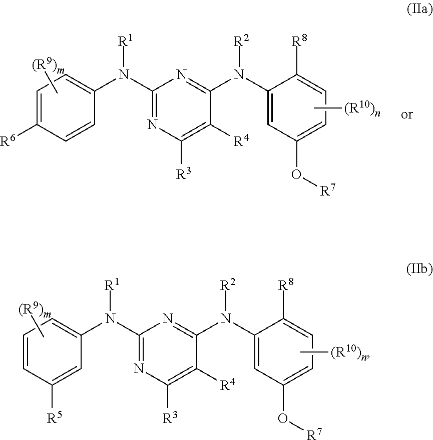
or a pharmaceutically acceptable salt, tautomer, or isotopically labeled derivative thereof.

30. The compound of claim 28 or 29, wherein the compound is of one of the following formulae: 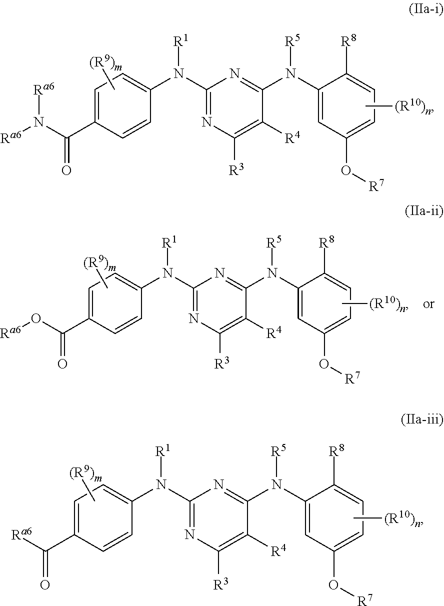
or a pharmaceutically acceptable salt, tautomer, or isotopically labeled derivative thereof.

31. The compound of claim 28 or 29, wherein the compound is of one of the following formulae: 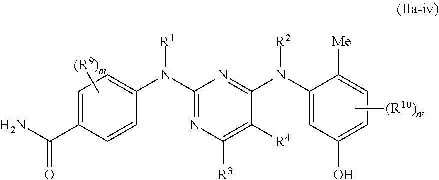
or a pharmaceutically acceptable salt, tautomer, or isotopically labeled derivative thereof.

32. The compound of claim 28 or 29, wherein the compound is of one of the formulae: 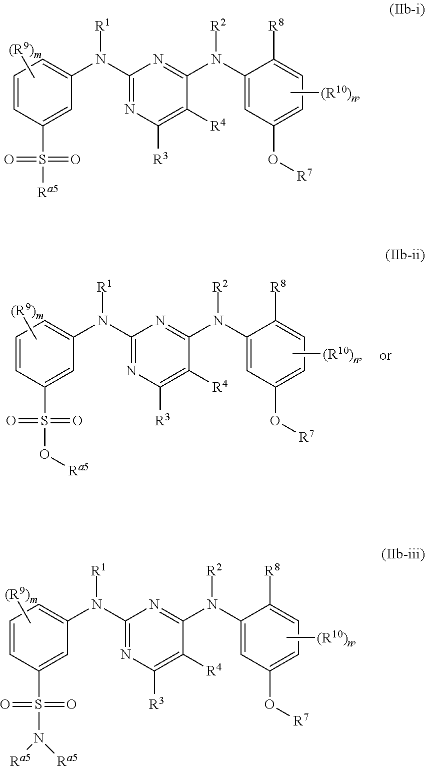
or a pharmaceutically acceptable salt, tautomer, or isotopically labeled derivative thereof.

33. The compound of claim 28 or 29, wherein the compound is of one of the formulae: 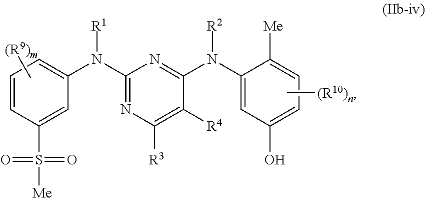
or a pharmaceutically acceptable salt, tautomer, or isotopically labeled derivative thereof.

34. The compound of any one of claims 28-30 or 32, or a pharmaceutically acceptable salt, tautomer, or isotopically labeled derivative thereof, wherein R⁸ is optionally substituted C₁₋₆ alkyl.

35. The compound of any one of claims 28-30 or 32, or a pharmaceutically acceptable salt, tautomer, or isotopically labeled derivative thereof, wherein R⁸ is methyl.

36. The compound of any one of claims 28-30 or 32, or a pharmaceutically acceptable salt, tautomer, or isotopically labeled derivative thereof, wherein R⁷ is —H, optionally substituted C₁₋₆ alkyl, or an oxygen protecting group.

37. The compound of claim 36, or a pharmaceutically acceptable salt, tautomer, or isotopically labeled derivative thereof, wherein R⁷ is —H.

38. The compound of any one of claims 28-30, 32, 34, 36, and 37, or a pharmaceutically acceptable salt, tautomer, or isotopically labeled derivative thereof, wherein R⁷ is —H, and R⁸ is optionally substituted C₁₋₆ alkyl.

39. The compound of any one of claims 28-30, 32, 34, or 36, or a pharmaceutically acceptable salt, tautomer, or isotopically labeled derivative thereof, wherein R⁷ and R⁸ are both optionally substituted C₁₋₆ alkyl.

40. The compound of any one of claims 28, 29, 34, or 36-39, or a pharmaceutically acceptable salt, tautomer, or isotopically labeled derivative thereof, wherein R⁵ is —S(═O)₂R^{a5}, and R⁸ is optionally substituted C₁₋₆ alkyl.

41. The compound of any one of claims 28, 29, 34, or 36-39, or a pharmaceutically acceptable salt, tautomer, or isotopically labeled derivative thereof, wherein R⁶ is —C(═O)N(R^{a6})₂, and R⁸ is optionally substituted C₁₋₆ alkyl.

42. The compound of any one of claims 28-41, or a pharmaceutically acceptable salt, tautomer, or isotopically labeled derivative thereof, wherein R¹ and R² are both —H.

43. The compound of any one of claims 28-41, or a pharmaceutically acceptable salt, tautomer, or isotopically labeled derivative thereof, wherein R¹ is —H and R² is -Me.

44. The compound of any one of claims 28-41, or a pharmaceutically acceptable salt, tautomer, or isotopically labeled derivative thereof, wherein R¹ and is -Me and R² is —H.

45. The compound of any one of claims 28-41, or a pharmaceutically acceptable salt, tautomer, or isotopically labeled derivative thereof, wherein R¹ and R² are both -Me.

46. The compound of any one of claims 28-45, or a pharmaceutically acceptable salt, tautomer, or isotopically labeled derivative thereof, wherein R³ and R⁴ are both —H.

47. The compound of any one of claims 28-46, or a pharmaceutically acceptable salt, tautomer, or isotopically labeled derivative thereof, wherein n and m are both 0.

48. The compound of claim 28, having the structure: 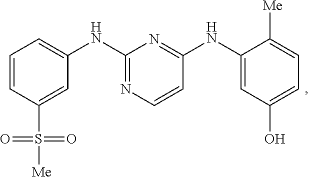 [SMILES: Cc1ccc(O)cc1Nc1ccnc(Nc2cccc(S(C)(=O)=O)c2)n1]
or a pharmaceutically acceptable salt, tautomer, or isotopically labeled derivative thereof.

49. The compound of claim 28, having the structure: 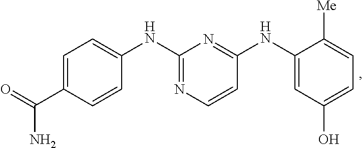 [SMILES: Cc1ccc(O)cc1Nc1ccnc(Nc2ccc(C(N)=O)cc2)n1]
or a pharmaceutically acceptable salt, tautomer, or isotopically labeled derivative thereof.

50. A pharmaceutical composition comprising a compound of any one of claims 1-49, or a pharmaceutically acceptable salt, tautomer, or isotopically labeled derivative thereof, and a pharmaceutically acceptable excipient.

51. The pharmaceutical composition of claim 50 further comprising an additional pharmaceutical agent.

52. A pharmaceutical composition comprising a compound of any one of claims 1-49, or a pharmaceutically acceptable salt, tautomer, or isotopically labeled derivative thereof, or a pharmaceutical composition of claim 50 or 51, for use in treating an inflammatory disease, joint disease, proliferative disease, fibrosis, or pain.

53. A method of treating an inflammatory disease, joint disease, proliferative disease, fibrosis, or pain in a subject in need thereof comprising administering to the subject a therapeutically effective amount of a compound of any one of claims 1-49, or a pharmaceutical salt, tautomer, or isotopically labeled derivative thereof, or a pharmaceutical composition of any one of claims 50-52.

54. The method of claim 53, wherein the inflammatory disease is osteoarthritis.

55. The method of claim 53, wherein the inflammatory disease is rheumatoid arthritis.

56. The method of claim 53, wherein the fibrosis is hepatic cirrhosis.

57. The method of claim 53, wherein the joint disease is traumatic cartilage injury.

58. The method of claim 53, wherein the pain is associated with allodynia.

59. The method of claim 53, wherein the pain is associated with hyperalgesia.

60. The method of claim 46, wherein the proliferative disease is cancer.

61. The method of any one of claims 53-60, wherein the compound of any one of claims 1-49, or pharmaceutically acceptable salt, tautomer, or isotopically labeled derivative thereof, or pharmaceutical composition of any one of claims 50-52, is administered intraarticularly.

62. A method of preventing articular cartilage degradation in a subject comprising administering to the subject a therapeutically effective amount of a compound of any one of claims 1-49, or a pharmaceutical salt, tautomer, or isotopically labeled derivative thereof, or a pharmaceutical composition of any one of claims 50-52.

63. A method of inhibiting DDR2 activity in a subject, in vivo, or in vitro comprising contacting a DDR2 protein in a subject, in vivo, or in vitro with a compound of any one of claims 1-49, or a pharmaceutically acceptable salt, tautomer, or isotopically labeled derivative thereof, or a pharmaceutical composition of any one of claims 50-52.

64. A method of inhibiting DDR1 activity in a subject, in vivo, or in vitro comprising contacting a DDR1 protein in a subject, in vivo, or in vitro with a compound of any one of claims 1-49, or a pharmaceutically acceptable salt, tautomer, or isotopically labeled derivative thereof, or a pharmaceutical composition of any one of claims 50-52.

65. A method of inhibiting p38 MAPK activity in a subject, in vivo, or in vitro comprising contacting a p38 MAPK protein in a subject, in vivo, or in vitro with a compound of any one of claims 1-49, or a pharmaceutically acceptable salt, tautomer, or isotopically labeled derivative thereof, or a pharmaceutical composition of any one of claims 50-52.

66. The method of claim 65, wherein the p38 MAPK is p38-alpha.

67. A method of inhibiting cell migration comprising contacting a cell, tissue, or biological sample with a compound of any one of claims 1-49, or a pharmaceutically acceptable salt, tautomer, or isotopically labeled derivative thereof, or a pharmaceutical composition of any one of claims 50-52.

68. Use of a compound of any one of claims 1-49, or a pharmaceutically acceptable salt, tautomer, or isotopically labeled derivative thereof, or a pharmaceutical composition of any one of claims 50-52, for the manufacture of a medicament for treating an inflammatory disease, joint disease, proliferative disease, fibrosis, or pain.

69. A compound of any one of claims 1-49, or a pharmaceutically acceptable salt, tautomer, or isotopically labelled derivative thereof, or a pharmaceutical composition of any one of claims 50-52, for use in treating or preventing an inflammatory disease, joint disease, proliferative disease, fibrosis, or pain.

70. A kit comprising:
a compound of any one of claims 1-49, or a pharmaceutically acceptable salt, tautomer, or isotopically labeled derivative thereof; or a pharmaceutical composition of any one of claims 50-52; and
instructions for using the compound, or pharmaceutically acceptable salt, tautomer, or isotopically labeled derivative thereof, or pharmaceutical composition.


SEQUENCE LISTING (2 sequences)

SEQ ID NO: 1 - protein, 14 aa, synthetic construct
KKKSPGEYVNIEFG

SEQ ID NO: 2 - protein, 13 aa, synthetic construct
KKSRGDYMTMQIG